SEQUENCE LISTING (172 sequences)

SEQ ID NO: 1 - DNA, 131217 bp, Japanese Macaque Herpesvirus
cccggggcgcgaccgagggccccgggagaacgggggatcgggaaaacgcgaggggtggcgggggacagaggggaacgtgtgcgtgcttgtggaacactatggcctgctggcctgctcgcttgcttggtagcttgcaagcttgctgctgagggtctttagagggacagtaggcctgcttgctggcctgcttgctggcctgcttgctggcctgcttgctggcctgcttgctggcctgcttgctggcctgctttgctggcctgcttgctggcctgcttgctggcctgcttgctggcctgcttgctggcctgcttgctggcctgcttgctggcctgcttgctggcctgcttgctggcctgcttgctggcctgcttgctggcctgcttgctggcctgcttgctggcctgcttgctggcctgcttgctggcctgcttgctggcctgcttgctggcctgcttgctggcctgcttgcttgctgctgaggggacagtagggctgcctgcttgcttgctgctgaggggacagtagggctgcctgcttgcttgctgctgaggggacagtagggctgcctgcttgcttgctgctgagtaggactgctagggctggtagtagtggggcagccggccagctgcgtgcgagggcgttgcagggccacacgaggacacgggacccgagccctccccaggaccgccgggcaagcggcccagatctgcccggcaggtcgctcggggagagcggacggaaagggcggtgggggctgggagcatagacgcggggctgggaacggggagtagggggatgggaaggggtgcagacgtggggccgggggacggggcgtggtttgagaccccaagctgatgactcaaaaatgggctaaaccacgcccacctcatttttttcccccggccctatcccaagcaacgctcccccaactaaaactccctcttgtcggcagctaagggtcgcctgccttcttcccattccccacccatccgctacaataaaaccgcctgcctgccgctgacggaaaaaggggaggtgtgtgtatgcttttgtaactttccagcggagggggtagagtgcacggccaatttccaagcacctgcttaactcgctttggctttatgtggttttgttgctatgtactacacacagtaattcattgcatggcgcctgcatacgctccacagcatccgaaacaagttcatttctctagactaactttagtgttcttgttggctcataccattttacaaagtttgctcctttgaataataagaaatccgttgtggttacaatacacctgccaatgtattggatggtttcttttagcatagaccttgacttgcattttaacattgattttttcccattagaccctcgtttcttcatgcaattaggtatgtctttgcctgtgtttactttgctctcctccgttctgtggaaatgtttgtgttgcttatatttatcttattgcaacctgcgtcgttggagcttttgcccgcaaaattaactgctgttccaacgtggtgtccaccgcatcccggagatacctacttgctaacctgccgcggaacatctacggccagagaccagcgcagcacacaatggtttcgcaacaacacgcttatgcatgggagtaatttctacggcaggctggtatctgtaactcccaattctacaatatctgactggtatgcgtgtcaaacaaaaacaacaacgcggagcaatagcatcgattttcgggtaaggtcatcgcgcctcacgctccaagaacgttgctcttcatacggctattctaacgcaaataacacaagggtattgaggtgttactctggtggaaacgtaactttaagaaacgttgtttttcatttaaatggtacagcggtcattaacggtactacaacagacatatatacatttgttttaacggaaaagacaggcggaacatattactgttctgcgtttattgggactgaaaaactctattctcaaaaaattaatgtgttttttacttcatttacctttaaacatacagacaatgttcaaaatgggtcagaatttaataaaactgagcaaattcaacaaacagctaatgtacaacacactgcaaactacgttgtgttctctgttcccgttttttctattggtgttttaacaggtattgcaatatcgttgattatgtgttggttatttacgttgcgctgcaacaagaactctgaatcctcgaataataggcatgcacaccaaacgagctacattcaaccctcccataatcagcattcccatacaagtgaaagtactacccatacctatagaaatgatcatcaggaagagagtattgaagaactaccaaaccaacacacacgtaaaactaattcttgtcaaacagttttacttgaagtgaaaaatgtagccttcgatggaccacaggggaacttgcacaatacaaacgacgaagtgatggaacagtatgatgatgtggttgtagaaaatatagaacaaacatcatatgataataatattgaacagatggactatagtgacattatacgccccaattttaattactacagtggactaatattggaagaagttgatgaggttttttacaatgaactagcaaatcaatatcatggattaatactggagaatttagatcacgatgagtacaaccatttaaataaattaaacatgatagaacagtatgattggttagaataagtcacttgtgggttattcttaaaataaacaaaacataaacaacattaaaaaaagttttattgtgtaactgctttatttattaaccttttttcatatacttgaaacttatatttaattccattttcttcaaagtttgtgtctttacctggtaattctgataacatggtatattcggtaaaattaattgatgggaaaaatacatcacaatcaaaactctccataatgcgcgtaatatatagttttaaagaacactcgtagttaagaacactctcataaacagattttcctccaataacccaaaccgtgtttaattgtttggttagtttatattgtctatagaaattaaacgcatcgtctagcgttcttgctagaaagtgcgctccctttggtggttcacgtaattctctgctcagaatgatgttaattctgttcgccagagggcgcttcttttcagggatggaaaaccatgttcttttccccataataaccacattcttttcacctataaccgatggcgtagaagtcattttctgaaaatacatcatttcgtttttaaggtacggccaaggcatggttccgtttttaccaattcctaattgtttatcaaccgcaacaatacagttaactgtaatgtccatggttttggttgcccctaacgggtttaacacagaaatatttcacttataaacaaatacccacgtgacatgtacttactaatgtgagtacttaacataatatagttaacaatattcgctttcacataatggcgttgcatatataagcgacgtggacttgtatgtttttatgagattaatcatgttttggttccccgttaccgtgtgctttgtatgccactttatactggtaaatgcgcaaaatgaaacaaatgttacctgtgataagcctaattttgatagatttatggttgcaatggaaaaaaaagaaaaatacgtccttggagataaagttgaattaacttgtcggcctggatatacattacagggaaaagtttacgtgcaatgccttcaaagcggaatgtggactacaccaaacgcagaatgtcatagaaaaaaatgcacaaattatggagatattctaaacgggcaggtaattgtaccagattctgacaatgcttttaaatttggaacaaatataacatataagtgcaatacaggatatctccttcttggcagtatggtccgcacgtgcctgctaactggtaattcaaacaccgttaattggcaacccgccgctcctacatgtgagattgaaaaatgtaaaaaacaaccggatattgaaaatggcaaatactatccagttcaagacttttacaactatctggaaacaattacgtttacgtgtaataaagatttttctctaattggaaatacaacaacaacgtgtatgacaaatggcacgtggagcagcccggttccaaaatgtgaacagataacttgcagcgcccctaatattgaacatggaacgctattagtgggttcttcgcgtgtatacaagcacggtcaatctattaccattggttgtgaaaatggatttaccttaaatggacataaaatgtgtacatgtgaatattcatcgtggaatccaccgcttcctacatgcgtgcctataaataagactgttccaactccatcggaagtacccagtccagggaccaataagcaggaaaggcccacaccagaaaatccaaagtcccatgagtcggaaactacaacagaaacaccaaaaacaggaacccataaatcagaaacaccatcaaaaaaaatacctaacccagagacccacaaaccaaccaccccaaaatctggaacatcagaacaaacaaccaataggccatcaaaagcaccgtcgcaaaatccaccaatggaacccccgatgagtaaatggaaaaggcatgttgtgttagttctttttgcaagtgttgcatccctgttattcgtgctcgttaccctttattgttgttttctaaaataacttttttgttttcaaatgcaggttcgccaggcaaactcgcacgcattaaccaatctgccgaccgccgttgattccggaattaagtttacattattcaaggttgtcaataaaggtggtttataaatattctcttgtgtttattgttttgtgttaacttgttatagctatcacgccaccttgtggggctacattatagagcattattattttcaactttatactttcacgtcctatgaatcatactcatccacatcacgtggcatgcgattgtggccgggactgaaattaacacaaagggtacataatccatccaggcggcacacattagacgcggtttataaaactaatatcccatgcgccaacgatcactgtcgctggcgacgctgctagaaaaacattttaacgttgtttagcagaattaaataacacgtaatggcatccaaaggcaacgccggacaacccctggaagataatcaggggtctcgtgccccaataggcgcttgcggatacgtatacgcgtattcgaaacaagactttcccttcgctgaggcgtcaatactcggcaacaggccatctggatctggcgtcttttcgttacccatcctttacggacttaccgtcgaacacgaattccctctcaccgtaaaagccgcacacaaaaaagttgacaccacaacgctcgccgtcaaggtgacgtgctttcacagagaggttattgtttttcacaacgcaaacctattcaggccggtgtttgaaggcaccggtcttaacgaactatgcgaggaagccagggctctcttcgggtatacgcagtttatagaaccgggtccacctcacggcatatggaaccctctggaatgtccgcagttaccggacaaggatgagatgttccttggcgttgtcgtgacggaagggtttaaggagagactgtggaggggctgtctcgttcccgcagtgtttcagacccagcaggtgcagatcgccgggcgtcaggcgttcaaagtgccgttgtacgacgaagacctgttcgcacctcacgggcataggatgccgcggttttaccataaagacgttagcgcgtacctctacaactccctttttaccagtatcgcccaggccctgagactcaaagacgtgacggcggtcatccacgccacagaaaaacaattcatgcaggaccattacaaaattgccaagatagtgcaggcaaaacagttttccacgacgctgccgaaaacggcagacgggtcgtcacacatgatcgtcgacagcgttgttgccgagctcgcccttagttacggctgtatgtttctcgagtgtccacaggacgcgtgtgagttgctgaactacgatagctggcccatatttgacggttgcgactcatcagaggatagggttaacgcgttagagcgctggtcggccgaacaggccgttcacgtggcgggtcaactgttcgctgccaattcggtgctgtacctgaccagagtgcaaaagcaagcacccaggggacaaaagggagacgtgaacgtgtacaactcctttttccttcaacacggactggggtttctgaacgaggcgacgattaaggaaaacggcagcgaagcctttaagggcgtgccctcaaacgccctcgatggttcttcgtttacgccgtatcatctggcctatgccgcgtcgttctcgccccatctgctggcaaagttatgttattacatgcagttcttgcagcaccacaaaagctccacgaaccaaacgttcaacatagtccactatgtcggcaccgccgccaactcagaaatgtgcacgctgtgtcacggcgacacgccgggaacgtgcctcaacacgctgttctatagactgaaggataggtttccagccgtaaccacccctcagcgcagggacccctacgtggtgaccggaaccgccggaacctttaacgacctggagatcctgggcaacttcgcgagtttcagagaccgcgaggaggacggaaaccctgccgacgagcacccaaagtacacgtactggcagctatgtcagaccgtgacggaaaagttatccgcgatcggaattaccgaggaccaggataaccacgtgaacctcatcaccaacatccaaagttttctcagggtgttcaagggtatcgactcggtggtggacggagaggtcatgaaatttattaactcgatgattaaaaataactttaattttcgcgagcacgttaaatcggtccaccacatacttcagttctgctgcaacgtgtattggcaggcgccgtgcgcggtgttcctgaatctgtactacaaatccctactgtggatcatccaggatatctgcctgccgtactgcatgatctacgaacaagataatccggcgatgggcatcctcccctccgagtggctaaagatgcattttcaaacgttgtggacgaacttcaaagcggcatgtctcgaccgcggggtcctcacgggatgcgaactgaaaatagtacaccgggacatgttctgcgacttcttcgacaccgacgccgggtccaacggtttactggccccctttaaaatgcaggttagaatagccagagctatgatggtcgttccgaaatcgattaaaataaaaaatagaattattttttccaacaccgcgggttccgaggcggtgcagtcgggattcgttaaaccaacgggaaccagggacacctacgtagtggccggaccgtacatgaaatttctaaactcgctccatcgcgcgctattcccaaacaccaagaccgccgcgctgtacctgtggcacaaaatctcccagaccaacaaaaccccagtcctgaaagacgtcccggacgacgagctggcggagctggtgtcgtacgtcaaaaccaacagcctcgcgttcgaggaaacgaacgtgctggacgtggtcccggattccctcatgtcgtacgcgaggattaaactgaacggggccattctgagggcgtgcggtcaaattcagttctacgccacaacgctgcattgccttacgccggtgttacaaactatcgacgccgaggaatacccccacgtgctaggctccgcggcgattgccacgccggtggcttacctgtccgaaatacgcggccgcaccgccctcaccgtccaaacgacggcgcgtcagccggtcgccgccacggggcgtctgcgccccgtgataaccgtacccatggtagttaacaaatacacgggagttaacgggaacaacaacgtttttcactgcgggaacctggggtacttcgcggggcgcggcgtggaccgcaacctgtggccggaaagttccccctttaagaaaacgggcgtcagcgcaatgctcagaaagagacacgtcatgatgacccccatagtcgaccgcctgataaagcgagccgcgggacagacgatcagcacgttcgaggcggagagcgtcaaaaggagcgtgcaggcgctgttagaggacaaagacaaccccaacctactgaagtcggtgatcttggagcttatacgacatctgggaaagggctgccaggacttaagctcggaggacgtgcaatattacctcggtgactattgcatgttgacggacgaggttttatttacgttggataatatagcacagtcaggcgtgccgtggacattcgaggacgcgggtgccctgatagaagatcgccagaacacagacgatcttcagttcgtggacagcgacgatatcgccaccgcttcctgccagccccccgaggaacagctaccgacccctagcgccggcgccctactggccgggaagaagcgtaaaattaacgtgctactgagtgatttagacctctaggaaaaccgtggtcggcgaacaacgatggccagggaactcgcggccttatacgcgcagctgtcggcccttgccgtcgatctgagtctggtggtctttgctgacccgcgaagtatcgacggttcccgcattctaaaaacaaaaacacagatagaaaacctgaaccgcgacctcctgccgctactacgcaagcaaaactcggtagagacgtccagcctgtcgctcgaagtggagcacctggccaaaaacatcgaggacaaactcggggagctagagcgcagtttgcggcaaagatattcgagccgagagcattttgagacactacacctgaggcccgagtgtcactatcacgctacggttacctttgagttttacgggggtgggctaatagatgtaaacatgtgcctaataaacgatgtagaactgctgtgtaaaagactagggagtgtgttttattgcatcggtgcgaacgaagccctgtccggattggaccgggttttggcgtttctgtccacactgcggggtatctcccccatcccgcacccagacctatacgtcacgtccgtgccttgcgtacagtgcctgcgggaaatcgaactagtaccaaatcaggggtccagtttactcgcggtgttggcagaccgacactgcgatcacctctgtaagaaggttagggcggagccaatacacggcctgtttgagacagaactcagccagttgggtctaaaagtagcaaaaggttcggacgccacgcagcacggcgtccggtcctcggcagatcagttaagggagtcgtcgctggcggccatacaagatcacaatatattcaagcgggtgtccgcgtcaatcatggaactatccaatctaatttattggaacgccgggcaaaccggcctccagaccgggacagaaaacgagtgctcccaaatggccaaactgctaacacacgaggccgagatgcacgagcaccgtgcgctaataacacccaaacaaagcgcggctcacttctacgactgtttccgaccggatcccatagagtccctgttctgcggcggtctttttaactctatagacgataccattaacgcactgagccgggattgttcggtaacgttctttcaacaggcaaactataccaacgtcatgcgaaaacaaaacgagctgtttaccagactcaacagcatattgtgtcaggggagcgcgggatcgcaaaaaccggccaccccctcggagccacggaccgccaccgtcgcggcgaccgcggcaagcgacgtcattaaagacgcacaatatcgcaaagaacagtacatgaaaaaggtggccagggacggctttaaaaaactaacagagtgtctgcagacgcaaagcgcggtgttggcaaacgcgctctgcatgcgcgtatgggggggcgtcgcatacggcgaggcgtccgagctggtgaaccattttctcctcaggcggcgcttcgtcgcgcttccctgggaagcgcgctgccgctcgaatcagattttattcgaaaactcaaagtacattaaaaactcactatattcccagcgcctcagtcgcgaacacgtagagattatcacactgcagttttacggcctgataaccggccccctaacgcgccagagcgatctctttcccggccccgccaacgtcgtgctggcccagtgtttcgaggcggccggaatgcttccgcatcacaaaatgctggtgtcagagatgatatggccccagattcaaccgaaagactggatagaccagacatttaatcgtttttaccaacttcccgaaggtgatctcaacgcggtacaaaagtccgcctggtgcttcatacgagagctcgtcctctcggtggcgctttataatcgcacgtgggaaaagacgctgaggatattttccctggcgcgcgagaaaccctccatctccgacctagacgttaaaagcctgacgcccggcctgtatctaacgtacgagcaaaacacgccgctcgttctaatttctcaaaataccggctggatatttaaagacctgtacgcccttctgtaccatcacctgcaactgtcagacggccatgatgataactaaccggacgcgtcgtctgctgcgggcgtgggtcgtgataatcgcgattggcgcggcggttggcgaaaacgtcaccaccccaaagggcgtgaccaccactgcaaagtccacgccgggcccgtcgacgcccacacctcccgaaaacccacccagggccgaggcgttcaagtttcgcgtgtgcagcgcctcggccaccggcgaactcttcaggtttaacctggaaaagacgtgtcctggcaccgaggacaaaacgcaccaagaaggcattctaatggtgtttaaaaaaaatatcgtcccccacatctttaaggttaggcggtaccgcaaggtggccacctcggtaaccgtctatcgagggtggaccgagaccgccgtgaccggtaagcaagaggtcattcgaccggtgccgcagtacgagattaaccacatggacacgacctaccagtgtttcagctccatgcgcgtgaacgttaacggcatcgaaaacacctacacggacagggacttcactaaccagaccgtgttcctgcaaccggtcgaggggctcacggataacattcagcgatacttcagtcagccggtgctgtacacgacaccgggatggtttcctggaatctacagggtccgaaccacggtcaactgcgagatcgtggacatgatcgcgcgttcggcggaaccgtactcttactttgtcaccgccctgggagacacggtagaggtatcgccgttctgcttaaacgactcgacgtgctccgtcgctgataaagccgaaaacggcctcggcgtgcgcgtgcttacaaattacaccattgttgacttcgctacccgcacgcccaccaccgaaacgcgagttttcgcagactcgggagaatacaccgtatcgtggaaggcggaagaccctaagtcggcagtctgtgcgctgacgctctggaaaaccttccccagggccatacagacgacgcacgaagccagctaccacttcgtggccaacgacgtgacggcaacctttacgtccccgctctccgaggtagccaactttactggcacgtactcctgccttgacaatgtaattcagaaaaccctcaacgccaccattaagaagctgtctgatacacacgcggcaaacgggtcggcgcagtactacgagacggaggggggactgtttctactgtggcagccgttaacgcaactgagcctcgtcgacgagatgcgcggcttaaatgacaccacgccggcaccccccaccacttcaacagccagtcgcgtgcgtagaagcgtcgatacaaattcgcaaacggccgaggacctcgcagcgccacagcttcagttcgcttatgacaaactacgcgcaagcattaacaaggtgctggaggaactatccagggcgtggtgccgagaacaggtgagggacacctacatgtggtacgaactcagtaagatcaaccccaccagcgtaatgacggcaatatacgggcggccggtgtcggccaagtttgtgggcgatgccatctccgtgacggactgcgtaacggtggaccaagggtccgtcagcattcacaagagcctccgcacgtccacccctgggatttgctactcgcgccccccggttactttcaggtttcttaacagcaccacgctgtttaagggccagctgggacccaggaacgagatcatactgaccgacaaccaggtggaggcgtgcaaagagacgtgcgaacactatttcatcgccagcaacgtaacgtactactacaaagactacgtatttgtaaaaaaaattaacacctccgagatatccaccctcggtacgttcatcgccctcaacctgtcgtttatagagaacattgactttagggttatagaactgtacagccgtgcggagaaaaagctgtccgggagcgtttttgatatagaaaccatgttcagggaatacaactactacacgcaacgactggcggggctccgagaggacctggacaacacgatcgacctgaaccgcgaccgcctggcacgcgacctgtccgatatagtcgcggacctgggcgacgttggccgcacggtcgttaacgtggccagtagcgtgataaccctgttcgggtctatcgtgagcgggttcattaactttataaagagtccgttcgggggcatgctcatgatcctggtgatcgtggcggtcatcctaatcgtgttcgcgctaaatcggcgcaccaacgccatcgcccaggcccccattaggatgatctaccccgacatagacaaaatgcagccctctggcggtaaagtcgaccaggagcagattaaaaacattctcgccggcatgcaccagctacaacaggaagagcgtaggcggttagacgaaaagcaaaggtcggcgccctcgcttttccggcgcgcgtcagacggactaaaacgtcgttttaggggatataaaccactggaaaacgaagaggctcaggagtatgagatgaacaaataaccacacccatacgcctgtacttgccgcccgccagagccgcgcggccaatcgattcgcgcacccggccggtcccaacaccctctggcgcccccgggcgtcagcgttctgtatcgagcatggatttttttaacccgtacctgggtcctcgaggaccacgccccccctcacacaaatgcaccgatgcgcccgcccccgccggcgccgttcagccgccaccagacgtctgcaggctcattcccgcctgcctccgaacgccgggggcaggcgggatgatcccggtcacgatcccgttcccgccgacgtactttgaaaacggcgcgcgcggagacgtgctgctcgcccacgaacggtccatgtggacggcgcgcggccaacggccagtcgtcccggacccccaagaccaatccattacgtttcacgcgtacgacgtcgttgaaacgacgtacgcggcggacaggtgcgccgaggtaccgagccgcttccagacggacattattccgagcggaaccgtgctcaagctcctggggcgaaccgagaacggcaccagcgtgtgcgtgaacgtgttccgtcaacaggtatatttctacgcgaaggtcccagccggcgtcaacgtcacccacgtcctccagcaggccctcaagaacaccgccggcagggccgcgtgcggcttctcgaccagacgagtaaccaaaaaaattctcaaaacgtacgacgtcgcggagcatcccgtcactgaaatcacgctatcgtccggttccatgctctcgaccctcagcgaccgcctcgtcgcgtgcgggtgcgaggtgttcgagtcaaacgtggacgccgttcgccggttcgtcctggatcacgggtttaccacgttcgggtggtactcgtgcgcgcgtgccacgccccgcctagcggccagagatgccagaacggccctggagtttgactgcagctgggaggacctcagcgttcaagcggaccgcagcgactggcccccgtaccgcatcgtggccttcgatatagagtgcatgggagaggcgggattcccgtgcgccacgcgcgacggcgacgcggtgatccagatctcctgcgtgttctacacgaccagggaaggcgcgcccaatccgccaaacatactgttcagcgtcggaacgtgcgaccccatcccggacaccgacgttctggagtttccgtcggaatatgacatgctggtgtcgttcttcgccatgctccgagacttcgaggtggactttttaaccggctacaacatctcaaacttcgatctcccttacctgattacgcgagcgtcccaggtgtacaatctccgattaaacgaatacacgaaaataaagaccggctctatctttgaagttcacgaaccccgtggcgggggaggggggttcatgaggtcggtctccaaaattaagatagcgggcatcgtccccatagacatgtaccaggtgtgtcgcgaaaagctcagcctctccgactacaaactggacacggtggccaggcagtgtttaggcggaaaaaaagaggacgtatcgtacaaggatatcccccctctgtttcgctcaggtcccggtgggagggctaaggtgggcagctattgcgtaatggactcagtcctggtgatggacctcttaaaaatgtttatgatacacgtggaaatctcggagatagccaagctggccaaaattcaggccaggcgcgtcctaacggacggccaacagcttcgcgtgttctcctgcctgttggaggccgcggccagggataactttatcctcccggtcccaacgcccgagggacaggggggttatcagggcgcgacggtgattaaccccatcccggggttttacgacgagccggtcctggtggtcgattttgccagcctgtacccaagcatcatccaggcgcacaacctgtgctactccaccatgatacacggacgagacctgcacctgcaccccaacctgacgccggacgactacgagacgttcgtgctgagcggcggaccggtgcattttgtaaaaaaacacaagcgggagtctctgctgggaagactgctaaccgtgtggctagaaaagcgaagggcgatccggcgcaccctggcggcgtgcgacgacccgtcgctgaaaaccatcttagataaacaacagctggccatcaaggtgacctgtaacgcggtttacgggttcaccggggtggccagcggactcctcccatgcattaacatagcggagaccgtgacgctccgggggcgcacgatgctggagatgtcaaagtcttacgtggaggccctaacgacggacaacctgcgaacgcgcctcggtcgcgaggtgaccgcctgtcacggtgcgcggtttcgcgtcgtctacggtgacaccgactcgctcttcatcgcgtgcgacggttactccgcggaagccgtttccgctttctgcgacgatctggcagccaggatcacggcggacctgttccccccaccgattaagctagaggcggaaaagacgttcaagtgtttgctgctgctgacgaaaaagcgctacatcggggtcctattgaacgacaaaatggttatgaaaggggtcgacctcattcgcaaaacggcctgcaagtttgtccaggagcgatgccgctccatcctggacctggtgctccacaatcaggaggtcaaggcggcggcgcggctgttgtgcaagcgaccgccgcacgcggtatacgaggaggggttgccggcgggctttataaaaattgtggaggtcctcaacgcgagctatgtggaccttcgaaacagcgtcgtgcccatcgagcagttaacgttctcgaccgagctcagccgccccgtctgcgattacaagaccaccaacctgccccacctggcggtgtaccagaagctggcgagcaggtgcgaggagctgccccaggtgcacgatagaatcccctacgtgttcgtggacgcgcccggggccctaaagtcggacctggccgaacacccggattacgtcaaacagcaccagatccccgtcgcggtcgacctgtatttcgacaaactggtgcacggcgcggccaacatcctccagtgcctgtttggcaacaacgcggacaccacggtggccatcctctacaattttctcaacgtcccgtataagctgttctcgtgaacgccgatagaaaaacgccagcataaaacgccgcgccgcggagtttgtggggaaagctcgcgcggcgaggaacgaccagcggagaccgccaccatgctggttaacgagctgtcgatggtcctcggcgactgggaggtgacctttcaccggggtaaattcagcttcgtcaacctagcccgcctgcagacgtttaagggccacgggggctacgccaaaatccgtctccccttctcgctcgaccagttactccaccaacacttcgcgttcggactcgtgacgcgtctcaacgaactgccccccttctccgactgcgtggccctcatcgccccacgggattccggcggcgacgcggacgcggcgcgcgtggcccccgggttcgtgctagactcctctcgcccgctaaccgtgtgggtgaacgcgaacgggcggcacacggtccggttctgcctcctcttcctaaagccgatcgacctggagcgcgcggttacgtacgtcttcggcgagaacggcggcgcgcgctcggaaggcgccccaaagcccacatgcaataccgaaagcctgcccggcggacccctgcgcgtctccggcgaggcgtctcagacgtcgccccactctttcgtcgcgtattttcccacggccgatccggtggcctgcctgagcctgttgcggttacaggtgaggccgttttcggatgacgcggcacacagggacgcgcggatctccccgaaatacgtcacgttcagtaacgccgggggtaacgtctgcaaggcgtccgttcacacgctgtccccgtcgcggtgtaaaacggcgcaaatggaaatcatttacgctccgggggaccccaacgccgagatagtcctgggccagtccggacccgtcctgcccacccacaccggcggccgcgtattgggggtgtacgccgacgccgaaaaaaccatccaacctggaagctccgcggaagtccgggttcagttaatcttccaacagggagcggccgctcggggcgatctggcgtttctagttgcgggcgtggcaccggagcccctattcatcgtcaccccaacacttttgctttccggttgcacaacacacctgcgcctattcaaccccaacggtacccccacgactataaaaagagacaccctggtggccgccgccgcgccctgccccgtggtgcgagtaagctccgccgacgacgcgccgcgggatctcgtcgcgtcaccagacaccggggcgctctccattgacgcgttcacaatcccggtcggtctccccggggtggtctcggcggagtgtcacgtgtctatgcgcgacaacggggtccacgaacgcatgagccattaacggcgacaatgggaacaccggtgcgtttctttcgcggcgagtggcaaacctcgagtctggtggacaacggcacgccacggtacagctccctggtgtgggccgccactatccacgacggctacctgacactggtgaacaggtcggagctgtgcgtcacggagcggtctccgtgtctgccggcgtgccccagcatcgggagactggtcgggaagaggtttcccggcttcgcctttgccagcgccacgctgggcgatcggggaacacgcaccgtgttctacgcgttcggtcaccgcgacaaccccctggacatcgtacccgccgtggtcgagcgcgcggaccgcgagctggtgctgcgagttcacgcgccgcgaacgacgcgggtgtcgcgatacggacttaagatatttgtggcgatcgtcactgtggtgcgcccgcccggggtgtttctacacttcccacaagaccgcgttccgatagcgctgacggacgcgtgcagccaggagggctccaggctaacctctgaagagccgtggataaaaattcaaggctttcccgtcctgtcggacgagaccgcgcatccgtttctcttaacccagaagaccaagccctttaccgagcgcaagttctgccgcctgatcatggacgacaaccagctcagcgccgtcaacaccgtctacctgggaaagcagcacgtgagggtaaccgtgacccgccccccggaaacaatcgtcaccgacggccccgtaacggcaaccctgtccctcaccggtaatgctccaatcgcctttcgccacaacccatactttgaactcccgtggtcgtccacgacggcgatattcactcccgtggtgtacgtgggcctgaccgtgtgcatcccacccaactgtagcaaatttgtgaggtacggtaacacctacgtctcggcatttaaccgcaagctgacggcgattattagcaaccacgcccacgacggcgggtttcgcattcaggactgcgagtggccaccgaaccgggagatagagattttggtaaccaacgtaacccaggccccggtgtatgtcagcactggaacgcagctggggcgagccatctttgtgttcgcgccgcggttcggcggcccggcaaaactgcggcagcttctcggccaccgatcgcgcgcgctggagctgcctggcggggtaaccgtggacagccaaaaactgtgtaggtttgaaaccatgtacctgttttccacgtaaattacgaataaaccggtcgttcgtattactcacacaacgccaaaccgtctttcattctcggggtcgcgcgtttcgcaaacacacgaggtttcccagacaccccctcccgtaccctcgccacacaaagccaaagccagtcaacggctttcgttaaatgcagagtttatttattattttcttacatcatagctattgcgcggcgcccgtcccgcaaaaacatctgtagatattccagtagacgaaacgcgctaagaataacgtccccggggctctgctctgctaacgcgcgcacaattttatcgttagactccacgccacctatcttgttatccacgggcagctcctcgattaatgagttgagggcggacagcaccacggtcaccgcggagccgatggccgcggcgcccgcgtttccgggcgcgtcgtccagcagcatcttcgagcgcgtcaggtaactctcgtaggcacggagcccgcgaaccatggcgttcatacactcggccccgcgaaactcgcggcgctggcactcgacgttagacattaatatagggaagttaataaacgacatgatgcgcgggtactcacgcacccgacagagagtggagtggtagcacaggtaaacgaaccagcgctgtatgtttaaccccagctcggacaagcgccccctggtccggtcaagagaggcgcggttaccggcccactgggggagaacgttaattccagcggcagtcgggggaggcgccagcgtagggctggccgcccaacacgacaggtaaaacaagacgaaccagacaggaaacataaccggttaaattactcgctcacaatcgcgggcggcggccggttaacgccaggtccataaaaacacacctgccgccaacccccaaagcgggaccgcgccctggaacgcggttccttccaatcgcaaagaaccgcgtcacaaaaagggctcgtttggaaccccttttgtgccagtttccaaagacggaaacggctcgttcgaaaatacaaaacggggtgctcaaacggccatctccatctctatgtgggggtgggggtcgtaaccctcgagactcaggtccgcacgcgtaaagtcctccagacgcgtcacctttctcagaatcttcaaccgcgggaaaggacgcggggtcctccccagctgaagcagcaggggcccaacgtggttgttgtaaacgtgggcatcccccagggtgtgtacaaagtctcccggggtcaggcccgtgacgtgcgctattagatacgtcaggagggcgtagctggcaatgttaaacgggaccccgaggcccatgtcggcggacctctggtacagctggcatgacagctccccccgagccacgtaaaactgacacaaaacgtgacacggcgggagggccatcctcgcaaggtccgcggggttccacgcgcacatgacaattcgccgatcgtggggccgtctgacgatcaggtccaccacgtagcgcagctggtccaccccttgaccttcgtagtctgcatcggcccccctgtactccgccccaaagtgcctccactggaacccgtacaccggccccaggtcgccctcgcggcggtccccaaagccctgcgccgccaagaaggcgcgggacccgtgcgcgtcccaaatttttacgccgcggcgcgacagttcggtggagtcggtggagcctctgataaaccacaacaactcctccacgacgcccctccagaacacccttttggtggtcaacaggggaaactcgtccctaaggttatatcgggcctgaagcccaaacacggacctggttcccacgcccgtcctgtcatccctctgaacgccgtgtctgatgattaaatccaggtgcgccaggtactgcagctcgccgtgctcgccgcggccgtccggggcacgcgccgcggcaacctcggatccgcgagaaacgcgacaacgcacggccgctattccagacaagcaagcgggtataatttttttaacgtagcagataccgagatgcaccaggacaatcataattatgaccgaggcgcttcacaactacagcccgtaaacggctgaaaacgagtctcattttaaaggcagcttgggcggggtcaggccacaaggcgcggcccggctattagcgagtagtaaacccacgtggtccctccccagcgtctcccccagagtcccccccggcgtctcccccggagtctcccccggcgtctcccccggagtctcccccggcgtctcccccggagtctcccccggcgtctcccccggagtctcccccagcgtctcccccggagtctcccccagcgttcattaaccaaacacgcatagtacaggaaccacaaaatacgtttatttttagtgtcatcgtggaagcgcccccgtggaggcacatagtaatagagtgcgattcggcatcacgcggaaatataaacggagggtcggtacgtataacgtcgtaaataagcagttgcgcgttgcttgcaaattccagcggcccgcggcaagcgttacgggcccaaccgcgttggccccgggggtctttttagaaaccgctgccagactccaggccttcgtcggaagcatcgtcaagcaggctgcgccgggttcttctgcccttgttgcgcttgggacgattgtccaccttctgaagcagcttacgcaccccgtcgtccccgggattggcgcacagcttctgaccgcggtgggtctctaatattacagcatccagcgtacactgtgacgaggtatagtagtaaagggccacggccccagaaggcggcaacttatttatataccctaaacagcaaacttcgggtgccggtccgctcatggagcccatgggaaaggcataatccaccacacacgcaaacaccgcaaaaaaggcgccaatcacaaggaccctcatggtgacaaaaaacacaaaacaacggagagacaaagttactcgtccgttaccaccagcgccgcgctcgccgtgcttggccacggatgacggcgtccattaatacccgcggcccgggtccgcctcgacggaaaccaggcgtggccagccacctggcgcacgtgcctcccgtccagatgtggcgtcgcgagtcccaaaacgggcgtgtctaactacggtcgcccccggggttcgaaagaacggggattccccaaacaggtaaacagcttttcgccttaacagagagccggtactgaaaggaatctccaaaacaagcaggggtcccgtgccagcgataaacgggaaatctcccccacctaaacacacgcgccgcataaactaataaaacacacaaaacttccccagcccgtgtgcaataaaaacacacaagggtcgtcatctcgcggggcccaaagggccccgcgcgtctgggggcgcgttgtctcgtcgtggctcgcaatcaccgccccgtctgcccgaaaacagggaaaccaccctcaggcctagtttttaaaacttaacactggcaagggaaaagggaggataggcgcggttaaaggaaaagggcaaccgccgggtcagcacgggcagcgaggggcaccaaccccaaaataacacaacgtgatagcgtaaaaccccgcgggcgttccgcgtgttttgcgggccggggtcggcacggccaatcccgcgttgggcgttcgcacaagcacgctttgctccgcacccaggcagggggcgccacgcggcggcgccggttcaggggtcaccagtgggtcagaaaaaatagtttccacggtcgcccggcgcgcgcgtggaaacatctttcggtgccctccgaacgacccgaaaaatccccgccgcaccgggtcgttcggagggcaccgaaaaatatccccaattgcaaccaagaacaacatgtccggggtttcggtgacgcgcggagcagaggcggccggtggtggcgtcggcaaaaatagtggccggagagcacgggtttcggtaatgacgcgcctggacggcggccacctggcggcgggccagagacgcgagcaccgcgagcggccggcaggtggcgccgcgagagaccgagcgccgcgatctgtcaagatggcggaccggcggcgccgattgggcgcagcggacgtcaatcacggcggagaggagcgccgttggtcgggttacacgtcaatcaccgagggccgcgctggttggcagataatatgaaaccggcgcgtccattggacgctggcggcagccaatgggcgcgttgggttttgtttacaagttccctatatatattatattataccttccctgttaagggaatcccatgttcttacatctaatcagctgggttacataattagaatgtcagtaattactattattactgtattatttattaaattatttattttattatttatttaataaattaattcttgcgttattatgtaatcatttactgagaaatctactaccaatttaataaaaataaataattaatacaattgtggtgggggttctaaatattgtgcggaaaaacaatattttttaaatctattcattaaaaatgcttctattttaattgtgacgtgcgtattaagaatgtgggagtggtttgtaaccatgttttcaaacaacaccattttgggccgtctgaaactcatataagcggaaagctcggcgttaattgccactggccgccaaccagtccaccttaccagttggagtttaccactgcttacggctttaccttggcacaggctgatttacgtaacacgcgttgggggtttaacccggttgaatttggttgtttgcacgtgcaaaaaagcccaccgcggcttgggtttaagcgtttaaagcgttgctggttttatgacatgctttttgatttaactgctatgggagctagctcgtagcccgcatgctttcggtttagtggttgtacaaaaacaatggctcgtaaaccgcagcttttggattgtcactggagccaaaaacggcgaacctacgcaacctgccccgcgtcgcggtttctgtttctagctcgctcgcactacattttaaattttaatttaaaatggagcaatggcacggcgtaaatttaagcacaagcacgttaagcttttagatgaactaacgtaaccagtacttttggcgacctggcgaatttaaaatttaatgccgtggtctttaacacaaagctgccagtttgcaatacgctgcatatatacagtgccgttctaattttatggttaaaaggattttaaaattgtgtgaattttgtaattgtgtacacgtgaatttttaaattgaatttaaaattgtaattatgtacgcgtgaattttaccattgtaattatgtattgaattttttaactgtgttatagttgtatttttgttaatttgtgtaattttgcttttagatttgttcgggtgtgaattccagacaggtaagattttagctcctaatgtttgccttgccgcctagctcctaatgtttgccttgccgcctagctcctaatgtttgccttgccgcctagctcctaatgtttgccttgccgcctagctcctaatgtttgccttgccgcctagctcctaatgtttgccttgccgcctagctcctaatgtttgccttgccgcctagctcctaatgtttgccttgccgcctagctcctaatgtttgccttgccgcctagctcctaatgtttgcccacgtttatgtttaagcacactaaaatttaaaaacgtttgtgttggtttttatgaccggcttggtacaaaacctgctggtgattttttacccaacaaaaataataaataaaaaagttaaaacttatttctgttgtctgtggtaattgcgttccccgagggtcccggtctccccgttccccgagggtcccggtctccccgttccccgagggtcccggtctccccgttccccgagggtcccggtctccccgttccccgagggtcccggtctccccgttccccgagggtcccggtctccccgttccccgagggtcccggtctccccgttccccgagggtcccggtctccccgttccccgagggtcccggtctccccgttccccgagggtcccggtctccccgttccccgagggtcccggtctccccgttccccgagggtcccggtctccccgttccccgagggtcccggtctccccgttccccgagggtcccggtctccccgttccccgagggtcccggtctccccgttccccgagggtcccggtctccccgttccccgagggtcccggtctccccgttccccgagggtcccggtctccccgttccccgctttaacgcaacgttaaagtggtttatttagcaaacacgtttttgctgcgtatccatgcaaaaaataaaaagttgggtttccgaagcgcatgggtgctttagtgtttcatagcacgttttcgtgtcacagtaggagtggttagttttttggtagcgtatgcaactaggtagctcgtctaaccgggaaaggaaacgttttaaaccgcagtgttaccgcggtgtggcatacggtttacgtgcaactgccggtacgcgcgtggtacctgaggttacgtgtagcaattatggcggctattcagggccctccgccgctccctgaggaggaaaatgaaaattctttgccagatgacgtgtatgctatagagggaatctttctttactgtgggcttggacaggcggagtacttgcaccatcccgtgtttagccctatcaaggaatttatcagctcctttctcaaggacagcgcgcgcctttacgaaaggcttttgcgccacaccgattaccgctctttgcgcggactaaacgccataggccaagggatgttgcatataaacacggacggacgccacaactggggtcgcgctttggccgtgttaggtcttggcgcctatgtggtggataaaattagagacgacgagcgtcttttaacgttcgctatagccgttctacccgtgtacgcgtacgaggcgctggagtctcagtggtttcgttcgcacggcggatgggagggactccggaattactgcgagcgaatactgaggcatcgccgcaatgcgaggagacacatgtgctacggggtcgcggctggtcttctggcgctagttgcgctgtttgccatcaggcgataggcgtgcgtttaaccgcgtatccccctaccctaaccttaacttagccaattgtatataaaaccaataaaacaaaacaagccaacgtgttattcgttagaacatctttttatttgagcagttcctcgcagaacattttctgtatttgtgacacgggggccggctgcacgctggcctccaccggggcccgcgcggactgtccgcagtctgaatcgaggggcgggcacgcacccgcgcggggggcagaggcggaaggaacggcgtttgacattgccgattcctgggcggcgggggctggagccaccggctgcgtggcaaggccggcgtggtggctcgccggctgtgggggcggcggctgcgtttgctgtatgccctgtgcgatctggtacggctgtgaggcgtggagatgggtgtccgttcctggataccactgcggctggtacgctaggtacggcgcagacgcgtaggtttgcggccacgagaactgaacggggggtggttgtggtcgttggtcggtgtccttttctccagaagtctgcgaagcggctcgtttcaggtccctaagatccgcctgaatgtccaatatgtttttggacagcgccatgacgtccttgtgtatgccgacctcctcgcctggaaacacgggctcgtcggaaagttccacgtcccgtttgcgcttctggtggcgagacggtgcctgagcgtagccgtacgtgggagaatagtacgtggcggcatacggagagggttggggggcaaacgccgactgaaaagggaacggtggcgctacgggcggcgcgtacgacgggacggtcagttctggtggcggtatccacgagggatacgccgccgttgccggcatcgagggtggcgcggaaacgtacgcgcgttggccctggttgcgcatgtgatcgagactgctctgcaccatggttagaaaggtgctcttgggaatggtgatgttgtcatccgggagggcgctcatggtttgggtgtcgcggttaccgccgtgctgagaggagggagactggctggcctttaaatacgtacttctcgcgactctggccacgcctttatctgttttgagcaggtctgtgcgatctcggataaatccggcgtcgatcgccttggccatcagtgtttcaagaggggctgcgaattctggcggtgtcacctgacgctcgcaaagatctaaacacttggacgttattttaccggcctcatctcgcgtaagagaatcaaatttggagacaacccaggtagggtctggtccgtaaacggcgatggaccccctacgcctccccagtgcgcacagagagacgtgctgaaacgcgggtgggtgattggggtcttgcagctcttctgggtgaagagacgagagggagagctctgggagccatgtgtgtaacatctccagcagggggtcccgaggcatggggttaagctggatttgggccgctcgcgagtcccccgcgagccgtgacgccagggccaaaaaatcggggctcgttagcttccccaggcaaaatatcccgtgtgttacttgaaagagaccgagcgtgtgacctacgtgggcttctgggacgtgctctatgtttatcggtagagggttcgtatacgggagcagggtcgccacgattgagggttctaaatacaggtccttttctatctttggtaggctgaccacgtccacgtatccaccgacgtacacggaagtcatgttgggtcggtgtttgcgtgagccgaaggagatgagcgccggccttcgggggctgatgtggcgcgttatccgttgtgagaatttaaatacgtttctcccgggcgagctcaggtttcttcacctggtgctgtgcgaaatgtacaactacggtcttaatgtgtacctgctgaaggaggcgattgccaataccggaacgagggacgatattgtcctcgggcgtaaggttccggtggagttctggaaaatcatttacgatgggttaagggagatgggggtttctgacgcaacgttgctctcagaaaccaaaaggggggctttgtggctttacttcaacgggcgtccgtgcctgttgaaaggtttaggcgactacgttttttgtcgactggggttaagccacagcgtacgcgtagtgccggaaaacctaacggatgggaattatctctataacctgggcagcgttataccatgtcgcctgctagtggcgctgagctactgcttggcgttttgggggcacgccgaccacgagccctgggtcaggttgttcgccggcaagatttttatactgtacttaataatatccggccacattatgccacgaaagtcaatcttagaacaggtgggaacgtccgggtacggtggctttgtcgaggctgtgtgtcgcgacgttcgcgcggtccacggcattccggcgtgggattttgcggcagcggctccggcgttaacgtctcgacagacggactacctgtttgcgttcaataacagcgttgtgtagggtcgtctcacactaccgacgtggcgaccggcaggtatcccaggaccaaaaaataaactttgtcgtagtcgtcgtaaactccgaattggctcttcattatctcgtgtgttaaaccggggggtgatgggtgcgagaggatggcggccaggccttcctcggccgttaaacgtagcgacgtttgggcctgaattaggctacccgcgtccctgaatgtaagcttttggttcaggatgtcaaaaatctcgttgaatctggagccggtcaggttaacgaacggacgagtgtgttttaaatgggaaatggcctttgcctccagcgccgttagatacgcaccagggaacaaaaacgacgtgggggcgtcgggcgcgcgcgaaagaagcggggagatgtaattttctgtcaggaaatcaaaaagttgtccctttttaaggtattgtttggaccccgacgggtcttccaccttggaaaatctaccacccaggagcgaggccaggttgaggcgccggtggggcgatctgggtgtgatttgggccagttccaataacacgtaaaccaaaactagacagcccagcgccgataggccggcgcgaaggttccattggtacgcttgaaagggaggcacgtcctcgctaaacaaagtggtcgcaaggatgtatacgagatcggggccgggtggctgcccggtacctgggtttttggaaccgggcagcagtccgctctgtgccaacgtcgcgtatatcacgtggtttgtaaagaagtccgcactgtactttgtttctttgtttaggggcgctagagaggcgcgttgcttcggattcgaatatgtaaactcaaaacccaggcccttcgtgttttgttttaggtgagagataagcgtcgccagctttgtgccgaggttatcccagaggccgtctcgagttggtggcaacggggtacccgtccggtgacagtagtacccgttgaggaggcacagcgcggcctgcgtgtccgatacgttgcggacatccgcgtaaaagtcttgtgtaatcgaggcggctgttgagataacagcagacattagttttagggacgtggagttttgaaagtttgtcgtgcctcgcagatctctggggaacaccctgcgttgcatggcgttgtcggtaaggctactgtaccacggaccgtattgtggcagccattggttttggttcatgtacaggctgggcaggagctccagacggagatcctggtttaccgtgaagcccgggtctccgggggctatcgtgacaacaaacgcgtctccatgtgtctgaggggcgattttgggtcgtgggtttattgacgtttgggtatcggcggtttccctggtcggggaccgcagatccccgaccatgttttgaattgtgtctaggtcgcgcatgaccatttctgtttcccgttggtgcgtctgcagcacgttgtctagctctagacggagcaggttttttcgcagcgtgtgaacccttagcgccagctgttgatttcgcatcacaaacgcgtttcttctttcttctatcagcgatggtttgtgaatgaacagaacgttgtttggatgtggggcccagaaagttgcggtaatttgccgcgctggttttcgtggatatctcatacagcatttttctgacgtgcgcatcttcccattggctaaaaaacccgttaagcgccaccgacgtggtgttgataaatgtaggcggaaacgctgatgactcagtatgaattgttttaagtgttttctggttcttaaatagcagccagggcctcaccgaccagttttcgtgccctctcggcgcggctctgcctatatatgccacggaatctgtcagttggtgtaagccctgccatcgctgtgatatttttattgcgttaccgggaaatacggccgcgctcaggcacgttttgagttcaatcacgtagcacgttttttgatgttcgctatcgacggtcagcacacaatctgctattcgcctgcccagggtaacctcaaaaaaaatcctaaatttaacgtctttggttgcttcgcgtgttgggcggattgaaagaaactttaaaatatcatcaagcgaccggtgtttaattaaccgtcggtatacggctaagtgggcgcgaattccagcccgtttgcgtgaggcaggaagggcttcgagggtcgcggcgtgtttacattgattcgcgaccgtcatggccgaaggagggtctggctttggcgacgagcttgtccgtcagatgcgggatcggaagcctagatgggacgaatcttccgacgataccgatgacgttgacaccgaaagcaccgatctcgagtatgatgatgtgttccccgtggttgatacgcacggcttaacgagccctggaagtcaaaactatgacgtacccacgtctccgtccggaacgccgtgggaattactgcacccggacgctctgtatgcgcagccgaggtgcccacctaaaagggcggccgtgccgggcggtggtgcgcggcccaaggtgtccgcgttctcggctagacttcaatatgttggacgacagagttttggggatagagagacgcggcagcttaccggggcccagttttcttccgagagcgagcacgaatatgcggaaataccggagcgtactacacgcccggttgaaagcggggacaagagacattttacctctgggcgtaggggtggaatttcgggaccctcgtcgtcaaaacctagtaacggtgcggggttaacacgtaagactaagacgtcgctgagtgttagtcttaaaaacttgctgcggataaaggacgatgatgttaaggttgatgtacccaggccggttaccgtgccggttcatcttatgcagccgcatcctatgacggaatatagaaatgcctttttaatctatctagagggggttatgggtgttggaaaaacaacgctattgaattcaatgactggcatggtgccgcaggaaaacgtcttaagctgtccagagcccatgaaattttggacgtgtgtttattcaaattgccttaaagagcagcgcagcatagttaagcaaggcacccacgggaaatcgatcacttctgctcgcgtatacgcgtgtcagagcaagtttgcgctaccttttcgtgcaacggccgccggcatcggtcgcaacctgcaaccgtggctagtgggaaacggtagcacaaagccagcgaattggattgtttttgacagacacctactgtccgccaccgttgtttttccgctggttcacgtaaagtacaacaggctaacgccggatcatttgtttcaaatcctatctcttttctcggcacacgacggtgatgtggtcgttttgctaacgctcaacagttcggaggcgcacaggcgcattcaaagtcgaggccgcaaggaagaaaaaggaatcacgcaaaactacttgcgacaggtagcgtgggcgtaccacgccgtgttctgtacgtgggtgatgatgcaatatctcacaccggaacaaatggttcagttgtgtgtacaaactgtgtccatagaggacatatgcaatatgaattccagattgactcatcggtttctaaccctaacaaagttgcatgaacagagtatgataccaatggtggtagaaatgttagcagcggttaaagaacacgtgaccttaatggaggtctgtttgggcctctttaaagagctacgaaagcttcagattttaattgttgacgcgggagaacatttagatgatacgtgtggcctttggggaaatatttatgggcaggtaatgtcaaatgaggctattaaaccacgagccgtgaactggccagcccttgaaagctacattcaaacgctaaccagcttggaaagcaatgcagccaattaaccacggtttgtgtttgttggtaattacattctctgtaattaatggatatgaatataatgaagaaaatgtacctggacttgaaatagttctttttacccctgccacaacaacaccagttaaaagcgatatatcaactgtggaactggaatttaacagaacacggtatcgtattaattggaaaaatgtaagtgaagtgttaacttctcgtgtaatacaagatgcttgggctagttcgaaaattctggaaacgttgcaagaaacgttacaaaaaagaaaaggtattttaaaattaaacatggcttatgttagtaataaagaaattaatttttgtaaaagtttttctagttccgaaataataaacattacctcagatttcaactacagttccttgcctggatttcttggatcgtttgatgttattaatcatgaattaacgcacggagttttagtatcaaaacgtgaattatttacaaatgccttggatataatgggacttttttatgcaatcaaattaaatgcagagacgtttaaaataacgtttgatgaatcaaaagttattatttcaggtattatgacagccgattggattttagtttctattgcaaattcaagttcagaagttaatggtcagtgtgtggcgttgctattcggtgatccggaaaaatttccaccgctaaaaggatatgtttcatatcgtgatttagtggtcgtaagaaatgataattatgcaatagcggtaattgcacctatgtctcatggtaccatgggattaaactttttgcctcaaaatttaaccgacatatttttttccattttgaatagtccacttaaagtaataaattatttgaaaggcaaactttttgaactggaatcaaaaggagcatgccagaatccttcaaacgaacaaaacatattgtctttattttttgaagtaactgcaattcactttttatatgtaagaaatttaaataaagaagaaccagtaaacgttggatgtgttgtaaaacacgtggcagccttaaaaagtttggaaaggttgtttaagttgtgcttcccggcatttgaattacattcacttaacttgtggacattgtcgcatcttgcagcatcacagatagctaatttaccgccaaataatgtaatgtcgttaactatggatgaacaagaagttgtattttctatgttcaaactggcagataataacaacgtaaatgaaatcattttaaacgaaattataaacataagtgatcaaatgtatactatgtatagtgacatatatcaactatcaaacacgtacagacaaacagttatggatatttatgaagtgttaacaacagtatccttaactaacgtaggtgcacgtgcagtctatccatacatattgttcacatccatgtgtaataatgtagaaatatcatatatgattaatcaaatatcaaaacccgatgacattactatatttcgcgtgttttcgccttgttttttaagtttaaggtttgatttggatgaaaacaagctacggtccgacgcaccgcaaacgtcaaagagaactggctcggaactggcacaaggagcgtctggtttttggcggttgttgcatgcttttcacgccacgcgtataaacgagttttccgtcataaattgcaccagactggcgtggaagcaagtaaccgcgctaatgccgcttacgaacatcacgtacgtcataagctccgtgcgcccggatcacgctcgcgtttacgaggtttcggaggtgttccttaacagcgcgatgtttgtctcggccgtttatccaaattgttctcattttacaccgcccggtaccgccctacacattccgatattgtataacttttccgccccgaggataggatgcccgctgtgtgattcgattgttttgagctatgacgaaaaccagggcttgcagaccatgatgtacgtgtcaaacccccaagttcaagccaaccttttttccccttattcgcccttttttgacaacgataattttcatatccattatttgtggttaatgaacaacggtaccgtcgtggaaattcggggcttgtacagaagacacgcactcagtgctattgcatttgtatttgcttttattggaacaatgtctgctctgtattttttgttcaagctgttttccatcctggcctaaactgttaataaagcgtaaaactttaaaaggttgtttcccgtttctttctgaatagtcacgcgggggttcaaattagcgagaacctcaaatagaggcgtaccgtcacatattgattcaatcacctctgcgccaacttgaatggccagatctcgattttggggaggaaacattagaaaatatttgtcggttgcggttaataattgctgaggagcccgcgacgtaactttaaaatccttctgaaagttgtgggactctatggagtttaaacaaaacaccatgtcttccaggcgctgttttacgcgcgaaagcatagattcgaagtattctctttgatacggggccgccgccttacacgtattcgcgtatagcattttaaagtgaaacagaatattttccaaacccaaacgctgcaataggacgtgctttaaaaaaaggtgttggataggctttagttttaaattaggtaagaacattatagagttgttggcgcgttgcatgattgtcacggtgttaaatgcggtaattggactctcgcctatttgtggctggcgcgaacccatggcaaaaaatatgtcggccagtaaacagtccgactgtacatctgagtcgctggtcgctggcggctgcttgagaatggatcgtagttttttatcctccgatgtaaaaggcgcgctacacaaggttggtggcgcatttggatcgtattcttgcgtggtgtttgccagttcgtttatggttatctgtttgttatttgtaagagttaccggtggggttttgacggtcacatggacggaaaatggcctgtagtgcaaaaaaagagagttactcgcgatcactaattttgagtcttacggtaattggcgtgtcatcgaagtcagaacttctaattactgggtctctgaacaatgatacgcctctctcgccttcgtccgttcccacgcacaagcagctgtttagtgcgtagggatggggtgacaccataattcgcaacatcgataattccggacgtttgggggcatacaatggcatcatgttagaatagatggtgccgaattttttaaacagcaggggaaggggtacggccggtcccggaagtccgaccatcgcggccagtgccgacgagtacacgacatgacagacgtactcgccgttggatagctgcactgcccccccgactaagcgagctttcaaatctggaatttttatcattgattttcctttggtagttttaggaaccgcggcggccctggcttcgtggttatatttttgctttatgtctccagtgaagtcccaggcgtctaatagtgtgacgaattacctgtggttttaagtttgaaaacgcgtgaccggtcgttgctatgaaagacataaaattggcgttgcgtcctaactcgtctaaccgtatttttttacttcgcacgtatatctttggaatgaggttgcattttttatatcccagaatagcgcattcgtgtttgcttttggtgtgactaatgatgcgttttgaaaagtcaaacgcgccaaggcgggcggctcggttcagggctcccaggtccaaacgcaaaaacgaacaacattccaccaaacactcaatgaaacgcttgtgcagagtttgaatttgcgctttatttctcgtctccagcgttgtgaatattgtgtttttgacctggtctattctagcgtatgggacgctcaatattgggtccaattcatccgaaagttttttaagtaagggcctgccgtatattttagaaattgttgcagtggctgccttaaaccaggaaacattgcatccgtctttactagaaaccacggaagggaaattcgtggtccagaaaacgtctcgccgcccccgtctaactatgtattggttttcgagcccgtggaggtctaacgtagagttccagttcgcaatcgaaaccgggaaaatgtgcttcaccggcaaaaagcacctcaggtttcccatagcgaacataaagtgcagcatgtccccgcttatgttaacgttaatcgcagccccgttgcacaggttgtcggagtagaacacgctcacagtgaatgacggttctttcacggagaattttttcattacaaagttatttgtgagcctggggatgtcgatgattgactcgaaggtaccggtgagagaccggcacgtgattttggtattaattaccatattggtattgaaaactgagattttgaagttgtgtaaacttaggtgctttactggatcgaacatgtctggaagcggttcggtctggtggtcgtggtgcacttgtttatgtcgatctgggttttgattggcgtcaaaaataaaattgtgatctctatcttttggagcgtattgaattcgcgaaaacatggcctctagaacgcgaggcgtttcgccgggccccaaaaactcggtgttgggatacagcctgtcgcacgaatgacacacgtgttccgcgacagacatgcacattgcgacgcggaatatcggtacctctggtggaaacgttagatagtacgttgactttggcgtcacaacaactaccttgtacggttttttgccaggatcttttttgttaattttcacctgtagatttacgccaagcgattgattgactatgttttggtagtcgatgattaattcgctcgccactctgatgtcctgaaatttgttttgttttgataggttttttaaaaggaccgggttggattcaatgttgtaaataattaccgtttggacgcgatgcagttcgggatgtgagatccaatccgaagtgaaacccaaaatgttaaattttaagtaattcagatgcagttttgcgcacgggggcggacaccgagctagcgtgagggttttgaacggctctaaaagtttaaagtaattctgataacacgtgccgatcagatgttccggcagtgtacgcgtgaaggcggcaaagcattcctcgaattcccatatttgaaggtaacgacagagaattcgaaacatgctttgataatttacgccacataccattaatggcgaacagtacagggaccgtaggattcgttcgtcgcgttgaacccaaaccgtgtcggacagttcgtaggtgaagacggtgggtaaaaacgtatgcgtttcgatgtgggaaaatgtacatccgatagcatcatcaattattaaacccggtcccgcgtactcgttaccggtcttgccaacgataccaccttggcggtgtataaaagggtatagtccttctcccaataccagacgccggagcgcacggcacgcgtaaaaggtggtctctgtttccatagccctgtccaagacctctagcgagacgttttcaagaatctcgttattggtggccaggcagcaaaaaattgccagttgagttttgaaatcggaattggccggtgacggggtgtcagctgtcgccgtagcgggacacgcgggcagaagaacagggccctgcaacagcatcatggaggcggctctggaggtacgaccctttccatatatggccacggaggccaacctgcttcgtcaaatgaaagagtcggcagccagtggactttttaaaagttttcaactgctgttgggaaaagacgccagggagggaggagttcaatttgagggtcttttgggcgtgtataccaacgtaattcagtttgtgaagtttctggaaacgtcgctagcggttgcgtgcgttaacaccgagtttaaggacctcaaacgaatgacggatggaaaaattcagtttaaggtatctgtgccgaccattgcgtatggggacggaaggcggcccacaaaacaaaaacaatacattatcatgaaggcctgcaataagcatcacatcggtgccgagatagagctgtcgacggatgacatagagctgctattcattgacagagaaaccccgctcgattacacagaatacgccggggccgtaaaaacgattaccgcctctctccagtttggcgtggacgcgctggagaggggcctggtagataccgtattgaatgttaagcttaggtccgccccgccgatgtttattctgaaaacactatcagatccggtctacaccgaacggggtttaaagaaggctgttaagtcagacatggtgtccatgttcaaaagctacctcatggacaactcgtttttccttgacaaatcggacatcgccgttaagggcaagcagtacgtgctgtcggttctctccgacatggtgggggcggtgtgtcacgaaacggtctttaaggggacgaacacgtatctgtccgcctcgggagagccgattgccggagtcatggagaccacggaaaatgtaatgcgaaaactgttaaacatgctaggtcaggttgacggtggcatgtccggtccggcgtcttacgccaattacgtcgtcaggggcgaaaatctcgtaaccgccgtaacgtacggtcgcgtcatgcggacgtttgaccagttcatgaaacgcatcgtggaccgtcccaacgcgcagcccaacgttgacgatgatcgggacgcggtggcggacgggcaggactccctcgccaaaacaccgatcgcggcggctgttatccagatcggagataaactggtggcgttggaaagtttacagcgaatgtacaacgagacccagtttcccttcccgttaaacaggcgtatgcactatacttattttttccccatcgggttgcacatgccacgtcctcagtattccacgtcggccacgattaaaggggtcgagaatccagcggaacaatccgtcgaaacgtggattgtaaataaaaacaacgtcctgctgagtttcaattatcaaaacgcgctaaagtccatctgtcatcctcgcatgcacaacccgatcccgtgtgggcaagctttgggtcaggcgtttcccgatcctgggcacgttcacaggtacggtcaaaaatctgagcaccccccgaacatgaacctatacgggctggtgtacaactattaccagggcaaaaacgtggcgcacgttccggatatcgccctgaaggcgactatgaccacggatgagttgctgcacccgacctcgcacgagacgcttcgtttggaggttcatccgatgtttgatttttttgttcatcagcagccaggtgcgcaagccgcgtatagggctacccacaggactatggtgggtaacattccgcaaccactggcgcccaacgagtttcaaaacagcagaggcctgcagtttgacagagcggcggccgtggctcacgtgctggaccagtcaaccatggaaattatccaagatacggcgtttgacacgtcatacccactgctttgttatgtcattgaatgcctcgtccacggacaggaagacaaatttttaattaactctcctttgattgcattaaccattgaaacctactggaacaacgccggaaaactggcgtttattaacagcttccctatgctgcgatttatctgcgttcacctgggcaacggtagtatttctaaggacgtgtacgcccattaccgaaaagtttttggcgagctcgttgttttgcaacaggcgctctcgaaaattgctggccacgaggtggtgggacgcaggccagcgtccgagctgattaactgtcttcaggaccccaatcttttgccgccctttgcttacaatgacgtttttaccaacctgcttaggcagtcctcgcggcaccctatggtactcataggagacgaggggtacgaaacggaaaatgacagggatacgtacattaacgtcagaggaaaaatggaggacctcgtcggtgacatggttaacatttacgaaaccagaaacaacgcggatcatgacggccgccacgtccttgacgtcggtccctttaacgaaaacgaacagcacatggcggtgctggaaaagctcttttattacgtggtcctgccagcctgtgccaacggccacgtctgcggcatgggcgtcgattttgacaacgtggccctggccctgacgtacaacggcccggtgtttgcggacgtcgtgaaccccgacgatgagattttggaccacctggagaacgggacgctccgcgagatgctcgaggcctcggatatacaccccaccgttgacatgattcgaactctttgcacgtcgtttctcacctgcccgtttgtcacccaggcctcccgtgtcgtgacccagcgggaccccgcgcaactgttaaccactcacgacgacgggagatacgtgagccagactgtcctcgttaacgggttcgcggcgtttgctattgcagataggtctcgtgacgtcgccgagaccatgttttacccggtgccgttcacgaagctgtacagcgatcccctggtggcggccacgctgcacccgctggtcgcaaattacgtaacgcggctgccggcccagcgcgtgccggtcgcgtttaacgttcccccggccctcatggccgagtacgaggagtggcacaagtctcctatattggcctacgctaacacctgcccgattacgcctacgtcgttgagcaccctaacgagcatgcacatgaagctgtccgcgccggggttcatctgccacgcaaagcacaaaattcacccgggctttgcgatgaccgccgtccgaaccgatgaggtgttggcggaaaacttgttattcagtgccagggcctcgacgtccatgtttttagggcagccgtcggttatgcgtcgggaagtcagggcggacgcagtcacgtttgaggtgaatcacgagttggcatcactggacatggcgctcggttattcttccaccatcacgcccgcccacgttgcggcgattacctcggacatgggcgttcactgtcaggacctgtttctcatgtttccaggggactcgtaccaggacaggaccctcaacgactacgtcaaacaaaaagccggatgccaacgattcggtggtccgggccagattcgcgagcccgtcgcttacgttgcgggggtaccgcactcggacaacataccgggactcagccacggacagctggccacgtgtgagattgttttgacgccagtcactgcagacgttacctattttcagacccccaacagtccccggggacgggcgtcctgcgtgatctcgtgtgacgcgtacaacaacgaaagcgcggaacgtttgctctttgaccactccatcccggattctgcctacgaataccgcactacggttaacccatgggcgtcgcagcagggctccctcggagacgtgctgtacaactcaacctcgcgccaggtcgcagtgcccgggatgtacagtccgtgtcgccagtttttccacaaggacgctattttgcgtaacaatcggggcctgaacacgctggtcacggagtacgcggcccgcctcacgggaacgcctgccaccagcgcgacggacctgcagtacgtggtggtcaacggaacggatgtgtttttggaacagccgtgccagtttctacaagaagcgtttcccacgctcgccgccagtcacagggctctgttggacgaatatatgtcgaataagctcacgcacgcccctgtgcacatgggccattatatgattgaggaagtggcccccatgaaaagactattaaagattggaaacaaggtcgcctattagtttagctcagacggtctggagctaactagagatggccctcgataagagcatcgtggtctcggtgacgtctagattattcgctgacgagatagccaatcttcagtcaaaaataggatgcattttgcctctcagagacgcccaccgtctgcagaatatacaggcgctgggtctggggaacctgtgctccagagattccgcggtggattttattcaggcataccactatctggacaaatgcacgctcgccgtgttggaagaggtcggtcccaacagtttgcggctgacgcgcatcgatcccatggacaattaccaaataaaaaacgcgtaccaaccggccttccattgggataactactcagaattggtagttataccaccggtctttgggcgcaaagatgcgaccgtctcactggagtctaacgggtttgatgtggtctttcctgccgtggtgccagaaccgctggcgcaaacagtgcttcaaaagctgttgctgtataacatttactaccgagtggcggaaacgacgcctaccgacgttaacctagccgaggtgacgctgtacacgaccaatatcacttacatgggtcgtaactacgccctggacatggaccccgttgggtcaagctcagctatgcggatgctggatgacctatccatttacctgtgcgtgttgtccgcgttgattccacgtgggtgcgtaaggctactgacctcattggtgcgccacaacaaacacgaattggttgagattttcgagggggtggtgccacctgaggtgcaggctctggatctcaataacgtaagcgtggccgacgacataacgcgcatgggtgccctcatgacctatctacgaagtctcagttctatatttaatctgggccgcagacttcacgtttacgcgttctcatcggacacgaataccgcttcctgttggtgtgcatataactaaaaacgggcctccctgtgctttgacatgtcaattcccaaaattatgacggtgtcccgggacaacgagggtacggtgtgtgaggtcgcggtggacaacggacggcacagagcgatgatttattatcccaagattacaaactcgagcaacgagcgcgcggcacagcgcgcggacgtcgtcaaggaagcgtttgataccgaaaccccagtggacatcgtaaagcaaattgtgaacgagggtttagctatatccaaaagaaattgcgtccgtttggcattgtacttatatttttatctgcagtacgtgtgcttcgctatcctcatcacttggcaattaaacccgcacatagacccaccgggtctggtgtttgcggttaacccaatgggtccaaaacatgtgtcgaaactgccgcatccggctattgttgcggtgggttgtgggacagacgccatctgtaagaactgtagcgtccccgatataaagactgagcttggagtggtctaccacaacgggtctagcgattctggtcagagcgcacactatgggctggccctgttaaaggcggcctggcttgttatgggaaatgtgtgtcccgagccagtagtgcggcaaggcgctgaattacttggtccgtggaaccggacggcgtggttggattttaaatcggcgatggcggcaaccacgttttgcggatccagaggcgtcctctggtcaccgattcatgaaaaaaacctctgccgccccacctggaatgatgtaattaacacatcatccttttttacaaatgaatcactctgtccaaatgtacctggggtgtccgaaattgtaatagtgcttaatggtgatgcataagaacaataaacgtattcccacgcactctatacgttgtttttattggtctggtgaggtatagacaggaatgggaggttccgatggtgcctataaagcaaaatggtcttagagtaacaatattgcggatagaaaagtggtttgtaaatattttcacccgaacaatatgactgcacacacgaatggggttctaaccacgacgggcgtttccacaagtcagccggaatcgtttcaaatttctccattttttcgcgtaattacaaaaccgccaattatgggcttgtttttctgcgtggctatgtgcattatcgcgttggtgtggtacgtaatgcgtagggtgtattgcaaggggcgcgttgttgccgattcgtgtcgcgacccgcgtcgacccgcgtatgaaatgttgaatgttagattgcgtccgcacgggaccaatccataaagaactttcattgcgatagttaaaccgcacggcgttctctcttgctcgtttattctagcgatttaaaaacaaatttatcgttagttgccatgtagtgggccatgacggtggcaacaagtacgtcgtccgacatggttttttgttttgcgcagtacgtgtgacctccgtcttttagtggaatgcaacgtattgccttgatttgatctattagatacgctacgggatcaaacgatagcttgatagtgtgagacaccacagtttggctggcgctaaacgttcccgagttcagtgcgtaaataaaagattcgaacgccttggctttctctggccccaacatgtaaatgggcgaacgtataagagtgtttttgtccgcgtggtgtaaaaaactaagagggaccgagcatatctcgtttaacacagtcgctatggccactccggcatcttgactgctgttgccttccactgcgacgttaacgtgcaggatctgaggatggagggtaacaatcgctcgaatcaacgcagctgcacatgacgctatctggtacgcggcagtgccggttagatctctcaggaaaaaatgttccacgcctaacaaaatgcatttaatcaccttgtgatttaccgcgatcaccgcaccgatgccggtgccggacgcgtcggtgttgttggtgtatgccgggtctatgtaaacgtggagaaacggcttcatcgcgccctgtatatcttgtgacgtgctctctacgcggcacagatccaactgtgacagcgaagagtcgctgacaatcttatgcatactttgcgcggacgtggcagcgtcccccattagctccgtggaaaatgcgccatccagaaaaagattagttgtgctccgtaccgtctcatcgatcgttatataggccgggatatgcaggcggtagcacgggcacgccacaaccgtgtcttgcaagttaaaatcatctttatgatccggacatacgtagctcaccacgttaagcatcttttcgtgggcgtccttcaaattaagtaaaaaactagttgatttgtcagatgagttactcgaggatataaaaatcagttttgcgtccttttgtagcatgaaacccagaatcgcggggagggcatccttcttaatgaagttcgcctcgtcgatatataggaggtggaacgtctgtcccctgatgctctagaggcaaagggaaacacaaaactagttacgcgggcatggagaacgaaacgcctaaggatagaatctcggaagctgactttcaacagtgtcaggaattttttcaccgtcccattagagaactaatttcctctggagctgaggccctgaaccactttagcctatcggaagcagacggacatagattggaacgaattgtccttctcctcgacctggtggggacagaatgtctttcctataccacaatcgctgcaaagagtgtcaaatgacgcgcgtcaacagtccaatatgtcaatttcataacgtatctagcttataccagtgtttggattgtaaacgctatcacgtatgcgacgggggacgagactgcgtcattgtatacactcgcgagaatctagtgtgtgatttaacgggaaactgcgttttggataacgtgcaggacgtatgttcgtacggtcctccagagcgccgcgcacccgacgcattcatcgatcctcttgtgtcgcacggcacgagggagagtctcaaaagcgatatattgaggtacttcgagacggtaggtgtgaaatctgaggcgtattctaccgttgtcaaaaatggacaactgaatggcatcataggtagattaatagacgctacgtttaatgagtgcctgccggtgatgagcgacggcgaaggcggcagagatctcgcggcgagcatttacatccacataattatctccatatactccactaaaacggtgtatgataatcttctatttaaatgtacgagaaataaaaaatacgaccacattgtaaaaaccatcagagcgcaatggatgcgcatggtctcaaccggcgatccgtcgcgggtcaatgcgacgggttgtttcacgtgatacttccgcgagggtttattctcgctaacaatattacgtgcggtggacggcaacggttttttgcgcacactcggttcgctgcatctgaacgcacgtctaaaactttatacgtgtggggacgggtatttcaaaacaccgaccctggcagcggagacggtccatccgggccgtggtccggactggcgattagtctgcctctgtttaccacgaatggaaaatttcatccgtttgatgtagttatactcagggccgagacgcctggctctggaagctcgtggaccgtgaagtttttgtatatgtcattaattgcggcttacagaaacgcaatgcgcggtttaaaagataaagtttcacaatccaccgatgccgccgttgacggtgaggttcatcctctgaccgtcttaaaagaagcattggtctcaccggacacggctacgcgaccagtgtccgcgtgcaaccctctacagatgttgaccggactcttacagtctagggtacgggacgattacgtgacacaccaccgtgcgctcgaacgcccgggaaatgtgaggggacaagtaagcgccccaacacgcaccgagatgcccaacggatcgccaagtcgtgtaaggcttggattccgccctcccaaacaagccaactatccaaagacgtgggcgcaggcgcggcacgttttctcgtctcgcacatattacgtgtgcgtctatgacaacgaggaattagataccaagtggcagcgacaagatccgcggccgttatctctagactggtccgatccggtcgcgtacctgttagaaggagatttgtttttaggagccaaacaaaatgcgtttgtggattctctagaaaaaacgtgcaggtgtcagaactataccattaagcaattttttccggtttcgataaatagggacaacgacaccgtcgatttaattaaggaacattttatagaggcgtgcttggtgattagaaaccaggcgtcagagaggagtgcttgggtgaaggcggcgctgtttcgcaacgatagtaacacgtattggaaggatgttttgggattatgggagcatgggcctcataaactgggtacggctataaaacgaccaacatcagagccttgcaatgccgacatagactggagttggctcctgtgtgacgcggatataactaggtcaattaacgggcagtctacggtctgcttagttgtctcacctgccctaatcgcctggctggtgctcccggggggctttgttattaaaggccgctacgacctatcaagtgaggatttaatgttcgtggcttcgagatatggccacccagcggcgtgacattcttaaatcgtttttaaacaaagaatgcatatggttgcgacacccgggtacgtccgcgtttgttcgagtttacaccgcgaccacggcgcattccgccgtctttgacccgccagtaactagcgaagatgcgatgtcacataactgtttaaatgtcatgattatgctcatgaaaccaaaggagtttggaccgtgcgttaccgtgtacataaatggagatattctagatttttgtgctacggaatatgtcgccataagggaagtgcctggtagggcggacctatgtttaattcgttttggtaccctttccaatgcgccgaggagcgttccgatacccgggccattaaacccacatccgcgagaaaacgtgcccgggctaacaaaacaggaaattatatacacttcccaaaccgtgcccagagcgcagatacaagatgccatcaaggggaaagcgttcaaacaaataaatccgtttgtgtggtttgatggaggagcgttttggcaactgttcctctctgtggattatatgctgctctgtcccgccctcgagatagttccgtctctggccagaatcgttggcctcctcacgcagtgcgataagagcacgtgtaagatatgcacgctggcccacgtacacgttaacgcgtatcgcggatacacgcctcctgactcgcaagggacctccccctcgtgcccctgccttatctcgtgcggggccaggcacgcgacggatgtcctggttaccggacacgtaaatctcttgggcctgctctttgatccaaaagtactccccaaagtgtccagactgcgtttaaaaagaaacccccacccggtgccgatagaggacgccatgtctggcgttacggccgaagggaccgaggtgctacccacttcgcaaccgtgggccctcattcgcctgccggatttagccagtcgtgtaatgctatacggctgccagaatttaaaaacaatctgcttacgttcttattgaagcacgttgcacacatcagcgaactcggtcgccgtcctgggctcgcgtacacgatggttccgttttccttctttatgttcaggttcttcgccggaaaccacctggatagcgtggcgatgacctcggagaaaacggcattggcgacgtgtttttggtgggccacgtaccccacgtgaacgttttcaaccgacgaaagcagaacgctgatgatggcaaccacgatccatgttttcccgtggcgacgtgggataacgaagacgctggccttctgcttaaacgtctgtaacacgtcgtccgtggtttcaaataaaccaaagtgttgcttaaacgtggcaaataactggtgtgtcttttcgggtgctttgatagacgcgataaaataaaacgtgtgcatgattaactgttgctgaaagggctcgaggcaacaaacccctggcacgtaactcccatttaaaaatgacgacaggttggctaaaaattgccgcagttctgcataaacaggacactctaggaaagcttcgtgggtccgttgggccgattggtattccatgtgctggtccttggcgtctgccattcggacccgacaacatgtctgtctaaggtagttcgtgagctcctcggttaaccggggcaacgtagcgtgactagaaattcggggcttccccaacgtagggggtggcatgggttcttgaagtctgcggtaggtttgaagataatcgtctaaaactgaactatatgcattaactgcgtgaataactcccaaacatgggtgggccattcgttcggttttgtcgttgcccgatataataacgggcggttgacgaaaccagttttcgcacccaccgtccgtgaccgcgcgcaggttattttgaaggcgttcgcgatagctggttaacaacatgttcccttcgagttttttgaataacgaaaaccccgaaacgggacgtcgctttgtcaaaggcgttcagttagctttagacctgtgcgacaacactcccggacagtttaaactagttgaaacacctcttaatagttttctgctggtatccaacgttctgccggaatcgcgcccggttagagactgtccgcaggcggaagggtttgactttgaacacattcacctcccgaaactgacacgcatgcagcgtgtcctggggagatactgcgaccatgttaacaacgacgacgatatgtgcgttaacgtaaaggccaggtcctcgaatgcacagggtgccttgttttatttgccgtatggacaggacgagtggaattgggcgctcacgttaaggaaagacaagttggtgaaaatggctgtagagggcttgtcagaccccacgacctggaaaggtttggagcccgtggaccctttaccgctcatatggcttctgttttacggtccccggtcgttctgtcgggaaccagaatgcctatatgaacgtaattttggtatgaagggacccatactcttaccgccacatatgtatgccccccgaaaggacgtaatgacttttgtccatcacgtaattaagtacgttaaattcttatacgtgaacgccggcgggggtctcgaaactgaactgtccccgccgtttgaggcctcgcggttgcgctccgccattgctcgtctcggggacgtggaagcggatgacgcatacctgtccgcaaagtgcatgttgtgccacctgtacaagcaaaacgatacaatttcgattcatgaaacacacgtgggcggagtcatcgccttaggcggagacggtgcgagatatataacgtctagtgttcgaactcaacggtgcacgagtcggggagatttcgttttaatcccgttgtacaacattgaagggctcgtaagcatgataagggaacatggcctcagcaacagctaaaaaattgttaattaagtccgagcttgagtcggagatcaacaaaaaactgtccatctccgtctttgacaggtttggggccgacagtgctgtgtttaacgcgcagtataagggaaccagggaatcgctgcggtcgtacgacagcctgaaaaagaaggacaatctggcgaccgttgtcggaacgctagaaacgtcgctgcgcgaaaaacaaagcgaattgggattactaaaggggtttaacagaaaaaaaattgaagagtttgacgctgtgacggacgcggttcgcgacctcaaggacgagctgtacggagaactggagattctaggtacgctcaacgatgaatctgttcccgtggaagaagagtccccaaaggaccacattattagatggaaattggagcgtctgccaagagtgtgccccaaaagcccttgatcccattcccaaggttcagactgaccttgacagaaccgcactgtcccatataaccgtcattcgaacccgcaagacgctcgcccaacttaagatacctaacacctggagccaatgtagtcaccaggcgacggactggaccgccgtgctcggacgcggctcgtatggtgtggtgaggtccatgtctctaggccgctgcgttaagcattttggcagccggcgtgagtttttttacgagtgcatttttaacgatatcgtacgcgcccgccgggagaaacatcccctgaaccgcgggggtgaccgtatactgtgtttcctggagccgtgcgtaccatgtcgcgccctgatattcccgcagttaaccgggaatctgctaaacgcggattttaaacacgtgaaccctgaacggctggccgttgaattctctgagctgagggaaggcgttagctttctaaacaatatatgtggtattgttcactgtgacatcagtccagaaaatatacttataaaaggggaactgacaactgcgtactggagacttatgatcggagatctagggtccgcctctttacacacgggaaccccttggaccggagtgatggtgacctccaaactcgggttcgtgcagcacacgtatcattttaaggcaccggccagatttatctgtaagcacatgtaccggccgtcgtgtctcctctaccggtgtttgctgtcgtgcgccgggggcccgcaggcgcgtatgctagatcagccgttccagatcactccacaactcggcctcacgattgacatgtcgtccctgggttatagtttgctagcatgcctagagaaatatcttcagccagctgacccatttccccagcagggagcgttggcggacgcttcctccgaatccgcccacccattgttctatttgcgttgcatggtgccaagagtagtcatcgccgaaattttttctgtggcctgggacgttccactcgatttaggtattgactcatctggccacgcgccagctattcccttgagagaagcgtacaggcggttttttgccaaccagtgtagtttatatagggcacaatacaaagaggatgcgttagaaaacgcatcctcgcggctgtgcaactcaaaacttaaactagttctccaaaagctgttggtcagggactactttagtcactgcggaaactgcggagatcatggattttttctcagatgagcctatggtacaggagatggcgctgcttgacattgatgaacagcagcgccatctttcgaaaatgagcctggccaactttttaaaacatgagcgcgttagggcgttttttaacgataacaaaaagaaaataagcatgccggccatacggttcgtgtataatttttatctgttcgccaaggtgggagattttatcggcaacaccgaagtgtacgatttctacgttagctgcgtgtttagggggaggcggctaacgctcctgtcggacgtgtacgatgcgtgcctaaacatgcacccgcacgaccgacaccacgtgtgtgcattgatagaacaggtaacgcgcggccaaaacatcaatcctctatgggacgctttgagggacggcataatttcgtcttcaaaatttcactgggccataaaacaacagaattcgtcaaaaaaaatttttaacccgtggcctatagtcaacaatcactttatcgcgggcccgctcgcgtttggactgcgttgcgaggatgtggttaaaaagatactggcgacgttgctgcatcccggcgaggcgcactgtgaaaactacggatttatgcagagtccccataacggcgtgtttggggtctccttggattttggaattaacgtcaagtctaatccaaaagacggcttggagtttcacccagactgcaagatttatgaaataaaatgccggttcaagtacaccttttccaagatggagtgtgacccaatttacgcagcgtatgccaaactttatcaaaagcccagcatgcagacgcttaaggggtttttgtactcaatatctaaaccggcgattgagtttgtcggagaggacaagctccccagcgaagcggactatctggtggcatatgacaaagaatgggaggtgtgtccgcggaaaaagaggcgcttaactgcagtacaccatctagttaaaaagtgcatgattcacaactctacggcgccttctgatgtgtatatattgtcagatccgcaggaaaccggaggccaaattaatattaaagctcatttaagcgccaacctatttataaacgtcaggcatccgtattattatcaagtgttgctgcaatcccttgttgtgcaggagtacatcagtctctccaagggaactaaaaatttgggaacccagaaaaactttatagcgactggattttttagaaaaaggcagtttcaagacccgagcaactgcacgatcggcgaatttgccccgttggatccacacgtagagataccgaccctgttaatcgtaacaccggtgtattttcccagcgtggccaaacaccaactggtaaagcaggccaccgaattctgggcggctagtgctagtgaggcctttcccgagctaccatgggatttatcctctctgtgtgccaacgccccaccaacgccgtagatgtaaagggggaacctatagatgtctccaaagaattcgatcctatcattggagacgaaagcattgtcttgttaacggcagatggaactgcccccgcggcgctgtacaaacccaaaaccaagccgtccaaacataaaaaaaataaattatcagattttgtttaattatgcgtatctttatttgaatagggtggatgggagggaatagagggaatggaattaagggaaggatgggatgctaaaatcatatgtatttcggatatgcatcctcgtcatcgctcacatcttcttctgtttcatataacgcgttcagttccggggagtacttaacttttgtcgttttctctttagagccttgaggaaggggtgcgtattcgggcgcctttgcgcaccatttccaatcgtttctgataattctcaagataatggctaggatgcacgtgattggaatcatagagatgttaacggctatgactttgtgcaggctggcagatatgaataccttgtcgtatcgaacgactgggagaagcagaatcaggtaactgacaaaaacgccaacgtagaatccaatctggtgctttaggtactttactagaaatagttctgtgttgatataccaagctactgtaaggacaaaaaacaagtttattgaaccaaaaacaatatcggaaacgagcatatagaaactgtttccgatggccatcatcgttccgagggagaatactaacatctccatggctattaaggacagataaagattggcgccaatcggttttccgtaacgcagcagggtgtctagcaagctattctcgggaatctgctgctctaaaacgcgtagagaccaggtggccgaattgcacgatatcatagcctggacgtgcgtaaacgataggcaaaaatgtatacagtatacaaacgaggccaaaagtatatgtttgtacgacaacacgtgaataaacaactgaatggtccacagccgcaaaatagacataaaaactacgctgtgagaacctatgagcacgatccaggtctggaggctggtcattgtcgtcgcgtgcacgcgcttggccttgaatatggccaggatggcccacgcgtagtaacacagcaaatatccgtccaccaaaaacgcccacgagatgtaaacaaacatttctggtgcttccaaaaacagcgtaggcgtcaggtgtttagcagaacttctgaccgttaggtttagtgcgctgtagttaaccagtgtgttaaagtagcacggaaatccaagtcccgggaatgtggcggtcagtggcactaccgctgacattataaacataagtccaagaataaccagtagttttacccatgacgataagataaacgagtcgctccgtgaaattttcatagttcagagaatccgctcgctggcgcgttcggcaaactagcagtaaaatcagcaagtggcttacatgttacggatttaaataggctccgcttttaaatatcacattctgtgcaaaggagtcgagccacggcaccatgaacgcccgggaggtggcactcacgggacatgttttgcacatatcgctgcatagcacgcacgagcgcgagaaattaataatctggcaggttcatttacttgtatgtcaacaatgcggaattcaaggagatgccgcatatctatttgtcaccgaaacattaagcaatactgactggggaaatataccggcgataaaccgtcacgcaccgtctatgaacgagcatggccgtaattatatgcagtgggaactccgtactcgtttacggaatcccatcattcaatcgttaagccgccagcccggtgcggttaacgtaagggtcagcgagccaaacatggtgatagtgagctgcgaacgagcgttgcatcactcgtgttcggtgcgcgtgactggcgcctatcttcattgcaataccactatggactttaggttggattctaacgtgtccccaacccgggaattttggttctcggagatgttttctaaatgtttagtttccaacatcgaggtctaccttaaaacaacgggcgggttatactatagggcatcgagtgccacgcaatgccggaaaagggcgaaagatggcgcattgggtattctcgatatctttaattgcgaatctcgtgaaatacaagttgccgggcagaagtacaacttgagtatcgccaccgcaacctttcacgttctttgggtggacgaggcgtgtatgtggaacggggccctggccgaatttttcagggcgctgcacaataagttgtttggcgaccgggaaggcgtagcgccaacgttaacgtacgtgtgtccgggggccactccggagggaacccccttccccccctacttttccgcgtttccacacctcctgcttgtgtttggaagaccgcgccggctcgacgtaaccgcggtccaagaactcccaaaagcacaaatcgcggtacactggcccccgtttaaaaattcaatcttaggggatcagcttctcatacctggcatttcacctaaaaagccaggtaccgtacccgttcgttggccgctctgggtgcaggatgttaacttgagtctctgtgagacgaccgaaagcgtcgcccgcatagttgacccacattctatagtaatcataaaaatttcaacactgttgtgccagcacctaaaatgccaccgtgcgtttgtcaaaaacgagttagaatacatagcgaccatctgttccagcgaccttcgccttttcatccaagaggaatacaaccggttacttgccacaatttttacgtgggccgcggcgagtgggtatacctgggcggccattgataaaacaacagtattcatcaaagctcctcagctcagcgcagctgtaagtggttcctgcccatcactaaatagttgccgtaggaaacaatattacaaaggtttaaaaataacagttcatttcctttcacaggaacagcaaaaggtcgtgacccgtcttgaggcgcagttgggacttcccgtacaggaaacttcccgcccacctgactggctcaagtatgaggtttgctccgcgtccgtgtttttaaaaataccagccggggttttatatgccggacttgcaaaagaccccgttagtgaagcaaaacgggactcgtggctggactgtctggtagaaggcgcgacgttatcgcttaacaactcagtgccaccaattggggcgctggcgggtatcttacccaccctttttgccaaaaggcggtgtgttaatttttggctcctgccacgcgagtgggtaaaatcggcgcctatatgccctcccctaccgattgactgtgttacgcctcaacagtttgtcgtaaccaagcgtggaccgatttgctggtataaagaatggccgttaccagtggacgttgattttatgtactacctacaggaggcactatgtgtttttagtgttgtgtccaacggggagggtacggagagtcacgcggacgatatacgacaattagagaagtttgaaaaggtactatgtttattttaaaacaaagtctgtctggggaggctgtgtaggctggttatttgaaaatattttaataccgatgcgttgtgaaggtgtctggtaaagatcgtgccgcaataaaatatattaaaagtctccccgtggctatcttcttctacgttttctgtgagacgaaaggattccaaataaatgggttttagtaagcctttcatgtcgtctgcgttttcgtccaacagcttatggagtttaaaccgcactaggggcggaacgcgcaaaactccttggggtgggtttaactcatggtatgccgccaaggtcgacgctatcacttcgatcccatacaatataaacgcggaatcattacacgtatacctctcgtgtaggtacctggcccggtcaagcgcggacaggataaatgataggggcggcttatcgtagttttccagcatagacagaatgcagcagagttctgttacgctcgcgcttgccggcttaaacagctgcacgcggtcagaaaaaacgtgcggatatatgtccagtgtgttgaccatgtcctcgtgctcgaccacacacgcaaacacaaacccggtaaacgttgccgactgaagcctggtgcggacgtgcacgcctaaatcgggaacgttcagtcgtcctcgaacacacagcttaggcgtattggaagccatgcaaatgctgttttttttgtgtacttccagcatcaggcgcggaagaactggataattatagggccctcttgatgcgcattctgtttcaggcattattgcctccgatcggtgctgctcccccattaaacgcttcagtatttggtccatccgttccaggctggataatttgttcgcaatatttgtggcctatgtcatttatatatacaaacagcctgctgtccacatacagctggtctgcgatatcgtgtaaacttaaggaaatgagcgagtgatttattaggtccatgtgagtcaatttggcgattatgtacgtaaacgggcctaagagaatggaaacagtgtcttgcgagtaggcgatgctcgttcgctgcccctgattgtcgaccacgggtgtgaggttgtacgtttgaaacatttccccctcccacaggctagttagctctctgagcatctccataaacggaggaacgtattgtgaaaaaaagctgtttaccacggagctgcctttagggatgtgacattcggataggttaatggttgggtcgcgcaaaccggatagagcctgcaacgtattctgagtgagtatattaatatcgtgtctgggcctgcctccctctggcaaactctgaagcaactgcgtttcgatgcattgaatcttctttacgtagtcgtctcgttctttctcaaggttagaaattgtatcgaactgttcgttaatctggttcgtcaggcacttgaacacggtatttgaaacctgtttccggagaccctgggtcgtactttccgtaccggtcccaaagagcttgtttttatccacgatgttctgagagacgtctgtgacgaaatcctcaacgacgtctgttatgccgctcactgttttgttctccgcaagctttatcagcaagtttaagagctccctcttggggtcggttttttgagacgattgctctaagcgagccagtatgtctttatacatgtcgttgcacgtgctgccgaccagcgcttttagaggcgcggtatttagtaactgacataactttgcgtgttccgcggtcctgtgacatgtcataatcgcggtaaataaccggtacagggggctatcaaaggagacgcgtccattgtaaattataggccgtgaaacaacctcaactgttatgtctttttgtctgtaggtgccgagataagcggccttatctccgttaaattccacagacacctcagcgtagtctggaatataaatagacgtcacatcggtcattatcgcggtaatctcctgtgccagtttgtctacgattcgcgatatcgtcacgtctccggaagcgtgcgttttcctcatctgttgacagtgtgcttgtatcctggcgatcgtttcaccgtcgggggcgcgcctgacgattggcaccaaccccagggcgaccacccagtcgacgtatcgttcgtacgataaaacagtcctggtgtgtagtacctgattaatagtgttcagtaacagttgatcgactgtcagacgaacggtacgggcccacgattcaaacgtggaccgtttaaattccggcgtcagatgctccacgccgcgccaacgagtcttcagggtggtctcgaagttggtccagtcctttaagacgttgtcgtatgtgcagctccccaatgccgtcttgtacaggtgtataaacagctgtcttccaaaaatccctggatttcgaagactggagtgtagcgtttgaccgcgaacgtacgcgtacttgccctgtaggatctcgaaaagcgagatggagagttccgtcggatggaccaaacacgtcgaacccagtccggggttcatcttgaacatgacgtccgatgccaaagtcaggggtgtcgtggatcacgtcagtcgcctctcaaatataactaccagcccaccggaaatgggttggtacgacctggcctttgatccggctgaagactccgggccgttcttgccgtttaccgtttatctaattacgggaacggctggcgctgggaaaagtaccagcatatcggccctgtaccaaaatttgaactgccttatcacgggtgcgaccaccatagccgcacagaacctatcgcgtcgcctaaaaacgttctgtcccacaatctttagcgcttttggctttaagagccgacacatcaatatcgccgtcagaaaagcgcatcagaccggagcggtatccattgagcaagttcagcaacaggagctatcgaagtattggccggttatagtggacatcatgaaagaggttatggcgaaaaaacccaacggcatgtacgggactatatccaacgcggattttgaaaccctctcgagaatgacaggaccgtgtttatggacttccaatattattgtgattgacgaggccggaaccctgtcctcttacatacttaccaccgtcgtgttcttttactggttcctgaacagttggctaaatacccctctttaccgccagggggcggttccgtgtatagtatgtgtcggttcgccaacgcaaaccaacgcgttccagtcaacttacaaccacgggacgcaaaagacggagatatcgtcgtgcgagaacatcctaacattcatgatagggaaaaaagtcgtatctgagtacgtgaacctggagagaaactgggcgctgtttataaataacaagcgctgcaccgatctgcagtttggacaccttttaaaaattctagagtacaatcttcccattcctgacgaagtcatgagttacgtagacaggtttgtcgttcctaaaagtaagattatggatcctttagaatacataggctggacccgactttttttgtcacatagcgaggtaaaggcgtatttgacaaatctacacacatgtctaacgctagggggcgataccagggacacgaagctctttacctgtcccgtggtgtgcgaggtgttcgtgaagccgtttgaggaatacaagcgggccgtcaacctcacccacctcaccgtaaccgagtgggtgacaaaaaacctctttaaattaagcaattattcgcagtttgtggaccaggacatgtccatagtcgccacggaatccactgaacgttcgacccaagttacctttatcactaagtttgttaaaaacagccacgtgtctttaaatggaaaaacaaaaaaatgcatatgtgggtttcagggtacgtactttgagttcaagagaatcctagacagcgaactttttgtggagacacattcgcaagaccgtccggaatacgtatacggcttcttaaacacgctactgtacaacgccatgtactcgtttcacgcgtacggcgtgactagggcacacgagaaatatctggaagatctgaaatttgcaccactccccaccgctctggcgaccgggcgtgtagactttcaaacggtccgtgaagagttaaacctggaagacgacatcttctaccacgtgtgcagtcccccaccacccgcgggtatcacctctctccaggtcttggtcgacacgtactgcgccctgaaggacgtgttcgcctccaggataaaggtggcgtgtcgctggtttggcggggagtttgagaaagaaacgttttccgcgtttacggttaacatggtcgtaagggacggggttgactttgtctccccttcagaacgtctaaacgggttgttggcgtttgcatcgaccgttgaatcgtataaaattaaggggtacacgtttttaccgatagcgttcggtcgctgccagggattgccactcagtgatgacctcaggaagaagatgccatctctggtcgtgcaggactctagcggttttatcgcgtgcctagagaataacatatccaaactgaccgaaaccatggatgatgggagcgtttttcaagtgtgttgtgcgggggactatggggttagctcaaatttagccatgaccatcgtaaaggcccaggggatgtcgttggagcgagtagccgtagtattcggggcccacaaaaacgtccaaacaagccacgtgtatgtagcaatatcaagggctgtcaactcaaattatctggtgatggacagcaacccccttaaaaccctcctcagagaaccagtcgataacacctccgccaagcatatagtccgcgccctccacaacccaaacacaaccctaatctactaaataaacaaaaacaagcaaatggaaaacactgtgtctttattcagtccagccacggccacggatagttgtcatttccacacaccgggggggtggccgccatagtttgacgaccagaaacgcccggaccctctgggttagaagtagccccgccccccgtggtcgaggtcatacgtctcttaggggcccgggttgtacccaaataaatcacatcgtcgtcaccgttgcagtccctgcgcgatgcgtgcctcttacaggcggcacggacaccgccgcttgtaacgtgatctgcatcgtcgtcgctgtctgaagacgaacacaggtcaatcacatccgtggcgcccgtgccctcatcggcgtctgattcgtcccatgtctcaacagagccacggtcgtccgtatcatcgtcatctgatataataatggtctcctgttctacagctgatccagtaacggcaggttcttcttcttcattggacgaccatccgccccctgccggtgatctagatgtatcacctgaccaggcaggcgaagcccgtggcgaaaagccatcagcatcttcgccactctcatcagtgtcctcatagtcatctataaaaaaatcgtctccgctgtctgatgtagatgacgggggtgcgcggtgttggccagtaaccaccgcgacgggccgttgaccaccaaaagcggtcagtggcgcaggcgcgcttggttgtgcggcgggacctgccgccgtaatgacggtaggtcgccagtcgctctggttaaaggactcaccgtcccgtgcgtgcggatctatcctaggtggggaggagaccggttgttcgtcttcttcatccatgtcactgtagttgctgttctccgaatgagagtcggtttcgtctggcgtttttggagtggtgggaggagaatctgcatcaaaaacatcgttaggtgcagtcatgtcaatcacaggggggcctccgtaaccgggcggtaccggcaaccccgtacgggacccggcgaacagaaaccgtcttgttctctttttcctaggtgcaccgggaatcggcaacatcctgggagtggccggcggggtccttggagggtccgacaagaacatagccatgaccaaaacgttcacctgtaaaacgcgccgcctatccccgatcagttaatattccaatcaacggcgcccctctgattttgaactagatagtcattcgcaagtttaaaatggttgcagcctagaaactgcggccagggagaaccggtggccgcgtgagcggcagctaggggagacggatggcgtgccttgaggacgagatgtctctggccgtttatcaacaccgccttgtctatggcttttcttccccacagcataaaaacgcaccgctgaagcttggcagaaaggcagcttattatgtagctggtgaaccaatcccagccaaggttggagtgtgaccccgccttccctctctccaccgtcaggatggtgtttaaaagcagaactccccgtttcgcccaacagtctaagcacccgtgagaaggagcggtgaacccaggtacagtgttggctatttctttaaaaatatttttgaggcttggaggtatactgtagtccggagcaacgctaaaggcgagtccggtggcttgaccgcgatggtaaggatcctggccaagaatcacgactttaatatcctccggttcgcaacaatacgaccaccacataattctgtctataggtggataaatcacggttgctttgctcatatccataacgcgcctcaacagagccgcaagcttcttttttaaaaatggagaaagatttaaaaatttaagccatgagtcactaataagtaacatttgcgtgggtgtttcatttggtcctgtcatggacattttgtgccaaactactgtctcgagccaagtatccataggtccccacgcctggcacctactttacgtgtgcgaaaagctgcactaccttttttcgttaagtccggcaccgctggtaaattagttacactttcgctattatcatgcgtgtccttaaaggcagacaaaaaatcttccaaactaatgcgaacgtaattcagcaagtcaagttcctctcgatgaacgctaaaatcaagcccacttaaaattcttataaaaaatgccctaaggtgaaaaccattcacacataactctgtagtaattgttccaactttatatcgcaaggttgcaacgcaccgtccttggcaccatgccaaatctgtaaagtggaaagaaacaatttcaaatggtgcaggaatattaactccagttagcggcacaatgtcacaacatggatttggaattaaaccactagagtagccaatatatagtatgaaataagttgtaaaaaaacttaatgtatacatatttcttgtatttcttacttaatgtaaatgtgaacttcgtacttatatatacattgtaaactgcatgtatattgtataggttaatagatttaaaaagaaagtacctaaaaatacgtactttcggatatccgcccagctgttaacccgaaaatacagaaatgactacacaaacacacatgaaaccaattttattcttaacacatgcaacaatatttcaatgtaacaccatgttaataaaaatacgcagcatgcacatttttagctaagctcctcaaatacaatttcctcttcaattgttgactcatcaacctcggtttccgtgtcaatgtcggaaatatgtgagtccaggataccctcatcatcgtcacaaattaattccagggcttgtaaaatatcatccagtgaatcagctgctagtgtcaaggatgttgtttctaattttagcccatcaaaacggtgagtgggtgacattttgggacaagaccccgtgaaattggcacgctcaacacaaacggaaggatacatttgattattcacgaggggcagcatatttatttttataacatcaggaaccggacccgccaatgacgcacatatgtccattaaaatcggagtcgtgacacgggttgaattgacggtgaaaaaaaatgtattagctgcacatgcgcaactattttttaaccacacgagggcggaataaaacgggtcccgggtgctaaggccgcggattcggcaggcacggctcattgccatctctagatttgttaaatctgttcgaatatacgagtctaaaattaagtataaccacgacaagttcatgcaggaggcaacgcggctggtttccacccggacatccccgctcacaaacccgaactctctaggaacgcacatcaaagcgttcaggcaaaattccgaaaccgaaaccctagttctgagcaccatctggttataaactgcggttaagaggcgagcggccccacactgctgcttgtgaggattcaacttaggtggcctgcaggcttgctgacagtcccgtagctgcctggcggcgctcgtgcagctctgacagccgcacgccagttccaaagcgaggcagaagcgctctttgcagtcgcgccatatctcggtcacaggagattcgctcgcatgatcttttaagtgcatatatatctgttgggtaacaaacctcactccctgtaaaaggggaataatgtccgttctttctatcgtcttctctgagtctatagctacatctttaagtataaccaaagacgctaaaaacccaggatggtcgacccctttaaaaaatcgcaaaagcgaccggatggctctatccgcgtttccatgttgttcaaaggtagtcacgatggatctccagttagactcggtctccctgtcaacaccctgaactggaatggaaacagccatgacaccgtgaacttcctgatgtctcctaaaactaaccccgaaacagagctaaataccaatgactgtcacccctaccccaagccacgcccccgtactattagaccagggtgagtaaccacgctatcctttaaaaacccatacgtggagtttgtaaggtaaacagctcgtgtatatttcagacgcctgtcataaaatggataccgacgacaatcaggtaattaaacttttttttattcaagattcaggagggcgtgtttacagtggtgtaggtgggagcatatctcgccgatgggaatggctgatgaatccacacttagtgctcggatcaggtgctctgtgtattttactagtgcagctgaacctaaatttggcgtgtttaggtcgtaccaaccaaaaaactgcctggcctcgccttttaggagctctatgcaagctttaatggtgtccaatagcttgtctttaaaaatgcaactctggtacatctttatgacagtggtccaaaaaaaacaaagatttaaaaacacgttaaactgcgtgtcctggtagtcctcgtatatcacctcctcaacaagaaaaaattttttaaccaaactcgccaggtactgaaacgatggtacggacaggtcgtggaaagtgtctatcatttccttcacctcccctttaaaggttttggttacaccgaccatgtgagataggcaccagctcaggggcgaggtcggatcgtgagggggcaacaattcgtggacgtgggggtactccagttttaggatctccggcaggatgcgtacgagttcttcgtcaaatcctacgcagcccgcatccaccaggggcagtactgagttgataagtccccgaattctatcgttcgtaattttgtggagctcctttagacagtaaaccatgttgctccggcactgcggcacaatgaaccgctctccttgtctgtgctgcataagcacctggagtatcttagacaggtatagcgcccgttccactctcagggttgccgcttggtagagcggattgcgggcattgacggtcagtgtgttcatctcaccgactatcatgccaagcggcggatagtggaacttatacatatgatttaaaagatggtctttcccgtaatggcgactcatggccgtgttttaacgttcacagggtgttaaaaaactcagaaggtcctgcgtggacaacttttgcggactctccgaaccgctcagggataagctgtaccacgcggtgcttcttataaacgaagcatacgtcaagaaacatgacccacgtgacctaaataagctcacagaagaagtctgtcgatgtattgtaatggaatgtgcctccctgggtcccatctccgggctcattgcggatttgaatctctttaacctgttctgcctttaccggggttcccgggttaaaacccgcggagccgccacgtgtaacgtcccgtgcgcagaatgcgcacagggcgtcgtgagaatcctgaccgagagggctctctgttgtaccgaaaagatgttcatagcgtctgcctgtagcggggttgttttaccgccccagctggccaaagttttccacgacgtctacgcggagatgaaggccaagtgtttaggggcgtggcgtcgtctcatatgttgcaggagacctatcatggctattgccgactcggttctcgtcacttataataccctggatgccgagggaaaactggaccttaaactcaaagcactgtgcaaacttgtttttcaacccatctttcttcaaagaatcttagcccctatgcagctactggccaacgggaaaatggtccccgacaattattttaccattaccggcacggccgagaagaggcgccctgtcgtgactggaagtactagcgggatgacgtgtccgggaagcagccttgtccccgattccttaatcctgccagtatgcgaaccggggcttctcccggcacccctggttgacctcagtaatgtcttagaaaatccagaaatcatcctcagcgccccacccctgagtcaatttgtcatcaccaacacgcaccccagtctgcctcagtcagtcagcattattacgccaactcagggcgttgttcctggccaatgttttatggacacgtggaaagcggtgtcacagagcattcaccatcaggcacatacgcctatttttgccgccacgctggccggttcaacatccgcggcccccggcccgcatatcgcatgttccccagttgccggcacgtctcggcaggtggaagcgtccgcgggcgttgactgcgggaaacccgtatgcgcgccgcagcccgatataccgccccatgtccccaccaaaaggatggaaacggtagcacagtcgggaaacgctcccgtaaaaaacgtccacatcggaggccgcgtatacgcgccactggttaatataccaataatagacttaacgtccccgtccggctccggacagagtccggtcaatatcgccaacactccagagtcccgcatggcggccggctctccgccctttgccgaaacggccgcaacggtccccgctaagagaaagcagccacgcgaggacgtggcagacaaaagatcgaagggcgacggtcggggcgccgcaacagtaaaccccccattcccgggaccgcccgcgatgcgcgttcgcgagcagggcttattcgatttaatcgaaagctccacggatgtaaccgcgaacgcatcggggccaaaaaatgacgacgacatgttagcggctatcctacaggacctatatggcctacaatccccccctgctattgattccccctccagcaactcggataatgaggacatatttccagaggtcagtccgccatctagcggtcacggatcgccttgaaggatgaccccaagacacaagatcacattaccgacaccttcccacgcaacccggcggatgtctagccgtaaaaaatatcaccggttggaaagtccctggtgcgacaaacataagcttcaaacttacccattttctgtgctatttcgggacactaaaaaattgtcgccactggcgacaattttttagtgtgtctaagtgtcactcattttcaagtttctgtcctagaattttcacatccagtttcatatcatataaagacatctgttttgctttcgtgaacctggcgtgtttttattttgaaaggcgccctaaaaacatgcctcgtgtgaaaacacaacccaagagaccccaagtgtttgactttatgccactagatctccacggtgaaacacgcgcggagatggattctcaaagcctgtgtcctgacggtcaggatctgcttgggtcttatatctatacggagaataacggcccgttttcccagatgatgcacaatggacagagcaataacgggacaggtgaaagcttcggcagctatgctgccggcgacggttttctgggcggttctgtgtcagggatgcatggaaacggcaccgtagatggcttgtgttctaaaaaacaatccgcgtgccgtaaacgctcggctgcactaatccacgcagcgtccgaagcgtctgtggccgagcaaggcacctcacaggggacaaatgccgtatctgaccggataggcagagacggtggcattggcaataaactactcaaggtgagtgcgcggctgcctgataaaacaaaaacgcttccggatccatccttgcactgctatttttgatgtttaacacgtaatgtctgtttatgttaaccccgccatagcccgaacccaacattgtgccatccgattataaaacgcaacactcggacacggcagatggacgcacaaaacgcgcgcggcagtgcaagacgtcaggaaaaaataaagtccaacagctgcagcaggtacaataagcaaatttagtaatctgtattttttaagtctccgaggttttcccgctggcatgcggcaccgtgtccgtgctttagttgcaataccttctcttaacaagcacgtattccaacaggagctggaaagtaaaacaactcaactcaccgtcctcaccgctcaacacgaagcccttcaaaagcacgcatcatttttacagaagatgatgatattgatgtgcaaaaacgggaacagcagtaaaaaaagcacgtcatgatttaaagccaccccgggtcattagtttaaaactgcctaattgattctattcacccttcaataaaataaaattttaatacgtttgtggtttgcccatttcctgtcttaaaaatattaaacaattcatttgggaatgaggatttatgggattaagggattaaatttttttgggatcatgggattttggaacatacctctgggatgggggttatgcttcatggtctgggtggcgtggattgcacggggacggtcggtgtgcccaacctggcacctgacggatgggaaatacgaggcggtatacaggcactacctcgaagagtgtcgcaaacatgaaggctcggggagcccggacggttccggaaagaccgaggggtctggaaccaaagcaaccaccgaagcgaatatatcgatacgacctaacgttgttacgtcaggtcaagataaagagccgatgaagacagcaccgagggccgaatcatcacacgacctgccacgcatcgagcaggttaacgctctccacttatcaaccccggaattggcgcaacccctcccggtagtagaatcgactccacgcgagtcacagtcaggtgggacaccctgggacgcgcgcccccacgcgttcattatgcacacaaacgacatgctcaacccatctgtggtcctgtctttcagagccgtccgtgcgcgctccatacgcgataccgagcagtccattcgcgatcggaacacggtcacgaccagctatcgtacccctggccgcccttccctcgttcaagccagaccctcgtcttacggtgcgcgtctacccccttcgccccgaacaatggcaagatacgccgagacgcgaccaacagacgaccaaaactgaacacaaagaagaccgcgctgtccattcgacgcagtgcccgcacgggcgctgcgcgggacctcgggcgtacgtgtcagacccgcgcctccctccgacaacgcaatggcacctggttaaccccctgtgggccatggccggaatattgggcctcacgatcgcctggtacacaatatatcaaatatatgcacatagaaactacacgacgcttcactgcgaataaaggtttatttatttcacacgctaatcagcgtcgttatttctggtcctggactgggcgcgccgcctcctgagcgccccgtccgcactggccgtgattccgcctctggtggtctcgtccatcgacacgtccacgcggatgttaattccggcgagggcttcttccacctgcccgcgcgtcacggccttggccgtgaccgccctcaccttagcttcaattttacgggttgccacgttacataaggccgaaacggcggccgcgattttggcctccttttcgctgttagagatgacggggtcggagccggacctggtgggatccccagaattaatcagctttttgagctgcttgttttcaacggacaatttttgcacctgagcggctaactcctccatcgttagctcttttttgggggcgcgcgtcttgggacgcgtggaagacatggccgctactacgcattacagactacagcggtacgtttaaatacttttaggcggccctgaggttactcgtgcgccacagtttcaacaaatttaaagaagcacatatatgtcaggtacagaaatgttgcaaccgcaaccagaacagagttgatcaccgcccatatgctcgaaaaactagacagcacgggctggtacgtgtccgccgagcaatcatagtcataaaaagagggcgttacggagggagccgcggagggggttgtagtcacaggaggcgtgcccgttccggtagtcgaggaggtggtgactgtggcgcacaccggcagacacagatataagcacgccgtcagggctaacgcgagcacaattgaccccgtcattttaaagccggcgttcggcagacaaccctccactttggcacacgagctctccttatatactatcagttaaacattatggcagaagtaaccgcccatacggtcgcatacgcctttgactcctgtaagtttgaaataataccaaaaaacaactcatctcgtatagcgttacgcaacaaatttccagtcgtggtaaaaccgggagaaccgctggtcgtgcccctggggctaagaattatccgcgcgccacagtgcgcatttttcctcagcggagcgccgacggatgaggtgtattaccacaccggacttatagatcaagggtatcgcggagaaataaaactcatcgttctcaataagactaaacagagcgtcacactttaccggggagaagtcaacgtctcactcatcgcgtttatgtacgcctcgcccgggcccctaaaatgtccgatactgaacctaccacattactctctggacgctggatttgatgtcacgtcgccacatgccatgaccatccccccgactgaccgaaccccgtttacgttgagtttgtattataagagtccgcagctcagcaccccccacgtcccactaatcgtggggcgctctggactggcggcaaaggggctgaccgtcgacgcgaccaaatggacccaatcgttggtccatttgaggttctataacttcaccaaagagccaatagatatacccgcaaacagccgcatatgtcaggtagtgtttattcacgaagaccacgtcccaagcgggtggaacattctgagatcccgcgtacaactcggcagcaccctccagatatcgtgggccaaaatcaggttcaccgacgtggccacgcttcccgcaacccacccgctcaactcccgccccactcaaagccagaccgaaccagagaccaaccgcggcgcaaaggggttggggtcgtcagggttataattttaaaacatggtattattttaaagttgtttatttttaataaaacaatccaaattaacattaaccatcagcctgggcatcttgtattattttgtctggcacgatattatccgccaccgccaactggggcgtcggttgtcgaatcaccggatgggaggggttggaggacgcttcggtatctaccagcacattattaatttccgcagcccacgtgacagagtcatcggacgcaattgtcgtagccagcttttccatttccaggagtgacacggtctcgtcggcccgtttattcaaaagcgcgtgaaaggcgctctcgtccaccgtaatatcccctatagccagggacaacatggcgttccagataacgttctgtatagactttagcttagatatttgaatcctaaccgtctcctggcaccccgcgggtaccctggccgcgtcaaaacgacgaagatactcggtaattttattggattgaacggccaacagataggtctgtcggagggcggcggtgcacatctctgcctgcttcacatcgcccatatccactcccgggggcagacccatggctatctcggtccgccagtgagtaggacactcgttaagaggatctggcagcggacgataccgacatcccacgcgtccaaacggactaaacccgcaacacgtccaaaatgagccaggtgcggccattgccagacgaacgggtaaatgaaattagggcaattttctccactagtggagatatggccgaagtaattacggatatactaactggaacgcaagcaacggcgtccttcttctgcgtgcttcacgatcggggcaacgtgcctataaatactccacacgccgtgattaaactctgcctgcccgccagacgcccaggcggcgggccaaggtgtttaccattgatggtgctgaatctaccggcgtggcaggttcatctattcctaacaggtgacgcaccattgacctcggataacattaaagaccgcattgatctggcccagaccgaggagatactcgagcccatattaagcgtactggcatgcaaacggtcggcgcagcagaccaaacatgactcgtttaaatctaaggtggcctggtttagggcaaagttcgtctcggccctaagaaaagtttacaaaatgaccccatccccatattggatgataacgctgttgggctcgttcgaggcatcgtttgtgctagccggcacgttttattttttccagtcttatacctgtacggcggagacgctggtgcatctgacaagactattcatttccagccagggccaaagcttggtcaccgtaaacacctatgacgaattggggcgtgtattcgggcggtcagattttcttgaaattgttccaaatttttgggcatatctcaaatacaagatgcagcaggacgacgtggagtccaaggccattgatcaaacaatcaactccatacgtggggggttgatgctgtcacctcaggacctcgtacacttcatctacctgtcattttacgagtgcatgaacgcacagacgttcctgtcgtactctcgtaccacggccagtttaccaacccccgcgacggttaacccgccgcagctgtgtcggcgcttagaagcggattttaaggaacacgtgatggcgtattacaataaagcgagctacctgagtacttacataaccattttaaccgtgcccgctcctctcccggaaggatacgaaaacttccaagagctagcatgtcaatactggtgtggacaatcgagagacgtggcggaaatcatgactagaattaatgaccagtatccacagctgaatctaacaaaagatttatccggtctccttgacctagcggccctagatcaggattccggaggtcccaaggaaaacctttttacggtggcatctagaattcccacatacaggtgcgagtttttgaataaacagtattttgttctaatgcacacagactgtatagacgcgtactggaaacaaaacattatcgtgcccgaagacgcccaattgcaaggcctgacggatcaagatctcacctccaggatattttactgcgacctcggcctatctctacctacgtttaaacaacaaattatggtttcgcgtcacgagtatttcaacccaagactcccagtttacagatgggtgctggattttgatctaaaagtaaccgaaggtagacggactctaaacgatatatacaacatatgcgtaacgctgcgacaggtcatattagaaacgttgcagttaataggtccactaaaaccccaccaccccgtgtatttttttaagtcggcctgtccagccgtaacctggccggatgacatttcagataccgcgttttgtcactgcgacacaaaaatagggatgagaatcgtaaccccgtttccgatcggatactgtttggtggggtccgctccacttgtgtccctgaccaacattctaaaccgcgtggttaaactggacacgcggctagcctcagagtatcctgggatcttagaagataaaggaccatttgactccggcatttacgccaaggggcgatgcgtgcgcgtaccccactgctataaggtgggtccgggtggcgagctgtcacggctcctaaaaattattatctgtcaccccgaagagtcagataaatcggcgtatttgaaaaacgcgtttaaagtttctaatctgctacatcacgccccaggagattctgtcaccaaaaacggccacctggtgtacgcaatcgctgacgaaaacgagggctttctagaaagcaaaaccaaaaataatcttccaaagacaatcacggatttggccgaaaaaatcgagcgaaccacagaaaaaccactaattgattgggcagcaaccgcagtgtggccaaaactacacgataccatacagcgtttctttccggatgaccgcatcggccaatttgcatctgtgagcttcatgcactctggagacaacattatacaagttagaccacaaaaaggaaacaactttttctgtattaatcataagcaccgaaaccacactcagactgttcgtgtatttttaaccctacattccacaaaagaaagcgaggtcacggtgacctttatgagtcagtgcttcgctgcaaaatgtaaccataatagcccaactgcacatttttcatttatggtacccataacctgcacgtaagacacacaaataaaatcaccagtgatgggccagctctttagctgctttgtattaatttttacaccgaactaccgcggttttccttcgcgaacgtaaacatggcacaggcgatggtaagcctggaatacatgaaggatattttggacggtaagaagacctcttatggctcttacaacttttatctaaaaccgcaacttaccaagcggctcgttttatacgctttacatgcggtacgtgtttcacgcactaatttgttttatagggggcatatcatcatcggacttcgacgactcctcatcggacgagatggacgacctgtccccgacgccggagccagaaccgtccacggcaccgcacagctttcctgaaggacccaaatcaaaagtggtcgcgttacctaaaattcgtaagcgatctaggtctaagacgccggtaaaaattgagcacagatctccgcttaaccgctcgcgatctcgatccagaacgcgatccggatccggtcaaagaacgtcaagcacatacgtcaagagatttaaaccaacggttgatgcgccccgttctcgggaaccgtggcaccgaggcggtaagggaaaggccccgtttattcgcagaaacgccctggctgaacgcggacggcgcacatacggcaacgagcatcgcgggaaatcagctttaacgcggaacattaaagagtctattaaaaaaatgcaccttccatccaccatgctctctcgtccacacgataaaaaggtattcgagggactgctgccacgacacctggaacagtgctttcaggtgtgcctgccggcgccgccgccgctgcgacccgaggtgttcacggatcgacagctcaccgctatagtcaagtctggcggacgcagagacgcgctggtggcaaaaaaagtcagcctggctaaagtcatgagcatgtataagcctctgctcacgttcgtgacgggaagaaacaaccaggcccactggttggcgacccgcaaaaacacgctggcgtctgcgggactcgaggctctggcggcctttattgaggaaggtctggcatgggcccaggtgtgcgtatctcaaaacagatcgttgaacgacagcaatctagatatcatcctagacagtagtcagagcgtgtgcacgtggttcatctcaaaaattaggcatttgcacatacagtgtttcttggaaaatcagggggagattagcttggttaaacagctaacctacctcgtgtgtataaataaccgcctggcggaggcggcaaacctggcgggggaggtgaaactgaattttaaattcgcgatgctgattgggtttgccctaaccttgccggcgcttctcgcagagcagaagttgtctggcgagagcctgtacctgtttaggtcttttctggagaaatatagaccgggagatgtcatggggctcttaaattcaatagtggtggagcactacacgaaatgtcgcagcgcggagtgtgttattacgacccgcgccatggtagggtctggtgaaaacaacaagggactttttttctttccagcgtaatataacccatgtgcgtagttacgttatattatttgagggcgtttatatgcaataatccgcataacaaaataaaagatgtgtaaaaccaaacacgcgtcaagccttttctgcaagggttctgtgcgcgcaacatctaggcagagttcgccacctagcggcacgtcttatatagcctttaaatacgcggatgttatttacggaagtgccacatcgaaccgtggccgggcgcgtaacagtaaacctggtccgtgacagtaaaccgggtccaatcgcgcaatttacacagcacataaaataggcgacagtttaacgctgtttatttaaacatacaacacacatttcaggagtttcggtctttttaaaaatttactttcattcaaagtgccgatatatttcacgttggtggacaataatgcatattggcgcttgtgataggtcctcctcctcgtatagggaccaggccgccacccaaatctgtgcaaacggatcgggtggaccgggatcagagggtcggggcgactgggctaactttgttatatagtcatcagtatcaaaaatgcgatgtcccacccccgatggcacgcgtgttggttccaaggtgtttaaggtattgccacgaaaccacaaattaagaaatccatatccattcatcataatcccatactgacaactgctcaaaacaatgccgtcctccagggcatttaacgcgttcacaagtctgctcaacataacatcgtcatccatatgaggtgccggaggcaaccacaactgaagcggcccgtgagccatacaaacgtggtgttcatttttatccgttggtcttgtggataaacgaacgcccataccggttctggtaaaaacctgtgattgcagaaacccataataatacacctgaatatgcataaatccccacgggtcgtcaacagatggcgccaaacacgcagaaagaccgcttgatacagatagcggcggagtgtgttcctcgggtaacgcaaccgcgcccccttcactctcgtcctgcctaccgtcttcaccggcggtggttgcggacgcctcggtggccggggcggtcatggtggaggcaccggcgtgacgggtacgaagcagcccgagcagctgctttaccgtgtattcagttaccctgcccacatcgtcggcgcccttgcgcttgaacatccgaaacctcaagccccgcagttctgccgctaccccagaagcccgagggcacaacctgcaagacatttcttcattggagcgcaacctgaagaccactgttgggaatgaaaaactccgtctcaaaaagttcttttcttctacgaaataggcactttttctaatcgcgcccaataggcgcgccttggcctggcacagcctaggcctcccggacggaagggggatgtttctttggttgcagtagtcctcgtacaccttcccatcccgagggtaatcaaattccacagtccacgctgtcgtagttgccagcacgaccctggtcttttcgtcgtctgcccaaaacagtccagaatacgttccaagatttgcgtgagtgacgatccattctcgaagtcccatggcggacggaccaccgctatgaggtgggcaccaggtagccatggctgtatatgtcaggttgatactcaccgctttcagttcatgttaaaataaccaaactccgcccatgcggctacccgtgggcgcagatgtgtgcagccgtcacgaaataatcccacctgcggcaactaattccctcagcgcccagtcagctggctaccggttgatttcactcacgagcacgccaccccatcatcttcaccgctcgtgcccgtgtccggattgtccattgaatctggtccggagtcgcccagctctactgccgggtcaagcgattgactcgtcccctctccgggatcttccatatggaaggaaccggcgacacgagcctctgatgttccaattgtcccggggtaaaaacaaccctcggtgccgcacacagacgaggtttcgcacacgtaacgcagtcttatggcgatgggtgcatgggcgggattcggcgactgaaccgacagacacagggcgatttgtgcataatctctaggtgggtcgtctccaggcttcggggaccttgcaagagctctaagataatccaccatatcaaacacctgaacgggctgcctggacggcaggtccttatactcctcctcattgttagtaagcactttcatttcccggttctccaggttctttacggaaatgccagtatccgtaagggcaatcaacagacccttctcgcactttttgtgcatgccccttagatcttctctggcgaaactacagtcaacgacccccggtggcggtaaaaacaaagtcctctcgtcttgaaaacaacacgcgtgaccagcgaggactggatatggtcgactgcagacccgtgttccagcgtgaacacgttcgaggccaacggtcgatccaaagtatagaaacgttaactggatggcccctggcagtcgatgctcgcgcacacgcttgttcttgtccatccaactccgagccaatactgtcgcggtaaaaacccgtctcctttcacgtccaaacccaccccattggtcgtatcggaactcactagttaaaccaaggcactgagtttcggtggaagacatcaaattacacatcgcacaacttaccaccggttccggcaacaggcgaaacagtctatatcgagcctccgatcgcccataggccttattaataaatttgagctctcgtatacttttgtggtgtctcatgtcgtacaaaaacctcattttcctgaatctgccatagtggggcggtttttcgccctgacatatgcctcgcatgttgcagtagtcatcaaaaaacttctcgctatcggaccgagctgcagttactttgttccacgggacccggacaacggtcttgtcctcattctcccaaaccaatccgtggtactcgctcgattcgaccgcggcaacaagccaggccttaatatcaactccacgacccgccattccgcgtattgggaccttgacggatcagctcttaacacaaatgaaactcgggcgttcgcggcaccagttaatgaacgtatctaaatgacggagtcagtgccctaaacatatggctaagtctactgagactgtgcttaggggaagagtgacaaacacctggcactcaataaaattttgagggaggtcattgacccgtgccttcctctggttagtattaaccccattgccaccatattcaaggaacagaactccaaatacaaaacactttattacagataaaaggcgctcgccggcctcacaaaaccggtcgcgcgcacaggagacattaaacattggcagcggacaacgcctgccacagacactcgtgccacagctgaataattaacggtacagtgctttgcaccccaccctcgggtgtaggtacgcccccaaaatataggtaaacacaagcttgtggaggcgccgtcccgtcccgaggtgttctggccatgcctttcatatagtccaccaagttaaatgcacgacacgcgttggcgcacgacaggaccacaatcgcccccggagattcgacgttcccttgaacaaacgccatggaatgccccaaccacctaatgtaaatgccggaggggtcggcccaaaacgccaacccgcgcaaaagatctttatccaggaagcgcaaagcctcggcctgaaactccactaagtggcttggaatctccggaaccagtgcttgctcggcaatcggcgcaacgcatatgtgcccttgagcgcgacgctcggacaatgggagcacgcggatgccgttgggcgattctgtcaccacttcaccaaatctgtctccataatagtaaaacgttatacgtagcatccaacactgctcacccccggctcccaccgcaccagtgtactgaacaccggcaccaaccggaccaaatttattcacggcctcctcgcgcaacccccgtaacatagcagtagtaccggaggcctgatcgcaacgagcacacgacctcacggtaattggtaaaaggcgaatggttcggcatctgttcggatatggagtcgaaaggtcatcattagtcgacacgtccgacacggtttgactatgccgtatggcgctggatagccagtttttgcattccttagcggtcaattccctgccagccgcgtgcaagatgcccctggatcgacagtagtcaataaagatgtttttttccgactcctcaacgcctctgcttcctctgtcgtgattccacggtatgcggattaaagtccgttcatcgtcgatccaccgcatcccgggatgacgacccgtctcgcaacattccaccagccatcctcgaaggctggatggtttggtcactcggaccggccgctccatatcctcctcgccgcgtgcacgctaatgccaaaccttgtccaagttacctcctgagaagcacctgcgaccctcacagagctacgcgtggcaaacgggaacttgtggtctagtctgctgggacagtacctcggcgttatttttatctatcgtgcactcctgtggaggaagtttgtggtttatgcgcaccaccgggaaggattcatctgtccctgcaccaactcgtcctgacagaccaccacagaaataggaacttgtgggctctcttgggcaccaggttgctgtgagagatacagtgttgccccgttgcacggcgcatggctctctggtaccggggagcgggcaagctttctcatgaaacccatgaaataaaaaatctgctgtttcgagaggaacatgacctctcgttgaataatatcaccattacagtcgttacccgcagcgtaaagcgtttccctgttcctattatcgacgtacactccggtttcattgccgtgtacgtagatgccctcgttatacgtgaccagcattgtgtttatttgcgttatctgatctggagtcagttttccaggatctggcgatggaaaccaaacggtaaacccggtgccgtaaaaacacagatgaccgggacgtggaaccgggcgcggatgcaaacgaacacctcttcgatcttgggacgtatgatccagtgccaaaacgccaaaataaaacacctgaattcgtaaccgccaaaacggcacaatttgatgaggcagtggcgcttctgggaccccgagacgatgtgctcgagcgtatgctgatctcgttggagggaccaacgtaggcaagggggcgggcagcggcattcctggttctaacccaatgccacacacctgctccaaataattgataatcccttcagagttctcatcaagaatacacgcaaaacatccagaatccatcaaaggctttacccggaaggcggtccagcctcccccgtgcgtccgcacgaccccgtgctcgcattcttgaaatccagcattctccctcaaacccctcaaaaatcgtctcttgcactcaaacacgtttccatcaatcccaacctgcaggcctctctgcgcgcagtaggcgacaacggctggagttcctgccgctcccttcttccaagacagcttaaaggtttttttctcctgatcgcaccaagtcaggtctgaataggttttctcgttgagatggtaaagcgtccacgatcgcaaatggctgtagttagaatctcgaatcgccatggcagtccacacctatacaataaggcgcaatgcgagtggactttactcccctgatgatattgttactgctatatctccttaaaacgtgcacctggcaaatcaagcggctctatttctgccacttcctcatttataaaaccatattgtttctggtctcacgtttttagctcacctgcaacgacacggtgaatgagcccgagtttcgctcctcccgtgctctgaaaaacactggcccagagctaaaaagtgaagcaaatggcatagctttcgatttcagtcaccggcagtgagtggtggacattagaaagtgttttgcccgcacattcaactgcgcactcaagcccaaccaatatggcacgtgaccattcccccatcacaacactagtacagaaaaacgaaaccatacgcgaggtgacattctgcaaggtagaaatttattaatccggtgcatccatctaacaaaacagcaaatgtttattcaaagtgcctatagatttctgtttggtgaacagtcatagataagggagccctcgaaacgtttctctcctcacccaatgaacaaccagaaacccaaagtttaacaaaggccgccggaggatccgtgtcgtgcggggagggggacctggcaatatcaagtaagtaacgatcagtgtcaaatgcccacacgtgcatcccggtataggcacgatgtggaaaattgcgcggttccaccgtatttccctgaaaccacacgttaggagcccctgtaatcgtaaacacaattcccctgctggtgctgcaaaaaattattccatccacaagccatttcaaggcgtcctcaagtctctgccccatggaagtgtcagttatctgtggaatcggaggaaaccaagcctggagaggcccatccaaaacacacacgtggtgtcctgacttatttgtcggtcttgaagatagtctaatgccctgcccggtggcagttgacagctcagcctgtaattgcccatagtaataaatttttatatgcatgtgaccccacgggtcgccggcagatggcgcagcacacgccataagtctggcgcgcaggacccccgacgaatatggtgcatcgtcgtcccgcgcgtcgtctccaccttcctgaccttccaaatgccgtgcctcgtgatcacctactgacgattctggaaaagcgtgaagcatcccgcgcccgagcatcgcaaagcgcatattgcgcaattcaaggcgaagcgcgtccacgcgcggacacacaggacaagacatctcttcaaatgaacgcaacctgaagaccaccgcagtaaacggaaattggtcggtagggaagtccttatcttcatcaaagtacttgctttttcttatggctcccaacaagcgtcccttggcttgacacaacctgcctcttccagacggcaaggggatgtgtcttaaatcacagtaggcatcatagtgttgaccgtcgcgttgataatcgtagttgggcaacgacggtgatgtggctgccaacacaagcctggtcttgtgctcgtctgcccaaaacaatcccgggtagaagccactgtcggctttggatatcagccattccctgagggccatgccataacgagatgggccccttctcgattcaccagcggccatgattctgtcagcaacgtttaaaacccatgtgaagtttcagttcgatttaaaatgacacaactccgcccctactgtgacaaacatagagaaagtggcacacgtgtgctgctttggccgttctgtcagatgaaccccaccggtaacatctacttcctccaatacggcgcgaaagtagaaacagaggtgctccctgttgttcccgaagcaccgccacgcactccagacggtccctcgtccccgctgttactgcccgtggcactgtcaccggtctcgacggatacatcatcgcacacgtagcgaagtttcagggcaatgggagcatcccatgtattttcggactgagtagacgggcacaggaaaatccacgcacagtcacgcggtgggagatcgccaggagtcggcgatctagccagtgcccgcaaataatgcaataaatcaaacacctggaactgctgcgtaggctgcagttcatagtaattttcagaatagttggtcagcgccgtcatgttgcgttcctccaggtttttcacgcagattccagactccaccatcacacaaatcagacccctctcacatgttttctgcagggcctgcaaatctgccctggcgaactcacatggaaccaactgcgccgacggcagaaaacgcgtcaatggattatcggcacaacacaggtgtccagggcgtttgggatccggaaggccacatattcgcacaccgcaaccggagcgcaaaatttcaaccccaacgttctctccaaagtaaaaaaatgatacctcaatagctccgggtgcagcctgcgcgggagagccctggagcctggctttccgtctctgaatggaaactctcaatggcaacctagcccttggccgccgcgcctgttcaaagaaatcatctcggacaccactgcccatggaacagctatgcaacgatgtggtcgcaatgaggttacacagatcgcagccatttgtaacctcaggaagcaattgaaagagtctgtatctctccccagagacgcccccggccttgctcggtggcgtgagttccctcaagtacctgtgagatcttactgcgtataacattctaattttcttaaatttattggcatgtgcagtcgaaccgctctgacacacgccacgtgctaagcagaaatcgtcaaatatcttgttatattcttcgtcgatgcgactgtcggtggatctgttccacggaatacgtataatggtcctgtcatcgtcatcccataccactccggggtatcgacgagactccacagcatcgatgaaccaggcttttaaatccatgttttgttccgccatctcgcgtgttcaccacctctaccgagcgggtcagttaaaataccgagcccacaggaccacaagtctccgtgaccaaaagaatctagatctgaaagaagcctacctatatacgcccacagggcagctctctaacaaataaaaaaaattttctcagaaaacgaatagaggaacactgacgacacgcaatctagtcaaacccaaacctaaaagccttcactgacccatatcttccccttcccgagcgagtgcggtttctcacatgccataagcaaacgaaaacgctacgcataatgaaacgtgagagcaaaaaaacactttattgcagggagaaggtgcttgcaaacagtgcacggtatgcacggtaacagtatgtgttacatggcagcatccccacacgcccgccacagacactcgtgccacatttgaatggacaacgggatcgtgggacacggctcgccttccggagtgggaacgcctcctatgtgtagagacgcataggcggccggaaacagcgttccgtgtggcggggtagctgacaccgcgctcagatattgaacaagcgaaaatacgcgcgcaactcgcccactatttatcatatccaccggctccggcgaacctccattacccaacaaaaaggctggaacgtgacccatataccgaacgtaaatcccggacccgtccgcataaatcattagtcctcgtataatctccttcgccagccacctcagcatgcttgtttggtaaggtagtaaattatgtgggttatgcggcaacatgacctgttctggcttgatgcctgcacaaacgtgtccttgcaagggttgcttgggagctggcaacaggcgaacgccgttgggtgacccaacctccatgttaccaactagccgcccatagtaataaataaccaaacgtatcatccaacaccgcaccccgtcgcttcccacggcaccttcgtactccctcccggcaccaagatacaggaatctcgcggtcacctcgttgcgtaggtccaggagcatgccacccgtggcatccgcttcgttgcagagcggacaggaccggacaaagattggtaacaggcgaataacgcggcacctgtaaggcgcaggcgccgacaggttttcctcagtggacacatcttcaacggtctgactgtggcgtatagcgctggccaaccagtttttacattctctgaggctcaatggtctgttaccggtgtgtcgaatgcctttaaattgacaataatccagaaagatgtttttctcattttcagaaacgccacctgcgccctttaaatgattccacggtagacgaaccagagttttttcttcattcagccaatgcatccccgggtgtttaccattatcgcaacaatcaagcaaccaggccctcaggccagacggccggtttacacgaattgaccccgcgcgtccttccgccatctctgagggtccgctcacctaaactggcttataaaatgtatttccctttggcacaagtattttacaaagttacacctccagcacatacccttggctggtagggaaggcacctagtagctattttcatctccagtgccgcctactagcggaaacctgcggtctacaaaatccaccgagccggccgaatgtcaccatgagtcaattcatcctgacagatcaccaaaatgacagggacctggggtcttttacgggagccagggcgacccccaaggtaaagagtgactgcgttgatgggaaccgggtcgccgggaacgggggaaagggcgagtttgcgcagtagacccataacataaaaaatctgaaatttggcacaggatcgcacaaaacgctgtgcgtggtttccctctgcgtcgttcccggcgcaggaaagagtgtgccttgttctgttatccacatatatccccagctcattgccatgcaaaaagataccctcctcacaataatttatgatctcacacacggtttctctctgctctggcgttaatggtccattctcatgcggcgttggaagccacatcttaaacgctgtcccataataacacgcatggccagcttttgggtcttggcgcttatgcaagcgtacccctctacgatccacgcccgtgtgttccgccaccagcgatccaaaataaaaaacttgtatacgtagccgccaaaatggagtgatctgctccgggtgtggtggcgcggcaattgccaacctggcggctctggcataaacggaccgcgccgcaccactagcctccgccggagcttgaggcggtaggggcgtccaaggctcaagtccatgaacgcaggctgcctctaagtaatttatgacggtttccgaatgactatccaaaatgcaggcataacacagtggatctacggcaggttttaagtaaaaaaatctttctccgccagaatttgtcgtcgttgccctggtatcgtcctgttcaaaccccgcgttttcccgaagcacccttagcagtcttttccgacaatccgacacgttatgctggcgtccacactgcagatccctatccaggcagtacgcaaccaccggttgcataccctgcatacctcggtgccatgatagcttaaagctcctcttttcctcgtcgcaccaacgcaagtggtctggatacgtgttcgcttccaaattggaaataatccatcttcgcaagtgattgtgagtgatttctgactcggccatcgtgaactaagttcacggaaccgtcgcggaagcactcagcaatcccccaaactgtaactgaattttatgtccaccttataggatgcacgtgtatgtatatgccccataaaaaaacttgcacttcctcataattcacccccaaagcgtttcgggtcacccaccttaactaacccacacattacacggttcagagtgtcccaggtttcaccactcccccaccgctacagagaaacgaaacgttcaatttaaagggcaacaagctcaactttatttaaagagaaaaaaaccaccgccattagaaggtttccgcaggcacacacccccaagactattcgtggcgagttcgcggcacgctgtagtttttttctaattatttgtaattcgcatccaatcgccccgaacatagtcagtaaaaaaaccgtaaacacacacactctggtgaacaaattatgcgtccctggccccaaaacgtgcgtgagcgtcagcagcacggccgctgctaaaacataaacaagggctaaaacgttcgaccgaaacacgagtccgatgcaacaaaaaagcccaacgtggaagacaaaaaaatagtgtacaagtccgaggagcagaggcgtcgtcaggccaacagacagcgtacccgcggccgcccctccaattaaaacagttacgagataaaagtcccatccacacacctcaagcgcttgctttatagagtatgtgatgtaccgcctagaggtgagcgaaaaaattgcatggcgtcgttgcaggcccgcctcgtaaagcggatgtgaccgcagcgctctgagcgaacaaacgccgcctactataaaggccaccaggggcagcaaaaacaccggggctagaaaaaggttcttcagaaccaagaaatagtacaccgagaacgctgcgaaaaatccaagttcgtaatatctatgggcgattgtggggcacagatataccacgtcattcgccacaacaacacagagcagcaggcacaggcacgaccccacaaaaaaagaaacgtaaacccacggaaggtcaacgccaagggtggacgccatggaaccggcggtgataagaatcgccgaaatggtgcacggtacatccacctgtcgcgagagccaggccggaggcccaactacacaaactagcagaagcagcccgtgtaagcccaccgcgccccaggagaatataatgttctcaacgctattcaggggctctaaaaaatacagcgagtgataaataaaacacaaaataaagggggaagccgcgaccgtcccgctcataaaagatagccaggcgagcgacgcctccgacgtgtacgtgcccatggcgcggtgtctaaaacaacgggttgaacgttgcgcgaggcgtcttggtttttgcttttccgcgactgggtgagctgctgtgctttcgcttgccgtgtttcttttctggtcgagagccgctcgtccgcagtgatttggcgcccccgtccgtcggctcggtgccgtcctcggcgttcggaacgcactgctcttttaagtccaaatcgggcgttcttggaggggacgggagctcaggaaccccctcgggatcggcaccttccgagagattgcccggaacggcattattccggtccgtcggatctacctttaatagcattacgcccaacttgacgtcagcaaggctatctgtcagcggcaggccctcgacttccagaatctcgttggcgtgccagcggaaaccaggcaaaaaaaatcccggaatcttacatgcgttcaatgcggccactagagactctaaactcacatgagtcgtcaggtcgctagaaaccacacccacgtccagctttcttcctttagcgcaagtccacttggcgtccggggtgtcaggtttaaaatcaataattttactgaccccgtcgaccgtaaacgtggcaactgccagcgtttcgcttaacgaaacggtgaccacctgcttggccctggttttctgctgccttagccattttgacaacaaggtcagcgtcttgaccgtgaggaacaggcgattccccgtcccggcctgctctagtctgtcgtggaacgagggtaggttgggttcgtccacaacgctcttgtgcacggtgctcgtgtgattgtccccgtaagcgatggtcgtcctaacaaactgcggctttccttccgcgttcgactcgtccccgtggtggtaaaacgccacgttcatctccgtgatgttaactccaaacatctcacgggtgtgggtgaacgtgttcccaagggaggtgttccgaaagctaaagctaacctcgtccgtcatgtcagaatacaccatcagcggcgccagcgcatttttaagccggagaccgaggacgcccgcgtctccaacgctggagagcacgctgaggacgggggccctgccctggccgctgatctgtataacgccttttttaacgatgcccttaatgtggtcgtaaaccttactaatgttaccaagcgcatcaacgtccacgcgggcgccgtaatgaaatgagaccggcatggctaaggcgaacctaacaccccagtacgacgagaagagaattggcaaagacaacagacaaagcactcaatgttcaaccttttaatagagcctctggcccggaaaatgagtaacatgggacacatggggactacagatcattatgaacggatgtgacatattcagaattatcacgctcaaaaaagttcacactcttggcgctcgtatacgacaggggacacgcgtttggggggcgggcaccgaacaggggcggtacgtttattgacttcaaaatcctgtcggctgtggcttgcaaaaactgctcaatgtctccgacgttcaccatagtcacgcctcgactcttagaacgaataaacagagtctctatctccacggcttcgcgaaaaaggccgtggatgtatgcgacggagggggactcgtctcgggagatcatggtgttgtacagcaagccggcggccctggtgtgcagcagctcgtccctggagatgtagtcgttagccaggcacaccccgcgcatgactccgcgaaggcgaaaaagtcctatagagtaaaatgaacttataaagtatatcccttcgataaccagaaacagcagcaccttttcggcccgcgtggtggccttgcgcactcgggaatgcagccagtcaatcttctttgccagggtagcgtcctcgactatttttttggcataagtcatcaagtcgccccgattaccgccaaagaataggttcaatatgttggcatacactttgccgtggatgttttccatggctacctgttcggcgtaatagtgggtcacgtcgtgattgctgaactctttaactaggtcctcgatgttaaagtttaccaaactctctgccatacccaaaaatgtgaagagaaactggtaaaactccctatcggtgggactcaatagtcgcacctcatcggagtcctcgtgcagggaaatctgactggggaaccatctgttctgccatgtctcctgggtgagtgctaaaaagccagggtggtcgctcgcgtacagaaaccgatcgggggactccatagcggcggttactattgacaggccagacatacctcggcctgctcaccgtcggccttttgtacgcggttagactccgccccttctgaagaggtgttgtcacccacgacctgatctaagcactgcagggcattaagttttgtctgtttctgaatccgacaataatacagcatggtttttaaccctagcctgtatccggtcaggagcaagtctcgaatataactcgcccgcgtggcctgctcctcctttaaaaagaaattaagcgactggctctggtctacaaacggagcccgcgcaccggcccgctccaactgtttgattggacagtaatcgaaggccgttaaaaaaatcttatatctgtccttcaggggttctggaaacgaagccacgtcgcctccgtaacggcgcaccgttcgcaggtcacccggcttcactcgcttaaaaaaagttacgtttggttttagaatctcttctttgctggtaaccttcgatgcgatattggcaaagaacggataaaatgcttcggtgtaacccgtaagctgagaggttccggccgttggcatcagcgccaaaaactggctgttgaaaattccatgctgggcgatgctgcgccccagctgctcccatctttccaagggcgggtgggacggcttaacgccgtcccacgtttgccagtgaaacacaccctgagccaatcgactccgctcccatccatgaaacggagtcccctcgccaagtaaaacaatctcgtgactcgtgtgcaccgccgcaaaatacattgcctgaaaaatctccacgtccagcttggcgctctcggcgtcaaggtagccaaagccgagttcggcaaacacgtcggccaatccttggacgccaatgcccattgatcgttccttctggccgcgcctaacgctctcggtaggcgccgtcccgcccaaaatgcacgcattgacaataataactgcggcctccacggcgtcatcgagcagttcaaagccaaatgttacatcccccttcccgggctccacaccagactttcccctggggggtctgagacacttcggcaggctgatgtttgccaagttacacacagaagcctgaccctcgggttgctgcacgatttccgcacagagattagaacagtttatggcactgccttgcgtctcacaccagtggtgtttgttgagcgcctcctttagcaggacgtagggactgccggtcttaatgacggtgttgataagggcatacatcatcgattttaacggcaacgagctagagtgcttgccagcggccactagtctgttgtattcaatctcaaattcggcaccgtatagttttaggagattgggcgccacctctggcgcaaacaggtgccactggccgtctgggtttgtttcgtacagccggaaaaaaagctccggcacacacacgccctggaacaggttgtgacaccgctcctgattctccggcatcttagcgttcaaaaaatcacaaatctgatgatgccataattccatgtaggcactggcaccaacgggacggatattgttgtcgttgaagtaccctacgtgggcgtttattaattttaaacagctggtaatgttcttgtgttccgcaaaggacgagacatctatccccacgcccgacttgctagcgagaaggggggacatttcctcgtgaagggctttgagggttttgttttcggtcgccatggacggctttaaaataaaacagctagaaagctgacctccgcgaagcccggctgaccttaaaacaggcgttgcgcagcacacaatttgtgacgagatgtaatggaacgcgtaaccaaccaggtacatctcgtccagctccgtttcactctgcaccaggtgtctgagggtcttccgcaaacacggaaattttatgcactgacaggccacaaaaacagccaccctcataaacatctgggcaacgctctcaaaaatgggcgaagagccatgggttctcagcacgtacgtatcgtaaaacctgacggcagacaggtagccgcagttaacgagatttgcgtatgccttgctctgcttaaagtcctgcaaaagaccgtcaagggccgcttcgtgttttaacataaactcgcgaacctcgtcgcttagcttttccccgaataccgtgagatagtctttcaccgtaaccagacacctgtcctccattattctgtgccatagaagaccggttagggagttggcctctacgtcccatccaagggtcgcctttaaagaattcacaagtgattcggcgcatcgttcggggtcaaggcttgagctacaccccccggtttccgcgttggtaactagggttaaaggtttggcagatttagccgcggagaaagaagtttctgtgttcattgtttaaagaaatagcttccaagaccccggattggaaacaaactccgtaaatttcaacaccggtagagcagcgcctttaaagtgaaggctttgaaaagatggttgtgaaccggaaggcacgcttccaagtctgcaaactacgccaaacgcaagcctatttatatacaggtcattctgcagctgaatgtatttggtgcgaatcacgccgctgtaaaaatccctcaattgggcagctatttcacaatatcctttaccagacttaaaaaacccaagtcctagcgccggcctattatttgccctgatgtaggttaagcaaacataaatagacgagtgggcggggagaggttcgtcgcgcaggctggacgggcatgagtcatctaaatccacaaacatgtcactaggaagcgtaaggccaatatgtgtaacaacaggctctctggcgactacgtcgccctttaacgcagacgtcaccttggtgacaaacgtactgtggaccgtttgaaccaacggcccaaccggcgcaagaaactggtgaagcgaaccggtttccaacaattcttcaaaattgggtatggcatcgagtagaccgctctcgtggccgtaccaaacacacgctattctattggtctggggggcagagtccgcgtccatcctagacagtcgcgccagcgacgtaggcgtgaataacatgtcaatggaggaccccgtgtcggtttgtttaaaggaaaacaggtaggtgccccgaggttcctgtgaactcatcgtctgagaataaatcaaaaaatctccgtacgtttgacatttaggcgagacagataaaaatccatctttgatggcctccaccccagtggtggtcgacaccacatatttagagagcagatcacgaacacccttagaaaagtcgcgaccgcgagataacgaaacccggtgaggaggcggcggcagtaaacgcatcaagctatcattcagcttgttcacgtttgcatccctggtcttcatgacgtcgtcaatttctgccgcgcgggcggataacggtgacgagaacaccggtggactttacagattaattgacaacctcctaacgtgcaccggatctctacaacagcttaaactcctgatggagttccaactaaaaccactaccaaccgctcaccttctaagcatgcccaccgtgacccggtttttaaatactgcattcaaaatagacaaccccctggtttcatttattcaaaaacaccctgtgttttttttgatgagagtcgccaggcttccggatccagtcattacagaccaccaaagcgcagaaacgtcgacaggcatactatccgaggtcgtgaatgttcttaatatagctattcaaaaaccacacgagtccccagcggctaaagacgacgactatctcgacaaccgtgccatattgaccatgatcacagaatacatccatcacgtaacttcacgtacgccctcggggatcccaccgacaccgccaatgggaatcagccatttaccgtgcgtagagcagatcctacacgaaacccaccggcaatattggaacctggccctcccggagtcgctcttcatcgacaccggcgaggtcgcgtctccgcttcagacgtggctaatcatatcgtattgtaaaaaactgcaactggcaccaccgcccctattcccacccgccgacgaactggcccgtcgcctggtcaccggccaccacgaactgttcgtccccctatcaacgtcactggaaacgtacatcaccatgccagtgtcgaaacggcgagcgtttgagatatacagcgtttttgccaaatcaaaaaacatagtcgacggtacaccgattctagcgtttaccgacacggaacttaccacgttcaccccagaactgttgttcctttacgacttcgtaattgaatccctgtgcaaaaaccaagcatacgggtgttcgcgcaacgcaattgagcattttattaagaaaggtattgattttatggctgagttgggggcgttcatcgaaaaaacatgtggttatcgatcgaccgtaagcctgtccaacgtaagatccgtgaaggccaggctcgcgagctgtggactatccaaagaagcgtgtgaagatttccgcacaatgatactgatgacacctcacgagacgacgccaaagtgggaaaattttacggactttttagaaatggtaaaccagttaacactatacgggttctatttttacgagtgtcttaaccagtacagccccactagcatatcgctcgcgaaaattcaaaatatcttgaaccgagtcgatgccgaacagagcgatcgcgccctatggcgcactccattgatagggtctttcccgttcccctggaaattaaacaacgtgctggcgttttttaagcctagtgcccccgtggcaacgctacaaaaaatctacaaggcaataccgtcgtacctaatgaggtctttgttcgaaatagcggccaacaaatcgtggggtaacatcgcgcttgcggagagagccccgctaaccgacatacagaccgccgaaccagaccagggtcccgtccctacccaggttatcgcaaaatattgcagccgtctccagataagcgcgacagattatgacgcagttatcgtctccagcccgggatttgccaccgagttcattagaacaaaactgtatccgatccttagcgaggtgctccgaaacacgtccaaaaagaatcgatcgttattccaactcagatggctcatcgtcttcgcggcagaggacgccaaagacctagcccctatcagacgctcgttggccctggcgtactttcaaataatggacattttggaagaaaagcattctccggagtcgttttacaacctgctggactatcttcaggacacatttaggtgcatacgacaggtgataccggaagccacgtgcccacaagaatttctacaatacctgtttacttttcaaaaccttccaatagcggctagctttattcagacatccatgacctttgtagacgacctgaacaacggcatccccggcatactcgaccttgtctccttaggtgccgcgttttataacatgaaactactgtacgattcaacgctagacaccgtcgagattccaacggaagaagggcaacccatcgtcgtgagcatgttcgtattcaaatccacgattcgcatcctcgaaaagctcttgcaggaagccgttatcgcgttagctcaaacgtctgaaccgatgtacgcagcgcacatccgtttgatgcagcacctcacgtacatgcaaaagatcgccggacacgaaataatgaccacacaacttccatccgtgtttcacgaaatacacgaggggtatttgcagtgttttaagcgttttaaacgtcttatgttacacgttacgggcagctgctgctactcactgacgcgatactttggattcctataccagccacccctaatacccgataccatcgtacaaaaaattttaaactttaacgacaaaacggacaccaccgacgacatcttaaagagtctatcgcagcccgtgggacaagaacctttatcggctgaaaacgaaagtagcagtcgactctcaaaaaacgacgttgagctgcttcaaaaactatacgacgactttcggaacggttccacaaacgataaccccacctctattaaacttgaatattcgggtaattataacgaaacacaagtgtccgtggattggagcacatataacctggtgacatacaccgcacccgacgatacgttaaaattcaccccggtcaacacagaggcactagatcgcatgcttgcggaataaacaatggaattgccgccaattttttcaaaatttaaaatagagggcgtggcaacgacacaccaggccgactgcaggttcggacaatacgccggatcgcagtgcctaagcaactgcgttatctacctcgcacaaagttatttcaatagagaatcccccgtgacagacacgaacgaccttgatgacgttttacggcaaggggcgacgttggactttatactaagacggtccgggacgctcggctacaaccaatatgcccaactgcaccacatacccagctttattaagaccaacgagtggtccgcggccatcttccaatcgcaggagtactttggcctgattggactcgacgcggccatccgcgaacctttcattgaatccctaaaatcaatcctgacgcgaaattacgccggcaccgtccaatattttctgtttatatgcggcgacaaagccggagccgtaattatcaaaaacaaaacgttttacctgttcgatccccattgcgtaccccacgtaccaaatagtccggcacacgtaatcagttcggcagaccccaccgccatactagagtacgtgtcaccaccagacagagaatacacgggcagttttctatacatcatgcccagcgaatatgtcaatccagagcactacattaccaaccactatagaaccataacgttcgccaaagtgcacggcccgcagatagatatatccaccggcatagagccgtgcaccatcgaagacatcccaagcccgccgcgatcgccggatgtgacgtcaaaatcatccaacctcgcacgcgtaccaaaaaccaccaccgacacgtctagcgccaaaaccacaccggcaccgctatccggtctactgggcgtcgaaccaccgacaagctacccagacccggcagccgacgacgcggacacaaaactcttaactcccgctccagcgcgaaccgccgtcgaccaccccgaattccaaacaacaccgggagccacgctacttctttcagaactgtcggcatccaggggtcggaaacgcaagctgtccagccttcagcgatattcggattccgacgaagcgtcgtctgacgacgaaggggccccacgtaggcgcgcacacgacgatgcgatatccgccgaggtgatctggatggacgacgatatatctcctctatattcaccgtcggcgactccatcgtttgacgacgtgtttgacagccccccgatgagcccggagttcacgtacacgaacgcgacagaggacacggatggagcgtttctagaacagatcgttcgagacgcggaaacgccgttctctgcgttcaacgaccttatatcggaccacgacttttcttccctcgataaaaaaatagaacagttaatcaaatacgaagcaccctcgcagcacctaccgaacatttcggacaaacgaaacgggcgagccgtccgagaagcggcggccctccaggcgatggacaaaattatgattaatatcatactcgaacacggtctaattacagacgcacaggcccggggaccgtccgcctgcaaaaacgttcttcaattttttatcctgtggggagaaaaactcaacataccaataagcgacgccaagcaggtcctagaactcgatctgcaactgataccgttacacacggcactcagcgaaggaaaattcaaacagggggcgttcaaaaaacacctaacaactaaaattaaccggtgtctggcgtcaatgagggccacacacgccaatgcgcacaaaaaactggcatcggcgttcaacatagaaggctcacagatctcgtccagcgaaacaaaaatatcagtccgggcgctaaaggaacagatcgccaaccacctaagtccaggctttttagcggtctactccgcggacgagctaaaacatctacgggataaaattcgggacctaaaaacaggcatcgagcagcgcaacagagaaatccaacaggaggaactattctttggcgccgtgctcacagccctggacacgttccaaccccctccgaaaccggcatatccaatggagatctttccgcaccgtaaaaccgacgttatgctcaaccacctagcgtccataaccaccaggttaaccgatgacgccaccgaagccctcaacaattacctagagaccccaccagatcaagggacgcacatcaccaacatcccaaacttttcatccatcgtggcaaatatcatatccacgataaaaatcctaacgtacgcagagaacgacatgcaattaaacgtaacgcccatggcaacgtacaggcgtcagctgttgtacctcggaggcgagttagcaaccatcttcaacctagagtggccatacgaaaccgtgccgccggtccaagaactacccctcgtagcgcgggcaaaagccaaaatggaatcggtaacaaagatggaaaagaaccaacaggctctcgaccagatactaggagacgccgaaacgttactggacactataaccgccacatccggaaatgagaacccggtccgcgccatgtccataccgatattggagacctatattacaaacgcgggcgccctgataggcacttctcggaacccgcggttcgaaaaactcaaggccgccatccacgacctggcgtcatcggagtcgttcattataatgctgctaaacaacacacggctcgataacatatcagacaatctggccaagatcgacggcatcctgaccaacaacacgcgttttctttcgaccgccactgttacgaaaacgctccaaacgctgggaagcagcctgatacgcgaatgcgtagaagcgctagataaaaggagcccatcttccctgaccgacgcgcgtctcctcgcggtccaaaccatactagggcacgcgtccgttcccgatcacgagacgctaacgcgcattgtgtccggtgttgccagcgcacaaaaggaatccgctggcgatgatccagacaggtggacgcgtgtgaccggtcacctaaacgagctgaagctcgcagctacccaatcgaacgtcgacaaagccaccaggcgcaaactgttaatgataataacccgtgacctcaaggaggcggaggtgtctcaggaaacggtcctggaaacacggtggcaagaaaacgtgctgaagtttcaaccgtcgacgtccaaagaaatcgaagactttttacagtcggcaccgtcagcgaaggcccgaaaatttgcagaaaaacacttacggacgctgatcgcccaattcgacggccacgagcaacggccgtccgaggcaaccgccgttcccatggactacacgccaacgcccatacccacgccacaggccgtttctacggctaccgcggaaaagggaaaggccgcgtggaataaaattcaacaggcctttcaggatttcaactttcacctcatcgacgcctcggattggcaagaaatggcctcagaatactccagacacggctcgtcccttccgggtacggttggaccgaagctggtacgtttcatggagagcatctcgaacaccctggacgacatcctcacgcaaaaactagcatctctgcttccaaacggtcccgcattcagacccccggcgttcgactggatcgcgccttatcaaacacgcgtaaacgcgtttctaaagaccataggcctgccaatggtgcgcgacctggcggacaaaatccatcaccaatgccaaactgtcagtcacgcggtgcaatccgcagaccttcaacaggccacggtggggacgagtttagaacgacccgcggctgagtactgtcgaatactctctgacatgcaagtcgctttcaacgaccacggaatcgccgtgagatcggaggccgcggcgtacacggacgcaatcaactcgcctgccaatgtcgcgactcccccgaaacccaacctagaagcccccaagaagctaatgacggcaacggacgtcctaaccgtcgaagactttccagagtttctgaaaacgtcaattctccaacaggaacagcgactcatcgcgcttcaaagagctgaatttcaacaactagaggccagcatctcggcggccgaacggctccgccaatccacgcgtaacgagatcgcaggcaaaatggcaaccgctatcacgcaactcttaccccgggcccccgtcgcgatatcatcgagaccgttaaacttatcaaaacctatagactttttgagttcaacggtatacgacaagattctggacaaggagccttacgagacagccatagctggattcgcgtggctagaaaccgcgacaaaatccgtaatggtctacagtcaccaaaaccaaacgcaacagttaaacgtactgctgagcgaggtcgaaaaacagagcaccatcgcccagcgtctacacgacctggaagtgtcggcgagaaacacggacgacgtaaaggtgctgaagcaggcgctagacgaactcgcgcccctcagggtaaagggcggaaagaccaccgtagacgcgtggaaacaaaaactggaaagcatagaatccctgcttcgcgccacgaggacggccggagaaatatcctcagagctcgaacgcatcggcgcgcaggcggtcggcaccatcgccgttcgcgatctaggaacgctctccgatcaatgccgggaagccgcaaattttctcaaacaggccagtttaccggaaggcttctcggacataggcacaaaactcagcgagcttcaggcgtacattaagtacaaaaaacagtttctggagcattttgaaacaacccagcctaacgtctttcgacgctttccgctatcccaaaacataaccgaaaacgttccggcgcgcctcgcgatggactcgatggccagactagccaatcaccttcacgtgcgcggcagcgcgccccactttacaacgtggatagaaacgctgccaaccgtcgatcctgaaaaacccactcacgtcccagcccacggaggcgcccctctgcaccgccaaatcacgtactccaacgtcctagaggcgttgttttcattatgttccaccacgctgaccccggttccaaccgcccccggtctggaaatcgcaaccagggcacgccgcggggcagaggccgcaacgtggatggacagacagtggcccgatatagctcagacgctccaagacgtcctcgacacgtacgaacacaccaccacccacgcaaaccgggatgcgacatctaacgcgttcttggcgatgtgcgtgtttacacaaatcatcaggggcgccagcagagccgtgacgctcccaaagttaccgggtaccgccgtcgattttccagaagagatcgttctaacacccagggaatgcacaacactggtcaccgccatgtggcccaccctggcggccgcaatcttacgattaaaatcctactcggaagccctgggactaatgagtcgtttcctcccgctaatgttccaggcgctgccgcacctgacgctagaggcccaggtccaaaacggcccacataacactccgcctcaggtgagatgcttcgccagaacggaggcgattccgtatttcccggcgcaatggcagtcagcgaacctagagcagagcctgtggggacagacggactttttgcaaatctgcgataacaatcaacgcaaggccagggtggcggccgtcacctgggcgctcacaaccataaacggcgtggttttggaccaactgtggtctacatttaaacccatgacagccgcgtcagacgacacgtacgtcgacctagtcaagaccctacacctgaccacctttggtccgcgcggtccaacaccaaggcgagaaacgaccaccgagcacccgccgtacgagtacggacagcccacgggctactgcatctcgggccaatcgaccacgccggtccaggcttcaaacacgccgggtgtccgcttttgaggcggtgctcggagcgatggtgtttcacgtaccaatgagaatctttttggcggcacgcccagcgcctgggccaggcgcgcggcggcatggggctcctcacacccatcctggaatgcgtacccgacgtcgagccgtttaaaagcctgtataacgcaccccgcaaacccgtgcccattaacacgttacccgcatccctccacccccacgacgagcaacaggtttttctgagacaggcgcagtggctatcctaccgattcataccacacgaagccgcgcggtcgtcaagtccgccgcttctggtggtcatagacccagaaaacctcgtaacggcaacgtactccagtggcggacctgcaaattttgagagcaggccgttttacgtaatgcccggaccataccccacagactggcccaaaacgctgtcggtcacatcaaacacgtccgtgacgcacctcagccacgacgagatatgtaacctgtttactacgctatcccgggaacacgggaccgtgcaaggcagggatatcttcgcagcggctcccacaaacgtcacaccggaacgaaccgccaatcatccgacatgggaaacggaaaaccaattaacaacgcaaacagaacccgccgaaaaaacgcatgtaattccggcgtctcctaaagcgcgaacagatccaatagcggaaaccacggcccaccattcacaagggcaagcgtcgcaacacgcaaacaacgtaaaccagcccggtcaaattacttcacacgcctcacgtaacaaaccgtcaaccgcgcctcaggcgtcatctcgcccggagaaattaaacacgcagacggtgcctcgactaatatctcaaacgtcggaaacggcccatataaaccacccagcctccggccaagtcaccgaaccaaagggaatctttgggacgtacaaaccccgagtgctcaccgagcccgccaaacccgcacacgccagcatagcctctcgccaaccagaggcaaccaccacggtccccaagttaccgattaatgcacccaccgctaaggtctttataggcaccgcgtccaaattcttgccagacgtcgaagagagccacggcaccacacccggcgcgcatcaatcaaagatagatcaaaaacaatacggcgaaagtcagcctcaccgcaccccacaccttgaagaggttccacgggcgcctcacgtcgacactccaaccagcgcacacataaacgtcccctctagccaaggtccaaaaacaatacacgcgcgcgtaacacccggtattcaaacagtaactcccagcgcccccccaccggccggtcaaatccgcgcattcaatacacgcttcccagaaccgacggccggtcgctccacgacgaatcggatgtggaattgaccccaaccaatgcggtaaaagaattgcccaaaacacaaaatgtgtctcacaggcccgaaccggacgactataaatggaccgccccattcggtccaaccgtagagattcatcgactagaacacccccaaattcttgaaaatataacatcactcaccgtccccactcccagagtcaccccaatccctcccactgacgtctggatacccctatcccacgttaacatgcaacacgaagaaatttcgagagccaagaatgtgttaatgcgatttattcaaaacgtacgaagaaaaattcaagcgtcgtctgacgctctatccgaggctattgccagaataaagtttttatatctttaacgcgcccatctcacttgctctttttgttttgagacgagcgtcttgtgtccagagtagtcgcgtcgctaggtgataacgaagtggaccctatgccagaagacgccacgctcccgggtccaccgccaccgggcgtagggccaattgagcctattaatgaatggggcccgctggagatcgtagtgaagctatttgacccaagggtggaggccaccggggcaacgctcggggcggcggagccggacccggacaaaacgctcgagatactagaactagcgtcctttttgccgcgaaggccccggaggtggtctttgcgcaaaattccgtttttcttttgcgtatacatgtcgtaatgatgtttggccgttaaaaacaccagataattacgtttcgcaatggcatactgggcgggagacatgtcaccctggggaaggttgttcatctcggcaaccagcgggtgattcggatagtcgtgctccagtcgtccctgaacaatcggctccttaacccgcaatgacgacatatcttaactcctattgcactttccacaggacagggacgatatagacgaggtcaaataaaacagctcggccacgcgcaaatgctttaataggccggtcgcacagtccggcgaggccaaacagggcacaacaacgctaaccaaagagttcgcgtctcgtatggagtgtgccgcgttgttggcgagcacgcaccgtaaatagggatcgccaacacacgtgatctcgaatagagatataacccgcatgtgcgatccgccacaataagagcaatatacgcgcccggtggtcgcgcagatcgaaagctgcttttctttttggtcgcgactaaaaaacacgttggtgggtgggaaatttacggtttcaaatttaccccgtccgaaattcaaacagtaaccgcactcgaggcataccaccaccctcggagcgggaacggttttttccagaacgctcctagccaccacctgggaccaaacaggtagagaaatacacggaaacagtacgttgtacgccaatactttctgacccaggtcgcgggatatctccgtctcggttgactcccctatgggcagcacaacccgggacatgctcaacaggcccctaaacgtcaggctcctcagaagggcgttaaacgggttgccgcacgggacggttggcgccagttctcgcagcgaggccagcagtcccgcgtccgaagggcccaggccacgctcatccaggttagctcccaaacgtctggaaatggacggaacgttcagctgcatcgcgacacaaccggccccgttccatttcttccgcaaaacgggcaaatccagcgcgtgctgtggcgaacatgtaaccaggggaaaccgctggcgacagttgagggttttgcacacaaaacaacacgccctcggaaacgacacgaccagatacctggaccatgcgcccctgggaacggccgttaaacttagaccttcgttgtcggcatagcccacgccggtgcagatacacctcagcacccacgcgtacgcctccaacaacaaccggccagtatcgtgcagccgcgatttcacatcgccgtttttaggcgggttatccggcatccagccggtagcgatatcgttaagggcagtttgaaaggatggggcagaaattaacgtgcgcgcccattccgggggttgcccctcccaggcaccgagcccgtatgtcacaaaacacacgtagcattcggggcacaggccaattgcgcgcatagaggccgcgaaatctaagcccagccgagaacaatccagccaatgatggacaggataaggaacgcgcccatgacaggcagcttcgtcttcagccaagtcaggcaagcgccccgaggccatcccccaatagtaccgatggaatcggttaatcggtcattgccgcaccgctctccgctcccaatatccgtcaactccgaccccgaggggcgggccacggagcccgttcgcctcaacgcgccaaggccgtcggagtgccccgtaagggcaaaaaccgtcctaagaagggtttttaaccgtttagcgctcttcggagtcacaaccaaaaactgtaagacctgtcggtgctccgtaaagtaggtacggcatataaccatggagctgtaaacgtttaggtctccggagaaaaccagacgtgccttaaatttcataaaatcgtcctggcccaggaacacggacgagttcctctcgagatacacgtccgaatttataggcaggttttttccaaactgggcatcggcatcacgtggcttacacaaaaaacacttcagcgtggtggccaggccgttgttagtaattacaaaacacggggcaaacgggtaggccagcctctctagtttgtggagccaaaacttatacacaaactcgagatgataaacgcagccgtgctgcaggcgcacggtgcacaccgggaccgccccgcctttagcgtatacgggagccccgtcctgacacctctccaagtccagagaaattccggagggtcccaggtaagagacaactacatcgcacagctcgtcaactaaacgtttcccggaactcatacttgtaaaaatcttttaggtgctgtgcgcggctcccgtaaaaaccgcgtccgtgctgacgattttgtgaatgacctgttttacggcgttcaccttggcgtccagaaccatgcagtgctcacagtgagctgaccgcgtctgagcacgattacaaaggaaagtttttaaatactgacagtagttaatggcattgagcctggagtatatggtgggaaacataattttcatgtcttcgggcagcagggactcaaacgccaataaatcgtcaccggacatcacgtgagtggtaaagatgcttaccgcgggtaccgcttagctcacgaacatcaaaccccttcaaatattttagccattaaaaaactaacccaccctaaatccgtttaacacagactaaacgctcctaccgagcatgttcgtgccgtggcaactggagacccttatgaaacactggccctctctgcgggaactcgtagaacaatcgttccttcccggaacccccgatgtagcttttaacagcccagtattgattcacactcaggactctctacagcccgcctcatcgtgcagggtgtgtaacattctgttcactctggtccgaacattcccaccccccgactctttctttgaagactacggctggttgtgtctcacgtgcctatacgccccccgatcatggacggctacacttatggtggccgccgaccttctggaactaatacacatgtacttcccgcaacgcgtcaaagatgggccagtttacaccacccaaaacatcctcggaatcgacgtccagctccacttcttcgcaacccgctgcttccgacccattgacagagaacaaatactccacacatctcatttaaattttttacaaaccgagtttattaggggcatgttagaaggcacaataccgggatcgttctgttttaaaacgtcctggccgcgcacagaaaaggacgaccaacaacctaccgttgcgtgttgttccgttggccgcggaaatcacgccaaccgagataaccacctacccgatgacctagaagaggcgttcaactccacgaacgccaaggaaaagcccagcatcctcggcgtcttttcggcaacgtggtcagaatcccagcttctcggctccgacacacaacaggcaactatccattcacaaccctccaccttcccaaccccagaagatgctgaccaatcacagggcccctgcctaatgcacccaacactcaacctcaaaacaaaaaaccacaccgcatccatctgcgttctatgtgaatgtctggccgcccacccggacgccggtccggttctgagagatctgcgtcgcgacatcctggaaaacatggaaaacaacgttaagctcgttaatcgcatatcgtacatcctaaacgatccggactccctgtcacacgtgcgcgacgaacatctgcgcggcctaattaaacggtgctcggcgcaagaaatccacaagcattttttctgcgacccgttgtgcgtcctcaacacgtactcgcactgtcccgcggttttatttaaatgcccacctcccgaaaagtataaaaagctcaaagctcgtctggcgaccggagagttcctagactgtaacagaatatttgactgcgaaaccttacagaccctggccgtcctctttaaggggtctcaactggccaaaatcggcaaaaccacctcgctcgaaataatccgtgaactcggatttcaactccgtcgacacaacattcaaatcacccacccgtttcaaacctccaacctatacatttgattttcagaagcgcgtcagacaatgccaaaacagcccagacgtcgattggcgtctcgaacgccatacgcacctagcgtcaagcgaccggacgggccccagtccacgcaaccggcatccaggcacggcagctgcaaaagcgaaattatgcagtggaaaaagttagtttcagacacgcagtttttttccgccctaacgcgccgccacgagctgggggtggactttttaagagaaatggggaccccgatatgcacctcaaagtccgttatgttgccgttaaacctaaaaaccatcgccccgggtcggtgcgtgtctctctcatcattcggacactcgtcaaacatgggattcaactgttcgtcgtgcacaccaactgacaggtcggcggtgtctctggacgcaaacgcgctcggcgaagattccgccagaaaaaacagtgagctgtgttcagtggcgttaaccttttaccaccacgccgaaaaggtcgtgcagcacaagggattttacctgtctctgctcagccactccatggaagtcgttaggagaagcttcacgcaacccgggttactttacgcccacctagtcctaaaaacatttggccacgatccgttacctatttttacagtagatgccaatgagagacttgcactctgggcggtgtttcacactagagacctacacctgggggaaaccagtctgcgactcattatggacaaccttccaaattatgacataacggtggattgcatcaagcaaacatacataatgaagtttacaccctcgcgaccggacaacgcgaccgtaacggttccagttaatagcatctgcgaggccgtggccacgctagactgcacggacgagtttcgagaagaaattcaaaggggaacaaccataataaactctcagggctcattgtaattttcccctaactggcacaaatgtaaaacacctaaccaaatgtaatttttatccaaatgtaagttttttaattttgtacagcttaccaataaaaataacttgtgtttacacgaattgaaaagttttttaatatcacaataagtaaacacgggaatagtaagaacagaggtcaacgaaactaaaagcgttatctggataggatccctagaaagcaattaacgtgcgccggtatcaattaacgggaacgcacgttaactgctctccacggatcgcaacgccaaactccgcgttttaaggaccattcgatgccgttaaagattggcggtaaatcgcaaaaggtcgtacatcgtagtactgattttacgatgtgcacacctttccgcggttcaccgccaacaacaaaagcagttcacctgggcacccaaatcgcccgggccttttacgccaacgtacaaaagtgactggcgttctacgatcacgagcaacactattgctcgcaaacacaatagcgttgttggtgaccgcaaactgccaaatcctgatatgcagggacatggtcctcaaaatctaaaacggacttcccaaaaaaaaacatgcggcaataaacaacccctcgcggttaatgcacatcgcaacgaaacggaatccaaagaacacgcccgatcttaaaaccacgggcacgtgtctttgggttccgggggcgttacgatggaagttcgtcaccagatatccaacaaacctgttacaaaagggtaaaaataatcggttttaaaataataaacaataaaaaattagccgccgaagttacattccgacatacaagcggacccaaaaaaacaccccaacgcaaaacaactggagaatgtgtctttgggtcccttccacgtcgcaatgaaattcctggtgaacgtgtgctgcttaacgcgttacagttatattttatggctacgtaaacacaccaatctaaaatgctaacacgcgcaccgcgcttaggcgcatctgtgcaactaactgctgtcaggttactactcttatttttaacaagtagcacaccaactgcaattaattgcactaaacccggttctgtcttcttttggttaagttggcccaaagtctttatggagaaccattattcattcatctccgttaacagcgttagcattactgttgtcgctgcgggcaaaataagttcgaggggaaaaacagcaacaaattgttagtttaaaacaactcaagttctaaaatctcacccaattataaacaaaaacatcgcagttaaattgtttgcagcaaacataaccacacaagttttttaacacaaatgcggtaggtaaccattgttgccaggcgtgccatgtgttaagtgtttttaaattacattgtgcgtttaatacaccgagcagtaatctcgggaggacggttaacgagcgatatacatattccctaaacacgggaacgcgcgctgaccaccctccccaaatcacaacacgggcatgtgtacaaaacctaaattgttaatgctaattaagaaaaaaacaaaaaaccgttagttgtgcgcaaaaacttgcaaatgtacccaaggctttcccctcaactttaaaacagcggtaatttgtttaaacatactaagtaaacacgtagacacccgcatgtaaaactatataattgtttccgttcattaaactggaaatataaaggctttgcgtgaaaatctgctaacgcaaagggcacttaatttttcccatgcaaacttagaaatctacagctaacgttaataacagcaactttaggcagcaaaacagtgtccgttgcagaccctgttagaactcaaaactctatagcaaatgttaaacggcgactgtaagcagcgaatcagtgtccgtcgcagaccctgttaaatataaacacaacagggagcacgactaaacacagactaactgctcattgagccctgttgaagaactggcgttgcaatgcattagggaatctaaaaacgaaatcctgctcggtaacacggaaattgtctttagttccctacagcattacaacaatttcaacctataaacgtacgcgcgtagggtcagtgtggtgtgtagtgcttggtgcaactgttacacaagggggcttgtaaatgtggtgcttagtgcaacgtgcaggtcccccggtggggtgcaggtcccccggtggggtgcaggtcccccggtggggtgcaggtcccccggtggggtgcaggtcccccggtggggtgcaggtcccccggtggggtgcaggtcccccggtggggtgcaggtcccccggtggggtgcaggtcccccggtggggtgcaggtcccccggtggggtgcaggtcccccggtggggtgcaggtcccccggtggggtgcaggtcccccggtggggtgcaggtcccccggtggggtgcaggtcccccggtggggtgcaggtcccccggtggggtgcaggtcccccggtggggtgcaggtcccccggtggggtgcaggtcccccggtggggtgcaggtcccccggtggggtgcaggtcccccggtggggtgcaggtcccccggtggggtgcaggtcccccggtggggtgcaggtcccccggtggggtgcaggtcccccggtggggtgcaggtcccccggtggggtgcaggtcccccggtggggtgcaggtcccccggtggggtgcaggtcccccggtggggtgcaggtcccccggtggggtgcaggtcccccggtggggtgcaggtcccccggtggggctccgggtggctccgggtggggtggcggctccgggtggctccgggtggggtggcggctccgggtggctccgggtggggtggcggctccgggtggctccgggtgggcacaggttctaaaagctacagtaaatccaacggcaaatccaacgcaaaaaagtccgtgcgccagccgcgcagaagcgcgactgcttacctgtgcttccggcgccctttatataggccaaaacaccctcccaaaatgagtaacatagtgttattatgcaaccataatactaataaatgaatcatcaatttttggttagcgcaccagcgtccaaccgcccgccaaccgtgcagcgccgtatattcgaatatcgcgctacacgaatacgggacgcggcatggtagcgggagcttggcattgcgcaatacccaggtacagcgcgcaggtacattgggcccagctaacggggattccccaaacaaggtaaagcccacatgcttgctatgtgaacggtctattcggggattccccatacctcagacccgccgagagaatattcagcgggactgtaggctgggtcctcccaggatagaggtagggagttcccctttaattaattatttatatatttaaaaatttaggctggcatactataatggagaaccaggctgggaacctttgtaccggggattcccctaacaaggtataactaatgtaaacccatataaattgatttttttttgggaaatccccgcttgcttttacattaatatagaattacaggctgtagcattagtaatggagaactaggctggagacctaggtaccggggatccccctaacaagatgtaattaatataaacctatataatttcaccctgttaggggaatccccgtttgtacctgtattaatataggattacagcctgtagattcggtaatggataccaggctggagaaccaggctgagtgatttactaacatgggattcaattaacagggaaaaaatataatagaatatatataatataggggaacttgtaaacaaaacccaactcgcggattggctgtttgtttgggccaattggctgcgagaattccgtgattgacaggcggttgggccaatggcctgcgagcatttttgattgacagatcgcctagccaatcgcggctgggaactcggcagcgaagcaagaagccgacggttgcgacaaccaatcgccgccgcgcgtcggtttgattgacggctcggcgggccaatgggaaacgtccctgccggcggcccttaatcccccggcttaagggattacggccgtaaacatcgctggcgcggtgccacagccgtcgagccccgcgcccggctcggtgtgacctccccgcgaccccgccgccctcatgcgcaccatgggtacaatgggtaccgtgggcaacattttgcgctgaccgccggatgaccccggcgccaaagagcggccgatgggtacgcgcggcgaccattaaaacatgtttcggtgacccccgcctgaccccgcgccccaaattgcgggcacatggtggcgccggcgccccaccgagcccatttcccacggcccgcttatgggcaccaaataggcccatggtgatatgtttctgtgaccctcgggtgacccggagcgacattttgcgctccatggtcctgaattcgccccatgagtacatgtttcggtgacccgccggcgaccccgggcggcaaaaaccgccccgtgggcaccaaacggcaccatgggcacatcttttcgtgacccgcgggtgacccggcgatggcaccgcgggcgcggggggtgatatgttctgatgaccgccgccttacgttcgcagcggtgtcagcggaacatattctcccctgctcccgggctcgggacccgcggacccgagtcgagttattaaagcgacattagggattaattttaatccccgaccccctttaatggtgcggctaggccggctgcgacgttgacgccggcgggttcgacccattgaccgcatcctcggtggccgggcagaggccggccggccaaaggtgcgatcgcggggtctaacccacccgtgtgagagagccctccgttgcgttgtcttcggcgttcctgaccccctggctcaccggggatgtcttgtgagcactttcccccgggttacaatggacaggaaagtgctggcaagacatcgacggggttgccggtcggggaacgggaggcgcgccggtacgccgcttgcgtgagtgaggtggcgccgatgggccgtatgacgacccccgcaacgcgaagggccgctggcgcggacatttcccagaaccggccgcgccgccaagcggcgcggctgcctcccccaacgacccttattttagcttttaagttgttatttaaagctcggctgtttgtggcacgcgcttaagcgttgccacctgctgcggagatcccccgtgcgcatcgccgccctcctcgcctcagtgcacgctgccgagcgtgccacctgtttggttgtcatcccatgtgcgcatccccccattttgtcttcccgacccggcaacataaacgtcacgttgtttgtgaaaaataattgtttttttttatttttgttaacccctcaacctcaatacagtctctcgccttccacccgttgttttgttaccagcccccctcttggcgaccggcaccggcgttaaccgggtgcgttggcggccacaaccttggccacgtcgcgtctggagacggcctgcagcaggtcttttaaaaccaggtagtctgaggggctgaccagggcagccttttccatttgcgagatccattgaaaaaaggtgggtgactggttgcgtttgcgacccagctggttgcccgttaaaaaaactaaattttttacgtccctttctgagagctgacggttaatggagagcattaggtgtttgtacggactaaagtaactggggctggaccggagcctctcgacaataaatcctacgtctagtaaaaaaaaggtggagaccaggtccaatcgcccgacgagaaacaccgcctccagtaacaccggaaagggaataccgggttcccctgttggggggcacagcccgtttgcgaacccctcgggtgtattgtcggacaccggccggtcaaaaagccacagcacggcctctctgtcgtctgcctctaggtggcgaccaaggtccactatccgcttatgcgggaacatgggtccggtgcggcgcttgccaacaccctcttttacctacgctggagcgtttgagaaatcacgacgaaccgcgggcgttatttaaaatgcggtcttcgtctaattcccgcaaggtggtggcaaccgactcggccgcggccgtgagggttgcggccgaaacgccgatggcctccgccaatttgtccagaggcggcgcagcggcgccgaggctaaacagcgcgcaggcggccgccaccagggacggggaaagcccgccggtgaccgggttcacgatggctttgtgaacagactccaccacccggctgtgaagggcaaagagctgctcttttgtaaagccgcttttaaacaaggcgggcccgatggcgtccgttggtaaaactgcttctagtttccaccttaaagccttaaggaccgatttttcctgtttcagtaaatccgccacggaaaaatcatccgccgcgcaaaagcacaaaaaggcggccttaaaaggattcaggtctctaatcttgccggctaaaaataggcaggcggcccccagcctttgaaaccgccgacgggggatgctgcgacactctagatagcgatccagaatgctgacggccaggggaaaaacgctagcatccgcctggtgggccctggcaacagaacgcatccacgtccccaagataactcgcatgcccaaagtcacctccgtttgtactgtcccgtagcaggccgtggatgttacaaaggacgtttcgtgggccaggaggttagaaagggcccggtcctggtacaggacggggtcaatggtcccggtgggcactgggccaacaaaagccattattacggcaaggtaaagaaagcgagataagtcctacctgcggctcacggaggcttaataccaacgaccggcttaacacccgatcttctttgcgctctatgtccgctccacagcaatcccatgtcttgtgcagttcagccctgcctgttcccggaaagcggcggactagagtcaacgcggaagcggctgttcatttaaggtgagtcacaggcggggcaatttgccagccagtgacgcgtttcctgcagtctgggtttttacctggcaagcgccactgatacttggcagcctgccggtggggatttttttagtgctgaattggcagtcctctgtccatgcgcactatgctagcatggaagtggggggcggatgggtgaaattgttcccaccggcgacgggcttttttgacggccttggcggttccgcgactttgacagaacaccgtgatgcaaaaagccgggatgacgggacttccaggtgcgtccggcaaaacgcggacgctggatgctctaatcccgggaaactgtaggcaccttgccaggcgatacagtgaataggggtctgggttacaaaagatgactccgtgacaccacgggcccggtcgaggggggtcgaccctgtctcccaacgcctgacgataaaatcttctttgtgggcggtattttcttggtatctcctcctcggatgagggtacggtatgggaatggggtatcgcggcgggcgcggggggcgcgccggttgctgttgccgctcctccccctcttgctcccgttgtctttcaggaacctcgtcctcgtgggagttgtgactcgtgcatctgagacgtaaacaaggaactcctccgggacgcctgggtggaccccaaccccccacggtgtacgtgtgggatgtatggccaggaaacgggggtcctggccgagacggcccggcggggtggtctgtgtggctcaccgcctgttgcgtgttaggggatggggcctgttgaggcgttggcgattgctgtggtccaggtgagtcgggcggggattgcggagggggcgaggacgttggcgttggcgattgtggtggtccaggtgagttgggagggggcgaggacgttggaggcccaggcggttcaggtgagtcgtggggcaacggttgcaaaaacgacggaggctgcggatcgggcgatggatgatgcgacgacggcggtgcaggtgagaggggggcggggtgcgatgggggtgatggtgggggcgaatgcgatgcgggggatcccggggacgaccctgatgtgtttcgaggtgacaggtctgaagctaacaccggcgcagacccgcttggcgatggcgttggagatcccaacacgatgacggactcagaccccgaccccgaaggcgacccctgtgatgacccgtgcccggactcggtggccgtttcggtgtccgatccgtgcccttgttcgacaaccaccagaacctgtggcccaggcggcgttggggaagcggaggccgcggatgcggaggctggtgcggccgcgccacgccccctaccgcgactgcgccccccggcctgttctctagtgccgccgcgtcttccgcaatgtcccctgcaggaacttcgcaacccgtgtcccgatactatgccggaacgatgttgccggctgccccacatgatcgcggtttactgcgtgtaaaaaacgacagattaagtagattgccctttccacgacccgcagccacctgagcgcgcgcacgcagaacctatgtttagtgcgcttgcgattatgtccgctaggtggcgggctattttgatcaggccgcgcacgcattgaccgttaggtggcgcagagcgccgtaaaccgcagagagcaccgcacggcggttttagtttcgctactataacgtttagttaaaatctgtggccgatattgggtggcagtatgtaattcacgcgcgccgcggcccccttaccttgttgccggcgaaccgcggaaactccgttccgccgcgcagcgcgcggccgctgccgaaaccaacgccgcttgaatttcgcgggcggccgggcttggtgtcaatgattcacacagatgcgatgtgattgggcgtggtggttaacgtcacttgttaacccgtaaatctggaaattcttgtccgccgcggcggtgagccgcggccctaagccttatagtgctgtgcagcgatcctgaaactcgaaagctggccatataaaccccagtatggcgttgcgtgtcggtggcaacctttttgaaaaagatttattgccgccaggtgttaagcaccggcaccgaccgtgcgttttcaatcatgttgggaggaattacattaacgctgctgctggcgacgctcgccacggttcggtgcgctcttcaaacgcactatgcggcggtccccgtgcactctaccgcgtcccttgggtgcgtgttaacaacagcccacaacgttcttatcgttacctggcaaaaacaggaatcgcctagccccgttaacgtggccacatatagttccgaagcgggcacggtggttcagcccccgttcgccggtagggttgacatccccgaacacaagttgaccagaacaaccctgaagttttttaatgccactatggaggacgaggggtgctacctgtgtatctttaacgcgtttggagtgggaaagctgtcgggaaccgcctgcttgacggtttacgtccccctgtccatgtccgtcacgttttaccccccaattaacccgacgcaacttgtctgtcgggccgaggccagtcccgcaccgtctgttaactggaccggcgtgccacccgagctgtgcagcgaacccgaagtgtttccccgacccaacggaacaaccctggtcgtgggtcgctgcaacgtaacgtcggtggatcccgaagacctcagaaacgccacgtgcctggtcactcatataggaggtttggcctcggcgcggcccctgggccccgtgttttcggatcctctggaagggacgagccactacgtggtgggtgtggtggcagcggtcgccgttttaggcatttttttaacgggtgtttttttgtataggtctatgtgagcgcgcgtggcccccgtgtctagtgttttgttccccagtgagtgtctccatgacaaatacaaatttgaggctggcttttagggtggtttctcgtgcaacgcttcctgtgtgactgcatacaccggggtgtcaccaggaaaccgcgtctcccctttatgttcgctcgccctctctgagcgaaggtgagaatggtacccggggcgttttggcgttgagagagtcgggcgatgttgccgtggcggcgtctgcaaaggctcacccgcttttgttttttcccttttgccagacaccgacatggacgccttgaacaataaccttaacctgctgatggattttctttcaaactattcgaatagctacagcagttatgacgataatatatcttacaccttagacacggaatccacgctgtgtcggctgactataattttcccacctactatttatgcgattatatgcttttttattttttgcattacgctgctggggaacgctttggtgctatatatattttttaaatttaaagctctcgccaactctgtggacgtgctaatggcggggttgtgttgtaactccctatttctgtgcgcgtcgtttttgttcagctggctgctgtacgtggcgccgcagattctcacgcccgcaacgtgcaaggtggaaatctttttcttttacctgtacacttactttggcgtgtacattgtggtgtgtattagcctcatcaggtgcctgttagttgtgttttcccgccgcccgtgggtcaagcactgggcctccggctttctctgcgtgtgtgtgtccttaattgtggcgctggcgctgtctgccaacgcgagcctctatagaacggccctgcgccacccagagaccagcgagtggatatgctacgaagatgccggtgaagacaccgtcaactggaagctgagaatcagaaccatcagcgcgatttgcgggtttttggttccgtttgggctgttggtgctgttttatggacttacgtggtgtatagttaaaagcacgaagctggccagaaagggagccgtgaggggtgtaattgtgacggtggtggtgctgtttttaattttttgccttccctatcacctgtgcaatttttttgataccctgttgaggaccggtttcgtgaccgaaacgtgctacatcagggacgtaatcagcgtggccatgcacatatgctctctgctacagagcatgtacagcgcgtttgtgcccgttgtgtattctggtcttgggtctctgtttaggcgaagggttagggatacctggtccatgtttaggtgtttttccacctcgggtagtttatgagacactcaaaagacacttggtgggattgtttgtgtacatttattttttattttgtgtacatttgttttcattaaagcgatctgacctgcagaccttacctgacgtttactgcctgtttcttacgcaccagaggaacagggactggaaggccagcgcccacggggaaattgtcatgtctgcgagttcgggcggcacgtacgcccactgccaggggtggaacaccacggctgggtctaccagcgaggccaggtggcgcccgtccggtgacgtgatggcggccacggccgagttggcagacgggttgcgtgggtagtgctgcgcgaacattcttgggtcggggttgccagtgtggaagttcagggcgatgcgctgctgctggttcaggtggtactcaatggcatcgcgcgggtatctcacgcccaggtaccggccgttggcccacccggggaggacgaggccccgaaggaccctaaacattatgctgatggtggtctgggggatgtggaggtttagccagaggcactcgtggttccctgatgcgttctcctccaggtggatgtcccactggtcgggggttttgggcccgggcgtgtcgtgaggggtttctctgagtagaccgagcgcccccaggagctggaacccaaactccccgcagcacagtgaaaatgtatccgctcggcggaagaatgccataaggcccccatagcacccagggtcgttgagaagacccatgattgcgcatcgggcccccacgtagctgtcttcgatgcccacgtttccaccgatggttaagccggagaatccccggaggatgttccctcctctaaggtcgttggtggagacggccgcgacgtcgaacccgacgttagtgaatgcggccatcagcgccctggggagcggggcgccgggggcgatcagggcggccacggccggtggcctcgacggcaccgcaaatagcgtcagttcgctgtttctgcagacgtcggcgaggtggcccaggttgtgtcggttgcacccgtagtcctttctgtagagttcctgcgcgggcgtgaagctgggcccccaggagtaccactgttcgtccgagaacgaggtccagttagcggccaccgaagtgagggtctgggagtaaacttcctcgttgttgtgtgagatgacgattctttccgacagaccctcctggcccacggtgccgcacactgtggcctggcagtcaaagttctggcacgcctggcgcacctcgtcaacgtgctggggcaggatttcaaaaataacgcccggggtttcggacaccagccacggcaggggcgtttcctctgacggaatgcaaatctgcaggcctcggttaccggccagggccatctcaatagcggtgaccactacgccgccgtcgctgacgtcgtggccggacaccaccagaccccgggagagcatagactctgttagcataaagaggttggccaggtgtgtcgcctgaacgtccggtagggtgggactcggcagaagacacaggtgctcgaaggttgatccctgggtcaggtgcggtccggggaaggttactagcacgaggaggttacccgtggccttgaggtcaggtgtgacgcgtcgcctggacgactttacctcggcggtggccgtaatgataaccgcgttaaacggcacgggggccaccgtctgatgctgttgcgtggcgctgatcagttcttccgacaggcacggggtgctgccggccgacgtgatggcaaagtttacgctcaggtcccggcatagttccttgcaggcaaacagtgcgtgttgcagtagccaggcttggtcttcctccgggttccaaccgacagacgccgtcagcgtaatgtcactcaggcgccgcacgtgcgctagcatgatgttggtgaccgcctcgcagatggcgtatcttgcgcccactgccgcgtcgatgcccatcttgtacgcctgttctccgtgggccacgcattgtccgacccacctgagtctgcccctgccctgggtgacccaggtttccgggctacccaggcgttccgtcgcgtcctgcctggtgatcggttgggggcgatgtatgggcacccgcgccattctgtccgtaaacaccgccgtgtggttgacgatgtggtaatctgacagcggtcggcccaggggtcccacttcgcactgctgagctacgagtccgttggagcatctgtccacatgtcgcgtgacaaactctttgctgccgacggttggacatcgcagaagctgatcgaccacggaggccaggcggaataggccccagtctgtcggcgagggtgcctgttcgccgccctcctccggttcttctgggcgcaccagccagttggaggaaacgggctgctcgtcgaacccaaaagttgcgatcacttcggccgaggtcggctgtctgatcggccacgctcccgggttccacagctctaggtcgttcacgaactggattcccggctcttcgcaggtttccccgacgacggcaaaagggcatccacagatgcgtgcggcccttctgatggcatccaacgggcccggttccggctccgtgtcgcgcgggagaacggcaaagaccacaaaggccgacacgttgagaaacttgttaagtaggatgtcgtttacggtttcccgtgagatgtctggcgtggcggtcagttggcttagaatggtcggtggaagctgagagacgaagaggcgaaggccgccccgcgagaccagggctcccaggtggctgatggtagaggcgggttcgtgggcgcgattaatgctggtaaagaggggcccttccaaaatctcgtaagccagcgacaacgtttggttaatgcgattaatacctgcggcgctccccaggtaaagagagcgctggttgccatcgccgtcaaccggttcaaaagacccaagacagatgataggctgattgaataaagacctcctgaggaatgtgtatgcttgaggcacgcacgataacagcgatgtggtggtaataacgttcgcctcactggtggtggcgctgtggacggttttaagaaagccaccaaggacaggcgtgccggtgcgcgccagctgctggcagtggagtgtcgttgttgtgttaatagctctgtggcgtgcgcgagaaaatgcgcccactgacggcgtagtggcgtaaaatccacacagggccgatctaatgtcgcccccgggaagcagcgtggaatggaacaggatgtcctgcagcgcacctgcacctgtccgaatggcgctgtgcaggtgtgtgaatgagtcagctatgtgggttacgttttcgcacggaaggcgctggaaaacgcagccgtagctcctgcgggccatgacggcgtatctggacaccgtatagctgttgccattggaggcctgtacggccaacggtaatatgttccgttgtaacgggagcatcaccgcggcgcatattgggtctctctccggcaacggtctccaggcggtggattgcactgtaaccgggagctggcgctgcacgcgcacatcatattctactagtgcgccaagtgtcagggcggcaaatgtgcccgggggtacccggtcggcgacaaaccgtctagtctggcagctcagatgtctggccagttctaaccgcaacgctgtatcaaagccgctgtctctaaggcactgcactagttcgctagatcttgtggtaggttggcgcagtaggtctgggccatagggtagggatatagtatgatggccctgaaggtgcgccgaccgagtttccagaggtgcataattaaccaagggcgccaggagctgccagataacgcccagcgttgggtcgtcatcgtcgttgtcaaagttatcagaggggcgggttacaagccaaagtaaaaattcactaaagcactgttctggcacgagcgccaaaacgctttctgcatcagaattgtcatgaataaaagcctcctcgtctgcactaagggccgcggcggcccagcgagggttcgtgcgttgggccatgtttgctaagtctagccggtgcaaaagcaagcgtatgggactttaatagggcgtgtaagggttgttaggtatttggggggtatttgtttatgttagatgaagtacaaacctatgttatgttagttttggccaccttcacacactgcatcaaaaaactccccaatgttacagtaatcacttgacaattcgccctccaactcatagaagctattttggggagttgaagatacaggtaagggggggaggggaatgttaggtggcgagaggcatgacgctgaaggggaggggctgggaataacttggaggggttgacttacgcttattggatgatcaacggttatttctgaaagtgctacccgctggacggtaggtcgtgggttaggtaaaacagaaagcggtagggaaattgacctcagggccgcccctccgctggcgcgttgccttttaacatctagtgctgtatggatgagcggttcatcgcctgccccggagtttgtgggcgtggtccgagggaggatgttcaagtcaatgttaacttcctcttgagaccgtggacgggcccgtctgtagtgtcttgtggagggggttgtctcaacgtgtgttggtgtaaacaatccaggtaatcggtaaaggcacgcggttagtccaatttgcagaaaacctaaaaacacaaaataggctcaaaaaagtacttttgtggcggttaataaaattccggcgccaacgtctacacaaagcgaaactactcaccaattagtaaaatgccaactgaaagggtgataacacttagagcttctgttccgatcggttcgaatttcagaatggatagcaaatttgccccgctgccaatgcctggaaataagcggtgtgtattttacatgtggtacaggacaaaaaaccttgtaggtttcagttgagtcaccctgccacgtaaacccgcctagagtgcggcacgcgttgtcggttgcaggaagtacatactaattttaacttgaaaaggtttattgtagaattgtggtcaaaattatattatacttacataacatccaatgaacatttgcactccagaatgatattcggtttaatgcgtgataaaataagtttgcaacaaatgcagcgacgagtaggcccaggaatataaaaaacactgcataaaagataatgttagttagtgtttgtgtcacataaatgttttaaaataacagtgtgttttgtgatgttaccgagtttgctttctggcaatttagtgtgaataatatgcactccaatttctgcggcaacaacgtgtagtgcaagtacaattccttgtagcataataaacaagcaggttccatgatttgtgcttctccaggccttcttacaagcaatatacattgcaacaacagctgttaaaaataacacaattaatataataaactaacattaatatttggaatttacattattaaaacaaaaagacagtattaaaaaataccagtgtttgctgtaataatagagtatgctaatgccattgttaaagatgatgtagtacttgcttcagttactgcgtacatgccaatgagagctgtaacaaaaaataaacagttaatttcttcataaatattgcctctgtttattataaataacaacaaaattgcaacatacctgcatatattgcaaaccacgatccaattattcttacaatgtttgtttgtgaacttaaccatacatatccataaaatgccattgctataaatattggcggtattaagggtataattttgtagtgtccattccataatgttattgttataaaaatcataagtattgcccatagtggcaaaactaacaaacatggagtacttgttaaagaacagcgaggtgtttgaatagcaaaaagaaaagtgcctgtaaaataaacacacaatgctgttagtaaaaataataaaaggtatttcaacaaaatgtgttttataaataaaatttcttacctatataaatttgggatacaaaggcaatagcaatacacgctttcacaagtaaagttgacccatgtaacgaaaacccggacaacaaaacacagcaagctagaaaaacaaattatgtaagtattagtggttattatgtaataaaagaccgctttccgccaaattataatgtcatacctgtaatccacggtattagcttcaataaaggtgtgtttaaccgttgtgatctactctgataaaaatacaatccaagagatttccataggaaaaaaaaacaagcaactgtagacacagcaactatataaaggtctttgttattgtcacaggcatattgtgccagtgaaataagccaaaatgttcctgttaaaatgtttgaaaataaccaggtacaccacacaaaaaaggggcagttacacgccatgtttaaaaaatcagtaaaaaagtctacacaagttaaagggggactttagtttaaacgccttattacatcgtttccaggcagacttctcactgagtcacacaaaggtttctatgttcccctgtcgggaatttgccctagtgctgatttcgttggcaaacaaccatggaaaagcgaactgggaatatttgtttttgttagctggccttggatgcttgcaaacattcttgttattctaatgttaggccaaattgtgggaagcattaagaattgttattgaaaataattctatttaatcaaaccatgtttcaggtcccagaagggttgtagtaatgtgtaataggaaacaggcctaatacagaacattctgttgggtacatgaactaaaaatatgtttaatgagggctgaggtaaaagctagggtgagggctggggtgagctagggtgagggctggggtgagctagggtgagggctggggtgagctagggtgagggctggggtgagctagggtgagggctggggtgagctagggtgagggctggggtgagctagggtgagggctagggtaatggatacgtttaaaatattaggctacgtttttactgttatgtaatttgcgttactgttaccttgaatgtgaattaggcatagggttatagtttataaaccacacatatgtaaagtaaataaaaaataaaatgcgaaccaagctggcacaagtaactgcacgctaccctaaacgtacaatactgggactagaacccagaggtagttagagtatacggtagttacagaactttacagttcctttaggccagcagggctctgtggttaattaaacaaagcttaaagataactgaaaatttaggaagtgcgtatgagtgcaatgtgttccaaatcgggcaagggttacataaattgttgcctagcagcggcgccaggcgagggcccctagcggcggcgcccggcgggggcccccaggcgcccggccggcggcggcgcggggccgcggccgccatcttgcgcccggggcgagggtctcccgcgcggcccccgggcccccgagcggggcgccgccggcctcccccggctcccggccctccgcccggccggcccgggcccgcggccgccgcccccggccgccccgcgccgcgcgccgggccgctttcggttcgcggggccgggggtcccgcggggggccggggccgcccccgggtcccgctccgccgggccccgccgactcccgggagcgccccggtccggccgagcccggcggcgccgcgccccgacgccccccgagcccggggccccacaagccgcggcgcgcggctcccgatgccgggcggccgccgcccggcatggcggtcctccgccggcctcccctcccccacgtcgccccgaaaggtggtctccgcgccgccgggaggggggctggggcccggggcgcctcggcggggccggccccggggcgcgaccgagggccccgggagaacgggggatcgggaaaacgcgaggggtggcgggggacagaggggaacgtgtgcgtgcttgtggaacactatggcctgctggcctgctcgcttgcttggtagcttgcaagcttgctgctgagggtctttagagggacagtaggcctgcttgctggcctgcttgctggcctgcttgctggcctgcttgctggcctgcttgctggcctgcttgctggcctgcttgctggcctgcttgctggcctgcttgctggcctgcttgctggcctgcttgctggcctgcttgctggcctgcttgctggcctgcttgctggcctgcttgctggcctgcttgctggcctgcttgctggcctgcttgctggcctgcttgctggcctgcttgctggcctgcttgctggcctgcttgctggcctgcttgctggcctgcttgctggcctgcttgctggcctgcttgcttgctgctgaggggacagtagggctgcctgcttgcttgctgctgaggggacagtagggctgcctgcttgcttgctgctgaggggacagtagggctgcctgcttgcttgctgctgagtaggactgctagggctggagtagggggcagccggccagctgcgtgcgagggcgttgcagggtccacacgaggacacgggacccgagccctccccaggaccgccgggcaagcggcc

SEQ ID NO: 2 - protein, 98 aa, Japanese Macaque Herpesvirus
MRWAWFSPFLSHQLGVSNHAPSPGPTSAPLPIPLLPVPSPASMLPAPTALSVRSPRATCRADLGRLPGGPGEGSGPVSSCGPATPSHAAGRLPHYYQP

SEQ ID NO: 3 - protein, 425 aa, Japanese Macaque Herpesvirus
MFVLLIFILLQPASLELLPAKLTAVPTWCPPHPGDTYLLTCRGTSTARDQRSTQWFRNNTLMHGSNFYGRLVSVTPNSTISDWYACQTKTTTRSNSIDFRVRSSRLTLQERCSSYGYSNANNTRVLRCYSGGNVTLRNVVFHLNGTAVINGTTTDIYTFVLTEKTGGTYYCSAFIGTEKLYSQKINVFFTSFTFKHTDNVQNGSEFNKTEQIQQTANVQHTANYVVFSVPVFSIGVLTGIAISLIMCWLFTLRCNKNSESSNNRHAHQTSYIQPSHNQHSHTSESTTHTYRNDHQEESIEELPNQHTRKTNSCQTVLLEVKNVAFDGPQGNLHNTNDEVMEQYDDVVVENIEQTSYDNNIEQMDYSDIIRPNFNYYSGLILEEVDEVFYNELANQYHGLILENLDHDEYNHLNKLNMIEQYDWLE

SEQ ID NO: 4 - protein, 193 aa, Japanese Macaque Herpesvirus
MDITVNCIVAVDKQLGIGKNGTMPWPYLKNEMMYFQKMTSTPSVIGEKNVVIMGKRTWFSIPEKKRPLANRINIILSRELREPPKGAHFLARTLDDAFNFYRQYKLTKQLNTVWVIGGKSVYESVLNYECSLKLYITRIMESFDCDVFFPSINFTEYTMLSELPGKDTNFEENGIKYKFQVYEKRLINKAVTQ

SEQ ID NO: 5 - protein, 395 aa, Japanese Macaque Herpesvirus
MRLIMFWFPVTVCFVCHFILVNAQNETNVTCDKPNFDRFMVAMEKKEKYVLGDKVELTCRPGYTLQGKVYVQCLQSGMWTTPNAECHRKKCTNYGDILNGQVIVPDSDNAFKFGTNITYKCNTGYLLLGSMVRTCLLTGNSNTVNWQPAAPTCEIEKCKKQPDIENGKYYPVQDFYNYLETITFTCNKDFSLIGNTTTTCMTNGTWSSPVPKCEQITCSAPNIEHGTLLVGSSRVYKHGQSITIGCENGFTLNGHKMCTCEYSSWNPPLPTCVPINKTVPTPSEVPSPGTNKQERPTPENPKSHESETTTETPKTGTHKSETPSKKIPNPETHKPTTPKSGTSEQTTNRPSKAPSQNPPMEPPMSKWKRHVVLVLFASVASLLFVLVTLYCCFLK

SEQ ID NO: 6 - protein, 1132 aa, Japanese Macaque Herpesvirus
MASKGNAGQPLEDNQGSRAPIGACGYVYAYSKQDFPFAEASILGNRPSGSGVFSLPILYGLTVEHEFPLTVKAAHKKVDTTTLAVKVTCFHREVIVFHNANLFRPVFEGTGLNELCEEARALFGYTQFIEPGPPHGIWNPLECPQLPDKDEMFLGVVVTEGFKERLWRGCLVPAVFQTQQVQIAGRQAFKVPLYDEDLFAPHGHRMPRFYHKDVSAYLYNSLFTSIAQALRLKDVTAVIHATEKQFMQDHYKIAKIVQAKQFSTTLPKTADGSSHMIVDSVVAELALSYGCMFLECPQDACELLNYDSWPIFDGCDSSEDRVNALERWSAEQAVHVAGQLFAANSVLYLTRVQKQAPRGQKGDVNVYNSFFLQHGLGFLNEATIKENGSEAFKGVPSNALDGSSFTPYHLAYAASFSPHLLAKLCYYMQFLQHHKSSTNQTFNIVHYVGTAANSEMCTLCHGDTPGTCLNTLFYRLKDRFPAVTTPQRRDPYVVTGTAGTFNDLEILGNFASFRDREEDGNPADEHPKYTYWQLCQTVTEKLSAIGITEDQDNHVNLITNIQSFLRVFKGIDSVVDGEVMKFINSMIKNNFNFREHVKSVHHILQFCCNVYWQAPCAVFLNLYYKSLLWIIQDICLPYCMIYEQDNPAMGILPSEWLKMHFQTLWTNFKAACLDRGVLTGCELKIVHRDMFCDFFDTDAGSNGLLAPFKMQVRIARAMMVVPKSIKIKNRIIFSNTAGSEAVQSGFVKPTGTRDTYVVAGPYMKFLNSLHRALFPNTKTAALYLWHKISQTNKTPVLKDVPDDELAELVSYVKTNSLAFEETNVLDVVPDSLMSYARIKLNGAILRACGQIQFYATTLHCLTPVLQTIDAEEYPHVLGSAAIATPVAYLSEIRGRTALTVQTTARQPVAATGRLRPVITVPMVVNKYTGVNGNNNVFHCGNLGYFAGRGVDRNLWPESSPFKKTGVSAMLRKRHVMMTPIVDRLIKRAAGQTISTFEAESVKRSVQALLEDKDNPNLLKSVILELIRHLGKGCQDLSSEDVQYYLGDYCMLTDEVLFTLDNIAQSGVPWTFEDAGALIEDRQNTDDLQFVDSDDIATASCQPPEEQLPTPSAGALLAGKKRKINVLLSDLDL

SEQ ID NO: 7 - protein, 104 aa, Japanese Macaque Herpesvirus
MVLHELFFCGVDDRRHVFESQGLGDTGKKGVVEVRANVFMVKPRHPMPVRCEQVFVVQRHFERLTPGDLHLLGLKHCGNETAPPQSLLKPFRHDNAKEHLILVR

SEQ ID NO: 8 - protein, 88 aa, Japanese Macaque Herpesvirus
MAPFSLILAYDMRESGTTSSTFVSSNARLLVLTYDTSSASSSSGTSFRTGVLLVWEILCHRYSAAVLVFGNSARWSEFRNFMYGPATT

SEQ ID NO: 9 - protein, 86 aa, Japanese Macaque Herpesvirus
MQRCGVELNLTARPQNGPVQFNPRVRHEGIRDHVQHVRFLEREAVGFDVRHQLRQLVVRDVFQDWGFVGLGDFVPQVQRGGLGVWE

SEQ ID NO: 10 - protein, 83 aa, Japanese Macaque Herpesvirus
MFLARCSTSSDRLDVSTEFCLRSSGRRSRFRFSICVFVFRMREPSILRGSAKTTRLRSTARADSCAYKAASSLAIVVRRPRFS

SEQ ID NO: 11 - protein, 686 aa, Japanese Macaque Herpesvirus
MARELAALYAQLSALAVDLSLVVFADPRSIDGSRILKTKTQIENLNRDLLPLLRKQNSVETSSLSLEVEHLAKNIEDKLGELERSLRQRYSSREHFETLHLRPECHYHATVTFEFYGGGLIDVNMCLINDVELLCKRLGSVFYCIGANEALSGLDRVLAFLSTLRGISPIPHPDLYVTSVPCVQCLREIELVPNQGSSLLAVLADRHCDHLCKKVRAEPIHGLFETELSQLGLKVAKGSDATQHGVRSSADQLRESSLAAIQDHNIFKRVSASIMELSNLIYWNAGQTGLQTGTENECSQMAKLLTHEAEMHEHRALITPKQSAAHFYDCFRPDPIESLFCGGLFNSIDDTINALSRDCSVTFFQQANYTNVMRKQNELFTRLNSILCQGSAGSQKPATPSEPRTATVAATAASDVIKDAQYRKEQYMKKVARDGFKKLTECLQTQSAVLANALCMRVWGGVAYGEASELVNHFLLRRRFVALPWEARCRSNQILFENSKYIKNSLYSQRLSREHVEIITLQFYGLITGPLTRQSDLFPGPANVVLAQCFEAAGMLPHHKMLVSEMIWPQIQPKDWIDQTFNRFYQLPEGDLNAVQKSAWCFIRELVLSVALYNRTWEKTLRIFSLAREKPSISDLDVKSLTPGLYLTYEQNTPLVLISQNTGWIFKDLYALLYHHLQLSDGHDDN

SEQ ID NO: 12 - protein, 107 aa, Japanese Macaque Herpesvirus
MWGTIFFLNTIRMPSWCVLSSVPGHVFSRLNLKSSPVAEALHTRNLNASALGGFSGGVGVDGPGVDFAVVVTPFGVVTFSPTAAPIAIITTHARSRRRVRLVIIMAV

SEQ ID NO: 13 - protein, 828 aa, Japanese Macaque Herpesvirus
MMITNRTRRLLRAWVVIIAIGAAVGENVTTPKGVTTTAKSTPGPSTPTPPENPPRAEAFKFRVCSASATGELFRFNLEKTCPGTEDKTHQEGILMVFKKNIVPHIFKVRRYRKVATSVTVYRGWTETAVTGKQEVIRPVPQYEINHMDTTYQCFSSMRVNVNGIENTYTDRDFTNQTVFLQPVEGLTDNIQRYFSQPVLYTTPGWFPGIYRVRTTVNCEIVDMIARSAEPYSYFVTALGDTVEVSPFCLNDSTCSVADKAENGLGVRVLTNYTIVDFATRTPTTETRVFADSGEYTVSWKAEDPKSAVCALTLWKTFPRAIQTTHEASYHFVANDVTATFTSPLSEVANFTGTYSCLDNVIQKTLNATIKKLSDTHAANGSAQYYETEGGLFLLWQPLTQLSLVDEMRGLNDTTPAPPTTSTASRVRRSVDTNSQTAEDLAAPQLQFAYDKLRASINKVLEELSRAWCREQVRDTYMWYELSKINPTSVMTAIYGRPVSAKFVGDAISVTDCVTVDQGSVSIHKSLRTSTPGICYSRPPVTFRFLNSTTLFKGQLGPRNEIILTDNQVEACKETCEHYFIASNVTYYYKDYVFVKKINTSEISTLGTFIALNLSFIENIDFRVIELYSRAEKKLSGSVFDIETMFREYNYYTQRLAGLREDLDNTIDLNRDRLARDLSDIVADLGDVGRTVVNVASSVITLFGSIVSGFINFIKSPFGGMLMILVIVAVILIVFALNRRTNAIAQAPIRMIYPDIDKMQPSGGKVDQEQIKNILAGMHQLQQEERRRLDEKQRSAPSLFRRASDGLKRRFRGYKPLENEEAQEYEMNK

SEQ ID NO: 14 - protein, 97 aa, Japanese Macaque Herpesvirus
MGAWAMALVRRFSANTIRMTATITRIMSMPPNGLFIKLMNPLTIDPNRVITLLATLTTVRPTSPRSATISDRSRARRSRFRSIVLSRSSRSPASRCV

SEQ ID NO: 15 - protein, 1012 aa, Japanese Macaque Herpesvirus
MDFFNPYLGPRGPRPPSHKCTDAPAPAGAVQPPPDVCRLIPACLRTPGAGGMIPVTIPFPPTYFENGARGDVLLAHERSMWTARGQRPVVPDPQDQSITFHAYDVVETTYAADRCAEVPSRFQTDIIPSGTVLKLLGRTENGTSVCVNVFRQQVYFYAKVPAGVNVTHVLQQALKNTAGRAACGFSTRRVTKKILKTYDVAEHPVTEITLSSGSMLSTLSDRLVACGCEVFESNVDAVRRFVLDHGFTTFGWYSCARATPRLAARDARTALEFDCSWEDLSVQADRSDWPPYRIVAFDIECMGEAGFPCATRDGDAVIQISCVFYTTREGAPNPPNILFSVGTCDPIPDTDVLEFPSEYDMLVSFFAMLRDFEVDFLTGYNISNFDLPYLITRASQVYNLRLNEYTKIKTGSIFEVHEPRGGGGGFMRSVSKIKIAGIVPIDMYQVCREKLSLSDYKLDTVARQCLGGKKEDVSYKDIPPLFRSGPGGRAKVGSYCVMDSVLVMDLLKMFMIHVEISEIAKLAKIQARRVLTDGQQLRVFSCLLEAAARDNFILPVPTPEGQGGYQGATVINPIPGFYDEPVLVVDFASLYPSIIQAHNLCYSTMIHGRDLHLHPNLTPDDYETFVLSGGPVHFVKKHKRESLLGRLLTVWLEKRRAIRRTLAACDDPSLKTILDKQQLAIKVTCNAVYGFTGVASGLLPCINIAETVTLRGRTMLEMSKSYVEALTTDNLRTRLGREVTACHGARFRVVYGDTDSLFIACDGYSAEAVSAFCDDLAARITADLFPPPIKLEAEKTFKCLLLLTKKRYIGVLLNDKMVMKGVDLIRKTACKFVQERCRSILDLVLHNQEVKAAARLLCKRPPHAVYEEGLPAGFIKIVEVLNASYVDLRNSVVPIEQLTFSTELSRPVCDYKTTNLPHLAVYQKLASRCEELPQVHDRIPYVFVDAPGALKSDLAEHPDYVKQHQIPVAVDLYFDKLVHGAANILQCLFGNNADTTVAILYNFLNVPYKLFS

SEQ ID NO: 16 - protein, 186 aa, Japanese Macaque Herpesvirus
MLRDVVRFENFFGYSSGREAARGPAGGVLEGLLEDVGDVDAGWDLRVEIYLLTEHVHAHAGAVLGSPQELEHGSARNNVRLEAARYLGAPVRRVRRFNDVVRVKRNGLVLGVRDDWPLAARRPHGPFVGEQHVSARAVFKVRRRERDRDRDHPACPRRSEAGGNEPADVWWRLNGAGGGGRIGAFV

SEQ ID NO: 17 - protein, 102 aa, Japanese Macaque Herpesvirus
MHRSAAQHERLVVVRRQVGVQVQVSSVYHGGVAQVVRLDDAWVQAGKIDHQDRLVVKPRDGVNHRRALITPLSLGRWDREDKVIPGRGLQQAGEHAKLLAVR

SEQ ID NO: 18 - protein, 101 aa, Japanese Macaque Herpesvirus
MPLHPGPGAPQSGGQGGGAAVVQATAARGIRGGVAGGLYKNCGGPQRELCGPSKQRRAHRAVNVLDRAQPPRLRLQDHQPAPPGGVPEAGEQVRGAAPGAR

SEQ ID NO: 19 - protein, 416 aa, Japanese Macaque Herpesvirus
MLVNELSMVLGDWEVTFHRGKFSFVNLARLQTFKGHGGYAKIRLPFSLDQLLHQHFAFGLVTRLNELPPFSDCVALIAPRDSGGDADAARVAPGFVLDSSRPLTVWVNANGRHTVRFCLLFLKPIDLERAVTYVFGENGGARSEGAPKPTCNTESLPGGPLRVSGEASQTSPHSFVAYFPTADPVACLSLLRLQVRPFSDDAAHRDARISPKYVTFSNAGGNVCKASVHTLSPSRCKTAQMEIIYAPGDPNAEIVLGQSGPVLPTHTGGRVLGVYADAEKTIQPGSSAEVRVQLIFQQGAAARGDLAFLVAGVAPEPLFIVTPTLLLSGCTTHLRLFNPNGTPTTIKRDTLVAAAAPCPVVRVSSADDAPRDLVASPDTGALSIDAFTIPVGLPGVVSAECHVSMRDNGVHERMSH

SEQ ID NO: 20 - protein, 91 aa, Japanese Macaque Herpesvirus
MESAPVSGDATRSRGASSAELTRTTGQGAAAATRVSLFIVVGVPLGLNRRRCVVQPESKSVGVTMNRGSGATPATRNARSPRAAAPCWKIN

SEQ ID NO: 21 - protein, 409 aa, Japanese Macaque Herpesvirus
MGTPVRFFRGEWQTSSLVDNGTPRYSSLVWAATIHDGYLTLVNRSELCVTERSPCLPACPSIGRLVGKRFPGFAFASATLGDRGTRTVFYAFGHRDNPLDIVPAVVERADRELVLRVHAPRTTRVSRYGLKIFVAIVTVVRPPGVFLHFPQDRVPIALTDACSQEGSRLTSEEPWIKIQGFPVLSDETAHPFLLTQKTKPFTERKFCRLIMDDNQLSAVNTVYLGKQHVRVTVTRPPETIVTDGPVTATLSLTGNAPIAFRHNPYFELPWSSTTAIFTPVVYVGLTVCIPPNCSKFVRYGNTYVSAFNRKLTAIISNHAHDGGFRIQDCEWPPNREIEILVTNVTQAPVYVSTGTQLGRAIFVFAPRFGGPAKLRQLLGHRSRALELPGGVTVDSQKLCRFETMYLFST

SEQ ID NO: 22 - protein, 207 aa, Japanese Macaque Herpesvirus
MFPVWFVLFYLSCWAASPTLAPPPTAAGINVLPQWAGNRASLDRTRGRLSELGLNIQRWFVYLCYHSTLCRVREYPRIMSFINFPILMSNVECQRREFRGAECMNAMVRGLRAYESYLTRSKMLLDDAPGNAGAAAIGSAVTVVLSALNSLIEELPVDNKIGGVESNDKIVRALAEQSPGDVILSAFRLLEYLQMFLRDGRRAIAMM

SEQ ID NO: 23 - protein, 333 aa, Japanese Macaque Herpesvirus
MIVLVHLGICYVKKIIPACLSGIAAVRCRVSRGSEVAAARAPDGRGEHGELQYLAHLDLIIRHGVQRDDRTGVGTRSVFGLQARYNLRDEFPLLTTKRVFWRGVVEELLWFIRGSTDSTELSRRGVKIWDAHGSRAFLAAQGFGDRREGDLGPVYGFQWRHFGAEYRGADADYEGQGVDQLRYVVDLIVRRPHDRRIVMCAWNPADLARMALPPCHVLCQFYVARGELSCQLYQRSADMGLGVPFNIASYALLTYLIAHVTGLTPGDFVHTLGDAHVYNNHVGPLLLQLGRTPRPFPRLKILRKVTRLEDFTRADLSLEGYDPHPHIEMEMAV

SEQ ID NO: 24 - protein, 88 aa, Japanese Macaque Herpesvirus
MCLHGGASTMTLKINVFCGSCTMRVWLMNAGGDSGGDAGGDSGGDAGGDSGGDAGGDSGGDAGGDSGGDAGGDSGGDAGEGPRGFTTR

SEQ ID NO: 25 - protein, 120 aa, Japanese Macaque Herpesvirus
MRVLVIGAFFAVFACVVDYAFPMGSMSGPAPEVCCLGYINKLPPSGAVALYYYTSSQCTLDAVILETHRGQKLCANPGDDGVRKLLQKVDNRPKRNKGRRTRRSLLDDASDEGLESGSGF

SEQ ID NO: 26 - protein, 91 aa, Japanese Macaque Herpesvirus
MRRVCLGGGDFPFIAGTGPLLVLEIPFSTGSLLRRKAVYLFGESPFFRTPGATVVRHARFGTRDATSGREARAPGGWPRLVSVEADPGRGY

SEQ ID NO: 27 - protein, 112 aa, Japanese Macaque Herpesvirus
MLFLVAIGDIFRCPPNDPVRRGFFGSFGGHRKMFPRARRATVETIFSDPLVTPEPAPPRGAPCLGAEQSVLVRTPNAGLAVPTPARKTRGTPAGFYAITLCYFGVGAPRCPC

SEQ ID NO: 28 - protein, 102 aa, Japanese Macaque Herpesvirus
MSGVSVTRGAEAAGGGVGKNSGRRARVSVMTRLDGGHLAAGQRREHRERPAGGAARDRAPRSVKMADRRRRLGAADVNHGGEERRWSGYTSITEGRAGWQII

SEQ ID NO: 29 - protein, 187 aa, Japanese Macaque Herpesvirus
MAAIQGPPPLPEEENENSLPDDVYAIEGIFLYCGLGQAEYLHHPVFSPIKEFISSFLKDSARLYERLLRHTDYRSLRGLNAIGQGMLHINTDGRHNWGRALAVLGLGAYVVDKIRDDERLLTFAIAVLPVYAYEALESQWFRSHGGWEGLRNYCERILRHRRNARRHMCYGVAAGLLALVALFAIRR

SEQ ID NO: 30 - protein, 536 aa, Japanese Macaque Herpesvirus
MTSVYVGGYVDVVSLPKIEKDLYLEPSIVATLLPYTNPLPINIEHVPEAHVGHTLGLFQVTHGIFCLGKLTSPDFLALASRLAGDSRAAQIQLNPMPRDPLLEMLHTWLPELSLSSLHPEELQDPNHPPAFQHVSLCALGRRRGSIAVYGPDPTWVVSKFDSLTRDEAGKITSKCLDLCERQVTPPEFAAPLETLMAKAIDAGFIRDRTDLLKTDKGVARVARSTYLKASQSPSSQHGGNRDTQTMSALPDDNITIPKSTFLTMVQSSLDHMRNQGQRAYVSAPPSMPATAAYPSWIPPPELTVPSYAPPVAPPFPFQSAFAPQPSPYAATYYSPTYGYAQAPSRHQKRKRDVELSDEPVFPGEEVGIHKDVMALSKNILDIQADLRDLKRAASQTSGEKDTDQRPQPPPVQFSWPQTYASAPYLAYQPQWYPGTDTHLHASQPYQIAQGIQQTQPPPPQPASHHAGLATQPVAPAPAAQESAMSNAVPSASAPRAGACPPLDSDCGQSARAPVEASVQPAPVSQIQKMFCEELLK

SEQ ID NO: 31 - protein, 299 aa, Japanese Macaque Herpesvirus
MFIGRGFVYGSRVATIEGSKYRSFSIFGRLTTSTYPPTYTEVMLGRCLREPKEMSAGLRGLMWRVIRCENLNTFLPGELRFLHLVLCEMYNYGLNVYLLKEAIANTGTRDDIVLGRKVPVEFWKIIYDGLREMGVSDATLLSETKRGALWLYFNGRPCLLKGLGDYVFCRLGLSHSVRVVPENLTDGNYLYNLGSVIPCRLLVALSYCLAFWGHADHEPWVRLFAGKIFILYLIISGHIMPRKSILEQVGTSGYGGFVEAVCRDVRAVHGIPAWDFAAAAPALTSRQTDYLFAFNNSVV

SEQ ID NO: 32 - protein, 97 aa, Japanese Macaque Herpesvirus
MSPASGAELLLGVLGARRPRALGQVVRRQDFYTVLNNIRPHYATKVNLRTGGNVRVRWLCRGCVSRRSRGPRHSGVGFCGSGSGVNVSTDGLPVCVQ

SEQ ID NO: 33 - protein, 547 aa, Japanese Macaque Herpesvirus
MRTSEKCCMRYPRKPARQITATFWAPHPNNVLFIHKPSLIEERRNAFVMRNQQLALRVHTLRKNLLRLELDNVLQTHQRETEMVMRDLDTIQNMVGDLRSPTRETADTQTSINPRPKIAPQTHGDAFVVTIAPGDPGFTVNQDLRLELLPSLYMNQNQWLPQYGPWYSSLTDNAMQRRVFPRDLRGTTNFQNSTSLKLMSAVISTAASITQDFYADVRNVSDTQAALCLLNGYYCHRTGTPLPPTRDGLWDNLGTKLATLISHLKQNTKGLGFEFTYSNPKQRASLAPLNKETKYSADFFTNHVIYATLAQSGLLPGSKNPGTGQPPGPDLVYILATTLFSEDVPPFQAYQWNLRAGLSALGCLVLVYVLLELAQITPRSPHRRLNLASLLGGRFSKVEDPSGSKQYLKKGQLFDFLTENYISPLLSRAPDAPTSFLFPGAYLTALEAKAISHLKHTRPFVNLTGSRFNEIFDILNQKLTFRDAGSLIQAQTSLRLTAEEGLAAILSHPSPPGLTHEIMKSQFGVYDDYDKVYFLVLGYLPVATSVV

SEQ ID NO: 34 - protein, 350 aa, Japanese Macaque Herpesvirus
MTVANQCKHAATLEALPASRKRAGIRAHLAVYRRLIKHRSLDDILKFLSIRPTREATKDVKFRIFFEVTLGRRIADCVLTVDSEHQKTCYVIELKTCLSAAVFPGNAIKISQRWQGLHQLTDSVAYIGRAAPRGHENWSVRPWLLFKNQKTLKTIHTESSAFPPTFINTTSVALNGFFSQWEDAHVRKMLYEISTKTSAANYRNFLGPTSKQRSVHSQTIADRRKKKRVCDAKSTAGAKGSHAAKKPAPSRARQRAADAPTGNRNGHARPRHNSKHGRGSAVPDQGNRRYPNVNKPTTQNRPSDTWRRVCCHDSPRRPGLHGKPGSPSGAPAQPVHEPKPMAATIRSVVQ

SEQ ID NO: 35 - protein, 556 aa, Japanese Macaque Herpesvirus
MAEGGSGFGDELVRQMRDRKPRWDESSDDTDDVDTESTDLEYDDVFPVVDTHGLTSPGSQNYDVPTSPSGTPWELLHPDALYAQPRCPPKRAAVPGGGARPKVSAFSARLQYVGRQSFGDRETRQLTGAQFSSESEHEYAEIPERTTRPVESGDKRHFTSGRRGGISGPSSSKPSNGAGLTRKTKTSLSVSLKNLLRIKDDDVKVDVPRPVTVPVHLMQPHPMTEYRNAFLIYLEGVMGVGKTTLLNSMTGMVPQENVLSCPEPMKFWTCVYSNCLKEQRSIVKQGTHGKSITSARVYACQSKFALPFRATAAGIGRNLQPWLVGNGSTKPANWIVFDRHLLSATVVFPLVHVKYNRLTPDHLFQILSLFSAHDGDVVVLLTLNSSEAHRRIQSRGRKEEKGITQNYLRQVAWAYHAVFCTWVMMQYLTPEQMVQLCVQTVSIEDICNMNSRLTHRFLTLTKLHEQSMIPMVVEMLAAVKEHVTLMEVCLGLFKELRKLQILIVDAGEHLDDTCGLWGNIYGQVMSNEAIKPRAVNWPALESYIQTLTSLESNAAN

SEQ ID NO: 36 - protein, 118 aa, Japanese Macaque Herpesvirus
MTYPRLRPERRGNYCTRTLCMRSRGAHLKGRPCRAVVRGPRCPRSRLDFNMLDDRVLGIERRGSLPGPSFLPRASTNMRKYRSVLHARLKAGTRDILPLGVGVEFRDPRRQNLVTVRG

SEQ ID NO: 37 - protein, 82 aa, Japanese Macaque Herpesvirus
MSLVPAFNRACSTLRYFRIFVLALGRKLGPGKLPRLSIPKTLSSNILKSSRERGHLGPRTTARHGRPFRWAPRLRIQSVRVQ

SEQ ID NO: 38 - protein, 109 aa, Japanese Macaque Herpesvirus
MFNFKIPFLFCNVSCNVSRIFELAQASCITREVNTSLTFFQLIRYRVLLNSSSTVDISLLTGVVVAGVKRTISSPGTFSSLYSYPLITENVITNKHKPWLIGCIAFQAG

SEQ ID NO: 39 - protein, 726 aa, Japanese Macaque Herpesvirus
MQPINHGLCLLVITFSVINGYEYNEENVPGLEIVLFTPATTTPVKSDISTVELEFNRTRYRINWKNVSEVLTSRVIQDAWASSKILETLQETLQKRKGILKLNMAYVSNKEINFCKSFSSSEIINITSDFNYSSLPGFLGSFDVINHELTHGVLVSKRELFTNALDIMGLFYAIKLNAETFKITFDESKVIISGIMTADWILVSIANSSSEVNGQCVALLFGDPEKFPPLKGYVSYRDLVVVRNDNYAIAVIAPMSHGTMGLNFLPQNLTDIFFSILNSPLKVINYLKGKLFELESKGACQNPSNEQNILSLFFEVTAIHFLYVRNLNKEEPVNVGCVVKHVAALKSLERLFKLCFPAFELHSLNLWTLSHLAASQIANLPPNNVMSLTMDEQEVVFSMFKLADNNNVNEIILNEIINISDQMYTMYSDIYQLSNTYRQTVMDIYEVLTTVSLTNVGARAVYPYILFTSMCNNVEISYMINQISKPDDITIFRVFSPCFLSLRFDLDENKLRSDAPQTSKRTGSELAQGASGFWRLLHAFHATRINEFSVINCTRLAWKQVTALMPLTNITYVISSVRPDHARVYEVSEVFLNSAMFVSAVYPNCSHFTPPGTALHIPILYNFSAPRIGCPLCDSIVLSYDENQGLQTMMYVSNPQVQANLFSPYSPFFDNDNFHIHYLWLMNNGTVVEIRGLYRRHALSAIAFVFAFIGTMSALYFLFKLFSILA

SEQ ID NO: 40 - protein, 99 aa, Japanese Macaque Herpesvirus
MFIISFKMISFTLLLSASLNIENTTSCSSIVNDITLFGGKLAICDAARCDNVHKLSECNSNAGKHNLNNLSKLFKAATCFTTHPTFTGSSLFKFLTYKK

SEQ ID NO: 41 - protein, 187 aa, Japanese Macaque Herpesvirus
MGSRQPQIGESPITAFNTVTIMQRANNSIMFLPNLKLKPIQHLFLKHVLLQRLGLENILFHFKMLYANTCKAAAPYQREYFESMLSRVKQRLEDMVFCLNSIESHNFQKDFKVTSRAPQQLLTATDKYFLMFPPQNRDLAIQVGAEVIESICDGTPLFEVLANLNPRVTIQKETGNNLLKFYALLTV

SEQ ID NO: 42 - protein, 103 aa, Japanese Macaque Herpesvirus
MIVTVLNAVIGLSPICGWREPMAKNMSASKQSDCTSESLVAGGCLRMDRSFLSSDVKGALHKVGGAFGSYSCVVFASSFMVICLLFVRVTGGVLTVTWTENGL

SEQ ID NO: 43 - protein, 128 aa, Japanese Macaque Herpesvirus
MIKIPDLKARLVGGAVQLSNGEYVCHVVYSSALAAMVGLPGPAVPLPLLFKKFGTIYSNMMPLYAPKRPELSMLRIMVSPHPYALNSCLCVGTDEGERGVSLFRDPVIRSSDFDDTPITVRLKISDRE

SEQ ID NO: 44 - protein, 733 aa, Japanese Macaque Herpesvirus
MMLLQGPVLLPACPATATADTPSPANSDFKTQLAIFCCLATNNEILENVSLEVLDRAMETETTFYACRALRRLVLGEGLYPFIHRQGGIVGKTGNEYAGPGLIIDDAIGCTFSHIETHTFLPTVFTYELSDTVWVQRDERILRSLYCSPLMVCGVNYQSMFRILCRYLQIWEFEECFAAFTRTLPEHLIGTCYQNYFKLLEPFKTLTLARCPPPCAKLHLNYLKFNILGFTSDWISHPELHRVQTVIIYNIESNPVLLKNLSKQNKFQDIRVASELIIDYQNIVNQSLGVNLQVKINKKDPGKKPYKVVVVTPKSTYYLTFPPEVPIFRVAMCMSVAEHVCHSCDRLYPNTEFLGPGETPRVLEAMFSRIQYAPKDRDHNFIFDANQNPDRHKQVHHDHQTEPLPDMFDPVKHLSLHNFKISVFNTNMVINTKITCRSLTGTFESIIDIPRLTNNFVMKKFSVKEPSFTVSVFYSDNLCNGAAINVNISGDMLHFMFAMGNLRCFLPVKHIFPVSIANWNSTLDLHGLENQYIVRRGRRDVFWTTNFPSVVSSKDGCNVSWFKAATATISKIYGRPLLKKLSDELDPILSVPYARIDQVKNTIFTTLETRNKAQIQTLHKRFIECLVECCSFLRLDLGALNRAARLGAFDFSKRIISHTKSKHECAILGYKKCNLIPKIYVRSKKIRLDELGRNANFMSFIATTGHAFSNLKPQVIRHTIRRLGLHWRHKAKI

SEQ ID NO: 45 - protein, 88 aa, Japanese Macaque Herpesvirus
MYIFPHRNAYVFTHRLHLRTVRHGLGSTRRTNPTVPVLFAINGMWRKLSKHVSNSLSLPSNMGIRGMLCRLHAYTAGTSDRHVLSELL

SEQ ID NO: 46 - protein, 1378 aa, Japanese Macaque Herpesvirus
MEAALEVRPFPYMATEANLLRQMKESAASGLFKSFQLLLGKDAREGGVQFEGLLGVYTNVIQFVKFLETSLAVACVNTEFKDLKRMTDGKIQFKVSVPTIAYGDGRRPTKQKQYIIMKACNKHHIGAEIELSTDDIELLFIDRETPLDYTEYAGAVKTITASLQFGVDALERGLVDTVLNVKLRSAPPMFILKTLSDPVYTERGLKKAVKSDMVSMFKSYLMDNSFFLDKSDIAVKGKQYVLSVLSDMVGAVCHETVFKGTNTYLSASGEPIAGVMETTENVMRKLLNMLGQVDGGMSGPASYANYVVRGENLVTAVTYGRVMRTFDQFMKRIVDRPNAQPNVDDDRDAVADGQDSLAKTPIAAAVIQIGDKLVALESLQRMYNETQFPFPLNRRMHYTYFFPIGLHMPRPQYSTSATIKGVENPAEQSVETWIVNKNNVLLSFNYQNALKSICHPRMHNPIPCGQALGQAFPDPGHVHRYGQKSEHPPNMNLYGLVYNYYQGKNVAHVPDIALKATMTTDELLHPTSHETLRLEVHPMFDFFVHQQPGAQAAYRATHRTMVGNIPQPLAPNEFQNSRGLQFDRAAAVAHVLDQSTMEIIQDTAFDTSYPLLCYVIECLVHGQEDKFLINSPLIALTIETYWNNAGKLAFINSFPMLRFICVHLGNGSISKDVYAHYRKVFGELVVLQQALSKIAGHEVVGRRPASELINCLQDPNLLPPFAYNDVFTNLLRQSSRHPMVLIGDEGYETENDRDTYINVRGKMEDLVGDMVNIYETRNNADHDGRHVLDVGPFNENEQHMAVLEKLFYYVVLPACANGHVCGMGVDFDNVALALTYNGPVFADVVNPDDEILDHLENGTLREMLEASDIHPTVDMIRTLCTSFLTCPFVTQASRVVTQRDPAQLLTTHDDGRYVSQTVLVNGFAAFAIADRSRDVAETMFYPVPFTKLYSDPLVAATLHPLVANYVTRLPAQRVPVAFNVPPALMAEYEEWHKSPILAYANTCPITPTSLSTLTSMHMKLSAPGFICHAKHKIHPGFAMTAVRTDEVLAENLLFSARASTSMFLGQPSVMRREVRADAVTFEVNHELASLDMALGYSSTITPAHVAAITSDMGVHCQDLFLMFPGDSYQDRTLNDYVKQKAGCQRFGGPGQIREPVAYVAGVPHSDNIPGLSHGQLATCEIVLTPVTADVTYFQTPNSPRGRASCVISCDAYNNESAERLLFDHSIPDSAYEYRTTVNPWASQQGSLGDVLYNSTSRQVAVPGMYSPCRQFFHKDAILRNNRGLNTLVTEYAARLTGTPATSATDLQYVVVNGTDVFLEQPCQFLQEAFPTLAASHRALLDEYMSNKLTHAPVHMGHYMIEEVAPMKRLLKIGNKVAY

SEQ ID NO: 47 - protein, 107 aa, Japanese Macaque Herpesvirus
MSCCTRPRTRRFVWRFIRCLIFLFISSQVRKPRIGLPTGLWWVTFRNHWRPTSFKTAEACSLTERRPWLTCWTSQPWKLSKIRRLTRHTHCFVMSLNASSTDRKTNF

SEQ ID NO: 48 - protein, 146 aa, Japanese Macaque Herpesvirus
MIRVVSGFVNVNHVTDEVLHFSSDVNVRIPVIFRFVPLVSYEYHRVPRGLPKQVGKNVIVSKGRQKIGVLKTVNQLGRWPASHHLVASNFRERLLQNNELAKNFSVMGVHVLRNTTVAQVNADKSQHREAVNKRQFSGVVPVGFNG

SEQ ID NO: 49 - protein, 83 aa, Japanese Macaque Herpesvirus
MTGIRTLTSEEKWRTSSVTWLTFTKPETTRIMTAATSLTSVPLTKTNSTWRCWKSSFITWSCQPVPTATSAAWASILTTWPWP

SEQ ID NO: 50 - protein, 305 aa, Japanese Macaque Herpesvirus
MALDKSIVVSVTSRLFADEIANLQSKIGCILPLRDAHRLQNIQALGLGNLCSRDSAVDFIQAYHYLDKCTLAVLEEVGPNSLRLTRIDPMDNYQIKNAYQPAFHWDNYSELVVIPPVFGRKDATVSLESNGFDVVFPAVVPEPLAQTVLQKLLLYNIYYRVAETTPTDVNLAEVTLYTTNITYMGRNYALDMDPVGSSSAMRMLDDLSIYLCVLSALIPRGCVRLLTSLVRHNKHELVEIFEGVVPPEVQALDLNNVSVADDITRMGALMTYLRSLSSIFNLGRRLHVYAFSSDTNTASCWCAYN

SEQ ID NO: 51 - protein, 274 aa, Japanese Macaque Herpesvirus
MSIPKIMTVSRDNEGTVCEVAVDNGRHRAMIYYPKITNSSNERAAQRADVVKEAFDTETPVDIVKQIVNEGLAISKRNCVRLALYLYFYLQYVCFAILITWQLNPHIDPPGLVFAVNPMGPKHVSKLPHPAIVAVGCGTDAICKNCSVPDIKTELGVVYHNGSSDSGQSAHYGLALLKAAWLVMGNVCPEPVVRQGAELLGPWNRTAWLDFKSAMAATTFCGSRGVLWSPIHEKNLCRPTWNDVINTSSFFTNESLCPNVPGVSEIVIVLNGDA

SEQ ID NO: 52 - protein, 91 aa, Japanese Macaque Herpesvirus
MTAHTNGVLTTTGVSTSQPESFQISPFFRVITKPPIMGLFFCVAMCIIALVWYVMRRVYCKGRVVADSCRDPRRPAYEMLNVRLRPHGTNP

SEQ ID NO: 53 - protein, 348 aa, Japanese Macaque Herpesvirus
MLQKDAKLIFISSSNSSDKSTSFLLNLKDAHEKMLNVVSYVCPDHKDDFNLQDTVVACPCYRLHIPAYITIDETVRSTTNLFLDGAFSTELMGDAATSAQSMHKIVSDSSLSQLDLCRVESTSQDIQGAMKPFLHVYIDPAYTNNTDASGTGIGAVIAVNHKVIKCILLGVEHFFLRDLTGTAAYQIASCAAALIRAIVTLHPQILHVNVAVEGNSSQDAGVAIATVLNEICSVPLSFLHHADKNTLIRSPIYMLGPEKAKAFESFIYALNSGTFSASQTVVSHTIKLSFDPVAYLIDQIKAIRCIPLKDGGHTYCAKQKTMSDDVLVATVMAHYMATNDKFVFKSLE

SEQ ID NO: 54 - protein, 217 aa, Japanese Macaque Herpesvirus
MSFLYHNRCKECQMTRVNSPICQFHNVSSLYQCLDCKRYHVCDGGRDCVIVYTRENLVCDLTGNCVLDNVQDVCSYGPPERRAPDAFIDPLVSHGTRESLKSDILRYFETVGVKSEAYSTVVKNGQLNGIIGRLIDATFNECLPVMSDGEGGRDLAASIYIHIIISIYSTKTVYDNLLFKCTRNKKYDHIVKTIRAQWMRMVSTGDPSRVNATGCFT

SEQ ID NO: 55 - protein, 119 aa, Japanese Macaque Herpesvirus
MLAARSLPPSPSLITGRHSLNVASINLPMMPFSCPFLTTVEYASDFTPTVSKYLNISLLRLSLVPCDTRGSMNASGARRSGGPYEHTSCTLSKTQFPVKSHTRFSRVYTMTQSRPPSHT

SEQ ID NO: 56 - protein, 461 aa, Japanese Macaque Herpesvirus
MDAHGLNRRSVAGQCDGLFHVILPRGFILANNITCGGRQRFFAHTRFAASERTSKTLYVWGRVFQNTDPGSGDGPSGPWSGLAISLPLFTTNGKFHPFDVVILRAETPGSGSSWTVKFLYMSLIAAYRNAMRGLKDKVSQSTDAAVDGEVHPLTVLKEALVSPDTATRPVSACNPLQMLTGLLQSRVRDDYVTHHRALERPGNVRGQVSAPTRTEMPNGSPSRVRLGFRPPKQANYPKTWAQARHVFSSRTYYVCVYDNEELDTKWQRQDPRPLSLDWSDPVAYLLEGDLFLGAKQNAFVDSLEKTCRCQNYTIKQFFPVSINRDNDTVDLIKEHFIEACLVIRNQASERSAWVKAALFRNDSNTYWKDVLGLWEHGPHKLGTAIKRPTSEPCNADIDWSWLLCDADITRSINGQSTVCLVVSPALIAWLVLPGGFVIKGRYDLSSEDLMFVASRYGHPAA

SEQ ID NO: 57 - protein, 336 aa, Japanese Macaque Herpesvirus
MATQRRDILKSFLNKECIWLRHPGTSAFVRVYTATTAHSAVFDPPVTSEDAMSHNCLNVMIMLMKPKEFGPCVTVYINGDILDFCATEYVAIREVPGRADLCLIRFGTLSNAPRSVPIPGPLNPHPRENVPGLTKQEIIYTSQTVPRAQIQDAIKGKAFKQINPFVWFDGGAFWQLFLSVDYMLLCPALEIVPSLARIVGLLTQCDKSTCKICTLAHVHVNAYRGYTPPDSQGTSPSCPCLISCGARHATDVLVTGHVNLLGLLFDPKVLPKVSRLRLKRNPHPVPIEDAMSGVTAEGTEVLPTSQPWALIRLPDLASRVMLYGCQNLKTICLRSY

SEQ ID NO: 58 - protein, 122 aa, Japanese Macaque Herpesvirus
MASCICALGTVWEVYIISCFVSPGTFSRGCGFNGPGIGTLLGALERVPKRIKHRSALPGTSLMATYSVAQKSRISPFMYTVTHGPNSFGFMSIIMTFKQLCDIASSLVTGGSKTAECAVVAV

SEQ ID NO: 59 - protein, 129 aa, Japanese Macaque Herpesvirus
MRAHGCEVGSTSVPSAVTPDMASSIGTGWGFLFKRSLDTLGSTFGSKSRPKRFTCPVTRTSVACLAPHEIRQGHEGEVPCESGGVYPRYALTCTWASVHILHVLLSHCVRRPTILARDGTISRAGQSSI

SEQ ID NO: 60 - protein, 327 aa, Japanese Macaque Herpesvirus
MLLTSYRERLQNNLRAVTDGGCENWFRQPPVIISGNDKTERMAHPCLGVIHAVNAYSSVLDDYLQTYRRLQEPMPPPTLGKPRISSHATLPRLTEELTNYLRQTCCRVRMADAKDQHMEYQSAQRTHEAFLECPVYAELRQFLANLSSFLNGSYVPGVCCLEPFQQQLIMHTFYFIASIKAPEKTHQLFATFKQHFGLFETTDDVLQTFKQKASVFVIPRRHGKTWIVVAIISVLLSSVENVHVGYVAHQKHVANAVFSEVIATLSRWFPAKNLNIKKENGTIVYASPGRRPSSLMCATCFNKNVSRLFLNSGSRIALHDWLNPAGE

SEQ ID NO: 61 - protein, 88 aa, Japanese Macaque Herpesvirus
MLSGPNGRRQGPAHGIPIGPTDPRSFPRVSCLCRTAAIFSQPVVIFKWELRARGLLPRALSATVNHAHVLFYRVYQSTRKDTPVICHV

SEQ ID NO: 62 - protein, 328 aa, Japanese Macaque Herpesvirus
MFPSSFLNNENPETGRRFVKGVQLALDLCDNTPGQFKLVETPLNSFLLVSNVLPESRPVRDCPQAEGFDFEHIHLPKLTRMQRVLGRYCDHVNNDDDMCVNVKARSSNAQGALFYLPYGQDEWNWALTLRKDKLVKMAVEGLSDPTTWKGLEPVDPLPLIWLLFYGPRSFCREPECLYERNFGMKGPILLPPHMYAPRKDVMTFVHHVIKYVKFLYVNAGGGLETELSPPFEASRLRSAIARLGDVEADDAYLSAKCMLCHLYKQNDTISIHETHVGGVIALGGDGARYITSSVRTQRCTSRGDFVLIPLYNIEGLVSMIREHGLSNS

SEQ ID NO: 63 - protein, 148 aa, Japanese Macaque Herpesvirus
MWSFGDSSSTGTDSSLSVPRISSSPYSSSLRSRTASVTASNSSIFFLLNPFSNPNSLCFSRSDVSSVPTTVARLSFFFRLSYDRSDSLVPLYCALNTALSAPNLSKTEMDSFLLISDSSSDLINNFLAVAEAMFPYHAYEPFNVVQRD

SEQ ID NO: 64 - protein, 149 aa, Japanese Macaque Herpesvirus
MASATAKKLLIKSELESEINKKLSISVFDRFGADSAVFNAQYKGTRESLRSYDSLKKKDNLATVVGTLETSLREKQSELGLLKGFNRKKIEEFDAVTDAVRDLKDELYGELEILGTLNDESVPVEEESPKDHIIRWKLERLPRVCPKSP

SEQ ID NO: 65 - protein, 435 aa, Japanese Macaque Herpesvirus
MNLFPWKKSPQRTTLLDGNWSVCQECAPKALDPIPKVQTDLDRTALSHITVIRTRKTLAQLKIPNTWSQCSHQATDWTAVLGRGSYGVVRSMSLGRCVKHFGSRREFFYECIFNDIVRARREKHPLNRGGDRILCFLEPCVPCRALIFPQLTGNLLNADFKHVNPERLAVEFSELREGVSFLNNICGIVHCDISPENILIKGELTTAYWRLMIGDLGSASLHTGTPWTGVMVTSKLGFVQHTYHFKAPARFICKHMYRPSCLLYRCLLSCAGGPQARMLDQPFQITPQLGLTIDMSSLGYSLLACLEKYLQPADPFPQQGALADASSESAHPLFYLRCMVPRVVIAEIFSVAWDVPLDLGIDSSGHAPAIPLREAYRRFFANQCSLYRAQYKEDALENASSRLCNSKLKLVLQKLLVRDYFSHCGNCGDHGFFLR

SEQ ID NO: 66 - protein, 480 aa, Japanese Macaque Herpesvirus
MDFFSDEPMVQEMALLDIDEQQRHLSKMSLANFLKHERVRAFFNDNKKKISMPAIRFVYNFYLFAKVGDFIGNTEVYDFYVSCVFRGRRLTLLSDVYDACLNMHPHDRHHVCALIEQVTRGQNINPLWDALRDGIISSSKFHWAIKQQNSSKKIFNPWPIVNNHFIAGPLAFGLRCEDVVKKILATLLHPGEAHCENYGFMQSPHNGVFGVSLDFGINVKSNPKDGLEFHPDCKIYEIKCRFKYTFSKMECDPIYAAYAKLYQKPSMQTLKGFLYSISKPAIEFVGEDKLPSEADYLVAYDKEWEVCPRKKRRLTAVHHLVKKCMIHNSTAPSDVYILSDPQETGGQINIKAHLSANLFINVRHPYYYQVLLQSLVVQEYISLSKGTKNLGTQKNFIATGFFRKRQFQDPSNCTIGEFAPLDPHVEIPTLLIVTPVYFPSVAKHQLVKQATEFWAASASEAFPELPWDLSSLCANAPPTP

SEQ ID NO: 67 - protein, 378 aa, Japanese Macaque Herpesvirus
MKISRSDSFILSSWVKLLVILGLMFIMSAVVPLTATFPGLGFPCYFNTLVNYSALNLTVRSSAKHLTPTLFLEAPEMFVYISWAFLVDGYLLCYYAWAILAIFKAKRVHATTMTSLQTWIVLIGSHSVVFMSILRLWTIQLFIHVLSYKHILLASFVYCIHFCLSFTHVQAMISCNSATWSLRVLEQQIPENSLLDTLLRYGKPIGANLYLSLIAMEMLVFSLGTMMAIGNSFYMLVSDIVFGSINLFFVLTVAWYINTELFLVKYLKHQIGFYVGVFVSYLILLLPVVRYDKVFISASLHKVIAVNISMIPITCILAIILRIIRNDWKWCAKAPEYAPLPQGSKEKTTKVKYSPELNALYETEEDVSDDEDAYPKYI

SEQ ID NO: 68 - protein, 104 aa, Japanese Macaque Herpesvirus
MYTVYKRGQKYMFVRQHVNKQLNGPQPQNRHKNYAVRTYEHDPGLEAGHCRRVHALGLEYGQDGPRVVTQQISVHQKRPRDVNKHFWCFQKQRRRQVFSRTSDR

SEQ ID NO: 69 - protein, 667 aa, Japanese Macaque Herpesvirus
MNAREVALTGHVLHISLHSTHEREKLIIWQVHLLVCQQCGIQGDAAYLFVTETLSNTDWGNIPAINRHAPSMNEHGRNYMQWELRTRLRNPIIQSLSRQPGAVNVRVSEPNMVIVSCERALHHSCSVRVTGAYLHCNTTMDFRLDSNVSPTREFWFSEMFSKCLVSNIEVYLKTTGGLYYRASSATQCRKRAKDGALGILDIFNCESREIQVAGQKYNLSIATATFHVLWVDEACMWNGALAEFFRALHNKLFGDREGVAPTLTYVCPGATPEGTPFPPYFSAFPHLLLVFGRPRRLDVTAVQELPKAQIAVHWPPFKNSILGDQLLIPGISPKKPGTVPVRWPLWVQDVNLSLCETTESVARIVDPHSIVIIKISTLLCQHLKCHRAFVKNELEYIATICSSDLRLFIQEEYNRLLATIFTWAAASGYTWAAIDKTTVFIKAPQLSAAVSGSCPSLNSCRRKQYYKGLKITVHFLSQEQQKVVTRLEAQLGLPVQETSRPPDWLKYEVCSASVFLKIPAGVLYAGLAKDPVSEAKRDSWLDCLVEGATLSLNNSVPPIGALAGILPTLFAKRRCVNFWLLPREWVKSAPICPPLPIDCVTPQQFVVTKRGPICWYKEWPLPVDVDFMYYLQEALCVFSVVSNGEGTESHADDIRQLEKFEKVLCLF

SEQ ID NO: 70 - protein, 116 aa, Japanese Macaque Herpesvirus
MNTVVLSMAAQVYPLAAAHVKIVASNRLYSSWMKRRRSLEQMVAMYSNSFLTNARWHFRCWHNSVEIFMITIECGSTMRATLSVVSQRLKLTSCTQSGQRTGTVPGFLGEMPGMRS

SEQ ID NO: 71 - protein, 271 aa, Japanese Macaque Herpesvirus
MDQILKRLMGEQHRSEAIMPETECASRGPYNYPVLPRLMLEVHKKNSICMASNTPKLCVRGRLNVPDLGVHVRTRLQSATFTGFVFACVVEHEDMVNTLDIYPHVFSDRVQLFKPASASVTELCCILSMLENYDKPPLSFILSALDRARYLHERYTCNDSAFILYGIEVIASTLAAYHELNPPQGVLRVPPLVRFKLHKLLDENADDMKGLLKPIYLESFRLTENVEEDSHGETFNIFYCGTIFTRHLHNASVLKYFQITSLHSLPRQTLF

SEQ ID NO: 72 - protein, 82 aa, Japanese Macaque Herpesvirus
MGFSKPFMSSAFSSNSLWSLNRTRGGTRKTPWGGFNSWYAAKVDAITSIPYNINAESLHVYLSCRYLARSSADRINDRGGLS

SEQ ID NO: 73 - protein, 581 aa, Japanese Macaque Herpesvirus
MFKMNPGLGSTCLVHPTELSISLFEILQGKYAYVRGQTLHSSLRNPGIFGRQLFIHLYKTALGSCTYDNVLKDWTNFETTLKTRWRGVEHLTPEFKRSTFESWARTVRLTVDQLLLNTINQVLHTRTVLSYERYVDWVVALGLVPIVRRAPDGETIARIQAHCQQMRKTHASGDVTISRIVDKLAQEITAIMTDVTSIYIPDYAEVSVEFNGDKAAYLGTYRQKDITVEVVSRPIIYNGRVSFDSPLYRLFTAIMTCHRTAEHAKLCQLLNTAPLKALVGSTCNDMYKDILARLEQSSQKTDPKRELLNLLIKLAENKTVSGITDVVEDFVTDVSQNIVDKNKLFGTGTESTTQGLRKQVSNTVFKCLTNQINEQFDTISNLEKERDDYVKKIQCIETQLLQSLPEGGRPRHDINILTQNTLQALSGLRDPTINLSECHIPKGSSVVNSFFSQYVPPFMEMLRELTSLWEGEMFQTYNLTPVVDNQGQRTSIAYSQDTVSILLGPFTYIIAKLTHMDLINHSLISLSLHDIADQLYVDSRLFVYINDIGHKYCEQIIQPGTDGPNTEAFNGGAAPIGGNNA

SEQ ID NO: 74 - protein, 790 aa, Japanese Macaque Herpesvirus
MESSVGWTKHVEPSPGFILNMTSDAKVRGVVDHVSRLSNITTSPPEMGWYDLAFDPAEDSGPFLPFTVYLITGTAGAGKSTSISALYQNLNCLITGATTIAAQNLSRRLKTFCPTIFSAFGFKSRHINIAVRKAHQTGAVSIEQVQQQELSKYWPVIVDIMKEVMAKKPNGMYGTISNADFETLSRMTGPCLWTSNIIVIDEAGTLSSYILTTVVFFYWFLNSWLNTPLYRQGAVPCIVCVGSPTQTNAFQSTYNHGTQKTEISSCENILTFMIGKKVVSEYVNLERNWALFINNKRCTDLQFGHLLKILEYNLPIPDEVMSYVDRFVVPKSKIMDPLEYIGWTRLFLSHSEVKAYLTNLHTCLTLGGDTRDTKLFTCPVVCEVFVKPFEEYKRAVNLTHLTVTEWVTKNLFKLSNYSQFVDQDMSIVATESTERSTQVTFITKFVKNSHVSLNGKTKKCICGFQGTYFEFKRILDSELFVETHSQDRPEYVYGFLNTLLYNAMYSFHAYGVTRAHEKYLEDLKFAPLPTALATGRVDFQTVREELNLEDDIFYHVCSPPPPAGITSLQVLVDTYCALKDVFASRIKVACRWFGGEFEKETFSAFTVNMVVRDGVDFVSPSERLNGLLAFASTVESYKIKGYTFLPIAFGRCQGLPLSDDLRKKMPSLVVQDSSGFIACLENNISKLTETMDDGSVFQVCCAGDYGVSSNLAMTIVKAQGMSLERVAVVFGAHKNVQTSHVYVAISRAVNSNYLVMDSNPLKTLLREPVDNTSAKHIVRALHNPNTTLIY

SEQ ID NO: 75 - protein, 137 aa, Japanese Macaque Herpesvirus
MVSVSLDMLFSRHAIKPLESCTTRDGIFFLRSSLSGNPWQRPNAIGKNVYPLILYDSTVDANANNPFRRSEGETKSTPSLTTMLTVNAENVSFSNSPPNQRHATFILEANTSFRAQYVSTKTWREVIPAGGGGLHTW

SEQ ID NO: 76 - protein, 353 aa, Japanese Macaque Herpesvirus
MAMFLSDPPRTPPATPRMLPIPGAPRKKRTRRFLFAGSRTGLPVPPGYGGPPVIDMTAPNDVFDADSPPTTPKTPDETDSHSENSNYSDMDEEDEQPVSSPPRIDPHARDGESFNQSDWRPTVITAAGPAAQPSAPAPLTAFGGQRPVAVVTGQHRAPPSSTSDSGDDFFIDDYEDTDESGEDADGFSPRASPAWSGDTSRSPAGGGWSSNEEEEPAVTGSAVEQETIIISDDDDTDDRGSVETWDESDADEGTGATDVIDLCSSSDSDDDADHVTSGGVRAACKRHASRRDCNGDDDVIYLGTTRAPKRRMTSTTGGGATSNPEGPGVSGRQTMAATPPVCGNDNYPWPWLD

SEQ ID NO: 77 - protein, 87 aa, Japanese Macaque Herpesvirus
MRVGFVWRFWSGGRRICIKNIVRCSHVNHRGASVTGRYRQPRTGPGEQKPSCSLFPRCTGNRQHPGSGRRGPWRVRQEHSHDQNVHL

SEQ ID NO: 78 - protein, 255 aa, Japanese Macaque Herpesvirus
MDTWLETVVWHKMSMTGPNETPTQMLLISDSWLKFLNLSPFLKKKLAALLRRVMDMSKATVIYPPIDRIMWWSYCCEPEDIKVVILGQDPYHRGQATGLAFSVAPDYSIPPSLKNIFKEIANTVPGFTAPSHGCLDCWAKRGVLLLNTILTVERGKAGSHSNLGWDWFTSYIISCLSAKLQRCVFMLWGRKAIDKAVLINGQRHLVLKARHPSPLAAAHAATGSPWPQFLGCNHFKLANDYLVQNQRGAVDWNIN

SEQ ID NO: 79 - protein, 163 aa, Japanese Macaque Herpesvirus
MYTLSFFTTYFILYIGYSSGLIPNPCCDIVPLTGVNIPAPFEIVSFHFTDLAWCQGRCVATLRYKVGTITTELCVNGFHLRAFFIRILSGLDFSVHREELDLLNYVRISLEDFLSAFKDTHDNSESVTNLPAVPDLTKKGSAAFRTRKVGARRGDLWILGSRQ

SEQ ID NO: 80 - protein, 389 aa, Japanese Macaque Herpesvirus
MAVSIPVQGVDRETESNWRSIVTTFEQHGNADRAIRSLLRFFKGVDHPGFLASLVILKDVAIDSEKTIERTDIIPLLQGVRFVTQQIYMHLKDHASESPVTEIWRDCKERFCLALELACGCQSCTSAARQLRDCQQACRPPKLNPHKQQCGAARLLTAVYNQMVLRTRVSVSEFCLNALMCVPREFGFVSGDVRVETSRVASCMNLSWLYLILDSYIRTDLTNLEMAMSRACRIRGLSTRDPFYSALVWLKNSCACAANTFFFTVNSTRVTTPILMDICASLAGPVPDVIKINMLPLVNNQMYPSVCVERANFTGSCPKMSPTHRFDGLKLETTSLTLAADSLDDILQALELICDDDEGILDSHISDIDTETEVDESTIEEEIVFEELS

SEQ ID NO: 81 - protein, 83 aa, Japanese Macaque Herpesvirus
MVDPFKKSQKRPDGSIRVSMLFKGSHDGSPVRLGLPVNTLNWNGNSHDTVNFLMSPKTNPETELNTNDCHPYPKPRPRTIRPG

SEQ ID NO: 82 - protein, 81 aa, Japanese Macaque Herpesvirus
MDTDDNQVIKLFFIQDSGGRVYSGVGGSISRRWEWLMNPHLVLGSGALCILLVQLNLNLACLGRTNQKTAWPRLLGALCKL

SEQ ID NO: 83 - protein, 301 aa, Japanese Macaque Herpesvirus
MSRHYGKDHLLNHMYKFHYPPLGMIVGEMNTLTVNARNPLYQAATLRVERALYLSKILQVLMQHRQGERFIVPQCRSNMVYCLKELHKITNDRIRGLINSVLPLVDAGCVGFDEELVRILPEILKLEYPHVHELLPPHDPTSPLSWCLSHMVGVTKTFKGEVKEMIDTFHDLSVPSFQYLASLVKKFFLVEEVIYEDYQDTQFNVFLNLCFFWTTVIKMYQSCIFKDKLLDTIKACIELLKGEARQFFGWYDLNTPNLGSAALVKYTEHLIRALSVDSSAIPIGEICSHLHHCKHALLNLE

SEQ ID NO: 84 - protein, 85 aa, Japanese Macaque Herpesvirus
MGMADESTLSARIRCSVYFTSAAEPKFGVFRSYQPKNCLASPFRSSMQALMVSNSLSLKMQLWYIFMTVVQKKQRFKNTLNCVSW

SEQ ID NO: 85 - protein, 514 aa, Japanese Macaque Herpesvirus
MECASLGPISGLIADLNLFNLFCLYRGSRVKTRGAATCNVPCAECAQGVVRILTERALCCTEKMFIASACSGVVLPPQLAKVFHDVYAEMKAKCLGAWRRLICCRRPIMAIADSVLVTYNTLDAEGKLDLKLKALCKLVFQPIFLQRILAPMQLLANGKMVPDNYFTITGTAEKRRPVVTGSTSGMTCPGSSLVPDSLILPVCEPGLLPAPLVDLSNVLENPEIILSAPPLSQFVITNTHPSLPQSVSIITPTQGVVPGQCFMDTWKAVSQSIHHQAHTPIFAATLAGSTSAAPGPHIACSPVAGTSRQVEASAGVDCGKPVCAPQPDIPPHVPTKRMETVAQSGNAPVKNVHIGGRVYAPLVNIPIIDLTSPSGSGQSPVNIANTPESRMAAGSPPFAETAATVPAKRKQPREDVADKRSKGDGRGAATVNPPFPGPPAMRVREQGLFDLIESSTDVTANASGPKNDDDMLAAILQDLYGLQSPPAIDSPSSNSDNEDIFPEVSPPSSGHGSP

SEQ ID NO: 86 - protein, 224 aa, Japanese Macaque Herpesvirus
MSSLSELLEGESIAGGDCRPYRSCRIAANMSSSFFGPDAFAVTSVELSIKSNKPCSRTRIAGGPGNGGFTVAAPRPSPFDLLSATSSRGCFLLAGTVAAVSAKGGEPAAMRDSGVLAILTGLCPEPDGDVKSIIGILTSGAYTRPPMWTFFTGAFPDCATVSILLVGTWGGISGCGAHTGFPQSTPADASTCRDVPATGEHAICGPGAADVEPASVAAKIGVCA

SEQ ID NO: 87 - protein, 170 aa, Japanese Macaque Herpesvirus
MPRVKTQPKRPQVFDFMPLDLHGETRAEMDSQSLCPDGQDLLGSYIYTENNGPFSQMMHNGQSNNGTGESFGSYAAGDGFLGGSVSGMHGNGTVDGLCSKKQSACRKRSAALIHAASEASVAEQGTSQGTNAVSDRIGRDGGIGNKLLKVSARLPDKTKTLPDPSLHCYF

SEQ ID NO: 88 - protein, 244 aa, Japanese Macaque Herpesvirus
MRIYGIKGLNFFGIMGFWNIPLGWGLCFMVWVAWIARGRSVCPTWHLTDGKYEAVYRHYLEECRKHEGSGSPDGSGKTEGSGTKATTEANISIRPNVVTSGQDKEPMKTAPRAESSHDLPRIEQVNALHLSTPELAQPLPVVESTPRESQSGGTPWDARPHAFIMHTNDMLNPSVVLSFRAVRARSIRDTEQSIRDRNTVTTSYRTPGRPSLVQARPSSYGARLPPSPRTMARYAETRPTDDQN

SEQ ID NO: 89 - protein, 92 aa, Japanese Macaque Herpesvirus
MDCSVSRMERARTALKDRTTDGLSMSFVCIMNAWGRASQGVPPDCDSRGVDSTTGRGCANSGVDKWRALTCSMRGRSCDDSALGAVFIGSLS

SEQ ID NO: 90 - protein, 139 aa, Japanese Macaque Herpesvirus
MSSTRPKTRAPKKELTMEELAAQVQKLSVENKQLKKLINSGDPTRSGSDPVISNSEKEAKIAAAVSALCNVATRKIEAKVRAVTAKAVTRGQVEEALAGINIRVDVSMDETTRGGITASADGALRRRRAQSRTRNNDAD

SEQ ID NO: 91 - protein, 104 aa, Japanese Macaque Herpesvirus
MTGSIVLALALTACLYLCLPVCATVTTSSTTGTGTPPVTTTPSAAPSVTPSFYDYDCSADTYQPVLSSFSSIWAVINSVLVAVATFLYLTYMCFFKFVETVAHE

SEQ ID NO: 92 - protein, 290 aa, Japanese Macaque Herpesvirus
MAEVTAHTVAYAFDSCKFEIIPKNNSSRIALRNKFPVVVKPGEPLVVPLGLRIIRAPQCAFFLSGAPTDEVYYHTGLIDQGYRGEIKLIVLNKTKQSVTLYRGEVNVSLIAFMYASPGPLKCPILNLPHYSLDAGFDVTSPHAMTIPPTDRTPFTLSLYYKSPQLSTPHVPLIVGRSGLAAKGLTVDATKWTQSLVHLRFYNFTKEPIDIPANSRICQVVFIHEDHVPSGWNILRSRVQLGSTLQISWAKIRFTDVATLPATHPLNSRPTQSQTEPETNRGAKGLGSSGL

SEQ ID NO: 93 - protein, 140 aa, Japanese Macaque Herpesvirus
MSGSVYSRRPRPKRVEHSEIPRTTRQHPPDIVGQNQVHRRGHASRNPPAQLPPHSKPDRTRDQPRRKGVGVVRVIILKHGIILKLFIFNKTIQININHQPGHLVLFCLARYYPPPPTGASVVESPDGRGWRTLRYLPAHY

SEQ ID NO: 94 - protein, 210 aa, Japanese Macaque Herpesvirus
MAAPGSFWTCCGFSPFGRVGCRYRPLPDPLNECPTHWRTEIAMGLPPGVDMGDVKQAEMCTAALRQTYLLAVQSNKITEYLRRFDAARVPAGCQETVRIQISKLKSIQNVIWNAMLSLAIGDITVDESAFHALLNKRADETVSLLEMEKLATTIASDDSVTWAAEINNVLVDTEASSNPSHPVIRQPTPQLAVADNIVPDKIIQDAQADG

SEQ ID NO: 95 - protein, 838 aa, Japanese Macaque Herpesvirus
MSQVRPLPDERVNEIRAIFSTSGDMAEVITDILTGTQATASFFCVLHDRGNVPINTPHAVIKLCLPARRPGGGPRCLPLMVLNLPAWQVHLFLTGDAPLTSDNIKDRIDLAQTEEILEPILSVLACKRSAQQTKHDSFKSKVAWFRAKFVSALRKVYKMTPSPYWMITLLGSFEASFVLAGTFYFFQSYTCTAETLVHLTRLFISSQGQSLVTVNTYDELGRVFGRSDFLEIVPNFWAYLKYKMQQDDVESKAIDQTINSIRGGLMLSPQDLVHFIYLSFYECMNAQTFLSYSRTTASLPTPATVNPPQLCRRLEADFKEHVMAYYNKASYLSTYITILTVPAPLPEGYENFQELACQYWCGQSRDVAEIMTRINDQYPQLNLTKDLSGLLDLAALDQDSGGPKENLFTVASRIPTYRCEFLNKQYFVLMHTDCIDAYWKQNIIVPEDAQLQGLTDQDLTSRIFYCDLGLSLPTFKQQIMVSRHEYFNPRLPVYRWVLDFDLKVTEGRRTLNDIYNICVTLRQVILETLQLIGPLKPHHPVYFFKSACPAVTWPDDISDTAFCHCDTKIGMRIVTPFPIGYCLVGSAPLVSLTNILNRVVKLDTRLASEYPGILEDKGPFDSGIYAKGRCVRVPHCYKVGPGGELSRLLKIIICHPEESDKSAYLKNAFKVSNLLHHAPGDSVTKNGHLVYAIADENEGFLESKTKNNLPKTITDLAEKIERTTEKPLIDWAATAVWPKLHDTIQRFFPDDRIGQFASVSFMHSGDNIIQVRPQKGNNFFCINHKHRNHTQTVRVFLTLHSTKESEVTVTFMSQCFAAKCNHNSPTAHFSFMVPITCT

SEQ ID NO: 96 - protein, 91 aa, Japanese Macaque Herpesvirus
MLSPECMKLTDANWPMRSSGKKRCMVSCSFGHTAVAAQSISGFSVVRSIFSAKSVIVFGRLFLVLLSRKPSFSSAIAYTRWPFLVTESPGA

SEQ ID NO: 97 - protein, 89 aa, Japanese Macaque Herpesvirus
MAQAMVSLEYMKDILDGKKTSYGSYNFYLKPQLTKRLVLYALHAVRVSRTNLFYRGHIIIGLRRLLIGRDGRPVPDAGARTVHGTAQLS

SEQ ID NO: 98 - protein, 440 aa, Japanese Macaque Herpesvirus
MRYVFHALICFIGGISSSDFDDSSSDEMDDLSPTPEPEPSTAPHSFPEGPKSKVVALPKIRKRSRSKTPVKIEHRSPLNRSRSRSRTRSGSGQRTSSTYVKRFKPTVDAPRSREPWHRGGKGKAPFIRRNALAERGRRTYGNEHRGKSALTRNIKESIKKMHLPSTMLSRPHDKKVFEGLLPRHLEQCFQVCLPAPPPLRPEVFTDRQLTAIVKSGGRRDALVAKKVSLAKVMSMYKPLLTFVTGRNNQAHWLATRKNTLASAGLEALAAFIEEGLAWAQVCVSQNRSLNDSNLDIILDSSQSVCTWFISKIRHLHIQCFLENQGEISLVKQLTYLVCINNRLAEAANLAGEVKLNFKFAMLIGFALTLPALLAEQKLSGESLYLFRSFLEKYRPGDVMGLLNSIVVEHYTKCRSAECVITTRAMVGSGENNKGLFFFPA

SEQ ID NO: 99 - protein, 413 aa, Japanese Macaque Herpesvirus
MATWCPPHSGGPSAMGLREWIVTHANLGTYSGLFWADDEKTRVVLATTTAWTVEFDYPRDGKVYEDYCNQRNIPLPSGRPRLCQAKARLLGAIRKSAYFVEEKNFLRRSFSFPTVVFRLRSNEEMSCRLCPRASGVAAELRGLRFRMFKRKGADDVGRVTEYTVKQLLGLLRTRHAGASTMTAPATEASATTAGEDGRQDESEGGAVALPEEHTPPLSVSSGLSACLAPSVDDPWGFMHIQVYYYGFLQSQVFTRTGMGVRLSTRPTDKNEHHVCMAHGPLQLWLPPAPHMDDDVMLSRLVNALNALEDGIVLSSCQYGIMMNGYGFLNLWFRGNTLNTLEPTRVPSGVGHRIFDTDDYITKLAQSPRPSDPGPPDPFAQIWVAAWSLYEEEDLSQAPICIIVHQREIYRHFE

SEQ ID NO: 100 - protein, 81 aa, Japanese Macaque Herpesvirus
MAPNTQKDRLIQIAAECVPRVTQPRPLHSRPAYRLHRRWLRTPRWPGRSWWRHRRDGYEAARAAALPCIQLPCPHRRRPCA

SEQ ID NO: 101 - protein, 138 aa, Japanese Macaque Herpesvirus
MRLPVGADVCSRHEIIPPAATNSLSAQSAGYRLISLTSTPPHHLHRSCPCPDCPLNLVRSRPALLPGQAIDSSPLRDLPYGRNRRHEPLMFQLSRGKNNPRCRTQTRFRTRNAVLWRWVHGRDSATEPTDTGRFVHNL

SEQ ID NO: 102 - protein, 413 aa, Japanese Macaque Herpesvirus
MAGRGVDIKAWLVAAVESSEYHGLVWENEDKTVVRVPWNKVTAARSDSEKFFDDYCNMRGICQGEKPPHYGRFRKMRFLYDMRHHKSIRELKFINKAYGRSEARYRLFRLLPEPVVSCAMCNLMSSTETQCLGLTSEFRYDQWGGFGRERRRVFTATVLARSWMDKNKRVREHRLPGAIQLTFLYFGSTVGLERVHAGTRVCSRPYPVLAGHACCFQDERTLFLPPPGVVDCSFAREDLRGMHKKCEKGLLIALTDTGISVKNLENREMKVLTNNEEEYKDLPSRQPVQVFDMVDYLRALARSPKPGDDPPRDYAQIALCLSVQSPNPAHAPIAIRLRYVCETSSVCGTEGCFYPGTIGTSEARVAGSFHMEDPGEGTSQSLDPAVELGDSGPDSMDNPDTGTSGEDDGVACS

SEQ ID NO: 103 - protein, 91 aa, Japanese Macaque Herpesvirus
MGGIRRLNRQTQGDLCIISRWVVSRLRGPCKSSKIIHHIKHLNGLPGRQVLILLLIVSKHFHFPVLQVLYGNASIRKGNQQTLLALFVHAP

SEQ ID NO: 104 - protein, 351 aa, Japanese Macaque Herpesvirus
MERPVRVTKPSSLRGWLVECCETGRHPGMRWIDDERTLIRIPWNHDRGSRGVEESEKNIFIDYCRSRGILHAAGRELTAKECKNWLSSAIRHSQTVSDVSTNDDLSTPYPNRCRTIRLLPITVRSCARCDQASGTTAMLRGLREEAVNKFGPVGAGVQYTGAVGAGGEQCWMLRITFYYYGDRFGEVVTESPNGIRVLPLSERRAQGHICVAPIAEQALVPEIPSHLVEFQAEALRFLDKDLLRGLAFWADPSGIYIRWLGHSMAFVQGNVESPGAIVVLSCANACRAFNLVDYMKGMARTPRDGTAPPQACVYLYFGGVPTPEGGVQSTVPLIIQLWHECLWQALSAANV

SEQ ID NO: 105 - protein, 361 aa, Japanese Macaque Herpesvirus
MAIRDSNYSHLRSWTLYHLNEKTYSDLTWCDQEKKTFKLSWKKGAAGTPAVVAYCAQRGLQVGIDGNVFECKRRFLRGLRENAGFQECEHGVVRTHGGGWTAFRVKPLMDSGCFACILDENSEGIINYLEQVCGIGLEPGMPLPAPLPTLVPPTRSAYARAHRLGVPEAPLPHQIVPFWRLRIQVFYFGVLALDHTSQDRRGVRLHPRPVPRPGHLCFYGTGFTVWFPSPDPGKLTPDQITQINTMLVTYNEGIYVHGNETGVYVDNRNRETLYAAGNDCNGDIIQREVMFLSKQQIFYFMGFMRKLARSPVPESHAPCNGATLYLSQQPGAQESPQVPISVVVCQDELVQGQMNPSRWCA

SEQ ID NO: 106 - protein, 135 aa, Japanese Macaque Herpesvirus
MALWYRGAGKLSHETHEIKNLLFREEHDLSLNNITITVVTRSVKRFPVPIIDVHSGFIAVYVDALVIRDQHCVYLRYLIWSQFSRIWRWKPNGKPGAVKTQMTGTWNRARMQTNTSSILGRMIQCQNAKIKHLNS

SEQ ID NO: 107 - protein, 385 aa, Japanese Macaque Herpesvirus
MAAGESRRGPSRYGMALREWLISKADSGFYPGLFWADEHKTRLVLAATSPSLPNYDYQRDGQHYDAYCDLRHIPLPSGRGRLCQAKGRLLGAIRKSKYFDEDKDFPTDQFPFTAVVFRLRSFEEMSCPVCPRVDALRLELRNMRFAMLGRGMLHAFPESSVGDHEARHLEGQEGGDDARDDDAPYSSGVLRARLMACAAPSAGDPWGHMHIKIYYYGQLQAELSTATGQGIRLSSRPTNKSGHHVCVLDGPLQAWFPPIPQITDTSMGQRLEDALKWLVDGIIFCSTSRGIVFTITGAPNVWFQGNTVEPRNFPHRAYTGMHVWAFDTDRYLLDIARSPSPHDTDPPAAFVKLWVSGCSLGEERNVSRAPLSMTVHQTEIYRHFE

SEQ ID NO: 108 - protein, 81 aa, Japanese Macaque Herpesvirus
MTQLRPYCDKHRESGTRVLLWPFCQMNPTGNIYFLQYGAKVETEVLPVVPEAPPRTPDGPSSPLLLPVALSPVSTDTSSHT

SEQ ID NO: 109 - protein, 390 aa, Japanese Macaque Herpesvirus
MAEQNMDLKAWFIDAVESRRYPGVVWDDDDRTIIRIPWNRSTDSRIDEEYNKIFDDFCLARGVCQSGSTAHANKFKKIRMLYAVRSHRYLRELTPPSKAGGVSGERYRLFQLLPEVTNGCDLCNLIATTSLHSCSMGSGVRDDFFEQARRPRARLPLRVSIQRRKARLQGSPAQAAPGAIEVSFFYFGENVGVEILRSGCGVRICGLPDPKRPGHLCCADNPLTRFLPSAQLVPCEFARADLQALQKTCERGLICVMVESGICVKNLEERNMTALTNYSENYYELQPTQQFQVFDLLHYLRALARSPTPGDLPPRDCAWIFLCPSTQSENTWDAPIALKLRYVCDDVSVETGDSATGSNSGDEGPSGVRGGASGTTGSTSVSTFAPYWRK

SEQ ID NO: 110 - protein, 86 aa, Japanese Macaque Herpesvirus
MLRSSRFFTQIPDSTITQIRPLSHVFCRACKSALANSHGTNCADGRKRVNGLSAQHRCPGRLGSGRPHIRTPQPERKISTPTFSPK

SEQ ID NO: 111 - protein, 91 aa, Japanese Macaque Herpesvirus
MRLSVDLFHGIRIMVLSSSSHTTPGYRRDSTASMNQAFKSMFCSAISRVHHLYRAGQLKYRAHRTTSLRDQKNLDLKEAYLYTPTGQLSNK

SEQ ID NO: 112 - protein, 355 aa, Japanese Macaque Herpesvirus
MAEGRAGSIRVNRPSGLRAWLLDCCDNGKHPGMHWLNEEKTLVRLPWNHLKGAGGVSENEKNIFLDYCQFKGIRHTGNRPLSLRECKNWLASAIRHSQTVEDVSTEENLSAPAPYRCRVIRLLPIFVRSCPLCNEADATGGMLLDLRNEVTARFLYLGAGREYEGAVGSDGVRCWMIRLVIYYYGRLVGNMEVGSPNGVRLLPAPKQPLQGHVCAGIKPEQVMLPHNPHNLLPYQTSMLRWLAKEIIRGLMIYADGSGIYVRYMGHVPAFLLGNGGSPEPVDMINSGRVARVFSLVQYLSAVSATPPHGTLFPAAYASLHIGGVPTPEGEPCPTIPLSIQMWHECLWRACGDAAM

SEQ ID NO: 113 - protein, 364 aa, Japanese Macaque Herpesvirus
MAESEITHNHLRRWIISNLEANTYPDHLRWCDEEKRSFKLSWHRGMQGMQPVVAYCLDRDLQCGRQHNVSDCRKRLLRVLRENAGFEQDDTRATTTNSGGERFFYLKPAVDPLCYACILDSHSETVINYLEAACVHGLEPWTPLPPQAPAEASGAARSVYARAARLAIAAPPHPEQITPFWRLRIQVFYFGSLVAEHTGVDRRGVRLHKRQDPKAGHACYYGTAFKMWLPTPHENGPLTPEQRETVCEIINYCEEGIFLHGNELGIYVDNRTRHTLSCAGNDAEGNHAQRFVRSCAKFQIFYVMGLLRKLALSPVPGDPVPINAVTLYLGGRPGSRKRPQVPVILVICQDELTHGDIRPARWIL

SEQ ID NO: 114 - protein, 86 aa, Japanese Macaque Herpesvirus
MAMSWGYMWITEQGTLFPAPGTTQRETTHSVLCDPVPNFRFFMLWVYCANSPFPPFPATRFPSTQSLFTLGVALAPVKDPRSLSFW

SEQ ID NO: 115 - protein, 102 aa, Japanese Macaque Herpesvirus
MDLSLGRPYRLKLRRRLVVRRGPFMPEPPGWQLPRHHTRSRSLHFGGYVYKFFILDRWWRNTRAWIVEGYACISAKTQKLAMRVIMGQRLRCGFQRRMRMDH

SEQ ID NO: 116 - protein, 360 aa, Japanese Macaque Herpesvirus
MGTYTSEASLAWLSFMSGTVAASPFILCFIYHSLYFLEPLNSVENIIFSWGAVGLHGLLLLVCVVGPPAWLSRQVDVPCTISAILITAGSMASTLGVDLPWVYVSFFVGSCLCLLLCVVVANDVVYLCPTIAHRYYELGFFAAFSVYYFLVLKNLFLAPVFLLPLVAFIVGGVCSLRALRSHPLYEAGLQRRHAIFSLTSRRYITYSIKQALEVCGWDFYLVTVLIGGAAAGTLSVGLTTPLLLGLVHYFFVFHVGLFCCIGLVFRSNVLALVYVLAAAVLLTLTHVLGPGTHNLFTRVCVFTVFLLTMFGAIGCELQIIRKKLQRAANSPRIVLGVCACGNLLMAVVFFSLNKVELVAL

SEQ ID NO: 117 - protein, 394 aa, Japanese Macaque Herpesvirus
MPVSFHYGARVDVDALGNISKVYDHIKGIVKKGVIQISGQGRAPVLSVLSSVGDAGVLGLRLKNALAPLMVYSDMTDEVSFSFRNTSLGNTFTHTREMFGVNITEMNVAFYHHGDESNAEGKPQFVRTTIAYGDNHTSTVHKSVVDEPNLPSFHDRLEQAGTGNRLFLTVKTLTLLSKWLRQQKTRAKQVVTVSLSETLAVATFTVDGVSKIIDFKPDTPDAKWTCAKGRKLDVGVVSSDLTTHVSLESLVAALNACKIPGFFLPGFRWHANEILEVEGLPLTDSLADVKLGVMLLKVDPTDRNNAVPGNLSEGADPEGVPELPSPPRTPDLDLKEQCVPNAEDGTEPTDGGAKSLRTSGSRPEKKHGKRKHSSSPSRGKAKTKTPRATFNPLF

SEQ ID NO: 118 - protein, 80 aa, Japanese Macaque Herpesvirus
MLTPNISRVWVNVFPREVFRKLKLTSSVMSEYTISGASAFLSRRPRTPASPTLESTLRTGALPWPLICITPFLTMPLMWS

SEQ ID NO: 119 - protein, 314 aa, Japanese Macaque Herpesvirus
MSGLSIVTAAMESPDRFLYASDHPGFLALTQETWQNRWFPSQISLHEDSDEVRLLSPTDREFYQFLFTFLGMAESLVNFNIEDLVKEFSNHDVTHYYAEQVAMENIHGKVYANILNLFFGGNRGDLMTYAKKIVEDATLAKKIDWLHSRVRKATTRAEKVLLFLVIEGIYFISSFYSIGLFRLRGVMRGVCLANDYISRDELLHTRAAGLLYNTMISRDESPSVAYIHGLFREAVEIETLFIRSKSRGVTMVNVGDIEQFLQATADRILKSINVPPLFGARPPNACPLSYTSAKSVNFFERDNSEYVTSVHNDL

SEQ ID NO: 120 - protein, 788 aa, Japanese Macaque Herpesvirus
MNTETSFSAAKSAKPLTLVTNAETGGCSSSLDPERCAESLVNSLKATLGWDVEANSLTGLLWHRIMEDRCLVTVKDYLTVFGEKLSDEVREFMLKHEAALDGLLQDFKQSKAYANLVNCGYLSAVRFYDTYVLRTHGSSPIFESVAQMFMRVAVFVACQCIKFPCLRKTLRHLVQSETELDEMYLVGYAFHYISSQIVCCATPVLRSAGLRGGQLSSCFILKPSMATENKTLKALHEEMSPLLASKSGVGIDVSSFAEHKNITSCLKLINAHVGYFNDNNIRPVGASAYMELWHHQICDFLNAKMPENQERCHNLFQGVCVPELFFRLYETNPDGQWHLFAPEVAPNLLKLYGAEFEIEYNRLVAAGKHSSSLPLKSMMYALINTVIKTGSPYVLLKEALNKHHWCETQGSAINCSNLCAEIVQQPEGQASVCNLANISLPKCLRPPRGKSGVEPGKGDVTFGFELLDDAVEAAVIIVNACILGGTAPTESVRRGQKERSMGIGVQGLADVFAELGFGYLDAESAKLDVEIFQAMYFAAVHTSHEIVLLGEGTPFHGWERSRLAQGVFHWQTWDGVKPSHPPLERWEQLGRSIAQHGIFNSQFLALMPTAGTSQLTGYTEAFYPFFANIASKVTSKEEILKPNVTFFKRVKPGDLRTVRRYGGDVASFPEPLKDRYKIFLTAFDYCPIKQLERAGARAPFVDQSQSLNFFLKEEQATRASYIRDLLLTGYRLGLKTMLYYCRIQKQTKLNALQCLDQVVGDNTSSEGAESNRVQKADGEQAEVCLACQ

SEQ ID NO: 121 - protein, 138 aa, Japanese Macaque Herpesvirus
MTPWRPQLLLSMRAFWAGRRLPRALGAARRNDQWALASKDWPTCLPNSALATLTPRAPSWTWRFFRQCILRRCTRVTRLFYLARGLRFMDGSGVDWLRVCFTGKRGTALSRPTRPWKDGSSWGAASPSMEFSTASFWR

SEQ ID NO: 122 - protein, 97 aa, Japanese Macaque Herpesvirus
MERVTNQVHLVQLRFTLHQVSEGLPQTRKFYALTGHKNSHPHKHLGNALKNGRRAMGSQHVRIVKPDGRQVAAVNEICVCLALLKVLQKTVKGRFVF

SEQ ID NO: 123 - protein, 331 aa, Japanese Macaque Herpesvirus
MKTRDANVNKLNDSLMRLLPPPPHRVSLSRGRDFSKGVRDLLSKYVVSTTTGVEAIKDGFLSVSPKCQTYGDFLIYSQTMSSQEPRGTYLFSFKQTDTGSSIDMLFTPTSLARLSRMDADSAPQTNRIACVWYGHESGLLDAIPNFEELLETGSLHQFLAPVGPLVQTVHSTFVTKVTSALKGDVVAREPVVTHIGLTLPSDMFVDLDDSCPSSLRDEPLPAHSSIYVCLTYIRANNRPALGLGFFKSGKGYCEIAAQLRDFYSGVIRTKYIQLQNDLYINRLAFGVVCRLGSVPSGSQPSFQSLHFKGAALPVLKFTEFVSNPGSWKLFL

SEQ ID NO: 124 - protein, 939 aa, Japanese Macaque Herpesvirus
MTSSISAARADNGDENTGGLYRLIDNLLTCTGSLQQLKLLMEFQLKPLPTAHLLSMPTVTRFLNTAFKIDNPLVSFIQKHPVFFLMRVARLPDPVITDHQSAETSTGILSEVVNVLNIAIQKPHESPAAKDDDYLDNRAILTMITEYIHHVTSRTPSGIPPTPPMGISHLPCVEQILHETHRQYWNLALPESLFIDTGEVASPLQTWLIISYCKKLQLAPPPLFPPADELARRLVTGHHELFVPLSTSLETYITMPVSKRRAFEIYSVFAKSKNIVDGTPILAFTDTELTTFTPELLFLYDFVIESLCKNQAYGCSRNAIEHFIKKGIDFMAELGAFIEKTCGYRSTVSLSNVRSVKARLASCGLSKEACEDFRTMILMTPHETTPKWENFTDFLEMVNQLTLYGFYFYECLNQYSPTSISLAKIQNILNRVDAEQSDRALWRTPLIGSFPFPWKLNNVLAFFKPSAPVATLQKIYKAIPSYLMRSLFEIAANKSWGNIALAERAPLTDIQTAEPDQGPVPTQVIAKYCSRLQISATDYDAVIVSSPGFATEFIRTKLYPILSEVLRNTSKKNRSLFQLRWLIVFAAEDAKDLAPIRRSLALAYFQIMDILEEKHSPESFYNLLDYLQDTFRCIRQVIPEATCPQEFLQYLFTFQNLPIAASFIQTSMTFVDDLNNGIPGILDLVSLGAAFYNMKLLYDSTLDTVEIPTEEGQPIVVSMFVFKSTIRILEKLLQEAVIALAQTSEPMYAAHIRLMQHLTYMQKIAGHEIMTTQLPSVFHEIHEGYLQCFKRFKRLMLHVTGSCCYSLTRYFGFLYQPPLIPDTIVQKILNFNDKTDTTDDILKSLSQPVGQEPLSAENESSSRLSKNDVELLQKLYDDFRNGSTNDNPTSIKLEYSGNYNETQVSVDWSTYNLVTYTAPDDTLKFTPVNTEALDRMLAE

SEQ ID NO: 125 - protein, 1966 aa, Japanese Macaque Herpesvirus
MELPPIFSKFKIEGVATTHQADCRFGQYAGSQCLSNCVIYLAQSYFNRESPVTDTNDLDDVLRQGATLDFILRRSGTLGYNQYAQLHHIPSFIKTNEWSAAIFQSQEYFGLIGLDAAIREPFIESLKSILTRNYAGTVQYFLFICGDKAGAVIIKNKTFYLFDPHCVPHVPNSPAHVISSADPTAILEYVSPPDREYTGSFLYIMPSEYVNPEHYITNHYRTITFAKVHGPQIDISTGIEPCTIEDIPSPPRSPDVTSKSSNLARVPKTTTDTSSAKTTPAPLSGLLGVEPPTSYPDPAADDADTKLLTPAPARTAVDHPEFQTTPGATLLLSELSASRGRKRKLSSLQRYSDSDEASSDDEGAPRRRAHDDAISAEVIWMDDDISPLYSPSATPSFDDVFDSPPMSPEFTYTNATEDTDGAFLEQIVRDAETPFSAFNDLISDHDFSSLDKKIEQLIKYEAPSQHLPNISDKRNGRAVREAAALQAMDKIMINIILEHGLITDAQARGPSACKNVLQFFILWGEKLNIPISDAKQVLELDLQLIPLHTALSEGKFKQGAFKKHLTTKINRCLASMRATHANAHKKLASAFNIEGSQISSSETKISVRALKEQIANHLSPGFLAVYSADELKHLRDKIRDLKTGIEQRNREIQQEELFFGAVLTALDTFQPPPKPAYPMEIFPHRKTDVMLNHLASITTRLTDDATEALNNYLETPPDQGTHITNIPNFSSIVANIISTIKILTYAENDMQLNVTPMATYRRQLLYLGGELATIFNLEWPYETVPPVQELPLVARAKAKMESVTKMEKNQQALDQILGDAETLLDTITATSGNENPVRAMSIPILETYITNAGALIGTSRNPRFEKLKAAIHDLASSESFIIMLLNNTRLDNISDNLAKIDGILTNNTRFLSTATVTKTLQTLGSSLIRECVEALDKRSPSSLTDARLLAVQTILGHASVPDHETLTRIVSGVASAQKESAGDDPDRWTRVTGHLNELKLAATQSNVDKATRRKLLMIITRDLKEAEVSQETVLETRWQENVLKFQPSTSKEIEDFLQSAPSAKARKFAEKHLRTLIAQFDGHEQRPSEATAVPMDYTPTPIPTPQAVSTATAEKGKAAWNKIQQAFQDFNFHLIDASDWQEMASEYSRHGSSLPGTVGPKLVRFMESISNTLDDILTQKLASLLPNGPAFRPPAFDWIAPYQTRVNAFLKTIGLPMVRDLADKIHHQCQTVSHAVQSADLQQATVGTSLERPAAEYCRILSDMQVAFNDHGIAVRSEAAAYTDAINSPANVATPPKPNLEAPKKLMTATDVLTVEDFPEFLKTSILQQEQRLIALQRAEFQQLEASISAAERLRQSTRNEIAGKMATAITQLLPRAPVAISSRPLNLSKPIDFLSSTVYDKILDKEPYETAIAGFAWLETATKSVMVYSHQNQTQQLNVLLSEVEKQSTIAQRLHDLEVSARNTDDVKVLKQALDELAPLRVKGGKTTVDAWKQKLESIESLLRATRTAGEISSELERIGAQAVGTIAVRDLGTLSDQCREAANFLKQASLPEGFSDIGTKLSELQAYIKYKKQFLEHFETTQPNVFRRFPLSQNITENVPARLAMDSMARLANHLHVRGSAPHFTTWIETLPTVDPEKPTHVPAHGGAPLHRQITYSNVLEALFSLCSTTLTPVPTAPGLEIATRARRGAEAATWMDRQWPDIAQTLQDVLDTYEHTTTHANRDATSNAFLAMCVFTQIIRGASRAVTLPKLPGTAVDFPEEIVLTPRECTTLVTAMWPTLAAAILRLKSYSEALGLMSRFLPLMFQALPHLTLEAQVQNGPHNTPPQVRCFARTEAIPYFPAQWQSANLEQSLWGQTDFLQICDNNQRKARVAAVTWALTTINGVVLDQLWSTFKPMTAASDDTYVDLVKTLHLTTFGPRGPTPRRETTTEHPPYEYGQPTGYCISGQSTTPVQASNTPGVRF

SEQ ID NO: 126 - protein, 121 aa, Japanese Macaque Herpesvirus
MESPTVNIEEIYRRPSRSPRRISHRRVRAYVGPLRRQTTLRRNPNIAEGWTACVSDPWMPTVLKEVAWLPVLFGIRGGRRRFALERELRVLCPRRRLPGLGSLSVVRRPVDRIAVPVWFWR

SEQ ID NO: 127 - protein, 147 aa, Japanese Macaque Herpesvirus
MILIIILSIAWRAAASRTARPFRLSEMFGRCCEGASYLINCSIFLSREEKSWSDIRSLNAENGVSASRTICSRNAPSVSSVAFVYVNSGLIGGLSNTSSNDGVADGEYRGDISSSIQITSADIASSCARLRGAPSSSDDASSESEYR

SEQ ID NO: 128 - protein, 106 aa, Japanese Macaque Herpesvirus
MVWTARRRASVREDGLLLSSASTHSRIRLLPSVWSVFVTVAVERKRVLLVRMPSILARLSDMLSSRVLFSSIIMNDSDDARSWMAALSFSNRGFREVPIRAPAFVI

SEQ ID NO: 129 - protein, 92 aa, Japanese Macaque Herpesvirus
MSRCSCWRIDVFRNSGKSSTVRTSVAVISFLGASRLGFGGVATLAGELIASVYAAASDLTAIPWSLKATCMSESIRQYSAAGRSKLVPTVAC

SEQ ID NO: 130 - protein, 97 aa, Japanese Macaque Herpesvirus
MVPTACAPMRSSSEDISPAVLVARSRDSMLSSFCFHASTVVFPPFTLRGASSSSACFSTFTSSVFLADTSRSCRRWAMVLCFSTSLSSTFNCCVWFW

SEQ ID NO: 131 - protein, 113 aa, Japanese Macaque Herpesvirus
MGTGLRGALYRLLNGSTSGTHSRMGVRSPMPPRAWPRRWACRQKDSHWYVKHHRSEHRLKSGHPACLKPGPAWSIGPRCSSPWAVRTRTAGARWSFLALVLDRADQRWSGVGS

SEQ ID NO: 132 - protein, 433 aa, Japanese Macaque Herpesvirus
MGLLTPILECVPDVEPFKSLYNAPRKPVPINTLPASLHPHDEQQVFLRQAQWLSYRFIPHEAARSSSPPLLVVIDPENLVTATYSSGGPANFESRPFYVMPGPYPTDWPKTLSVTSNTSVTHLSHDEICNLFTTLSREHGTVQGRDIFAAAPTNVTPERTANHPTWETENQLTTQTEPAEKTHVIPASPKARTDPIAETTAHHSQGQASQHANNVNQPGQITSHASRNKPSTAPQASSRPEKLNTQTVPRLISQTSETAHINHPASGQVTEPKGIFGTYKPRVLTEPAKPAHASIASRQPEATTTVPKLPINAPTAKVFIGTASKFLPDVEESHGTTPGAHQSKIDQKQYGESQPHRTPHLEEVPRAPHVDTPTSAHINVPSSQGPKTIHARVTPGIQTVTPSAPPPAGQIRAFNTRFPEPTAGRSTTNRMWN

SEQ ID NO: 133 - protein, 140 aa, Japanese Macaque Herpesvirus
MLYFQEFGGVLVDESLRLDRMGRSIYSRPVRACETHFVFWAILLPHWLGSIPHPIRRGATGRRFWEACIECADLTGRWGGAGSYCLNTGCYARVYCFWTLARGDVYVCAGWSVDVRRPWNLFKVWGAVRLTFAVLFLIYL

SEQ ID NO: 134 - protein, 169 aa, Japanese Macaque Herpesvirus
MSSLRVKEPIVQGRLEHDYPNHPLVAEMNNLPQGDMSPAQYAIAKRNYLVFLTAKHHYDMYTQKKNGILRKDHLRGLRGKKDASSSISSVLSGSGSAAPSVAPVASTLGSNSFTTISSGPHSLIGSIGPTPGGGGPGSVASSGIGSTSLSPSDATTLDTRRSSQNKKSK

SEQ ID NO: 135 - protein, 86 aa, Japanese Macaque Herpesvirus
MPEDATLPGPPPPGVGPIEPINEWGPLEIVVKLFDPRVEATGATLGAAEPDPDKTLEILELASFLPRRPRRWSLRKIPFFFCVYMS

SEQ ID NO: 136 - protein, 448 aa, Japanese Macaque Herpesvirus
MASGRLPDLAEDEAACHGRVPYPVHHWLDCSRLGLDFAASMRAIGLCPECYVCFVTYGLGAWEGQPPEWARTLISAPSFQTALNDIATGWMPDNPPKNGDVKSRLHDTGRLLLEAYAWVLRCICTGVGYADNEGLSLTAVPRGAWSRYLVVSFPRACCFVCKTLNCRQRFPLVTCSPQHALDLPVLRKKWNGAGCVAMQLNVPSISRRLGANLDERGLGPSDAGLLASLRELAPTVPCGNPFNALLRSLTFRGLLSMSRVVLPIGESTETEISRDLGQKVLAYNVLFPCISLPVWSQVVARSVLEKTVPAPRVVVCLECGYCLNFGRGKFETVNFPPTNVFFSRDQKEKQLSICATTGRVYCSYCGGSHMRVISLFEITCVGDPYLRCVLANNAAHSIRDANSLVSVVVPCLASPDCATGLLKHLRVAELFYLTSSISSLSCGKCNRS

SEQ ID NO: 137 - protein, 135 aa, Japanese Macaque Herpesvirus
MGSTTRDMLNRPLNVRLLRRALNGLPHGTVGASSRSEASSPASEGPRPRSSRLAPKRLEMDGTFSCIATQPAPFHFFRKTGKSSACCGEHVTRGNRWRQLRVLHTKQHALGNDTTRYLDHAPLGTAVKLRPSLSA

SEQ ID NO: 138 - protein, 268 aa, Japanese Macaque Herpesvirus
MSSGKRLVDELCDVVVSYLGPSGISLDLERCQDGAPVYAKGGAVPVCTVRLQHGCVYHLEFVYKFWLHKLERLAYPFAPCFVITNNGLATTLKCFLCKPRDADAQFGKNLPINSDVYLERNSSVFLGQDDFMKFKARLVFSGDLNVYSSMVICRTYFTEHRQVLQFLVVTPKSAKRLKTLLRTVFALTGHSDGLGALRRTGSVARPSGSELTDIGSGERCGNDRLTDSIGTIGGWPRGACLTWLKTKLPVMGAFLILSIIGWIVLGWA

SEQ ID NO: 139 - protein, 104 aa, Japanese Macaque Herpesvirus
MMDRIRNAPMTGSFVFSQVRQAPRGHPPIVPMESVNRSLPHRSPLPISVNSDPEGRATEPVRLNAPRPSECPVRAKTVLRRVFNRLALFGVTTKNCKTCRCSVK

SEQ ID NO: 140 - protein, 86 aa, Japanese Macaque Herpesvirus
MSGDDLLAFESLLPEDMKIMFPTIYSRLNAINYCQYLKTFLCNRAQTRSAHCEHCMVLDAKVNAVKQVIHKIVSTDAVFTGAAHST

SEQ ID NO: 141 - protein, 457 aa, Japanese Macaque Herpesvirus
MFVPWQLETLMKHWPSLRELVEQSFLPGTPDVAFNSPVLIHTQDSLQPASSCRVCNILFTLVRTFPPPDSFFEDYGWLCLTCLYAPRSWTATLMVAADLLELIHMYFPQRVKDGPVYTTQNILGIDVQLHFFATRCFRPIDREQILHTSHLNFLQTEFIRGMLEGTIPGSFCFKTSWPRTEKDDQQPTVACCSVGRGNHANRDNHLPDDLEEAFNSTNAKEKPSILGVFSATWSESQLLGSDTQQATIHSQPSTFPTPEDADQSQGPCLMHPTLNLKTKNHTASICVLCECLAAHPDAGPVLRDLRRDILENMENNVKLVNRISYILNDPDSLSHVRDEHLRGLIKRCSAQEIHKHFFCDPLCVLNTYSHCPAVLFKCPPPEKYKKLKARLATGEFLDCNRIFDCETLQTLAVLFKGSQLAKIGKTTSLEIIRELGFQLRRHNIQITHPFQTSNLYI

SEQ ID NO: 142 - protein, 127 aa, Japanese Macaque Herpesvirus
MYDMRLTSLTLFSMFSRMSRRRSLRTGPASGWAARHSHRTQMDAVWFFVLRLSVGCIRQGPCDWSASSGVGKVEGCEWIVACCVSEPRSWDSDHVAEKTPRMLGFSLAFVELNASSRSSGRWLSRLA

SEQ ID NO: 143 - protein, 108 aa, Japanese Macaque Herpesvirus
MFDECPNDERDTHRPGAMVFRFNGNITDFEVHIGVPISLKKSTPSSWRRVRAEKNCVSETNFFHCIISLLQLPCLDAGCVDWGPSGRLTLGAYGVRDANRRLGCFGIV

SEQ ID NO: 144 - protein, 297 aa, Japanese Macaque Herpesvirus
MPKQPRRRLASRTPYAPSVKRPDGPQSTQPASRHGSCKSEIMQWKKLVSDTQFFSALTRRHELGVDFLREMGTPICTSKSVMLPLNLKTIAPGRCVSLSSFGHSSNMGFNCSSCTPTDRSAVSLDANALGEDSARKNSELCSVALTFYHHAEKVVQHKGFYLSLLSHSMEVVRRSFTQPGLLYAHLVLKTFGHDPLPIFTVDANERLALWAVFHTRDLHLGETSLRLIMDNLPNYDITVDCIKQTYIMKFTPSRPDNATVTVPVNSICEAVATLDCTDEFREEIQRGTTIINSQGSL

SEQ ID NO: 145 - protein, 83 aa, Japanese Macaque Herpesvirus
MLTRAPRLGASVQLTAVRLLLLFLTSSTPTAINCTKPGSVFFWLSWPKVFMENHYSFISVNSVSITVVAAGKISSRGKTATNC

SEQ ID NO: 146 - protein, 289 aa, Japanese Macaque Herpesvirus
MWCLVQRAGPPVGCRSPGGVQVPRWGAGPPVGCRSPGGVQVPRWGAGPPVGCRSPGGVQVPRWGAGPPVGCRSPGGVQVPRWGAGPPVGCRSPGGVQVPRWGAGPPVGCRSPGGVQVPRWGAGPPVGCRSPGGVQVPRWGAGPPVGCRSPGGVQVPRWGAGPPVGCRSPGGVQVPRWGAGPPVGCRSPGGVQVPRWGAGPPVGCRSPGGAPGGSGWGGGSGWLRVGWRLRVAPGGVAAPGGSGWAQVLKATVNPTANPTQKSPCASRAEARLLTCASGALYIGQNTLPK

SEQ ID NO: 147 - protein, 108 aa, Japanese Macaque Herpesvirus
MGNPRIDRSHSKHVGFTLFGESPLAGPNVPARCTWVLRNAKLPLPCRVPYSCSAIFEYTALHGWRAVGRWCANQKLMIHLLVLWLHNNTMLLILGGCFGLYKGRRKHR

SEQ ID NO: 148 - protein, 234 aa, Japanese Macaque Herpesvirus
MYSWGEFRTMERKMSLRVTRGSQKHITMGLFGAHKRAVGNGLGGAPAPPCARNLGRGVRRGSPKHVLMVAARTHRPLFGAGVIRRSAQNVAHGTHCTHGAHEGGGVAGRSHRAGRGARRLWHRASDVYGRNPLSRGIKGRRQGRFPLARRAVNQTDARRRLVVATVGFLLRCRVPSRDWLGDLSIKNARRPLAQPPVNHGILAANWPKQTANPRVGFCLQVPLYYIYSIIFFPC

SEQ ID NO: 149 - protein, 85 aa, Japanese Macaque Herpesvirus
MTPAPKSGRWVRAATIKTCFGDPRLTPRPKLRAHGGAGAPPSPFPTARLWAPNRPMVICFCDPRVTRSDILRSMVLNSPHEYMFR

SEQ ID NO: 150 - protein, 88 aa, Japanese Macaque Herpesvirus
MVPFGAHGAVFAARGRRRVTETCTHGANSGPWSAKCRSGSPEGHRNISPWAYLVPISGPWEMGSVGRRRHHVPAIWGAGSGGGHRNMF

SEQ ID NO: 151 - protein, 94 aa, Japanese Macaque Herpesvirus
MSCEHFPPGYNGQESAGKTSTGLPVGEREARRYAACVSEVAPMGRMTTPATRRAAGADISQNRPRRQAARLPPPTTLILAFKLLFKARLFVARA

SEQ ID NO: 152 - protein, 174 aa, Japanese Macaque Herpesvirus
MFPHKRIVDLGRHLEADDREAVLWLFDRPVSDNTPEGFANGLCPPTGEPGIPFPVLLEAVFLVGRLDLVSTFFLLDVGFIVERLRSSPSYFSPYKHLMLSINRQLSERDVKNLVFLTGNQLGRKRNQSPTFFQWISQMEKAALVSPSDYLVLKDLLQAVSRRDVAKVVAANAPG

SEQ ID NO: 153 - protein, 254 aa, Japanese Macaque Herpesvirus
MAFVGPVPTGTIDPVLYQDRALSNLLAHETSFVTSTACYGTVQTEVTLGMRVILGTWMRSVARAHQADASVFPLAVSILDRYLECRSIPRRRFQRLGAACLFLAGKIRDLNPFKAAFLCFCAADDFSVADLLKQEKSVLKALRWKLEAVLPTDAIGPALFKSGFTKEQLFALHSRVVESVHKAIVNPVTGGLSPSLVAAACALFSLGAAAPPLDKLAEAIGVSAATLTAAAESVATTLRELDEDRILNNARGSS

SEQ ID NO: 154 - protein, 436 aa, Japanese Macaque Herpesvirus
MWGSRQHRSGIVSGHGLRSSCRGHCGRRGGTREQAGGRSRGRGRGAAAPASASAASASPTPPGPQVLVVVEQGHGSDTETATESGHGSSQGSPSGSGSESVIVLGSPTPSPSGSAPVLASDLSPRNTSGSSPGSPASHSPPPSPPSHPAPLSPAPPSSHHPSPDPQPPSFLQPLPHDSPEPPGPPTSSPPPNSPGPPQSPTPTSSPPPQSPPDSPGPQQSPTPQQAPSPNTQQAVSHTDHPAGPSRPGPPFPGHTSHTYTVGGWGPPRRPGGVPCLRLRCTSHNSHEDEVPERQREQEGEERQQQPARPPRPPRYPIPIPYPHPRRRYQENTAHKEDFIVRRWETGSTPLDRARGVTESSFVTQTPIHCIAWQGAYSFPGLEHPASAFCRTHLEVPSSRLFASRCSVKVAEPPRPSKKPVAGGNNFTHPPPTSMLA

SEQ ID NO: 155 - protein, 126 aa, Japanese Macaque Herpesvirus
MGPVEALAIAVVQVSRAGIAEGARTLALAIVVVQVSWEGARTLEAQAVQVSRGATVAKTTEAADRAMDDATTAVQVRGGRGAMGVMVGANAMRGIPGTTLMCFEVTGLKLTPAQTRLAMALEIPTR

SEQ ID NO: 156 - protein, 108 aa, Japanese Macaque Herpesvirus
MTDSDPDPEGDPCDDPCPDSVAVSVSDPCPCSTTTRTCGPGGVGEAEAADAEAGAAAPRPLPRLRPPACSLVPPRLPQCPLQELRNPCPDTMPERCCRLPHMIAVYCV

SEQ ID NO: 157 - protein, 83 aa, Japanese Macaque Herpesvirus
MRARPDQNSPPPSGHNRKRTKHRFCVRALRWLRVVERAIYLICRFLHAVNRDHVGQPATSFRHSIGTRVAKFLQGTLRKTRRH

SEQ ID NO: 158 - protein, 86 aa, Japanese Macaque Herpesvirus
MALRVGGNLFEKDLLPPGVKHRHRPCVFNHVGRNYINAAAGDARHGSVRSSNALCGGPRALYRVPWVRVNNSPQRSYRYLAKTGIA

SEQ ID NO: 159 - protein, 253 aa, Japanese Macaque Herpesvirus
MLGGITLTLLLATLATVRCALQTHYAAVPVHSTASLGCVLTTAHNVLIVTWQKQESPSPVNVATYSSEAGTVVQPPFAGRVDIPEHKLTRTTLKFFNATMEDEGCYLCIFNAFGVGKLSGTACLTVYVPLSMSVTFYPPINPTQLVCRAEASPAPSVNWTGVPPELCSEPEVFPRPNGTTLVVGRCNVTSVDPEDLRNATCLVTHIGGLASARPLGPVFSDPLEGTSHYVVGVVAAVAVLGIFLTGVFLYRSM

SEQ ID NO: 160 - protein, 165 aa, Japanese Macaque Herpesvirus
MSDQARGVSEVFGIHRRYVAATHDQGCSVGSGKHFGFAAQLGWHAGPVNRRCGTGLGPTDKLRRVNWGVKRDGHGQGDVNRQAGGSRQLSHSKRVKDTQVAPLVLHSGIKKLQGCSGQLVFGDVNPTGERGLNHRARFGTICGHVNGARRFLFLPGNDKNVVGCC

SEQ ID NO: 161 - protein, 342 aa, Japanese Macaque Herpesvirus
MDALNNNLNLLMDFLSNYSNSYSSYDDNISYTLDTESTLCRLTIIFPPTIYAIICFFIFCITLLGNALVLYIFFKFKALANSVDVLMAGLCCNSLFLCASFLFSWLLYVAPQILTPATCKVEIFFFYLYTYFGVYIVVCISLIRCLLVVFSRRPWVKHWASGFLCVCVSLIVALALSANASLYRTALRHPETSEWICYEDAGEDTVNWKLRIRTISAICGFLVPFGLLVLFYGLTWCIVKSTKLARKGAVRGVIVTVVVLFLIFCLPYHLCNFFDTLLRTGFVTETCYIRDVISVAMHICSLLQSMYSAFVPVVYSGLGSLFRRRVRDTWSMFRCFSTSGSL

SEQ ID NO: 162 - protein, 1298 aa, Japanese Macaque Herpesvirus
MAQRTNPRWAAAALSADEEAFIHDNSDAESVLALVPEQCFSEFLLWLVTRPSDNFDNDDDDPTLGVIWQLLAPLVNYAPLETRSAHLQGHHTISLPYGPDLLRQPTTRSSELVQCLRDSGFDTALRLELARHLSCQTRRFVADRVPPGTFAALTLGALVEYDVRVQRQLPVTVQSTAWRPLPERDPICAAVMLPLQRNILPLAVQASNGNSYTVSRYAVMARRSYGCVFQRLPCENVTHIADSFTHLHSAIRTGAGALQDILFHSTLLPGGDIRSALCGFYATTPSVGAFSRARHRAINTTTTLHCQQLARTGTPVLGGFLKTVHSATTSEANVITTTSLLSCVPQAYTFLRRSLFNQPIICLGSFEPVDGDGNQRSLYLGSAAGINRINQTLSLAYEILEGPLFTSINRAHEPASTISHLGALVSRGGLRLFVSQLPPTILSQLTATPDISRETVNDILLNKFLNVSAFVVFAVLPRDTEPEPGPLDAIRRAARICGCPFAVVGETCEEPGIQFVNDLELWNPGAWPIRQPTSAEVIATFGFDEQPVSSNWLVRPEEPEEGGEQAPSPTDWGLFRLASVVDQLLRCPTVGSKEFVTRHVDRCSNGLVAQQCEVGPLGRPLSDYHIVNHTAVFTDRMARVPIHRPQPITRQDATERLGSPETWVTQGRGRLRWVGQCVAHGEQAYKMGIDAAVGARYAICEAVTNIMLAHVRRLSDITLTASVGWNPEEDQAWLLQHALFACKELCRDLSVNFAITSAGSTPCLSEELISATQQHQTVAPVPFNAVIITATAEVKSSRRRVTPDLKATGNLLVLVTFPGPHLTQGSTFEHLCLLPSPTLPDVQATHLANLFMLTESMLSRGLVVSGHDVSDGGVVVTAIEMALAGNRGLQICIPSEETPLPWLVSETPGVIFEILPQHVDEVRQACQNFDCQATVCGTVGQEGLSERIVISHNNEEVYSQTLTSVAANWTSFSDEQWYSWGPSFTPAQELYRKDYGCNRHNLGHLADVCRNSELTLFAVPSRPPAVAALIAPGAPLPRALMAAFTNVGFDVAAVSTNDLRGGNILRGFSGLTIGGNVGIEDSYVGARCAIMGLLNDPGCYGGLMAFFRRADTFSLCCGEFGFQLLGALGLLRETPHDTPGPKTPDQWDIHLEENASGNHECLWLNLHIPQTTISIMFRVLRGLVLPGWANGRYLGVRYPRDAIEYHLNQQQRIALNFHTGNPDPRMFAQHYPRNPSANSAVAAITSPDGRHLASLVDPAVVFHPWQWAYVPPELADMTISPWALAFQSLFLWCVRNRQ

SEQ ID NO: 163 - protein, 90 aa, Japanese Macaque Herpesvirus
MRNHGSSQRPWVLWGPYGILPPSGYIFTVLRGVWVPAPGGARSTQRNPSRHARAQNPRPVGHPPGGERIREPRVPLAKPPHPPDHHQHNV

SEQ ID NO: 164 - protein, 84 aa, Japanese Macaque Herpesvirus
MPTFPPMVKPENPRRMFPPLRSLVETAATSNPTLVNAAISALGSGAPGAIRAATAGGLDGTANSVSSLFLQTSARWPRLCRLHP

SEQ ID NO: 165 - protein, 107 aa, Japanese Macaque Herpesvirus
MTILSDRPSWPTVPHTVAWQSKFWHAWRTSSTCWGRISKITPGVSDTSHGRGVSSDGMQICRPRLPARAISIAVTTTPPSLTSWPDTTRPRESIDSVSIKRLARCVA

SEQ ID NO: 166 - protein, 131 aa, Japanese Macaque Herpesvirus
MAKFTLRSRHSSLQANSACCSSQAWSSSGFQPTDAVSVMSLRRRTCASMMLVTASQMAYLAPTAASMPILYACSPWATHCPTHLSLPLPWVTQVSGLPRRSVASCLVIGWGRCMGTRAILSVNTAVWLTMW

SEQ ID NO: 167 - protein, 160 aa, Japanese Macaque Herpesvirus
MPFCRRRGNLRRAGNPVRERPRAVEPGSVADQTADLGRSDRNFWVRRAARFLQLAGAPRRTGGGRRTGTLADRLGPIPPGLRGRSASAMSNRRQQRVCHATCGQMLQRTRSSAVRSGTPGPTAVRLPHRQPHGGVYGQNGAGAHTSPPTDHQAGRDGTPG

SEQ ID NO: 168 - protein, 102 aa, Japanese Macaque Herpesvirus
MFRCNGSITAAHIGSLSGNGLQAVDCTVTGSWRCTRTSYSTSAPSVRAANVPGGTRSATNRLVWQLRCLASSNRNAVSKPLSLRHCTSSLDLVVGWRSRSGP

SEQ ID NO: 169 - protein, 123 aa, Japanese Macaque Herpesvirus
MFVSFATMGNTYDFYNNNIMEWTLQNYTLNTANIYSNGILWICMVKFTNKHCKNNWIVVCNICRYVAILLLFIINRGNIYEEINCLFFVTALIGMYAVTEASTTSSLTMALAYSIITANTGIF

SEQ ID NO: 170 - protein, 85 aa, Japanese Macaque Herpesvirus
MACNCPFFVWCTWLFSNILTGTFWLISLAQYACDNNKDLYIVAVSTVACFFFLWKSLGLYFYQSRSQRLNTPLLKLIPWITGMTL

SEQ ID NO: 171 - protein, 144 aa, Japanese Macaque Herpesvirus
MPGGGRPASGAARRGLWGPGLGGRRGAAPPGSAGPGRSRESAGPGGAGPGGGPGPPRDPRPREPKAARRAARGGRGRRPRARAGRAEGREPGEAGGAPLGGPGAARETLAPGARWRPRPRAAAGRAPGGPRRAPPLGALAWRRC

SEQ ID NO: 172 - protein, 91 aa, Japanese Macaque Herpesvirus
MPGGRRPAWRSSAGLPSPTSPRKVVSAPPGGGLGPGAPRRGRPRGATEGPGRTGDRENARGGGGQRGTCACLWNTMACWPARLLGSLQACC

(19) United States
(12) Patent Application Publication
(10) Pub. No.: US 2004/0234953 A1
(43) Pub. Date: Nov. 25, 2004

(54) Japanese macaque herpesvirus nucleic acid and polypeptide sequences and their use

(75) Inventors: Scott W. Wong, Beaverton, OR (US); Michael K. Axthelm, Hillsboro, OR (US); Scott G. Hansen, Hillsboro, OR (US)

Correspondence Address: KLARQUIST SPARKMAN, LLP, 121 SW SALMON STREET, SUITE 1600, PORTLAND, OR, 97204, US

(73) Assignee: Oregon Health & Science University

(21) Appl. No.: 10/779,597

(22) Filed: Feb. 12, 2004

Related U.S. Application Data
(63) Continuation-in-part of application No. 10/276,524.
Application No. 10/276,524 is a 371 of international application No. PCT/US01/16274, filed on May 17, 2001.
(60) Provisional application No. 60/205,652, filed on May 18, 2000.

(51) Int. Cl. C12Q 1/70; C07H 21/04; C12N 7/00; C12N 15/86; C12N 5/06

(52) U.S. Cl. 435/5; 435/69.3; 435/235.1; 435/456; 435/364; 530/350; 536/23.72

(57) ABSTRACT

Japanese macaques can harbor a virus related to RRV, called Japanese macaque herpesvirus (JMHV). An isolated virus is disclosed herein (Japanese macaque herpesvirus, JMHV) as deposited with ATCC as Deposit Accession No. PTA-1884, deposited May 18, 2000, as are viral particles including this virus and host cells infected with this virus. The entire nucleic acids sequence of this virus is provided herein. Also disclosed are the nucleic acid sequences of unique open reading frames, and the polypeptide sequences encoded by these open reading frames. Pharmaceutical compositions are also disclosed that include the viral nucleic acid, a polypeptide encoded by the viral nucleic acid, an antibody that binds the JMHV polypeptide, or a polynucleotide encoding at least one JMHV polypeptide. Model systems for screening for agents of use in the treatment of MS are also disclosed.

### PRIORITY CLAIM

This is a continuation-in part of U.S. application Ser. No. 10/276,524, filed Nov. 13, 2002, which is a § 371 U.S. national stage of International Application No. PCT/US01/16274, filed May 17, 2001, which was published in English under PCT Article 21(2), and claims the benefit of U.S. Provisional Application No. 60/205,652 filed May 18, 2000. These applications are incorporated herein by reference.

### STATEMENT OF GOVERNMENT SUPPORT

This disclosure was made with United States government support pursuant to grant RR00163 and CA75922 from the National Institutes of Health; the United States government has certain rights in the invention.

### FIELD

This application relates to a Japanese macaque herpesvirus (JMHV), specifically to the nucleic acid sequence of the virus, open reading frames in this virus, and to amino acid sequences encoded by these sequences. Compositions and methods are provided for the production of animal models useful in assessing the efficacy of drugs for the treatment or prevention of conditions associated with infection by the virus, such as multiple sclerosis.

### BACKGROUND

An autoimmune disease is a condition that results from a pathological immune reaction against an individual's own tissues. Examples of autoimmune diseases, in which the immune system attacks otherwise healthy tissue include multiple sclerosis (MS), autoimmune uveitis, myasthenia gravis (MG), psoriasis, and rheumatoid arthritis (RA).

Multiple sclerosis (MS) is a chronic, neurological, autoimmune, demyelinating disease. MS can cause blurred vision, unilateral vision loss (optic neuritis), loss of balance, poor coordination, slurred speech, tremors, numbness, extreme fatigue, changes in intellectual function (such as memory and concentration), muscular weakness, paresthesias, and blindness. Many subjects develop chronic progressive disabilities, but long periods of clinical stability may interrupt periods of deterioration. Neurological deficits may be permanent or evanescent. In the United States there are about 250,000 to 400,000 persons with MS, and every week about 200 new cases are diagnosed. Worldwide, MS may affect 2.5 million individuals. Because it is not contagious, which would require U.S. physicians to report new cases, and because symptoms can be difficult to detect, the incidence of disease is only estimated and the actual number of persons with MS could be much higher.

The pathology of MS is characterized by an abnormal immune response directed against the central nervous system. In particular, T-lymphocytes are activated against the myelin sheath of the central nervous system causing demyelination. In the demyelination process, myelin is destroyed and replaced by scars of hardened “sclerotic” tissue which is known as plaque. These lesions appear in scattered locations throughout the brain, optic nerve, and spinal cord. Demyelination interferes with conduction of nerve impulses, which produces the symptoms of multiple sclerosis. Most patients recover clinically from individual bouts of demyelination, producing the classic remitting and exacerbating course of the most common form of the disease known as relapsing-remitting multiple sclerosis.

Although the immune system plays an important role in the pathogenesis of MS, epidemiological data and the inflammatory nature of the disease suggests that a viral or bacterial infection may trigger the autoimmune attack against nerve cells in genetically susceptible individuals. Many pathogens have been associated with MS, such as Epstein-Barr virus (EBV), Herpes simplex virus type 1 (HSV-1), Human herpesvirus-6 (HHV-6) and Chlamydia pneumoniae. While there is no solid evidence that supports any one infectious agent as the causative agent of MS, experimental models suggest that the disease may result from one of three different mechanisms including: i) immunologic response against bacterial/viral antigens in the central nervous system (CNS), resulting in demyelination; ii) an infectious agent in CNS that releases myelin antigens that initiate an autoimmune reaction against myelin, a concept referred to as “epitope spread”; and iii) a virus or bacteria that contains antigens with significant homology with myelin antigens and thus elicit an immunologic response against the infectious agent and the myelin antigens, a process referred to as “molecular mimicry.”

There is a need to develop animal models of multiple sclerosis. Specifically, these animal models can be used to evaluate agents that can be used to treat this disease. Any treatment that could delay the progression or recurrence of the disease would be enormously beneficial to people who have this disease, by reducing neurological impairment and disability.

### SUMMARY

Japanese macaques can harbor a virus related to Rhesus macaque rhadinovirus (RRV), called Japanese macaque herpesvirus (JMHV). An isolated virus is disclosed herein (Japanese macaque herpesvirus, JMHV) as deposited with ATCC as Deposit Accession No. PTA-1884, deposited May 18, 2000, as are viral particles including this virus and host cells infected with this virus. The entire nucleic acids sequence of this virus is provided herein. Also disclosed are the nucleic acid sequences of unique open reading frames, and the polypeptide sequences encoded by these open reading frames. Pharmaceutical compositions are also disclosed that include the viral nucleic acid, a polypeptide encoded by the viral nucleic acid, an antibody that binds the JMHV polypeptide, or a polynucleotide encoding at least one JMHV polypeptide.

In one embodiment, a method is provided for testing the efficacy of a drug in the treatment of a condition associated with infection with JMHV. In one example, the method includes administering the drug to a non-human primate infected with JMHV; and observing the non-human primate to determine if the drug prevents or reduces the presentation of one or more symptoms associated with JMHV infection.

In a further embodiment, a method is provided for detecting the presence of JMHV or a related virus in a biological specimen, by amplifying by polymerase chain reaction a JMHV nucleic acid sequence, or by using hybridization technology, if such sequence is present in the sample.

A method is also provided for detecting the presence of JMHV in a biological specimen, including contacting the biological specimen with an antibody that binds to a JMHV polypeptide, and detecting binding of the antibody to the biological specimen or a component thereof. Binding of the antibody to the biological specimen indicates the presence of JMHV.

Kits are provided that include an antibody that binds to a JMHV polypeptide or an oligonucleotide that hybridizes to a JMHV nucleic acid sequence.

The foregoing and other objects, features, and advantages of the disclosure will become more apparent from the following detailed description of several examples which proceeds with reference to the accompanying figures.

### BRIEF DESCRIPTION OF THE FIGURES

FIG. 1 is a schematic diagram of an ORF map of the JMHV genome. Map of the JMHV genome showing the ORFs that are homologous to JMHV, RRV, Kaposi's sarcoma-associated herpesvirus (KSHV) and Herpesvirus saimiri (HVS) (light gray arrows), JMHV and RRV (gray arrows), JMHV, RRV and KSHV (white arrows), JMHV unknown (black arrows), and JMHV unique (dark gray arrows). JMHV putative genes are numbered according to Table 1 from JM1 to JM171.

### SEQUENCE LISTING

The nucleic and amino acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. In the accompanying sequence listing:

SEQ ID NO: 1 is the nucleic acid sequences of JMHV.

SEQ ID NOs: 2-172 are the amino acid sequences of polypeptides encoded by JMHV.

### DETAILED DESCRIPTION

I. Abbreviations

CPE: Cytopathic effects

EAE: Autoimmune encephalomyelitis

JME: Japanese macaque encephalomyelitis

JMHV: Japanese macaque herpesvirus

MS: Multiple sclerosis

ORF: Open reading frame

PCR: Polymerase chain reaction

RRV: Rhesus macaque rhadinovirus

TEM: Transmission electron microscopy

II. Terms

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

In order to facilitate review of the various embodiments of this disclosure, the following explanations of specific terms are provided.

Amplification of a nucleic acid molecule (for example, a DNA or RNA molecule): A technique that increases the number of copies of a nucleic acid molecule in a specimen. An example of amplification is the polymerase chain reaction in which a biological sample collected from a subject is contacted with a pair of oligonucleotide primers, under conditions that allow for the hybridization of the primers to a nucleic acid template in the sample. The primers are extended under suitable conditions, dissociated from the template, and then re-annealed, extended, and dissociated to amplify the number of copies of the nucleic acid. The product of amplification may be characterized by electrophoresis, restriction endonuclease cleavage patterns, oligonucleotide hybridization or ligation, and/or nucleic acid sequencing using standard techniques. Other examples of amplification include strand displacement amplification, as disclosed in U.S. Pat. No. 5,744,311; transcription-free isothermal amplification, as disclosed in U.S. Pat. No. 6,033,881; repair chain reaction amplification, as disclosed in WO 90/01069; ligase chain reaction amplification, as disclosed in EP 0320308; gap filling ligase chain reaction amplification, as disclosed in U.S. Pat. No. 5,427,930; and NASBA™ RNA transcription-free amplification, as disclosed in U.S. Pat. No. 6,025,134.

Animal: Living multicellular vertebrate organisms, a category which includes, for example, humans, non-human primates, mammals, and birds.

Antibody: Immunoglobulin molecules and immunologically active portions of immunoglobulin molecules, for instance, molecules that contain an antigen binding site that specifically binds (immunoreacts with) an antigen.

A naturally occurring antibody (for example, IgG, IgM, IgD) includes four polypeptide chains, two heavy (H) chains and two light (L) chains inter-connected by disulfide bonds. However, it has been shown that the antigen-binding function of an antibody can be performed by fragments of a naturally occurring antibody. Thus, these antigen-binding fragments are also intended to be designated by the term “antibody.” Specific, non-limiting examples of binding fragments encompassed within the term antibody include (i) an Fab fragment consisting of the V_{L}, V_{H}, CL, and CH1 domains; (ii) an Fd fragment consisting of the V_{H} and CH1 domains; (iii) an Fv fragment consisting of the V_{L} and V_{H} domains of a single arm of an antibody, (iv) a dAb fragment (Ward et al., Nature 341:544-546, 1989) which consists of a V_{H} domain; (v) an isolated complimentarity determining region (CDR); and (vi) an F(ab′)₂ fragment, a bivalent fragment comprising two Fab fragments linked by a disulfide bridge at the hinge region.

Immunoglobulins and certain variants thereof are known and many have been prepared in recombinant cell culture (for example, see U.S. Pat. No. 4,745,055; U.S. Pat. No. 4,444,487; WO 88/03565; EP 0256654; EP 0120694; EP 0125023; Faoulkner et al., Nature 298:286, 1982; Morrison, J. Immunol. 123:793, 1979; Morrison et al., Ann Rev. Immunol 2:239, 1984).

Autoimmune disorder: A disorder in which the immune system produces an immune response (e.g. a B cell or a T cell response) against an endogenous antigen, with consequent injury to tissues.

Cell: A plant, animal, insect, bacterial, or fungal cell.

Conservative variants: “Conservative” amino acid substitutions are those substitutions that do not substantially affect or decrease an activity or antigenicity of a JMHV polypeptide. Specific, non-limiting examples of a conservative substitution include the following examples:

| Original | Conservative |
|---|---|
| Residue | Substitutions |
| Ala | Ser |
| Arg | Lys |
| Asn | Gln, His |
| Asp | Glu |
| Cys | Ser |
| Gln | Asn |
| Glu | Asp |
| His | Asn; Gln |
| Ile | Leu, Val |
| Leu | Ile; Val |
| Lys | Arg; Gln; Glu |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

The term conservative variation also includes the use of a substituted amino acid in place of an unsubstituted parent amino acid, provided that antibodies raised to the substituted polypeptide also immunoreact with the unsubstituted polypeptide. Non-conservative substitutions are those that reduce an activity or antigenicity.

Degenerate variant: A polynucleotide encoding a JMHV polypeptide that includes a sequence that is degenerate as a result of the genetic code. There are 20 natural amino acids, most of which are specified by more than one codon. Therefore, all degenerate nucleotide sequences are included in the disclosure as long as the amino acid sequence of the JMHV polypeptide encoded by the nucleotide sequence is unchanged.

Expression Control Sequences: Nucleic acid sequences that control and regulate the expression of a nucleic acid sequence, such as a heterologous nucleic acid sequence, to which it is operably linked. Expression control sequences are operably linked to a nucleic acid sequence when the expression control sequences control and regulate the transcription and, as appropriate, translation of the nucleic acid sequence. Thus expression control sequences can include appropriate promoters, enhancers, transcription terminators, polyA signals, a start codon (for instance, ATG) in front of a protein-encoding polynucleotide sequence, splicing signal for introns, maintenance of the correct reading frame of that gene to permit proper translation of mRNA, and stop codons. The term “control sequences” is intended to include, at a minimum, components whose presence can influence expression, and can also include additional components whose presence is advantageous, for example, leader sequences and fusion partner sequences. Expression control sequences can include a promoter.

A promoter is a minimal sequence sufficient to direct transcription of a nucleic acid. Promoters may be cell-type specific or tissue specific. A promoter includes necessary nucleic acid sequences near the start site of transcription, such as, in the case of a polymerase II type promoter, a TATA element. A promoter also optionally includes distal enhancer or repressor elements which can be located as much as several thousand base pairs from the start site of transcription. Both constitutive and inducible promoters are included (see for example, Bitter et al., Methods in Enzymology 153:516-544, 1987).

For example, when cloning in bacterial systems, inducible promoters such as pL of bacteriophage lambda, plac, ptrp, ptac (ptrp-lac-hybrid promoter) and the like may be used. In one embodiment, when cloning in mammalian cell systems, promoters derived from the genome of mammalian cells (for example, metallothionein promoter) or from mammalian viruses (for example, the retrovirus long terminal repeat; the adenovirus late promoter; the vaccinia virus 7.5K promoter) can be used. Promoters produced by recombinant DNA or synthetic techniques may also be used to provide for transcription of the nucleic acid sequences. A polynucleotide can be inserted into an expression vector that contains a promoter sequence which facilitates the efficient transcription of the inserted genetic sequence of the host. The expression vector typically contains an origin of replication, a promoter, as well as specific nucleic acid sequences that allow phenotypic selection of the transformed cells. In one embodiment, the promoter is a cytomegalovirus promoter.

Herpesvirus: Viruses that include large (genomes up to 235 kbp DNA) that are generally complex viruses such that the virus encodes about 35 virion proteins. Herpesviruses encode a variety of enzymes involved in nucleic acid metabolism, DNA synthesis and protein processing (protein kinase).

The herpesvirus particle is complex, and includes a core that has a toroidal shape, with the large DNA genome would around a proteinaceous core. The complex capsid surrounds the core. Outside the capsid is the tegument, a protein-filled region which appears amorphous in electron micrographs. On the outside of the particle is the envelope, which contains numerous glycoproteins.

Herpesvirus genomes can have a unique long (UL) and a unique short (US) region, bounded by inverted repeats. The repeats allow rearrangements of the unique regions and Herpesvirus genomes exist as a mixture of four isomers. Herpesvirus genomes also contain multiple repeated sequences. The JMHV genome, however, has a unique long region bound by inverted repeat, which is consistent for gamma-herpesvirues.

Homologs: Two nucleotide or amino acid sequences that share a common ancestral sequence and diverged when a species carrying that ancestral sequence split into two species. Homologs frequently show a substantial degree of sequence identity.

Immuno-compromised: Lacking a normal immune response. Immuno-compromised refers to a condition in which some or all of an animal's immune system is inoperative, leaving the animal with an increased susceptibility to infection or disease. An animal may be rendered immuno-compromised by a biological agent such as, in the case of non-human primates, Simian Immunodeficiency Virus (SIV). Many strains of SIV have been isolated and characterized; any SIV strain that produces an immuno-compromised state can be used in the present disclosure including, but not limited to, for example, SIVmac239 (Kestler et al., Science 248:1109-12, 1990), SIVmac251 (Daniels et al., Science 228:1201-4, 1985), SIVdeltaB670 (Murphy-Corb et al., Nature 321:435, 1986) and SIVmne (Benveniste et al., J. Virol. 62:2091-101, 1988). In addition, hybrid SIV/HIV chimeras as known in the field can be employed, as can HIV-2. Simian type D retroviruses (SRVs), which cause an AIDS-like disease in rhesus monkeys, can alternatively be used to immuno-compromise the animals in place of SIV. These viral agents are administered to the animal using conventional means, such as intravenous or intramuscular injection, or oral, intrarectal or intravaginal inoculation (also see Example 24). Either intact viral particles or viral DNA may be administered. As known in the field, plasmid constructs containing the entire SIV genome are infectious when inoculated into animals and so may be employed in place of purified viral DNA.

Alternatively, an animal may be rendered immuno-compromised by administration of agents that target the immune system, including but not limited to anti-CD3 antibody (CD3 being the T cell receptor) either alone or conjugated with a toxic moiety, or immunosuppressive compounds including prednisone, azathioprine, cyclosporine A, and cyclophosphamide. Where an immunosuppressive compound such as cyclosporine is employed, an allogenic stimulus (such as a blood transfusion) may be administered with the subsequent administration of RRV to activate infection.

Alternatively, other methods of rendering an animal immuno-compromised may be used, including radiation treatment and surgical intervention.

Immune response: A response of a cell of the immune system, such as a B cell, T cell, or monocyte, to a stimulus. In one embodiment, the response is specific for a particular antigen (an “antigen-specific response”). The response can also be a non-specific response (not targeted specifically to salivary polypeptides) such as production of lymphokines. In one embodiment, an immune response is a T cell response, such as a CD4+ response or a CD8+ response. In another embodiment, the response is a Th1 (a subset of helper T cells) response. In yet another embodiment, the response is a B cell response, and results in the production of specific antibodies.

Isolated: An “isolated” biological component (such as a nucleic acid, peptide or protein) has been substantially separated, produced apart from, or purified away from other biological components in the cell of the organism in which the component naturally occurs, i.e., other chromosomal and extrachromosomal DNA and RNA, and proteins. Nucleic acids, peptides and proteins which have been “isolated” thus include nucleic acids and proteins purified by standard purification methods. The term also embraces nucleic acids, peptides and proteins prepared by recombinant expression in a host cell as well as chemically synthesized nucleic acids.

Japanese Macaque Herpesvirus (JMHV): A virus having the virological, immunological or pathological characteristics of Japanese macaque herpesvirus (e.g. JMHV 17792). JMHV causes the symptoms of MS in Japanese macaque monkeys which are infected with the virus. In particular examples, the JMHV has at least 85% (for example at least 90%, 95% or 98%) sequence identity to the JMHV deposited with the virus deposited as ATCC Accession No. PTA-1884. In other examples, the JMHV has at least 90%, 91%, 92%, 93%, 94%, 95%, or 98% homology with an RRV (e.g. ATCC VR-2901). Without being bound by theory, the JMHV may be derived from an RRV (see the experimental examples).

JMHV 17792: Japanese macaque herpesvirus isolate 17792. A Budapest Treaty deposit of JMHV 17792 was made with the American Type Culture Collection (ATCC), Manassas, Va., on May 18, 2001. This virus may be grown on primary Japanese macaque fibroblasts, using standard virological techniques. Alternatively, it may be grown on commercially available macaque cell lines. Infection of a non-human primate with JMHV 17792 can be accomplished using any standard method, including intravenous injection. In one embodiment, infection is achieved using 106 plaque forming units of JMHV 17792.

Label: A detectable compound or composition that is conjugated directly or indirectly to another molecule to facilitate detection of that molecule. Specific, non-limiting examples of labels include fluorescent tags, enzymatic linkages, and radioactive isotopes.

Multiple Sclerosis: A chronic, progressive disease of the central nervous system. Currently, the exact cause of the disease is unknown and there is no cure. Multiple sclerosis refers to multiple areas of patchy scarring, or plaques, that result from demyelination (destruction of myelin, a fatty insulation covering the nerve fibers). When the myelin sheath is destroyed during the MS disease process, signals transmitted throughout the CNS are slowed or disrupted. In many cases, the body may compensate for the loss of myelin by increasing the density of the sodium channels so that action potentials can continue to be carried, in spite of loss of myelin. The nerves also retain the capacity to remyelinate. Unfortunately, the disease process often outpaces these corrective actions.

The symptoms, severity, and course of MS vary widely depending partly on the sites of the plaques and the extent of the demyelination. Experts generally group multiple sclerosis into four types: relapsing-remitting, primary-progressive, secondary-progressive, and progressive-relapsing MS. Relapsing-remitting multiple sclerosis generally occurs in younger people and is the most common form of MS. Symptoms flare up for several days and then go into remission over the next four to eight weeks. The latter three forms (primary-progressive, secondary-progressive, and progressive-relapsing MS) generally fall under the category of chronic-progressive MS. In chronic-progressive MS the symptoms of the disease continue to worsen slowly without remission. About 20% of multiple sclerosis patients (usually those patients whose first symptoms occur after age 45) have the chronic-progressive form of MS without first developing relapsing-remitting MS. Chronic-progressive MS may lead to serious speech problems and paralysis, and generally the symptoms continue to worsen over time.

Multiple sclerosis is defined as an autoimmune disease; that is, the body's immune system is damaged by genetic or environmental factors or both, causing it to attack its own tissues. In the case of MS, these tissues are the myelin covering the nerve fibers in the brain.

A symptom of MS is any physical characteristic associated with the disease process. For example, the first symptom of MS is often optic neuritis, the inflammation of the optic nerve. Vision, usually in one eye, becomes unclear or doubled, and there may be a shimmering effect. Pain or nystagmus, involuntary jerking or movement of the eye, may also occur. In 20% of people with this condition, MS develops within two years; in 45% to 80% it develops within 15 years. Other early symptoms of multiple sclerosis include fatigue, heaviness or clumsiness in the arms and legs, tingling sensations, and poor coordination. Another indication of MS is a reaction known as Llermitte's sign, whereby bending the neck produces an electrical sensation that runs down the back and into the legs.

As the disease develops over months or even years, other symptoms may include spasticity, imbalance, tremors, incontinence, constipation, sexual dysfunction, hearing loss, vertigo, facial pain, and difficulties in swallowing. Problems in speech may occur because of difficulty in controlling the quality of the voice and articulating words. About half of patients display changes in mental function, including problems in concentration and problem solving. In about 10% of cases, there is severe mental dysfunction which resembles dementia. One of the primary symptoms of MS is spasticity, which is characterized by weakness, loss of dexterity, and the inability to control specific movements.

Myelin: A coating of nerve cells (neurons) made from layers of cell membranes that are produced in the brain and spinal cord by specialized cells called oligodendrocytes. Myelin coats lie in segments along the axons, the long filaments that carry electric impulses away from a nerve cell. The segments are separated from each other by tiny clusters called nodes of Ranvier, which house channels for sodium ions. These sodium ions are important for boosting the electrical charge required to pass signals from one nerve to another.

Non-human primate: Simian primates including chimpanzees, orangutans, baboons, and macaques. Any non-human primate may be used to produce a KSHV-disease animal model or a JMHV-disease animal model by the methods disclosed herein. Thus, in addition to the rhesus macaque and Japanese macaque models described in detail below, pigtail and cynomologus macaques and baboons may also be used to produce KSHV-disease or JMHV-disease animal models by the methods disclosed herein.

Oligonucleotide: A linear polynucleotide sequence of up to about 200 nucleotide bases in length, for example a polynucleotide (such as DNA or RNA) which is at least 6 nucleotides, for example at least 15, 25, 50, 100 or even 200 nucleotides long.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

ORF: Open reading frame. Contains a series of nucleotide triplets (codons) coding for amino acids without any termination codons. These sequences are usually translatable into protein.

PCR: Polymerase chain reaction. Describes a technique in which cycles of denaturation, annealing with primer, and then extension with DNA polymerase are used to amplify the number of copies of a target DNA sequence.

Polypeptide: A polymer in which the monomers are amino acid residues that are joined together through amide bonds. When the amino acids are alpha-amino acids, either the L-optical isomer or the D-optical isomer can be used, the L-isomers being preferred. The terms “polypeptide” or “protein” as used herein is intended to encompass any amino acid sequence and include modified sequences such as glycoproteins. The term “polypeptide” is specifically intended to cover naturally occurring proteins, as well as those that are recombinantly or synthetically produced.

The term “fragment” refers to a portion of a polypeptide that is at least 8, 10, 15, 20 or 25 amino acids in length. The term “functional fragments of a polypeptide” refers to all fragments of a polypeptide that retain an activity of the polypeptide (e.g., the binding of an antigen). Biologically functional fragments, for example, can vary in size from a polypeptide fragment as small as an epitope capable of binding an antibody molecule to a large polypeptide capable of participating in the characteristic induction or programming of phenotypic changes within a cell. The term “soluble” refers to a form of a polypeptide that is not inserted into a cell membrane.

Polypeptide modifications: JMHV polypeptides include synthetic embodiments of polypeptides described herein. In addition, analogs (non-peptide organic molecules), derivatives (chemically functionalized peptide molecules obtained starting with the disclosed polypeptide sequences) and variants (homologs) of these proteins can be utilized in the methods described herein. Each polypeptide of the disclosure is comprised of a sequence of amino acids, which may be either L- and/or D-amino acids, naturally occurring and otherwise.

Polypeptides may be modified by a variety of chemical techniques to produce derivatives having essentially the same activity as the unmodified polypeptides, and optionally having other desirable properties. For example, carboxylic acid groups of the protein, whether carboxyl-terminal or side chain, may be provided in the form of a salt of a pharmaceutically-acceptable cation or esterified to form a C₁-C₁₆ ester, or converted to an amide of formula NR₁R₂ wherein R₁ and R₂ are each independently H or C₁-C₁₆ alkyl, or combined to form a heterocyclic ring, such as a 5- or 6-membered ring. Amino groups of the peptide, whether amino-terminal or side chain, may be in the form of a pharmaceutically-acceptable acid addition salt, such as the HCl, HBr, acetic, benzoic, toluene sulfonic, maleic, tartaric, and other organic salts, or may be modified to C₁-C₁₆ alkyl or dialkyl amino or further converted to an amide.

Hydroxyl groups of the peptide side chains may be converted to C₁-C₁₆ alkoxy or to a C₁-C₁₆ ester using well-recognized techniques. Phenyl and phenolic rings of the peptide side chains may be substituted with one or more halogen atoms, such as fluorine, chlorine, bromine, or iodine, or with C₁-C₁₆ alkyl, C₁-C₁₆ alkoxy, carboxylic acids and esters thereof, or amides of such carboxylic acids. Methylene groups of the peptide side chains can be extended to homologous C₂-C₄ alkylenes. Thiols can be protected with any one of a number of well-recognized protecting groups, such as acetamide groups. Those skilled in the art will also recognize methods for introducing cyclic structures into the peptides of this disclosure to select and provide conformational constraints to the structure that result in enhanced stability.

Peptidomimetic and organomimetic embodiments are envisioned, whereby the three-dimensional arrangement of the chemical constituents of such peptido- and organomimetics mimic the three-dimensional arrangement of the peptide backbone and component amino acid side chains, resulting in such peptido- and organomimetics of a L. longipalpis polypeptide having measurable or enhanced ability to generate an immune response. For computer modeling applications, a pharmacophore is an idealized, three-dimensional definition of the structural requirements for biological activity. Peptido- and organomimetics can be designed to fit each pharmacophore with current computer modeling software (using computer assisted drug design or CADD). See Walters, “Computer-Assisted Modeling of Drugs,” Klegerman & Groves (eds.), 1993, Pharmaceutical Biotechnology, Interpharm Press: Buffalo Grove, Ill., pp. 165-174 and Principles of Pharmacology Munson (ed.) 1995, Ch. 102, for descriptions of techniques used in CADD. Also included are mimetics prepared using such techniques.

Pharmaceutically acceptable carriers: The pharmaceutically acceptable carriers useful in this disclosure include conventional carriers. Remington's Pharmaceutical Sciences, by E. W. Martin, Mack Publishing Co., Easton, Pa., 15th Edition (1975), describes compositions and formulations suitable for pharmaceutical delivery of the viruses, nucleic acids and/or proteins herein disclosed.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol, ethanol, combinations thereof, or the like, as a vehicle. The carrier and composition can be sterile, and the formulation suits the mode of administration. For solid compositions (e.g., powder, pill, tablet, or capsule forms), conventional non-toxic solid carriers can include, for example, pharmaceutical grades of mannitol, lactose, starch, sodium saccharine, cellulose, magnesium carbonate, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

The composition can be a liquid solution, suspension, emulsion, tablet, pill, capsule, sustained release formulation, or powder. The composition can be formulated as a suppository, with traditional binders and carriers such as triglycerides.

Probes and primers: Nucleic acid probes and primers may readily be prepared based on the amino acid sequences provided by this disclosure. A probe is an isolated nucleic acid attached to a detectable label or reporter molecule. Typical labels include radioactive isotopes, ligands, fluorescent molecules, chemiluminescent molecules, and enzymes. In other embodiments, labels include co-factors, enzyme substrates; and haptens.

Methods for labeling and guidance in the choice of labels appropriate for various purposes are discussed, e.g., in Sambrook et al., in Molecular Cloning: A Laboratory Manual, Cold Spring (1989) and Ausubel et al., in Current Protocols in Molecular Biology, Greene Publishing Associates and Wiley-Intersciences (1987).

Primers are short nucleic acids, such as DNA oligonucleotides 10 nucleotides or more in length. Primers may be annealed to a complementary target DNA strand by nucleic acid hybridization to form a hybrid between the primer and the target DNA strand, and then extended along the target DNA strand by a DNA polymerase enzyme. Primer pairs can be used for amplification of a nucleic acid sequence, e.g., by the polymerase chain reaction (PCR) or other nucleic-acid amplification methods known in the art.

Methods for preparing and using probes and primers are described, for example, in Sambrook et al. (Molecular Cloning: A Laboratory Manual, 2^{nd} ed., vol. 1-3, Cold Spring Harbor, N.Y. 1989); Ausubel et al. (Current Protocols in Molecular Biology, Greene Publ. Assoc. & Wiley-Intersciences. 1987) and Innis et al. (PCR Protocols, A Guide to Methods and Applications, 1990, Innis et al. (eds.), 21-27, Academic Press, Inc., San Diego, Calif.). PCR primer pairs can be derived from a known sequence, for example, by using computer programs intended for that purpose such as Primer (Version 0.5, © 1991, Whitehead Institute for Biomedical Research, Cambridge, Mass.).

Probes and primers as used in the present disclosure typically comprise at least 15 contiguous nucleotides of the RRV genome sequence (SEQ ID NO: 1), or 15 contiguous nucleotides of a JMHV sequence. One of skill in the art will appreciate that the specificity of a particular probe or primer increases with its length. Thus, for example, a primer comprising 20 consecutive nucleotides will anneal to a target with a higher specificity than a corresponding primer of only 15 nucleotides. Thus, in order to obtain greater specificity, probes and primers may be selected that comprise 20, 25, 30, 35, 40, 50 or more consecutive nucleotides. In order to enhance specificity, longer probes and primers may also be employed, such as probes and primers that comprise at least 20, 30, 40, 50, 60, 70, 80, 90, 100, or 150 consecutive nucleotides of the disclosed nucleic acid sequences.

Alternatively, such probes and primers may comprise at least 15, 20, 30, 40, 50, 60, 70, 80, 90, 100, or 150 consecutive nucleotides that share a defined level of sequence identity with the disclosed RRV or JMHV sequence, for instance, at least a 60%, 70%, 80%, 90%, 95% or 98% sequence identity. Alternatively, such probes and primers may be nucleotide molecules which hybridize under wash conditions of 70° C. and about 0.2×SSC for 1 hour, or alternatively under less stringent conditions of 65° C., 60° C., or 55° C. with from about 0.2 to 2×SSC (with, for instance, about 0.1% SDS) for 1 hour with a portion of the JMHV sequence.

Protein Purification: The JMHV polypeptides disclosed herein can be purified by any of the means known in the art. See, e.g., Guide to Protein Purification, ed. Deutscher, Meth. Enzymol. 185, Academic Press, San Diego, 1990; and Scopes, Protein Purification: Principles and Practice, Springer Verlag, New York, 1982. Substantial purification denotes purification from other proteins or cellular components. A substantially purified protein is at least 60%, 70%, 80%, 90%, 95% or 98% pure. Thus, in one specific, non-limiting example, a substantially purified protein is 90% free of other proteins or cellular components.

Purified (Isolated): The term purified or isloated does not require absolute purity; rather, it is intended as a relative term. Thus, for example, a purified peptide preparation is one in which the peptide or protein is more enriched than the peptide or protein is in its natural environment within a cell. Preferably, a preparation is purified such that the protein or peptide represents at least 50% of the total peptide or protein content of the preparation.

Recombinant: A recombinant nucleic acid is one that has a sequence that is not naturally occurring or has a sequence that is made by an artificial combination of two otherwise separated segments of sequence. This artificial combination is often accomplished by chemical synthesis or, more commonly, by the artificial manipulation of isolated segments of nucleic acids, e.g., by genetic engineering techniques.

RRV 17577: Rhesus macaque rhadinovirus RRV isolate 17577. A Budapest Treaty deposit of RRV 17577 was made with the American Type Culture Collection, Manassas, Va., on Mar. 12, 1998, and has been accorded ATCC Accession No. VR-2601. This virus may be grown on primary rhesus fibroblasts, as described below (see Examples 1 and 14), using standard virological techniques. Alternatively, it may be grown on commercially available rhesus cell lines, including those available from ATCC, such as ATCC CRL-6306 and ATCC CL-160. Infection of a non-human primate with RRV 17577 may be accomplished using any standard method, including intravenous injection (see Examples 13, 23 and 24). Typically, infection is achieved by intravenous injection of around 106 plaque forming units (PFUs) of RRV 17577.

Rhesus Macaque Rhadinovirus (RRV): A virus having the virological and immunological characteristics of RRV 17577, which causes Kaposi's sarcoma-like disease and lymphoma in infected immunocompromised rhesus monkeys (see published PCT Application No. WO 00/28040 and U.S. patent application Ser. No. 09/831,000, filed May 2, 2001, which are both incorporated by reference herein).

Sequence Identity: The similarity between two nucleic acid sequences, or two amino acid sequences, is expressed in terms of the similarity between the sequences, otherwise referred to as sequence identity. Sequence identity is frequently measured in terms of percentage identity (or similarity or homology); the higher the percentage, the more similar the two sequences are. Homologs or orthologs of nucleic acid or amino acid sequences will possess a relatively high degree of sequence identity when aligned using standard methods. This homology will be more significant when the orthologous proteins or nucleic acids are derived from species which are more closely related (e.g., human and chimpanzee sequences), compared to species more distantly related (e.g., human and C. elegans sequences). Typically, orthologs are at least 50% identical at the nucleotide level and at least 50% identical at the amino acid level when comparing human orthologous sequences.

Methods of alignment of sequences for comparison are well known. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al., Computer Appls. Biosci. 8:155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations.

The NCBI Basic Local Alignment Search Tool (BLAST) (Altschul et al., J. Mol. Biol. 215:403-10, 1990) is available from several sources, including the National Center for Biotechnology Information (NCBI, Bethesda, Md.) and on the Internet, for use in connection with the sequence analysis programs blastp, blastn, blastx, tblastn and tblastx. Each of these sources also provides a description of how to determine sequence identity using this program.

Homologous sequences are typically characterized by possession of at least 60%, 70%, 75%, 80%, 90%, 95% or at least 98% sequence identity counted over the full length alignment with a sequence using the NCBI Blast 2.0, gapped blastp set to default parameters. Queries searched with the blastn program are filtered with DUST (Hancock and Armstrong, Comput. Appl. Biosci. 10:67-70, 1994). It will be appreciated that these sequence identity ranges are provided for guidance only; it is entirely possible that strongly significant homologs could be obtained that fall outside of the ranges provided.

Nucleic acid sequences that do not show a high degree of identity may nevertheless encode similar amino acid sequences, due to the degeneracy of the genetic code. It is understood that changes in nucleic acid sequence can be made using this degeneracy to produce multiple nucleic acid sequences that all encode substantially the same protein.

One indication that two nucleic acid sequences are substantially identical is that the polypeptide encoded by the first nucleic acid is immunologically cross reactive with the polypeptide encoded by the second nucleic acid.

An alternative indication that two nucleic acid molecules are closely related is that the two molecules hybridize to each other under stringent conditions, as described under “specific hybridization.”

Homologs of the disclosed RRV or JMHV nucleic acids typically possess at least 50% sequence identity counted over the length of one of the nucleic acids (the reference nucleic acid) using the NCBI Blast 2.0.6, gapped blastn set to default parameters. Nucleic acids showing substantial similarity when assessed by this method may show, for example, at least 50%, 60%, 70%, 80%, 90%, 95% or even 98% or greater sequence identity. When less than the entire sequence is being compared for sequence identity, substantially similar nucleotide sequences will typically possess at least 70% sequence identity over short windows of 30-90 nucleic acids, and may possess sequence identities of at least 80%, 90%, 95% or 98% or greater.

Homologs of the disclosed RRV or JMHV proteins typically possess at least 50% sequence identity counted over full-length alignment with the amino acid sequence of RRV using the NCBI Blast 2.0, gapped blastp set to default parameters. For comparisons of amino acid sequences of greater than about 30 amino acids, the Blast 2 sequences function is employed using the default BLOSUM62 matrix set to default parameters, (gap existence cost of 11, and a per residue gap cost of 1). When aligning short peptides (fewer than around 30 amino acids), the alignment should be performed using the Blast 2 sequences function, employing the PAM30 matrix set to default parameters (open gap 9, extension gap 1 penalties). Proteins with even greater similarity to the reference sequence will show increasing percentage identities when assessed by this method, such as at least 50%, at least 55%, at least 60%, at least 70%, at least 75%, at least 80%, at least 90%, at least 95%, at least 98%, or at least 99% sequence identity. When less than the entire sequence is being compared for sequence identity, homologs will typically possess at least 70% sequence identity over short windows of 10-20 amino acids, and may possess sequence identities of at least 75%, at least 85% or at least 90%, at least 95% or 98% depending on their similarity to the reference sequence.

When comparing degrees of sequence identity between similar proteins, the degree of identity will be equal to or less than that the degree of similarity, due to the fact the similarity takes into account conservative amino acid substitutions. So, for instance, the degree of sequence identity between to substantially similar proteins may be at least 50%, 55%, 65%, 75%, 85%, 95%, 98% or more.

One of ordinary skill in the art will appreciate that these sequence identity ranges are provided for guidance only; it is entirely possible that strongly significant homologs could be obtained that fall outside of the ranges provided. The present disclosure provides not only the peptide homologs that are described above, but also nucleic acid molecules that encode such homologs.

An alternative indication that two nucleic acid molecules are closely related is that the two molecules hybridize to each other under stringent conditions. In nucleic acid hybridization reactions, the conditions used to achieve a particular level of stringency will vary, depending on the nature of the nucleic acids being hybridized. For example, the length, degree of complementarity, nucleotide sequence composition (e.g., GC v. AT content), and nucleic acid type (e.g., RNA v. DNA) of the hybridizing regions of the nucleic acids can be considered in selecting hybridization conditions. An additional consideration is whether one of the nucleic acids is immobilized, for example, on a filter.

Specific hybridization: Specific hybridization refers to the binding, duplexing, or hybridizing of a molecule only or substantially only to a particular nucleotide sequence when that sequence is present in a complex mixture (e.g. total cellular DNA or RNA). Specific hybridization may also occur under conditions of varying stringency.

A specific, non-limiting example of progressively higher stringency conditions is as follows: 2×SSC/0.1% SDS at about room temperature (hybridization conditions); 0.2×SSC/0.1% SDS at about room temperature (low stringency conditions); 0.2×SSC/0.1% SDS at about 42° C. (moderate stringency conditions); and 0.1×SSC at about 68° C. (high stringency conditions). One of skill in the art can readily determine variations on these conditions (for example, Molecular Cloning: A Laboratory Manual, 2nd ed., vol. 1-3, ed. Sambrook et al., Cold Spring Harbor Laboratory Press, Cold Spring Harbor, N.Y., 1989). The hydridization conditions can be carried out over 2 to 16 hours. Washing can be carried out using only one of the above conditions, for example, high stringency conditions, or each of the conditions can be used, for example, for 10-15 minutes each, in the order listed above, repeating any or all of the steps listed. However, as mentioned above, optimal conditions will vary, depending on the particular hybridization reaction involved, and can be determined empirically.

Specific binding agent: An agent that binds substantially only to a defined target. As used herein, the term “JMHV peptide specific binding agent” includes anti-JMHV peptide antibodies and other agents that bind substantially only to the JMH peptide. The antibodies may be monoclonal or polyclonal antibodies that are specific for a JMHV peptide, as well as immunologically effective portions (“fragments”) thereof.

In one embodiment, the antibodies are monoclonal antibodies (or immunologically effective portions thereof) and may also be humanized monoclonal antibodies (or immunologically effective portions thereof). Immunologically effective portions of monoclonal antibodies include Fab, Fab′, F(ab′)₂, Fabc and Fv portions (for a review, see Better and Horowitz, Methods. Enzymol. 178:476-96, 1989). Anti-inhibitory peptide antibodies may also be produced using standard procedures described in a number of texts, including Antibodies, A Laboratory Manual by Harlow and Lane, Cold Spring Harbor Laboratory (1988).

Methods of making humanized monoclonal antibodies are well known, and include those described in U.S. Pat. No. 5,585,089; U.S. Pat. No. 5,565,332; U.S. Pat. No. 5,225,539; U.S. Pat. No. 5,693,761; U.S. Pat. No. 5,693,762; U.S. Pat. No. 5,585,089; and U.S. Pat. No. 5,530,101 and references cited therein. Similarly, methods of making and using immunologically effective portions of monoclonal antibodies, also referred to as antibody fragments, are well known and include those described in Better and Horowitz, Meth. Enzymol. 178:176-496, 1989; Better et al., 1990, Better and Horowitz, 1990, Advances in Gene technology: The Molecular Biology of Immune Disease & the Immune Response (ICSU Short Reports); Glockshuber et al., Biochemistry 29:1362-7, 1990; and U.S. Pat. No. 5,648,237; U.S. Pat. No. 4,946,778 and U.S. Pat. No. 5,455,030, and references cited therein.

The determination that a particular agent binds substantially only to a JMHV peptide may readily be made by using or adapting routine procedures. One suitable in vitro assay makes use of the Western blotting procedure (described in many standard texts, including Antibodies, A Laboratory Manual by Harlow and Lane). Western blotting may be used to determine that a given JMHV peptide binding agent, binds substantially only to the specific JMHV protein.

Supernatant: The culture medium in which a cell is grown. The culture medium includes material from the cell. If the cell is infected with a virus, the supernatant can include viral particles.

Subject: This term includes both human and non-human subjects. Similarly, the term “patient” includes both human and veterinary subjects. In one embodiment, the subject has multiple sclerosis.

Symptom and sign: Any subjective evidence of disease or of a subject's condition, i.e., such evidence as perceived by the subject; a noticeable change in a subject's condition indicative of some bodily or mental state. A “sign” is any abnormality indicative of disease, discoverable on examination or assessment of a subject. A sign is generally an objective indication of disease. Signs include, but are not limited to any measurable parameters such as tests for immunological status or the presence of lesions in a subject with multiple sclerosis.

Therapeutically Effective Amount: A dose sufficient to prevent advancement, or to cause regression of the disease, or which is capable of reducing symptoms caused by the disease, such as multiple sclerosis.

Transduced: A transduced cell is a cell into which has been introduced a nucleic acid molecule by molecular biology techniques. As used herein, the term transduction encompasses all techniques by which a nucleic acid molecule might be introduced into such a cell, including transfection with viral vectors, transformation with plasmid vectors, and introduction of naked DNA by electroporation, lipofection, and particle gun acceleration.

Transgenic Cell: Transformed cells which contain foreign, non-native DNA.

Variants of Amino Acid and Nucleic Acid Sequences: The production of RRV or JMHV proteins can be accomplished in a variety of ways (for example see Examples 17, 21 and 25). DNA sequences which encode the protein, or a fragment of the protein, can be engineered such that they allow the protein to be expressed in eukaryotic cells, bacteria, insects, and/or plants. In order to accomplish this expression, the DNA sequence can be altered and operably linked to other regulatory sequences. The final product, which contains the regulatory sequences and the therapeutic protein, is referred to as a vector. This vector can then be introduced into the eukaryotic cells, bacteria, insect, and/or plant. Once inside the cell the vector allows the protein to be produced.

One of ordinary skill in the art will appreciate that the DNA can be altered in numerous ways without affecting the biological activity of the encoded protein. For example, PCR may be used to produce variations in the DNA sequence which encodes JMHV proteins. Such variants may be variants that are optimized for codon preference in a host cell that is to be used to express the protein, or other sequence changes that facilitate expression.

Two types of cDNA sequence variant may be produced. In the first type, the variation in the cDNA sequence is not manifested as a change in the amino acid sequence of the encoded polypeptide. These silent variations are simply a reflection of the degeneracy of the genetic code. In the second type, the cDNA sequence variation does result in a change in the amino acid sequence of the encoded protein. In such cases, the variant cDNA sequence produces a variant polypeptide sequence. In order to preserve the functional and immunologic identity of the encoded polypeptide, it is preferred that any such amino acid substitutions are conservative. Conservative substitutions replace one amino acid with another amino acid that has some homology in size, hydrophobicity, etc. Such substitutions generally are conservative when it is desired to finely modulate the characteristics of the protein. For example, conservative substitutions generally maintain (a) the structure of the polypeptide backbone in the area of the substitution, for example, as a sheet or helical conformation, (b) the charge or hydrophobicity of the molecule at the target site, or (c) the bulk of the side chain.

Examples of amino acids which may be substituted for an original amino acid in a protein and which are regarded as conservative substitutions include: Ser for Ala; Lys for Arg; Gln or His for Asn; Glu for Asp; Ser for Cys; Asn for Gln; Asp for Glu; Pro for Gly; Asn or Gln for His; Leu or Val for Ile; Ile or Val for Leu; Arg or Gln for Lys; Leu or Ile for Met; Met, Leu or Tyr for Phe; Thr for Ser; Ser for Thr; Tyr for Trp; Trp or Phe for Tyr; and Ile or Leu for Val.

The substitutions which in general are expected to produce the greatest changes in protein properties will be non-conservative, for instance changes in which (a) a hydrophilic residue, e.g., seryl or threonyl, is substituted for (or by) a hydrophobic residue, e.g., leucyl, isoleucyl, phenylalanyl, valyl or alanyl; (b) a cysteine or proline is substituted for (or by) any other residue; (c) a residue having an electropositive side chain, e.g., lysyl, arginyl, or histadyl, is substituted for (or by) an electronegative residue, e.g., glutamyl or aspartyl; or (d) a residue having a bulky side chain, e.g., phenylalanine, is substituted for (or by) one not having a side chain, e.g., glycine.

Variations in the DNA sequence that result in amino acid changes, whether conservative or not, should be minimized in order to preserve the functional and immunologic identity of the encoded protein. The immunologic identity of the protein may be assessed by determining whether it is recognized by an antibody to an RRV (or JMHV) protein; a variant that is recognized by such an antibody is immunologically conserved. Any DNA sequence variant will preferably introduce no more than 20, and preferably fewer than 10 amino acid substitutions into the encoded polypeptide. Variant amino acid sequences can, for example, be 80%, 90%, 95% or even 98% identical to the native amino acid sequence.

Vector: A nucleic acid molecule as introduced into a host cell, thereby producing a transformed host cell. A vector may include nucleic acid sequences that permit it to replicate in the host cell, such as an origin of replication. A vector may also include one or more selectable marker genes and other genetic elements known in the art.

Virion: A complete viral particle including envelope, capsid (if any), and nucleic acid elements.

The present disclosure utilizes standard laboratory practices for the cloning, manipulation and sequencing of nucleic acids, purification and analysis of proteins and other molecular biological and biochemical techniques, unless otherwise stipulated. Such techniques are explained in detail in standard laboratory manuals such as Sambrook et al. (Molecular Cloning: A Laboratory Manual, 2^{nd} ed., vol. 1-3, Cold Spring Harbor, N.Y., 1989) and Ausubel et al. (Current Protocols in Molecular Biology, Greene Publ. Assoc. & Wiley-Intersciences, 1987).

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The term “comprises” means “includes.” All publications, patent applications, patents, and other references mentioned herein are incorporated by reference in their entirety. In case of conflict, the present specification, including explanations of terms, will control. In addition, the materials, methods, and examples are illustrative only and not intended to be limiting.

### Polynucleotides and Polypeptides

The sequence of a JMHV is disclosed herein (see SEQ ID NO: 1). Homologous nucleic acid sequences including an nucleic acid sequence at least about 90%, 95%, 98%, or 99% identical to the nucleic acid sequence as set forth in SEQ ID NO: 1 are also disclosed herein. Polynucleotides encoding a JMHV polypeptide (encoded by an open reading frame, or ORF) are also provided, and are termed JMHV polynucleotides. These polynucleotides include DNA, cDNA and RNA sequences which encode a JMHV polypeptide, or encode a polypeptide at least about 85%, at least about 90%, at least about 95%, at least about 98%, or at least about 99% homologous to the JMHV ORF (see Table 3). Specific, non-limiting examples of a JMHV nucleic acid encoding a ORF are nucleic acid 21845 to nucleic acid 22120 of SEQ ID NO: 1 (JMHV25), nucleic acid 22363 to nucleic acid 22701 of SEQ ID NO: 1 (JMHV26), nucleic acid 33254 to nucleic acid 33553 of SEQ ID NO: 1 (JMHV39), nucleic acid 35301 to nucleic acid 35687 of SEQ ID NO: 1 (JMVH41), nucleic acid 40188 to nucleic acid 40439 of SEQ ID NO: 1 (JMHV48), nucleic acid 45836 to nucleic acid 46195 of SEQ ID NO: 1 (JMHV54), nucleic acid 47768 to nucleic acid 48136 of SEQ ID NO: 1 (JM57), nucleic acid 57325 to nucleic acid 57573 of SEQ ID NO: 1 (JM71), nucleic acid 62823 to nucleic acid 63086 of SEQ ID NO: 1 (JM76), nucleic acid 65629 to nucleic acid 65880 of SEQ ID NO: 1 (JM80), nucleic acid 67920 to nucleic acid 68594 of SEQ ID NO: 1 (JM85), nucleic acid to nucleic acid of SEQ ID NO: 1 (JM87), nucleic acid 70328 to nucleic acid 70606 of SEQ ID NO: 1 (JM88), nucleic acid 75447 to nucleic acid 75722 of SEQ ID NO: 1 (JM95), nucleic acid 105581 to nucleic acid 106003 of SEQ ID NO: 1 (JM132), nucleic acid 117501 to nucleic acid 118265 of SEQ ID NO: 1 (JM152), nucleic acid to nucleic acid of SEQ ID NO: 1 (JM159), nucleic acid to nucleic acid of SEQ ID NO: 1 (JM166), and nucleic acid to nucleic acid of SEQ ID NO: 1 (JM167).

It is understood that all polynucleotides encoding a JMHV polypeptide are also included herein, as long as they encode a polypeptide with the recognized activity, such as the binding to an antibody that recognizes the JMHV polypeptide. The JMHV polynucleotides include sequences that are degenerate as a result of the genetic code. There are 20 natural amino acids, most of which are specified by more than one codon. Therefore, all degenerate nucleotide sequences are included as long as the amino acid sequence of the JMHV polypeptide encoded by the nucleotide sequence is functionally unchanged.

The JMHV polynucleotides include a recombinant DNA which is incorporated into a vector; into an autonomously replicating plasmid or virus; or into the genomic DNA of a prokaryote or eukaryote, or which exists as a separate molecule (e.g., a cDNA) independent of other sequences. The nucleotides can be ribonucleotides, deoxyribonucleotides, or modified forms of either nucleotide. The term includes single and double forms of DNA. Also included are fragments of the above-described nucleic acid sequences that are at least 15 bases in length, which is sufficient to permit the fragment to selectively hybridize to DNA that encodes the disclosed JMHV polypeptide (such as a polynucleotide that is encoded by SEQ ID NO: 1) under physiological conditions. These nucleic acids can be used as probes or primers for the identification of viral nucleic acid. The term “selectively hybridize” refers to hybridization under moderately or highly stringent conditions, which excludes non-related nucleotide sequences. The JMHV polynucleotide sequence disclosed herein include, but are not limited to, nucleic acid 21845 to nucleic acid 22120 of SEQ ID NO: 1 (JMHV25), nucleic acid 22363 to nucleic acid 22701 of SEQ ID NO: 1 (JMHV26), nucleic acid 33254 to nucleic acid 33553 of SEQ ID NO: 1 (JMHV39), nucleic acid 35301 to nucleic acid 35687 of SEQ ID NO: 1 (JMVH41), nucleic acid 40188 to nucleic acid 40439 of SEQ ID NO: 1 (JMHV48), nucleic acid 45836 to nucleic acid 46195 of SEQ ID NO: 1 (JMHV54), nucleic acid 47768 to nucleic acid 48136 of SEQ ID NO: 1 (JM57), nucleic acid 57325 to nucleic acid 57573 of SEQ ID NO: 1 (JM71), nucleic acid 62823 to nucleic acid 63086 of SEQ ID NO: 1 (JM76), nucleic acid 65629 to nucleic acid 65880 of SEQ ID NO: 1 (J(M80), nucleic acid 67920 to nucleic acid 68594 of SEQ ID NO: 1 (JM85), nucleic acid to nucleic acid of SEQ ID NO: 1 (JM87), nucleic acid 70328 to nucleic acid 70606 of SEQ ID NO: 1 (JM88), nucleic acid 75447 to nucleic acid 75722 of SEQ ID NO: 1 (JM95), nucleic acid 105581 to nucleic acid 106003 of SEQ ID NO: 1 (JM132), nucleic acid 117501 to nucleic acid 118265 of SEQ ID NO: 1 (JM152), nucleic acid to nucleic acid of SEQ ID NO: 1 (JM159), nucleic acid to nucleic acid of SEQ ID NO: 1 (JM166), and nucleic acid to nucleic acid of SEQ ID NO: 1 (JM167).

The JMHV polypeptides of this disclosure include polypeptides encoded by all of the open reading frames disclosed herein. Polypeptides least 85%, at least about 90%, at least about 95%, at least about 98%, or at least about 99% homologous to the JMHV ORF (see Table 3) are also encompassed by this disclosure. In one embodiment, these polypeptides retain a function of the ORF. The polypeptides also include amino acid sequences including at most one, at most two, at most three, at most four, at most five or at most ten conservative substitutions of the JMHV polypeptide. Fusion proteins are also contemplated that include a heterologous amino acid sequence chemically linked to a JMHV polypeptide. Exemplary fusion proteins include short amino acid sequence tags (such as six histidine residues) as well a fusion of other proteins (such as c-myc or green fluorescent protein fusions). Epitopes of the ORFs, that retain the ability to bind an antibody or to bind the major histocompatibility complex (MHC), and can be used to induce an immune response, are also encompassed by this disclosure. Specific examples of JMHV polypeptides are provided as SEQ ID NOs: 2-171.

DNA sequences encoding any JMHV polypeptide can be expressed in vitro by DNA transfer into a suitable host cell. The cell may be prokaryotic or eukaryotic. The term also includes any progeny of the subject host cell. It is understood that all progeny may not be identical to the parental cell since there may be mutations that occur during replication. Methods of stable transfer, meaning that the foreign DNA is continuously maintained in the host, are known in the art.

Polynucleotide sequences encoding an open reading frame of JMHV can be operatively linked to expression control sequences. An expression control sequence operatively linked to a coding sequence is ligated such that expression of the coding sequence is achieved under conditions compatible with the expression control sequences. The expression control sequences include, but are not limited to appropriate promoters, enhancers, transcription terminators, a start codon (i.e., ATG) in front of a protein-encoding gene, splicing signal for introns, maintenance of the correct reading frame of that gene to permit proper translation of mRNA, and stop codons.

The polynucleotide sequences encoding a JMHV ORF may be inserted into an expression vector including, but not limited to a plasmid, virus or other vehicle that can be manipulated to allow insertion or incorporation of sequences and can be expressed in either prokaryotes or eukaryotes. Hosts can include microbial, yeast, insect and mammalian organisms. Methods of expressing DNA sequences having eukaryotic or viral sequences in prokaryotes are well known in the art. Biologically functional viral and plasmid DNA vectors capable of expression and replication in a host are known in the art.

Transformation of a host cell with recombinant DNA may be carried out by conventional techniques as are well known to those skilled in the art. Where the host is prokaryotic, such as E. coli, competent cells which are capable of DNA uptake can be prepared from cells harvested after exponential growth phase and subsequently treated by the CaCl₂ method using procedures well known in the art. Alternatively, MgCl₂ or RbCl can be used. Transformation can also be performed after forming a protoplast of the host cell if desired, or by electroporation.

When the host is a eukaryote, such methods of transfection of DNA as calcium phosphate coprecipitates, conventional mechanical procedures such as microinjection, electroporation, insertion of a plasmid encased in liposomes, or virus vectors may be used. Eukaryotic cells can also be cotransformed with JMHV polynucleotide sequences, and a second foreign DNA molecule encoding a selectable phenotype, such as the herpes simplex thymidine kinase gene. Another method is to use a eukaryotic viral vector, such as simian virus 40 (SV40) or bovine papilloma virus, to transiently infect or transform eukaryotic cells and express the protein (see for example, Eukaryotic Viral Vectors, Cold Spring Harbor Laboratory, Gluzman ed., 1982).

Isolation and purification of recombinantly expressed polypeptide may be carried out by conventional means including preparative chromatography and immunological separations. The peptides can be chemically synthesized by any of a number of manual or automated methods of synthesis known in the art. For example, solid phase peptide synthesis (SPPS) is carried out on a 0.25 millimole (mmole) scale using an Applied Biosystems Model 431A Peptide Synthesizer and using 9-fluorenylmethyloxycarbonyl (Fmoc) amino-terminus protection, coupling with dicyclohexylcarbodiimide/hydroxybenzotriazole or 2-(1H-benzo-triazol-1-yl)-1,1,3,3-tetramethyluronium hexafluorophosphate/hydroxybenzotriazole (HBTU/HOBT), and using p-hydroxymethylphenoxymethylpolystyrene (HMP) or Sasrin resin for carboxyl-terminus acids or Rink amide resin for carboxyl-terminus amides.

Fmoc-derivatized amino acids are prepared from the appropriate precursor amino acids by tritylation and triphenylmethanol in trifluoroacetic acid, followed by Fmoc derivitization as described by Atherton et al. (Solid Phase Peptide Synthesis, IRL Press: Oxford, 1989).

Sasrin resin-bound peptides are cleaved using a solution of 1% TFA in dichloromethane to yield the protected peptide. Where appropriate, protected peptide precursors are cyclized between the amino- and carboxyl-termini by reaction of the amino-terminal free amine and carboxyl-terminal free acid using diphenylphosphorylazide in nascent peptides wherein the amino acid sidechains are protected.

HMP or Rink amide resin-bound products are routinely cleaved and protected sidechain-containing cyclized peptides deprotected using a solution comprised of trifluoroacetic acid (TFA), optionally also comprising water, thioanisole, and ethanedithiol, in ratios of 100:5:5:2.5, for 0.5-3 hours at room temperature.

Crude peptides are purified by preparative high pressure liquid chromatography (HPLC), for example using a Waters Delta-Pak C18 column and gradient elution with 0.1% TFA in water modified with acetonitrile. After column elution, acetonitrile is evaporated from the eluted fractions, which are then lyophilized. The identity of each product so produced and purified may be confirmed by fast atom bombardment mass spectroscopy (FABMS) or electrospray mass spectroscopy (ESMS).

### Antibodies

A JMHV polypeptide or a fragment or conservative variant thereof can be used to produce antibodies which are immunoreactive or bind to an epitope of the JMHV polypeptide. Polyclonal antibodies, antibodies which consist essentially of pooled monoclonal antibodies with different epitopic specificities, as well as distinct monoclonal antibody preparations are included.

The preparation of polyclonal antibodies is well-known to those skilled in the art. See, for example, Green et al., “Production of Polyclonal Antisera,” in Immunochemical Protocols pages 1-5, Manson, ed., Humana Press 1992; Coligan et al., “Production of Polyclonal Antisera in Rabbits, Rats, Mice and Hamsters,” in: Current Protocols in Immunology, section 2.4.1, 1992.

The preparation of monoclonal antibodies likewise is conventional. See, for example, Kohler & Milstein, Nature 256:495, 1975; Coligan et al., sections 2.5.1-2.6.7; and Harlow et al., in: Antibodies: a Laboratory Manual, page 726, Cold Spring Harbor Pub., 1988. Briefly, monoclonal antibodies can be obtained by injecting mice with a composition comprising an antigen, verifying the presence of antibody production by removing a serum sample, removing the spleen to obtain B lymphocytes, fusing the B lymphocytes with myeloma cells to produce hybridomas, cloning the hybridomas, selecting positive clones that produce antibodies to the antigen, and isolating the antibodies from the hybridoma cultures. Monoclonal antibodies can be isolated and purified from hybridoma cultures by a variety of well-established techniques. Such isolation techniques include affinity chromatography with Protein-A Sepharose, size-exclusion chromatography, and ion-exchange chromatography. See, e.g., Coligan et al., sections 2.7.1-2.7.12 and sections 2.9.1-2.9.3; Barnes et al., “Purification of Immunoglobulin G (IgG),” in: Methods in Molecular Biology, Vol. 10, pages 79-104, Humana Press, 1992.

Methods of in vitro and in vivo multiplication of monoclonal antibodies are well known to those skilled in the art. Multiplication in vitro may be carried out in suitable culture media such as Dulbecco's Modified Eagle Medium or RPMI 1640 medium, optionally supplemented by a mammalian serum such as fetal calf serum or trace elements and growth-sustaining supplements such as normal mouse peritoneal exudate cells, spleen cells, thymocytes or bone marrow macrophages. Production in vitro provides relatively pure antibody preparations and allows scale-up to yield large amounts of the desired antibodies. Large-scale hybridoma cultivation can be carried out by homogenous suspension culture in an airlift reactor, in a continuous stirrer reactor, or in immobilized or entrapped cell culture. Multiplication in vivo may be carried out by injecting cell clones into mammals histocompatible with the parent cells, e.g., syngeneic mice, to cause growth of antibody-producing tumors. Optionally, the animals are primed with a hydrocarbon, especially oils such as pristane (tetramethylpentadecane) prior to injection. After one to three weeks, the desired monoclonal antibody is recovered from the body fluid of the animal.

Antibodies can also be derived from subhuman primate antibody. General techniques for raising therapeutically useful antibodies in baboons can be found, for example, in WO 91/11465, 1991, and Losman et al., Int. J. Cancer 46:310, 1990.

Alternatively, an antibody that specifically binds a JMHV polypeptide can be derived from a humanized monoclonal antibody. Humanized monoclonal antibodies are produced by transferring mouse complementarity determining regions from heavy and light variable chains of the mouse immunoglobulin into a human variable domain, and then substituting human residues in the framework regions of the murine counterparts. The use of antibody components derived from humanized monoclonal antibodies obviates potential problems associated with the immunogenicity of murine constant regions. General techniques for cloning murine immunoglobulin variable domains are described, for example, by Orlandi et al., Proc. Nat'l Acad. Sci. U.S.A. 86:3833, 1989. Techniques for producing humanized monoclonal antibodies are described, for example, by Jones et al., Nature 321:522, 1986; Riechmann et al., Nature 332:323, 1988; Verhoeyen et al., Science 239:1534, 1988; Carter et al., Proc. Nat'l Acad. Sci. U.S.A. 89:4285, 1992; Sandhu, Crit. Rev. Biotech. 12:437, 1992; and Singer et al., J. Immunol. 150:2844, 1993.

Antibodies can be derived from human antibody fragments isolated from a combinatorial immunoglobulin library. See, for example, Barbas et al., in: Methods: a Companion to Methods in Enzymology, Vol. 2, page 119, 1991; Winter et al., Ann. Rev. Immunol. 12:433, 1994. Cloning and expression vectors that are useful for producing a human immunoglobulin phage library can be obtained, for example, from STRATAGENE Cloning Systems (La Jolla, Calif.).

In addition, antibodies can be derived from a human monoclonal antibody. Such antibodies are obtained from transgenic mice that have been “engineered” to produce specific human antibodies in response to antigenic challenge. In this technique, elements of the human heavy and light chain loci are introduced into strains of mice derived from embryonic stem cell lines that contain targeted disruptions of the endogenous heavy and light chain loci. The transgenic mice can synthesize human antibodies specific for human antigens, and the mice can be used to produce human antibody-secreting hybridomas. Methods for obtaining human antibodies from transgenic mice are described by Green et al., Nature Genet. 7:13, 1994; Lonberg et al., Nature 368:856, 1994; and Taylor et al., Int. Immunol.6:579, 1994.

Antibodies include intact molecules as well as fragments thereof, such as Fab, F(ab′)₂, and Fv which are capable of binding the epitopic determinant. These antibody fragments retain some ability to selectively bind with their antigen or receptor and are defined as follows:

(1) Fab, the fragment which contains a monovalent antigen-binding fragment of an antibody molecule, can be produced by digestion of whole antibody with the enzyme papain to yield an intact light chain and a portion of one heavy chain;

(2) Fab′, the fragment of an antibody molecule can be obtained by treating whole antibody with pepsin, followed by reduction, to yield an intact light chain and a portion of the heavy chain; two Fab′ fragments are obtained per antibody molecule;

(3) (Fab′)₂, the fragment of the antibody that can be obtained by treating whole antibody with the enzyme pepsin without subsequent reduction; F(ab′)₂ is a dimer of two Fab′ fragments held together by two disulfide bonds;

(4) Fv, defined as a genetically engineered fragment containing the variable region of the light chain and the variable region of the heavy chain expressed as two chains; and

(5) Single chain antibody (SCA), defined as a genetically engineered molecule containing the variable region of the light chain, the variable region of the heavy chain, linked by a suitable polypeptide linker as a genetically fused single chain molecule.

Methods of making these fragments are known in the art. (See for example, Harlow and Lane, Antibodies: A Laboratory Manual, Cold Spring Harbor Laboratory, New York, 1988). An epitope is any antigenic determinant on an antigen to which the paratope of an antibody binds. Epitopic determinants usually consist of chemically active surface groupings of molecules such as amino acids or sugar side chains and usually have specific three dimensional structural characteristics, as well as specific charge characteristics.

Antibody fragments can be prepared by proteolytic hydrolysis of the antibody or by expression in E. coli of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)₂. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly (see U.S. Pat. No. 4,036,945 and U.S. Pat. No. 4,331,647, and references contained therein; Nisonhoff et al., Arch. Biochem. Biophys.89:230, 1960; Porter, Biochem. J. 73:119, 1959; Edelman et al., Methods in Enzymology, Vol. 1, page 422, Academic Press, 1967; and Coligan et al. at sections 2.8.1-2.8.10 and 2.10.1-2.10.4).

Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

For example, Fv fragments comprise an association of V_{H} and V_{L} chains. This association may be noncovalent (Inbar et al., Proc. Nat'l Acad. Sci. U.S.A. 69:2659, 1972). Alternatively, the variable chains can be linked by an intermolecular disulfide bond or cross-linked by chemicals such as glutaraldehyde. See, e.g., Sandhu, supra. Preferably, the Fv fragments comprise V_{H} and V_{L} chains connected by a peptide linker. These single-chain antigen binding proteins (sFv) are prepared by constructing a structural gene comprising DNA sequences encoding the V_{H} and V_{L} domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are known in the art (see Whitlow et al., Methods: a Companion to Methods in Enzymology, Vol. 2, page 97, 1991; Bird et al., Science 242:423, 1988; U.S. Pat. No. 4,946,778; Pack et al., Bio/Technology 11:1271, 1993; and Sandhu, supra).

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells (Larrick et al., Methods: a Companion to Methods in Enzymology, Vol. 2, page 106, 1991).

Antibodies can be prepared using an intact polypeptide or fragments containing small peptides of interest as the immunizing antigen. The polypeptide or a peptide used to immunize an animal can be derived from substantially purified polypeptide produced in host cells, in vitro translated cDNA, or chemical synthesis which can be conjugated to a carrier protein, if desired. Such commonly used carriers which are chemically coupled to the peptide include keyhole limpet hemocyanin (KLH), thyroglobulin, bovine serum albumin (BSA), and tetanus toxoid. The coupled peptide is then used to immunize the animal (e.g., a mouse, a rat, or a rabbit).

Polyclonal or monoclonal antibodies can be further purified, for example, by binding to and elution from a matrix to which the polypeptide or a peptide to which the antibodies were raised is bound. Those of skill in the art will know of various techniques common in the immunology arts for purification and/or concentration of polyclonal antibodies, as well as monoclonal antibodies (See for example, Coligan et al., Unit 9, Current Protocols in Immunology, Wiley Interscience, 1991).

It is also possible to use the anti-idiotype technology to produce monoclonal antibodies which mimic an epitope. For example, an anti-idiotypic monoclonal antibody made to a first monoclonal antibody will have a binding domain in the hypervariable region that is the “image” of the epitope bound by the first monoclonal antibody.

Effector molecules, e.g., therapeutic, diagnostic, or detection moieties, can be linked to an antibody that specifically binds a JMHV polypeptide, using any number of means known to those of skill in the art. Both covalent and noncovalent attachment means may be used. The procedure for attaching an effector molecule to an antibody varies according to the chemical structure of the effector. Polypeptides typically contain a variety of functional groups; e.g., carboxylic acid (COOH), free amine (—NH₂) or sulfhydryl (—SH) groups, which are available for reaction with a suitable functional group on an antibody to result in the binding of the effector molecule. Alternatively, the antibody is derivatized to expose or attach additional reactive functional groups. The derivatization may involve attachment of any of a number of linker molecules such as those available from Pierce Chemical Company, Rockford, Ill. The linker can be any molecule used to join the antibody to the effector molecule. The linker is capable of forming covalent bonds to both the antibody and to the effector molecule. Suitable linkers are well known to those of skill in the art and include, but are not limited to, straight or branched-chain carbon linkers, heterocyclic carbon linkers, or peptide linkers. Where the antibody and the effector molecule are polypeptides, the linkers may be joined to the constituent amino acids through their side groups (such as through a disulfide linkage to cysteine) or to the alpha carbon amino and carboxyl groups of the terminal amino acids.

In some circumstances, it is desirable to free the effector molecule from the antibody when the immunoconjugate has reached its target site. Therefore, in these circumstances, immunoconjugates will comprise linkages that are cleavable in the vicinity of the target site. Cleavage of the linker to release the effector molecule from the antibody may be prompted by enzymatic activity or conditions to which the immunoconjugate is subjected either inside the target cell or in the vicinity of the target site.

In view of the large number of methods that have been reported for attaching a variety of radiodiagnostic compounds, radiotherapeutic compounds, label (e.g. enzymes or fluorescent molecules) drugs, toxins, and other agents to antibodies, one skilled in the art will be able to determine a suitable method for attaching a given effector molecule to an antibody or other polypeptide.

The immunoconjugates can be prepared by cloning techniques. Examples of appropriate cloning and sequencing techniques, and instructions sufficient to direct persons of skill through many cloning exercises are found in Sambrook et al., Molecular Cloning: A Laboratory Manual (2nd Ed.), Vols. 1-3, Cold Spring Harbor Laboratory (1989), Berger and Kimmel (eds.), Guide to Molecular Cloning Techniques, Academic Press, Inc., San Diego Calif. (1987), or Ausubel et al. (eds.), Current Protocols in Molecular Biology, Greene Publishing and Wiley-Interscience, NY (1987). Product information from manufacturers of biological reagents and experimental equipment also provide useful information. Such manufacturers include the SIGMA chemical company (Saint Louis, Mo.), R&D systems (Minneapolis, Minn.), Pharmacia LKB Biotechnology (Piscataway, N.J.), CLONTECH Laboratories, Inc. (Palo Alto, Calif.), Chem Genes Corp., Aldrich Chemical Company (Milwaukee, Wis.), Glen Research, Inc., GIBCO BRL Life Technologies, Inc. (Gaithersburg, Md.), Fluka Chemica-Biochemika Analytika (Fluka Chemie AG, Buchs, Switzerland), Invitrogen (San Diego, Calif.), and Applied Biosystems (Foster City, Calif.), as well as many other commercial sources known to one of skill.

Nucleic acids encoding native effector molecules or anti-JMHV antibodies can be modified to form the effector molecule, antibodies, or immunoconjugates. Modification by site-directed mutagenesis is well known in the art. Nucleic acids encoding effector molecule or anti-JMHV antibodies can be amplified by in vitro methods. Amplification methods include the polymerase chain reaction (PCR), the ligase chain reaction (LCR), the transcription-based amplification system (TAS), the self-sustained sequence replication system (3SR). A wide variety of cloning methods, host cells, and in vitro amplification methodologies are well known in the art.

In one embodiment, immunoconjugates are prepared by inserting a cDNA which encodes an anti-JMHV polypeptide scFv antibody into a vector which comprises the cDNA encoding the effector molecule. The insertion is made so that the scFv and the EM are read in frame that is in one continuous polypeptide which contains a functional Fv region and a functional EM region

In addition to recombinant methods, the immunoconjugates, effector molecules, and antibodies can also be constructed in whole or in part using standard peptide synthesis. Solid phase synthesis of the polypeptides of less than about 50 amino acids in length may be accomplished by attaching the C-terminal amino acid of the sequence to an insoluble support followed by sequential addition of the remaining amino acids in the sequence. Techniques for solid phase synthesis are described by Barany & Merrifield, “The Peptides: Analysis, Synthesis, Biology,” Vol. 2, Special Methods in Peptide Synthesis, Part A. pp. 3-284; Merrifield et al. J. Am. Chem. Soc. 85:2149-2156, 1963, and Stewart et al., Solid Phase Peptide Synthesis, 2nd ed., Pierce Chem. Co., Rockford, Ill., 1984. Proteins of greater length may be synthesized by condensation of the amino and carboxyl termini of shorter fragments. Methods of forming peptide bonds by activation of a carboxyl terminal end (e.g., by the use of the coupling reagent N,N′-dicycylohexylcarbodiimide) are known to those of skill.

Once the nucleic acids encoding an EM, anti-JMHV antibody, or an immunoconjugate, are isolated and cloned, one may express the desired protein in a recombinantly engineered cell such as bacteria, plant, yeast, insect and mammalian cells. It is expected that those of skill in the art are knowledgeable in the numerous expression systems available for expression of proteins including E. coli, other bacterial hosts, yeast, and various higher eukaryotic cells such as the COS, CHO, HeLa and myeloma cell lines.

Antibodies can be covalently or non-covalently linked to a detectable label. Detectable labels suitable for such use include any composition detectable by spectroscopic, photochemical, biochemical, immunochemical, electrical, optical or chemical means. Useful labels in the present disclosure include magnetic beads (e.g. DYNABEADS), fluorescent dyes (e.g., fluorescein isothiocyanate, Texas red, rhodamine, green fluorescent protein, and the like), radiolabels (e.g., ³H, ¹²⁵I, ³⁵S, ¹⁴C, or ³²P), enzymes (e.g., horseradish peroxidase, alkaline phosphatase and others commonly used in an ELISA), and calorimetric labels such as colloidal gold or colored glass or plastic (e.g., polystyrene, polypropylene, latex, etc.) beads. Means of detecting such labels are well known to those of skill in the art. Thus, for example, radiolabels may be detected using photographic film or scintillation counters, fluorescent markers may be detected using a photodetector to detect emitted illumination. Enzymatic labels are typically detected by providing the enzyme with a substrate and detecting the reaction product produced by the action of the enzyme on the substrate, and colorimetric labels are detected by simply visualizing the colored label.

### Diagnostic Methods

A method for screening a subject to determine if the subject has been infected with JMHV is disclosed herein. One major application of the JMHV sequence information presented herein is in the area of diagnostic testing for predisposition to a disease (such as for multiple sclerosis) that develops in at least a subset of hosts infected with JMHV. The nucleic acid sequence of the JMHV ORFs is also useful in such diagnostic methods. The method includes providing a biological sample obtained from the subject, in which sample includes DNA or RNA, and providing an assay for detecting in the biological sample the presence of any of the JMHV nucleic acids or proteins. Suitable biological samples include samples obtained from body cells, such as those present in peripheral blood, urine, saliva, tissue biopsy, surgical specimen, fine needle aspirate specimen, amniocentesis samples and autopsy material. The detection in the biological sample may be performed by a number of methodologies, as outlined below.

The foregoing assay may be assembled in the form of a diagnostic kit and preferably comprises either: hybridization with oligonucleotides; PCR amplification of the gene or a part thereof using oligonucleotide primers; RT-PCR amplification of the RNA or a part thereof using oligonucleotide primers; or direct sequencing of any of the JMHV genes present in a subject using oligonucleotide primers. The efficiency of these molecular genetic methods should permit the rapid identification of patients infected with JMHV. Thus kits can include containers with JMHV nucleic acid sequences (such as probes or primers) and/or containers including an antibody that specifically binds JMHV.

One embodiment of such detection techniques is the polymerase chain reaction amplification of reverse transcribed RNA (RT-PCR) of RNA isolated from cells (for example lymphocytes) followed by direct DNA sequence determination of the products. The presence of one or more JMHV genes is taken an indicative of potential JMHV infection.

Alternatively, DNA extracted from lymphocytes or other cells may be used directly for amplification. The direct amplification from genomic DNA would be appropriate for analysis of an entire JMHV nucleic acid sequence including regulatory sequences located upstream and downstream from the open reading frame. Recent reviews of direct DNA diagnosis have been presented by Caskey (Science 236:1223-1228, 1989) and by Landegren et al. (Science 242:229-37, 1989). DNA diagnostic methods to specifically detect an ORF, or to detect a particular polymorphism in an ORF, can be designed.

The detection of specific DNA mutations or alterations in gene sequences may be achieved by methods such as hybridization using specific oligonucleotides (Wallace et al., Cold Spring Harbor Symp. Quant. Biol. 51:257-61, 1986), direct DNA sequencing (Church and Gilbert, Proc. Natl. Acad. Sci. USA. 81:1991-5, 1984), the use of restriction enzymes (Flavell et al., Cell 15:25, 1978; Geever et al., Proc. Natl. Acad. Sci USA 78:5081, 1981), discrimination on the basis of electrophoretic mobility in gels with denaturing reagent (Myers and Maniatis, Cold Spring Harbor Symp. Quant. Biol. 51:275-284, 1986), RNase protection (Myers et al., Science 230:1242, 1985), chemical cleavage (Cotton et al., Proc. Natl. Acad. Sci. USA 85:4397-401, 1985), and the ligase-mediated detection procedure (Landegren et al., Science 241:1077, 1988).

Oligonucleotides specific to normal, mutant or alterative sequences are chemically synthesized using commercially available machines, labeled radioactively with isotopes (such as ³²P) or non-radioactively, with tags such as biotin (Ward and Langer et al., Proc. Natl. Acad. Sci. USA 78:6633-57, 1981), and hybridized to individual DNA samples immobilized on membranes or other solid supports by dot-blot or transfer from gels after electrophoresis. The presence of these specific sequences are visualized by methods such as autoradiography or fluorometric (Landegren et al., Science 242:229-37, 1989) or calorimetric reactions (Gebeyehu et al., Nucleic Acids Res. 15:4513-34, 1987). The absence of hybridization would indicate a mutation in the particular region of the gene, or that the patient is not infected with JMHV.

Sequence differences between disclosed and other forms of JMHV genes may also be revealed by the direct DNA sequencing method of Church and Gilbert (Proc. Natl. Acad. Sci. USA 81:1991-5, 1988). Cloned DNA segments may be used as probes to detect specific DNA segments. The sensitivity of this method is greatly enhanced when combined with PCR (Wrichnik et al., Nucleic Acids Res. 15:529-42, 1987; Wong et al., Nature 330:384-6, 1987; Stoflet et al., Science 239:491-4, 1988). In this approach, a sequencing primer which lies within the amplified sequence is used with double-stranded PCR product or single-stranded template generated by a modified PCR. The sequence determination is performed by conventional procedures with radiolabelled nucleotides or by automatic sequencing procedures with fluorescent tags.

Sequence alterations may occasionally generate fortuitous restriction enzyme recognition sites or may eliminate existing restriction sites. Changes in restriction sites are revealed by the use of appropriate enzyme digestion followed by conventional gel-blot hybridization (Southern, J. Mol. Biol. 98:503, 1975). DNA fragments carrying the site (either normal, mutant, or alternative) are detected by their reduction in size or increase of corresponding restriction fragment numbers. Genomic DNA samples may also be amplified by PCR prior to treatment with the appropriate restriction enzyme; fragments of different sizes are then visualized under UV light in the presence of ethidium bromide after gel electrophoresis.

Screening based on DNA sequence differences may be achieved by detection of alteration in electrophoretic mobility of DNA fragments in gels with or without denaturing reagent. Small sequence deletions and insertions can be visualized by high-resolution gel electrophoresis. For example, a PCR product with small deletions is clearly distinguishable from a normal sequence on an 8% non-denaturing polyacrylamide gel (WO 91/10734; Nagamine et al., Am. J. Hum. Genet. 45:337-9, 1989). DNA fragments of different sequence compositions may be distinguished on denaturing formamide gradient gels in which the mobilities of different DNA fragments are retarded in the gel at different positions according to their specific “partial-melting” temperatures (Myers et al., Science 230:1242, 1985). Alternatively, a method of detecting a mutation comprising a single base substitution or other small change could be based on differential primer length in a PCR. For example, an invariant primer could be used in addition to a primer specific for a mutation. The PCR products of the normal and mutant genes can then be differentially detected in acrylamide gels.

In addition to conventional gel-electrophoresis and blot-hybridization methods, DNA fragments may also be visualized by methods where the individual DNA samples are not immobilized on membranes. The probe and target sequences may be both in solution, or the probe sequence may be immobilized (Saiki et al., Proc. Nat. Acad. Sci. USA 86:6230-4, 1989). A variety of detection methods, such as autoradiography involving radioisotopes, direct detection of radioactive decay (in the presence or absence of scintillant), spectrophotometry involving calorigenic reactions and fluorometry involved fluorogenic reactions, may be used to identify specific individual genotypes.

If more than one mutation or alternative sequence is frequently encountered in one or more JMHV genes, a system capable of detecting such multiple mutations can be produced. For example, a PCR with multiple, specific oligonucleotide primers and hybridization probes can be used to identify all possible mutations or alternative sequences at the same time (Chamberlain et al., Nucl. Acids Res. 16:1141-55, 1988). The procedure may involve immobilized sequence-specific oligonucleotides probes (Saiki et al., Proc. Nat. Acad. Sci. USA 86:6230-4, 1989).

### Quantitation of JMHV Proteins

An alternative method of determining if a subject has been infected with RRV or JMHV is to quantitate the level of one or more RRV (or JMHV) proteins in the cells of a subject. This diagnostic tool would also be useful for detecting the levels of the JMHV proteins which result from infection by JMHV. These diagnostic methods provide an enhanced ability to diagnose susceptibility to diseases caused by JMHV infection.

The determination of JMHV protein levels would be an alternative or supplemental approach to the direct determination of the presence of one or more JMHV genes. The availability of antibodies specific to one or more of the JMHV proteins will facilitate the quantitation of cellular JMHV proteins by one of a number of immunoassay methods which are well known in the art and are presented in Harlow and Lane (Antibodies, A Laboratory Manual, Cold Spring Harbor Laboratory, New York., 1988).

Such assays permit the detection of JMHV proteins in a biological sample and the quantitation of such proteins. Typical methods involve: providing a biological sample of the subject in which the sample contains cellular proteins, and providing an immunoassay for quantitating the level of at least one JMHV protein in the biological sample. This can be achieved by combining the biological sample with a JMHV specific binding agent, such as an antibody (e.g. monoclonal or polyclonal antibodies that bind a JMHV protein), so that complexes form between the binding agent and the viral protein present in the sample, and then detecting or quantitating such complexes.

In particular forms, these assays may be performed with the JMHV specific binding agent immobilized on a support surface, such as in the wells of a microtiter plate or on a column. The biological sample is then introduced onto the support surface and allowed to interact with the specific binding agent so as to form complexes. Excess biological sample is then removed by washing, and the complexes are detected with a reagent, such as a second anti-JMHV protein antibody, that is conjugated with a detectable marker.

In an alternative assay, the cellular proteins are isolated and subjected to SDS-PAGE followed by Western blotting. After resolving the proteins, the proteins are transferred to a membrane, which is probed with specific binding agents that recognize any of the JMHV proteins. The proteins are detected, for example with labeled (such as horseradish peroxidase, HRP)-conjugated secondary antibodies, and quantitated.

In yet another assay, the level of one or more JMHV proteins in cells is analyzed using microscopy. Using specific binding agents which recognize JMHV, samples can be analyzed for the presence of one or more JMHV proteins. For example, frozen biopsied tissue sections are thawed at room temperature and fixed with acetone at −200° C. for 5 minutes. Slides are washed twice in cold PBS for 5 minutes each, then air-dried. Sections are covered with 20-30 μl of antibody solution (15-45 μg/ml) (diluted in PBS, 2% BSA at 15-50 μg/ml) and incubated at room temperature in humidified chamber for 30 minutes. Slides are washed three times with cold PBS 5 minutes each, allowed to air-dry briefly (5 minutes) before applying 20-30 μl of the second antibody solution (diluted in PBS, 2% BSA at 15-50 μg/ml) and incubated at room temperature in humidified chamber for 30 minutes. The label on the second antibody may contain a fluorescent probe, enzyme, radiolabel, biotin, or other detectable marker. The slides are washed three times with cold PBS 5 minutes each then quickly dipped in distilled water, air-dried, and mounted with PBS containing 30% glycerol. Slides can be stored at 4° C. prior to viewing.

For samples prepared for electron microscopy (versus light microscopy), the second antibody is conjugated to gold particles. Tissue is fixed and embedded with epoxy plastics, then cut into very thin sections (˜1-2 μm). The specimen is then applied to a metal grid, which is then incubated in the primary anti-JMHV antibody, washed in a buffer containing BSA, then incubated in a secondary antibody conjugated to gold particles (usually 5-20 nm). These gold particles are visualized using electron microscopy methods.

For the purposes of quantitating the JMHV proteins, a biological sample of the subject, which sample includes cellular proteins, is required. Such a biological sample may be obtained from body cells, such as those present in which expression of the protein has been detected. The expression of JMHV proteins in peripheral blood leukocytes is clearly the most accessible and convenient source from which specimens can be obtained. Specimens can be obtained from peripheral blood, urine, saliva, tissue biopsy, amniocentesis samples, surgical specimens, fine needle aspirates, and autopsy material, particularly cancer cells. Quantitation of JMHV proteins would be made by immunoassay and compared to levels of the protein found in non-JMHV expressing cells, or to the level of JMHV proteins in non-JMHV infected cells (cells of the same origin that are not infected). A significant (preferably 50% or greater) increase in the amount of one or more JMHV proteins in the cells of a subject compared to the amount of one or more JMHV proteins found in non-JMHV infected cells or that found in normal cells, would be taken as an indication that the subject may have been infected with JMHV.

### Screening Assays for Pharmaceutical Agents of Interest

The present disclosure provides a virus that is involved in the causation or progression of certain diseases, such as multiple sclerosis, and therefore provides an animal model and assays directed to identifying potential pharmaceutical agents that inhibit the biological activity of the virus. Drug screening assays which determine whether or not a drug has activity against the virus can include incubating a compound to be evaluated for use in treatment of the condition with cells which express the JMHV proteins or peptides, and determining the effect of the compound on the activity of the virus. In vitro assays in which the virus is maintained in suitable cell culture are provided as are in vivo animal models (such as a non-human primate model).

In vitro assays include infecting cells such as Japanese macaque fibroblasts, neuronal cells, peripheral blood leukocytes or susceptible B or T cell lines with the agent of interest in the presence of varying concentrations of compounds targeted against viral replication. These compounds include, but are not limited to nucleoside analogs, chain terminators, antisense oligonucleotides and random polypeptides. (Asada et al., J. Clin. Microbiol. 27:2204, 1989; Kikuta et al., Lancet 7:861, 1989). Infected cultures and their supernatants can be assayed for the total amount of virus, including the presence of the viral genome, by quantitative PCR, by dot blot assays, or by using immunologic methods. For example, a culture of susceptible cells could be infected with the JMHV in the presence of various concentrations of drug, fixed on slides after a period of days, and examined for viral antigen by indirect immunofluorescence with monoclonal antibodies to viral polypeptides. Alternatively, chemically adhered cell monolayers can be used for an infectious agent assay using indirect immunofluorescent antibody staining to search for focus reduction (Higashi, J. Clin. Microbiol. 27:2204, 1989, incorporated by reference).

As an alternative to whole cell in vitro assays, purified enzymes isolated from the JMHV can be used as targets for rational drug design to determine the effect of the potential drug on enzyme activity, such as thymidylate sunthase or DNA polymerase. A measure of enzyme activity indicates an effect on the infectious agent itself. Drug screens using herpes viral products are known and have been previously described in EP 0514830 (herpes proteases) and WO 94/04920 (U_{L} 13 gene product).

In particular embodiments, this disclosure provides an assay for screening anti-MS chemotherapeutics. Infected cells can be incubated in the presence of a chemical agent that is a potential chemotherapeutic against MS. The level of virus in the cells is then determined after several days by IFA for antigens or Southern blotting for viral genome or Northern blotting for mRNA and compared to control cells. This assay can quickly screen large numbers of chemical compounds that may be useful against MS. This disclosure also provides an assay system that is employed to identify drugs or other molecules capable of binding to the DNA molecule or proteins, either in the cytoplasm or in the nucleus, thereby inhibiting or potentiating transcriptional activity. This assay would be useful in the development of drugs that are specific against particular cellular activity, or that would potentiate such activity, in time or in level of activity. Also included are drugs identified by this assay which have an anti-viral activity, and have therapeutic value for conditions associated with JMHV infection.

Thus, an assay is provided herein for screening anti-multiple sclerosis (MS) therapeutics. Infected cells can be incubated in the presence of a chemical agent that is a potential anti-inflammatory agent or other agent of use in treating MS (such as an agent that alters the interaction of IL-2 with its receptor). The level of virus in the cells is then determined by IFA for antigens, Southern blotting for the viral genome, Northern blotting for mRNA, or PCR, and compared to control cell. This assay can quickly screen large numbers of agents that may be useful in the treatment of MS. This disclosure also provides an assay system that is employed to identify drugs or other molecules capable of binding to the DNA molecule or proteins, either in the cytoplasm or in the nucleus, thereby inhibiting or potentiating transcriptional activity. This assay would be useful in the development of drugs that are specific against particular cellular activity, or that would potentiate such activity, in time or in level of activity. Also included are drugs identified by this assay which have an anti-viral activity, and an effect against conditions associated with JMHV infection, such as MS.

Animal models are useful for resolving a number of fundamental problems of infectious diseases that include, but are not limited to, determinants of virulence of the organism, mechanisms of host resistance, mechanisms of pathogenicity, establishment and regulation of chronic infection, and antimicrobial and chemotherapeutic actions of drugs on infectious agents. Variables that are commonly manipulated to address fundamental problems include, but are not limited to, the strain of infectious agent, the infecting dose of infectious agent and the route of administration of the infectious agent, the species or subspecies of animal, the age of animal, and the genetic background of the animal (Viral pathogenesis, N. Nathanson, Lippincot-Raven, Philadelphia, Pa., 1997).

Multiple sclerosis (MS) is a chronic, debilitating inflammatory disease limited to central nervous system (CNS) white matter. Currently, there are very few effective treatments for MS. The disease may not be etiologically homogeneous, but rather a complex set of diseases that have in common pathogenic mechanisms that involve genetically predisposed individuals, and infectious agents as initiators and diverse mechanisms of inflammatory white matter destruction (Hafler, J Clin Invest 104:527-529, 1999). Genetic studies in families with MS-affected members have revealed that MS is a complex trait, that the contribution of individual genes to susceptibility is probably small, and that differences are possible between familial and sporadic forms of the disease (Kalman and Lublin, Biomed Pharmacother 53:358-370, 1999).

An animal model, including a Japanese macaque infected with a virus at least 90% identical to the nucleic acid sequence set forth as SEQ ID NO: 1 is provided herein. This animal model can be used to assess the efficacy of agents for the treatment of multiple sclerosis.

In another embodiment, in which one or more JMHV strains are employed for generating an animal model, the JMHV used may be naturally occurring variant isolates recovered from Japanese macaques and other non-human primate species, molecular clones generated from these naturally occurring variant isolates and recombinant viruses with introduced mutations, deletions or recombined genomes designed to address function of specific genes. For example, the JMHV can include a nucleic acid sequence set forth as SEQ ID NO: 1. To create the animal model, an animal, such as a non-human primate, is utilized. For example, a macaque monkey can be administered JMVH. The non-human animal is administered a JMVH by any route, including oral, inhalation, intratracheal, intravaginal, intrarectal and parenteral routes including, but not limited to intravenous, intraarterial, intradermal, subcutaneous, intramuscular, intraperitoneal and organ-specific administration routes such and intracerebral and intraocular administration. The effect of an agent can then be evaluated in the animal. The agent can be administered prior to infection with the virus, at the same time as infection with the virus, or after infection with the virus. A therapeutically effective amount of the agent is administered, and a signs, symptom, or parameter of viral infection (such as viral load or expression of a JMHV protein) is assessed. In one embodiment, the sign, symptom, or a parameter of viral infection is compared to a control, such as to an animal of the same species infected with JMHV that is not treated with the agent, is treated with a vehicle control, or is not infected with JMHV.

### Pharmaceutical Compositions and Use

Pharmaceutical compositions including JMHV nucleic acid sequences, JMHV proteins, or antibodies that bind these proteins are disclosed herein.

These pharmaceutical compositions include a therapeutically effective amount of one or more JMHV polypeptide, or a nucleic acid encoding a JMHV polypeptide, or an antibody that specifically binds a JMHV polypeptide, alone or in combination, in conjunction with a pharmaceutically acceptable carrier.

Disclosed herein are substances suitable for use as vaccines for the prevention of diseases associated with JMHV infection, such as MS, and methods for administering them. Particular vaccines are directed against JMHV or related viruses, and may include antigens obtained from JMHV or its related viruses. In one embodiment, the vaccine contains attenuated JMHV, or related viruses found in humans. In another embodiment, the vaccine contains killed JMHV. In another embodiment, the vaccine contains a nucleic acid vector encoding at least one JMHV ORF, such as a surface protein of JMHV. In another embodiment, the composition contains a JMHV subunit, such as glycoprotein B, major capsid protein, or other gene products found to elicit appropriate humoral and/or cell mediated immune responses.

Various delivery systems for administering pharmaceutical compositions including JMHV proteins include encapsulation in liposomes, microparticles, microcapsules, expression by recombinant cells, receptor-mediated endocytosis (see Wu and Wu, J. Biol. Chem. 262:4429-32, 1987), and construction of a therapeutic nucleic acid (such as an anti-sense molecule) as part of a retroviral or other vector. Methods of introduction include, but are not limited to, intradermal, intramuscular, intraperitoneal, intravenous, subcutaneous, intranasal, and oral routes. The compounds may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, the pharmaceutical compositions may be introduced into the central nervous system by any suitable route, including intraventricular and intrathecal injection; intraventricular injection may be facilitated by an intraventricular catheter, for example, attached to a reservoir, such as an Ommaya reservoir.

The use of liposomes as a delivery vehicle is another delivery method of the present disclosure. The liposomes fuse with the target site and deliver the contents of the lumen intracellularly. The liposomes are maintained in contact with the target cells for a sufficient time for fusion to occur, using various means to maintain contact, such as isolation and binding agents. Liposomes may be prepared with purified proteins or peptides that mediate fusion of membranes, such as Sendai virus or influenza virus. The lipids may be any useful combination of known liposome forming lipids, including cationic lipids, such as phosphatidylcholine. Other potential lipids include neutral lipids, such as cholesterol, phosphatidyl serine, phosphatidyl glycerol, and the like. For preparing the liposomes, the procedure described by Kato et al. (J. Biol. Chem. 266:3361, 1991) may be used.

The pharmaceutical compositions or methods of treatment may be administered in combination with other therapeutic treatments, such as with additional immunosuppressive therapies.

JMHV nucleic acids can be used to generate an animal model, or to express JMHV polypeptides to produce an immune response against JMHV. In an embodiment in which one or more JMHV nucleic acids are employed for generating an animal model, or for producing an immune response, the nucleic acid can be delivered intracellularly (e.g., by expression from a nucleic acid vector or by receptor-mediated mechanisms). In a specific embodiment where the therapeutic molecule is a nucleic acid, administration may be achieved by an appropriate nucleic acid expression vector which is administered so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see e.g., Joliot et al., Proc. Natl. Acad. Sci. USA 88:1864-8, 1991). Alternatively, the nucleic acid can be introduced intracellularly and incorporated within host cell DNA for expression, by homologous recombination.

The vector pCDNA, is an example of a method of introducing the foreign cDNA into a cell under the control of a strong viral promoter (CMV) to drive the expression. However, other vectors can be used. Other retroviral vectors (such as pRETRO-ON, Clontech), also use this promoter but have the advantages of entering cells without any transfection aid, integrating into the genome of target cells only when the target cell is dividing (as cancer cells do, especially during first remissions after chemotherapy) and they are regulated. It is also possible to turn on the expression of the RRV or JMHV nucleic acid by administering tetracycline when these plasmids are used. Hence these plasmids can be allowed to transfect the cells, then administer a course of tetracycline with a course of chemotherapy to achieve better cytotoxicity.

Other plasmid vectors, such as pMAM-neo (also from Clontech) or pMSG (Pharmacia) use the MMTV-LTR promoter (which can be regulated with steroids) or the SV10 late promoter (pSVL, Pharmacia) or metallothionein-responsive promoter (pBPV, Pharmacia) and other viral vectors, including retroviruses. Examples of other viral vectors include adenovirus, AAV (adeno-associated virus), recombinant HSV, poxviruses (vaccinia) and recombinant lentivirus (such as HIV). All these vectors achieve the basic goal of delivering into the target cell the cDNA sequence and control elements needed for transcription. The present disclosure includes all forms of nucleic acid delivery, including synthetic oligos, naked DNA, plasmid and viral, integrated into the genome or not.

Also contemplated are inhibitory nucleic acid therapeutics that can inhibit the activity of JMHV, for example in a subject with MS or other diseases associated with JMHV infection. Inhibitory nucleic acids may be single-stranded nucleic acids, which can specifically bind to a complementary nucleic acid sequence. By binding to the appropriate target sequence, an RNA-RNA, a DNA-DNA, or RNA-DNA duplex or triplex is formed. These nucleic acids are often termed “antisense” because they are usually complementary to the sense or coding strand of the gene, although recently approaches for use of “sense” nucleic acids have also been developed. The term “inhibitory nucleic acids” as used herein, refers to both “sense” and “antisense” nucleic acids, and includes small inhibitory RNAs.

By binding to the target nucleic acid, the inhibitory nucleic acid can inhibit the function of the target nucleic acid. This could, for example, be a result of blocking DNA transcription, processing or poly(A) addition to mRNA, DNA replication, translation, or promoting inhibitory mechanisms of the cells, such as promoting RNA degradation. Inhibitory nucleic acid methods therefore encompass a number of different approaches to altering expression of JMHV genes. Cleavage, and therefore inactivation, of the target nucleic acids may be effected by attaching a substituent to the inhibitory nucleic acid which can be activated to induce cleavage reactions. The substituent can be one that affects either chemical, or enzymatic cleavage. Alternatively, cleavage can be induced by the use of ribozymes or catalytic RNA. In this approach, the inhibitory nucleic acids would include either naturally occurring RNA (ribozymes) or synthetic nucleic acids with catalytic activity.

The inhibitory nucleic acid therapies can be used to target nucleic acids to sequences of JMHV for use in treating conditions caused by the JMHV, or proteins of the JMHV, for example for treating MS or an other immune mediated syndromes, such as an autoimmune disease.

Therapeutic, intravenous, polyclonal or monoclonal antibodies have been used as a mode of passive immunotherapy of herpesviral diseases, such as infection with CMV. Immune globulin from subjects previously infected with the JMHV and bearing a suitably high titer of antibodies against the virus can be given in combination with antiviral agents (e.g. ganciclovir), or in combination with other modes of immunotherapy that are currently being evaluated for the treatment of MS, which are targeted to modulating the immune response (such as treatment with antibodies that inhibit the interaction of IL-2 with its receptor). In one embodiment, antibodies specific for an epitope expressed on cells infected with the JMHV are utilized and can be obtained as described above.

Thus, pharmaceutical compositions which include a therapeutically effective amount of an antibody, and a pharmaceutically acceptable carrier or excipient. The antibody can be polyclonal or monoclonal.

A method is provided herein for vaccinating a subject against multiple sclerosis, comprising administering to a susceptible subject an effective amount of the peptide or polypeptide encoded by an isolated DNA molecule encoding a polypeptide or combination of polypeptides expressed by the DNA molecule, and a suitable acceptable carrier. In one embodiment, DNA is administered to the subject in an effective amount to vaccinate the subject against multiple sclerosis, or other disease associated with JMHV infection.

The vaccine can be made using synthetic peptide or recombinantly-produced polypeptide described above as antigen. Typically, a vaccine will include from about 1 to 50 micrograms of antigen, for example from about 15 to about 45 micrograms. Typically, the vaccine is formulated so that a dose includes about 0.5 milliliters. The vaccine may be administered by any route known in the art, for example parenteral, subcutaneous or intramuscular.

There are a number of strategies for amplifying an antigen's effectiveness, particularly as related to the art of vaccines. For example, cyclization of a peptide can increase the peptide's antigenic and immunogenic potency. See U.S. Pat. No. 5,001,049. More conventionally, an antigen can be conjugated to a suitable carrier, usually a protein molecule. This procedure can allow multiple copies of an antigen, such as a peptide, to be conjugated to a single larger carrier molecule. Additionally, the carrier may possess properties which facilitate transport, binding, absorption or transfer of the antigen.

For parenteral administration, such as subcutaneous injection, examples of suitable carriers are the tetanus toxoid, the diphtheria toxoid, serum albumin and lamprey, or keyhole limpet, hemocyanin because they provide the resultant conjugate with minimum genetic restriction. Conjugates including these universal carriers can function as T cell clone activators in individuals having very different gene sets. The conjugation between a peptide and a carrier can be accomplished using one of the methods known in the art. Specifically, the conjugation can use bifunctional cross-linkers as binding agents as detailed, for example, by Means and Feeney, “A recent review of protein modification techniques,” Bioconjugate Chem. 1:2-12, 1990.

Vaccines against JMHV can be made from the JMHV envelope glycoproteins. These proteins can be purified and used for vaccination (Lasky, J. Med. Virol. 31:59, 1990). MHC-binding peptides from cells infected with the human herpesvirus can be identified for vaccine candidates per the methodology of Marloes et al., Eur. J. Immunol.21:2963-2970, 1991. The JMHV antigen may be combined or mixed with various solutions and other compounds as is known in the art. For example, it may be administered in water, saline or buffered vehicles with or without various adjuvants or immunodiluting agents. Examples of such adjuvants or agents include aluminum hydroxide, aluminum phosphate, aluminum potassium sulfate (alum), beryllium sulfate, silica, kaolin, carbon, water-in-oil emulsions, oil-in-water emulsions, muramyl dipeptide, bacterial endotoxin, lipid X, Corynebacterium parvum (Propionibacterium acnes), Bordetella pertussis, polyribonucleotides, sodium alginate, lanolin, lysolecithin, vitamin A, saponin, liposomes, levamisole, DEAE-dextran, blocked copolymers or other synthetic adjuvants. Such adjuvants are available commercially from various sources, for example, Merck Adjuvant 65 (Merck and Company, Inc., Rahway, N.J.) or Freund's Incomplete Adjuvant and Complete Adjuvant (Difco Laboratories, Detroit, Mich.). Other suitable adjuvants are Amphigen (oil-in-water), Alhydrogel (aluminum hydroxide), or a mixture of Amphigen and Alhydrogel. Only aluminum is approved for human use.

The proportion of antigen and adjuvant can be varied over a broad range so long as both are present in effective amounts. For example, aluminum hydroxide can be present in an amount of about 0.5% of the vaccine mixture (Al₂O₃ basis). On a per-dose basis, the amount of the antigen can range from about 0.1 μg to about 100 μg protein per subject, for example about 1 μg to about 50 μg per dose, or about 15 μg to about 45 μg. A suitable dose size is about 0.5 ml. Accordingly, a dose for intramuscular injection, for example, would comprise 0.5 ml containing 45 μg of antigen in admixture with 0.5% aluminum hydroxide. After formulation, the vaccine may be incorporated into a sterile container which is then sealed and stored at a low temperature, for example 4° C., or it may be freeze-dried. Lyophilization permits long-term storage in a stabilized form.

The vaccines may be administered by any conventional method for the administration of vaccines including oral and parenteral (e.g., subcutaneous or intramuscular) injection. Intramuscular administration is preferred. The treatment may consist of a single dose of vaccine or a plurality of doses over a period of time. Also, the antigen could be a component of a recombinant vaccine which is adaptable for oral administration. Vaccines of the disclosure may be combined with other vaccines for other diseases to produce multivalent vaccines. A pharmaceutically effective amount of the antigen can be employed with a pharmaceutically acceptable carrier such as a protein or diluent useful for the vaccination of mammals, particularly humans. Other vaccines may be prepared according to methods well-known to those skilled in the art.

Those of skill will readily recognize that it is only necessary to expose a mammal to appropriate epitopes in order to elicit effective immunoprotection. The epitopes are typically segments of amino acids which are a small portion of the whole protein. Using recombinant genetics, it is routine to alter a natural protein's primary structure to create derivatives embracing epitopes that are identical to or substantially the same as (immunologically equivalent to) the naturally occurring epitopes. Such derivatives may include peptide fragments, amino acid substitutions, amino acid deletions and amino acid additions of the amino acid sequence for the viral polypeptides from the human herpesvirus. For example, it is known in the protein art that certain amino acid residues can be substituted with amino acids of similar size and polarity without an undue effect upon the biological activity of the protein. The human herpesvirus proteins have significant tertiary structure and the epitopes are usually conformational. Thus, modifications should generally preserve conformation to produce a protective immune response.

The disclosure is illustrated by the following non-limiting Examples.

### EXAMPLES

Japanese macaque herpesvirus (JMHV) was isolated from a monkey with encephalomyelitis and the complete nucleotide sequence was determined. DNA sequence and phylogenetic analysis revealed JMHV is a gamma-2 herpesvirus. JMHV was associated with demyelinating disease in vivo. The virus can also be propagated in vitro. As disclosed herein the entire genome of a novel gamma herpesvirus, referred to as Japanese macaque herpesvirus (JMHV) has been identified and sequenced. The virus was isolated from an inflamed spinal cord lesion obtained from a Japanese macaque with spontaneous MS-like disease.

Rhesus macaques naturally harbor a virus related to KSHV, referred to as RRV, for rhesus rhadinovirus. JMHV is related to RRV, which is described in International Application No. PCT/US99/26260 filed Nov. 5, 1999, which claims priority from U.S. Provisional Application No. 60/109,409 filed Nov. 20, 1998 and U.S. Provisional Application No. 60/107,507 filed Nov. 6, 1998, are also incorporated herein by reference

### Example 1

### Virus Isolation

Japanese macaque encephalomyelitis (JME) is a spontaneous demyelinating disease with clinical features such as ataxia and paralysis similar to those of MS. To investigate whether a virus is associated with the development of JME, a spinal cord lesion was obtained at necropsy from a macaque with JME, dissociated and cultured with primary macaque fibroblasts. Cytopathic effects (CPE) developed in the culture after 10 days and the culture was harvested for passage. Primary macaque fibroblasts were inoculated with filtered extracts from the original culture and observed for CPE. Cultures developing CPE were harvested and analyzed by transmission electron microscopy (TEM) for the presence of virus particles. TEM examination revealed 150-200 nm virus particles with characteristic dense cores resembling herpesviruses. Limiting dilution of the original virus isolate yielded a purified herpesvirus preparation, which was subsequently expanded for viral DNA isolated and identified by degenerate PCR (Wucherpfennig and Strominger, Cell 80:695-705, 1995).

### Example 2

### Sequence Analysis and Identification of Open Reading Frames

Sequence analysis of the degenerate PCR product targeting the DNA polymerase gene revealed that the JMHV was closely related to rhesus macaque rhadinovirus (RRV) (Searles et al., J Virol 73:3040-53, 1999). To further identify the herpesvirus, a shotgun subclone library of the JMHV genome was generated and the genomic sequence deduced as recently described for rhesus cytomegalovirus (Hansen et al, J Virol 77:6620-36, 2003). The complete JMHV sequence was compiled and analyzed using MacVector version 7.2 (Acelrys, San Diego, Calif.). The target search criterion was for open reading frames (ORFs) that encoded proteins of 80 amino acids or more. Putative ORFs were translated and homologous proteins were identified using GenBank's BLASTP search engine. The full length nucleic acid sequence is shown in SEQ ID NO: 1.

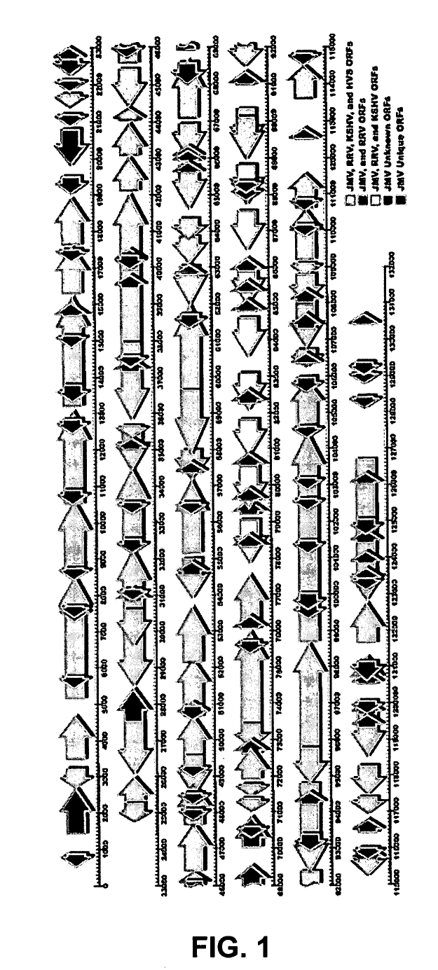

Analysis of the JMHV DNA sequence indicated the genome is similar to all herpesviruses, has a linear, double-stranded DNA genome that is 131,217 base pairs in length and encodes 171 potential ORFs (Table 1, see also FIG. 1). The ORF finder of MacVector was used to identify all 171 putative ORFs greater than or equal to 80 amino acids. Putative ORFs were numbered by the order in which they appear in the genome. In Table 1, ORFs that read left to right are designated W, whereas ORFs that read right to left are designated by C. ORFs from RRV strain 17577 (GenBank Accession No. AF083501) were used for comparison. The complete JMHV genome sequence has been deposited in GenBank.

TABLE 1
JMHV ORF Summary

| | | Position (nt) | | Length | | RRV | |
|---|---|---|---|---|---|---|---|
| ORF | Strand | From | To | b.p. | a.a | Homolog | Description |
| JM1 | C | 594 | 890 | 296 | 99 | | |
| JM2 | W | 1444 | 2721 | 1277 | 426 | R1 | IgG receptor |
| JM3 | C | 2778 | 3359 | 581 | 194 | ORF 2 | Dihydrofolate reductase |
| JM4 | W | 3526 | 4713 | 1187 | 396 | ORF 4 | Complement binding |
| | | | | | | | protein |
| JM5 | W | 5146 | 8544 | 3398 | 1133 | ORF 6 | ssDNA binding protein |
| JM6 | C | 5581 | 5895 | 314 | 105 | | |
| JM7 | C | 7410 | 7676 | 266 | 89 | | |
| JM8 | C | 7462 | 7722 | 260 | 87 | | |
| JM9 | C | 8541 | 8792 | 251 | 84 | | |
| JM10 | W | 8569 | 10629 | 2060 | 687 | ORF 7 | Processing/transport |
| | | | | | | | protein |
| JM11 | C | 10606 | 10929 | 323 | 108 | | |
| JM12 | W | 10616 | 13102 | 2486 | 829 | ORF 8 | Glycoprotein B |
| JM13 | C | 12565 | 12858 | 293 | 98 | | |
| JM14 | W | 13219 | 16257 | 3038 | 1013 | ORF 9 | DNA polymerase |
| JM15 | C | 13267 | 13827 | 560 | 187 | | |
| JM16 | C | 14809 | 15117 | 308 | 103 | | |
| JM17 | W | 15726 | 16031 | 305 | 102 | | |
| JM18 | W | 16351 | 17601 | 1250 | 417 | ORF 10 | |
| JM19 | C | 17226 | 17501 | 275 | 92 | | |
| JM20 | W | 17610 | 18839 | 1229 | 410 | ORF 11 | |
| JM21 | C | 19018 | 19641 | 623 | 208 | R2 | vIL-6 |
| JM22 | C | 19861 | 20862 | 1001 | 334 | ORF 70 | Thymidylate synthetase |
| JM23 | C | 20969 | 21235 | 266 | 89 | | |
| JM24 | C | 21386 | 21748 | 362 | 121 | R3 | VMIP |
| JM25 | C | 21845 | 22120 | 275 | 92 | | HHV2 RS1; collagen-like, |
| | | | | | | | myosin XV |
| JM26 | C | 22363 | 22701 | 338 | 113 | | Myosin IA, sphingosine |
| | | | | | | | kinase |
| JM27 | W | 22700 | 23008 | 308 | 103 | | Similar to RRV ORF RU1-R |
| JM28 | W | 25392 | 25955 | 563 | 188 | ORF 16 | Bcl-2 homolog |
| JM29 | C | 26062 | 27672 | 1610 | 537 | ORF 17 | Capsid protein |
| JM30 | W | 27545 | 28444 | 899 | 300 | ORF 18 | |
| JM31 | W | 28138 | 28431 | 293 | 98 | | |
| JM32 | C | 28452 | 30095 | 1643 | 548 | ORF 19 | Tegument protein |
| JM33 | C | 29590 | 30642 | 1052 | 351 | ORF 20 | |
| JM34 | W | 30641 | 32311 | 1670 | 557 | ORF 21 | Thymidine kinase |
| JM35 | W | 30825 | 31181 | 356 | 119 | | MHC psoriasis candidate |
| JM36 | C | 30863 | 31111 | 248 | 83 | | Possible transposable |
| | | | | | | | element |
| JM37 | C | 32279 | 32608 | 329 | 110 | | |
| JM38 | W | 32298 | 34478 | 2199 | 727 | ORF 22 | Glycoprotein H |
| JM39 | C | 33254 | 33553 | 299 | 100 | | Succinate dehydrogenase |
| JM40 | C | 34475 | 35687 | 1212 | 404 | ORF 23 | EBV BTRF1 homolog |
| JM41 | W | 34974 | 35285 | 311 | 104 | | Alpha-1A adrenergic |
| | | | | | | | receptor |
| JM42 | C | 35301 | 35687 | 386 | 129 | ORF 23 | EBV BTRF1 homolog |
| JM43 | C | 35737 | 37938 | 2201 | 734 | ORF 24 | EBV BcRF1 homolog |
| JM44 | C | 37346 | 37612 | 266 | 89 | | |
| JM45 | W | 37937 | 42073 | 4136 | 1379 | ORF 25 | Major capsid protein |
| JM46 | W | 39498 | 39821 | 323 | 108 | | |
| JM47 | C | 39842 | 40282 | 440 | 147 | | |
| JM48 | W | 40188 | 40439 | 251 | 84 | | ICHIT protein; mucin |
| JM49 | W | 42105 | 43022 | 917 | 306 | ORF 26 | Minor capsid protein |
| JM50 | W | 43047 | 43871 | 824 | 275 | ORF 27 | EBV BDLF2 homolog |
| JM51 | W | 44035 | 44310 | 275 | 92 | ORF 28 | |
| JM52 | C | 44360 | 45406 | 1371 | 349 | ORF 29b | Packaging protein (spliced) |
| JM53 | W | 45720 | 46373 | 653 | 218 | ORF 31 | EBV BDLF4 homolog |
| JM54 | C | 45836 | 46195 | 359 | 120 | | Collagen family member; |
| | | | | | | | ataxin 7 |
| JM55 | W | 46310 | 47695 | 1385 | 462 | ORF 32 | EBV BGLF1 homolog |
| JM56 | W | 47676 | 48686 | 1010 | 337 | ORF 33 | EBV BGLF2 homolog |
| JM57 | C | 47768 | 48136 | 368 | 123 | | Calcium channel |
| JM58 | C | 48218 | 48607 | 389 | 130 | | Protein kinase-like |
| JM59 | C | 48604 | 49587 | 983 | 328 | ORF 29a | Packaging protein (spliced) |
| JM60 | C | 49010 | 49276 | 266 | 89 | | |
| JM61 | W | 49586 | 50572 | 986 | 329 | ORF 34 | EBV BGLF3 |
| JM62 | C | 50504 | 50950 | 446 | 149 | | |
| JM63 | W | 50553 | 51002 | 449 | 150 | ORF 35 | EBV BGLF3.5 homolog |
| JM64 | W | 50908 | 52215 | 1307 | 436 | ORF 36 | Kinase |
| JM65 | W | 52196 | 53638 | 1442 | 481 | ORF 37 | Alkaline exonuclease |
| JM66 | C | 53882 | 55018 | 1136 | 379 | ORF 39 | Glycoprotein M |
| JM67 | W | 54536 | 54850 | 314 | 105 | | Histidyl-tRNA synthetase, |
| | | | | | | | ligase |
| JM68 | W | 55153 | 57156 | 2003 | 668 | ORF 40 | Helicase/primase complex |
| | | | | | | | component |
| JM69 | C | 56124 | 56474 | 350 | 117 | | Unknown |
| JM70 | C | 57153 | 57968 | 815 | 272 | ORF 42 | EBV BBRF2 homolog |
| JM71 | W | 57325 | 57573 | 248 | 83 | | Chromatin associated |
| | | | | | | | protein |
| JM72 | C | 57907 | 59652 | 1745 | 582 | ORF 43 | Capsid protein |
| JM73 | W | 59591 | 61963 | 2372 | 791 | ORF 44 | Helicase/primase complex |
| | | | | | | | component |
| JM74 | C | 61249 | 61662 | 413 | 138 | | Transcription factor AFX1, |
| | | | | | | | ChCMV UL7 |
| JM75 | C | 62004 | 63065 | 1061 | 354 | ORF 45 | EBV BKRF4 |
| JM76 | W | 62823 | 63086 | 263 | 88 | | Plexin |
| JM77 | C | 63107 | 63874 | 767 | 256 | ORF 46 | Uracil DNA glucosidase |
| JM78 | C | 63850 | 64341 | 491 | 164 | ORF 47 | Glycoprotein L |
| JM79 | C | 64600 | 65769 | 1169 | 390 | ORF 48 | EBV BRRF2 homolog |
| JM80 | W | 65629 | 65880 | 251 | 84 | | NADH-ubiquinone |
| | | | | | | | dehydrogenase chain 8 |
| JM81 | W | 65963 | 66208 | 245 | 82 | | |
| JM82 | C | 66000 | 66905 | 905 | 302 | ORF 49 | EBV BRRF1 homolog |
| JM83 | W | 66058 | 66315 | 257 | 86 | | |
| JM84 | W | 67096 | 68640 | 1544 | 515 | ORF 50 | Transactivator |
| JM85 | C | 67920 | 68594 | 674 | 225 | | Chromatin remodeling |
| | | | | | | | complex |
| JM86 | W | 68957 | 69469 | 512 | 171 | | BZIP transcription factor |
| JM87 | W | 70026 | 70760 | 734 | 245 | | Glycoprotein R8.1; mucin |
| JM88 | C | 70328 | 70606 | 278 | 93 | | Neurexin 1-alpha |
| JM89 | C | 71001 | 71420 | 419 | 124 | ORF 52 | EBV BLRF2 homolog |
| JM90 | C | 71483 | 71797 | 314 | 105 | ORF 53 | EBV BLRF1 homolog |
| JM91 | W | 71873 | 72745 | 872 | 291 | ORF 54 | DUTPase |
| JM92 | W | 72520 | 72942 | 422 | 141 | | Transducin-like enhancer |
| JM93 | C | 72806 | 73438 | 632 | 211 | ORF 55 | EBV BSRF1 homolog |
| JM94 | W | 73420 | 75936 | 2516 | 839 | ORF 56 | DNA replication protein |
| JM95 | C | 75447 | 75722 | 275 | 92 | | Cytochrome oxidase |
| | | | | | | | subunit I |
| JM96 | W | 76035 | 76304 | 269 | 90 | | |
| JM97 | W | 76162 | 77484 | 1322 | 441 | ORF 57 | Immediate-early protein |
| JM98 | C | 77857 | 79098 | 1241 | 414 | R6 | VIRF |
| JM99 | W | 78411 | 78656 | 245 | 82 | | |
| JM100 | W | 79163 | 79579 | 416 | 139 | | Ribonuclease III |
| JM101 | C | 79269 | 80510 | 1241 | 414 | R7 | VIRF |
| JM102 | W | 79521 | 79769 | 248 | 83 | | Hepatitis B core protein |
| JM103 | C | 80836 | 81891 | 1055 | 352 | R8 | VIRF |
| JM104 | C | 82068 | 83153 | 1085 | 362 | R9 | VIRF |
| JM105 | W | 82206 | 82613 | 407 | 136 | | RNA polymerase |
| | | | | | | | associated factor |
| JM106 | C | 83628 | 84785 | 1157 | 386 | R10 | VIRF |
| JM107 | W | 84837 | 85082 | 245 | 82 | | Transposase |
| JM108 | C | 84932 | 86104 | 1172 | 391 | R11 | VIRF |
| JM109 | W | 85290 | 85550 | 260 | 87 | | |
| JM110 | W | 85968 | 86243 | 275 | 92 | | |
| JM111 | C | 86479 | 87546 | 1067 | 356 | R12 | VIRF |
| JM112 | C | 87707 | 88801 | 1094 | 365 | R13 | VIRF |
| JM113 | C | 87763 | 88023 | 260 | 87 | | |
| JM114 | C | 88087 | 88395 | 308 | 103 | | |
| JM115 | C | 89047 | 90129 | 1082 | 361 | ORF 58 | |
| JM116 | C | 90140 | 91324 | 1184 | 395 | ORF 59 | DNA replication protein |
| JM117 | W | 91017 | 91259 | 242 | 81 | | |
| JM118 | C | 91455 | 92399 | 944 | 315 | ORF 60 | Small ribonucleotide |
| | | | | | | | reductase |
| JM119 | C | 92381 | 94747 | 2366 | 789 | ORF 61 | Large ribonucleotide |
| | | | | | | | reductase |
| JM120 | C | 92929 | 93345 | 416 | 139 | | |
| JM121 | W | 94175 | 94468 | 293 | 98 | | DNA repair and genetic |
| | | | | | | | recombination |
| JM122 | C | 94751 | 95746 | 995 | 332 | ORF 62 | Assembly/DNA maturation |
| | | | | | | | protein |
| JM123 | W | 95745 | 98564 | 2819 | 940 | ORF 63 | Tegument protein |
| JM124 | W | 98568 | 104468 | 5900 | 1967 | ORF 64 | Tegument protein |
| JM125 | C | 99388 | 99753 | 365 | 122 | | |
| JM126 | C | 99611 | 100054 | 443 | 148 | | Drug resistance protein |
| JM127 | C | 101108 | 101428 | 320 | 107 | | |
| JM128 | C | 102290 | 102568 | 278 | 93 | | |
| JM129 | C | 102857 | 103150 | 293 | 98 | | |
| JM130 | C | 104302 | 104643 | 341 | 114 | | |
| JM131 | W | 104555 | 105856 | 1301 | 434 | ORF 64 | Tegument protein |
| JM132 | C | 105581 | 106003 | 422 | 141 | | C-myc promoter binding |
| | | | | | | | protein |
| JM133 | C | 106220 | 106729 | 509 | 170 | ORF 65 | Capsid protein |
| JM134 | W | 106299 | 106559 | 260 | 87 | | |
| JM135 | C | 106733 | 108079 | 1346 | 449 | ORF 66 | EBV BFRF2 homolog |
| JM136 | W | 107289 | 107696 | 407 | 136 | | |
| JM137 | C | 107974 | 108780 | 806 | 269 | ORF 67 | Tegument protein |
| JM138 | W | 108002 | 108316 | 314 | 105 | | |
| JM139 | C | 108796 | 109056 | 260 | 87 | ORF 67.5 | |
| JM140 | W | 109190 | 110563 | 1373 | 458 | ORF 68 | Glycoprotein |
| JM141 | C | 109783 | 110166 | 383 | 128 | | |
| JM142 | C | 110578 | 110904 | 326 | 109 | | Aldehyde dehydrogenase |
| JM143 | W | 110585 | 111478 | 893 | 298 | ORF 69 | EBV BFLF2 homolog |
| JM144 | W | 112447 | 112698 | 251 | 84 | | RRV ORF RU3-R |
| JM145 | W | 113610 | 114479 | 869 | 290 | | RRV ORF RU4-R |
| JM146 | C | 114427 | 114753 | 326 | 109 | | |
| JM147 | C | 115214 | 115918 | 704 | 235 | | RRV ORF RU13-L; |
| | | | | | | | serotonin receptor |
| JM148 | W | 115671 | 115928 | 257 | 86 | | EBV nuclear antigen |
| JM149 | C | 115717 | 115983 | 266 | 89 | | |
| JM150 | W | 116366 | 116650 | 284 | 95 | | Lagopus leucurus gag |
| | | | | | | | polyprotein |
| JM151 | C | 116918 | 117442 | 524 | 175 | ORF 71 | FLIP homolog |
| JM152 | C | 117501 | 118265 | 764 | 255 | ORF 72 | Cyclin D homolog |
| JM153 | C | 118608 | 119918 | 1310 | 437 | ORF 73 | Latency-associated nuclear |
| | | | | | | | antigen |
| JM154 | W | 119237 | 119617 | 380 | 127 | | |
| JM155 | W | 119614 | 119940 | 326 | 109 | | |
| JM156 | C | 119824 | 120075 | 251 | 84 | | |
| JM157 | W | 120484 | 120744 | 260 | 87 | | |
| JM158 | W | 120572 | 121333 | 761 | 254 | R15 | N-CAM Ox-2 homolog |
| JM159 | C | 120689 | 121186 | 497 | 166 | | CD36; carotenoid uptake |
| JM160 | W | 121628 | 122656 | 1028 | 343 | ORF 74 | IL-8 receptor; G protein |
| | | | | | | | coupled receptor |
| JM161 | C | 122762 | 126658 | 3896 | 1299 | ORF 75 | Tegument protein |
| JM162 | C | 123126 | 123398 | 272 | 91 | | |
| JM163 | W | 123423 | 123577 | 154 | 85 | | |
| JM164 | W | 123810 | 124133 | 323 | 108 | | |
| JM165 | W | 124395 | 124790 | 395 | 132 | | K1 glycoprotein HHV-8 |
| JM166 | C | 124683 | 125165 | 482 | 161 | | Collagen family member |
| JM167 | W | 126063 | 126371 | 308 | 103 | | Myosin phosphatase |
| JM168 | C | 128122 | 128493 | 371 | 124 | | C-C Chemokine receptor |
| JM169 | C | 128789 | 129046 | 257 | 86 | | |
| JM170 | C | 129970 | 130404 | 434 | 145 | | Unknown |
| JM171 | W | 130383 | 130655 | 272 | 91 | | |

The overall G+C content was 51.9% and is distributed evenly throughout the length of the genome. The genes were named from left to right starting at the first ORF on the coding or complementary strand (e.g., 155). The JM prefix precedes each numbered gene to distinguish different viruses' nomenclature (e.g., JM1). The arrangement of JMHV genes is shown in FIG. 1 and was compared with other primate gamma-2 herpesvirus genomes. A phylogenetic analysis was performed using six ORFs from HSV-1, Kaposi's sarcoma-associated herpesvirus (KSHV), human cytomegalovirus (HCMV) and JMHV. The DNA polymerase, helicase, glycoprotein B, major capsid protein, single-stranded DNA binding protein, and uracil N-glycosylase were examined by bootstrap analysis with the maximum parsimony method. Alignments were performed using ClustalW. The KSHV proteins were used as the root for analysis. The phylogenetic analysis revealed that JMHV is a gamma-herpesvirus most closely related to RRV.

JMHV ORFs are arranged collinearly with other gamma-herpesviruses, including RRV and KSHV (Chang et al., Science 266:1865-9, 1994; Moore et al., J Virol 70:549-58, 1996; Russo et al., Proc Natl Acad Sci USA 93:14862-7, 1996; Searles et al., J Virol 73:3040-53, 1999). Most all of the conserved herpesvirus gene blocks are retained in JMHV in both position and orientation, coding all of the necessary enzymes and structural components. Of the 171 ORFs, 81 (47%) are homologous to known RRV proteins. However, the homologous genes are on average 88% identical with a high of 99% and a low of 36%. A protein/protein comparison was done. The percent identity of the two proteins and the scores (except value; calculated based on the size of nr database) for the alignments are shown in Table 2.

TABLE 2
Similarity between JMHV genes
with amino acid sequence homology to RRV

| | | | | | Score |
|---|---|---|---|---|---|
| JMHV | Length | RRV | Homolog | Identity for | (except |
| gene | (aa) | homolog | length (aa) | overlap (%) | value) |
| JM2 | 426 | R1 | 424 | 82 | 1845 |
| JM3 | 194 | ORF2 | 189 | 94 | 937 |
| JM4 | 396 | ORF4 | 646 | 51 | 1001 |
| JM5 | 1133 | ORF6 | 1133 | 97 | 5878 |
| JM10 | 687 | ORF7 | 687 | 90 | 3240 |
| JM12 | 829 | ORF8 | 830 | 89 | 3789 |
| JM14 | 1013 | ORF9 | 1015 | 93 | 4982 |
| JM18 | 417 | ORF10 | 417 | 91 | 1997 |
| JM20 | 410 | ORF11 | 410 | 97 | 2128 |
| JM21 | 208 | R2 | 208 | 85 | 915 |
| JM22 | 334 | ORF70 | 334 | 93 | 1637 |
| JM24 | 121 | R3 | 116 | 65 | 378 |
| JM28 | 188 | ORF16 | 188 | 76 | 734 |
| JM29 | 537 | ORF17 | 537 | 82 | 2211 |
| JM30 | 300 | ORF18 | 300 | 95 | 1527 |
| JM32 | 548 | ORF19 | 548 | 97 | 2796 |
| JM33 | 351 | ORF20 | 351 | 91 | 1700 |
| JM34 | 557 | ORF21 | 558 | 87 | 2532 |
| JM38 | 727 | ORF22 | 705 | 73 | 2638 |
| JM43 | 734 | ORF24 | 733 | 90 | 3477 |
| JM45 | 1379 | ORF25 | 1379 | 97 | 7077 |
| JM49 | 306 | ORF26 | 306 | 99 | 1556 |
| JM50 | 275 | ORF27 | 270 | 90 | 1327 |
| JM51 | 92 | ORF28 | 92 | 91 | 459 |
| JM52 | 349 | ORF29B | 349 | 94 | 1689 |
| JM53 | 218 | ORF31 | 218 | 90 | 1068 |
| JM55 | 462 | ORF32 | 465 | 91 | 2255 |
| JM56 | 337 | ORF33 | 337 | 91 | 1630 |
| JM61 | 329 | ORF34 | 329 | 92 | 1612 |
| JM63 | 150 | ORF35 | 150 | 94 | 702 |
| JM64 | 436 | ORF36 | 436 | 93 | 2196 |
| JM65 | 481 | ORF37 | 481 | 96 | 2496 |
| JM66 | 379 | ORF39 | 379 | 94 | 1844 |
| JM68 | 668 | ORF40 | 469 | 96 | 2399 |
| JM70 | 272 | ORF42 | 273 | 95 | 1376 |
| JM72 | 582 | ORF43 | 577 | 95 | 2810 |
| JM73 | 791 | ORF44 | 791 | 98 | 4092 |
| JM75 | 354 | ORF45 | 353 | 68 | 1227 |
| JM77 | 256 | ORF46 | 256 | 87 | 1226 |
| JM78 | 164 | ORF47 | 170 | 55 | 440 |
| JM79 | 390 | ORF48 | 390 | 92 | 1759 |
| JM82 | 302 | ORF49 | 302 | 99 | 1595 |
| JM84 | 515 | ORF50 | 515 | 92 | 2492 |
| JM89 | 124 | ORF52 | 124 | 79 | 532 |
| JM90 | 105 | ORF53 | 105 | 59 | 300 |
| JM91 | 291 | ORF54 | 291 | 97 | 1479 |
| JM93 | 211 | ORF55 | 211 | 99 | 1092 |
| JM94 | 839 | ORF56 | 829 | 95 | 4186 |
| JM97 | 441 | ORF57 | 443 | 83 | 1890 |
| JM98 | 414 | R6 | 416 | 81 | 1778 |
| JM101 | 414 | R7 | 416 | 90 | 1978 |
| JM103 | 352 | R8 | 352 | 92 | 1752 |
| JM104 | 362 | R9 | 254 | 89 | 1217 |
| JM106 | 386 | R10 | 386 | 86 | 1814 |
| JM108 | 391 | R11 | 391 | 77 | 1617 |
| JM111 | 356 | R12 | 356 | 88 | 1739 |
| JM112 | 365 | R13 | 365 | 84 | 1697 |
| JM115 | 361 | ORF58 | 361 | 74 | 1319 |
| JM116 | 395 | ORF59 | 395 | 94 | 1937 |
| JM118 | 315 | ORF60 | 315 | 98 | 1589 |
| JM119 | 789 | ORF61 | 789 | 96 | 4008 |
| JM122 | 332 | ORF62 | 332 | 98 | 1696 |
| JM123 | 940 | ORF63 | 940 | 92 | 4506 |
| JM124 | 1967 | ORF64 | 2549 | 92 | 9299 |
| JM133 | 170 | ORF65 | 170 | 66 | 557 |
| JM135 | 449 | ORF66 | 449 | 92 | 2212 |
| JM137 | 269 | ORF67 | 225 | 97 | 1120 |
| JM139 | 87 | ORF67.5 | 87 | 95 | 431 |
| JM140 | 458 | ORF68 | 458 | 94 | 2361 |
| JM143 | 298 | ORF69 | 298 | 97 | 1520 |
| JM151 | 175 | ORF71 | 175 | 91 | 836 |
| JM152 | 255 | ORF72 | 255 | 76 | 967 |
| JM153 | 437 | ORF73 | 449 | 36 | 162 |
| JM158 | 254 | R15 | 254 | 88 | 1190 |
| JM160 | 343 | ORF74 | 343 | 78 | 1387 |
| JM161 | 1299 | ORF75 | 1299 | 90 | 6241 |

Unique to JMHV are numerous small ORFs that have low homology to enzymes or structural components found in nervous tissue. Many of these putative proteins are less than 100 amino acids, which is less than what was reported in the characterization of RRV (Searles et al., supra). These potential ORFs are unique to JMHV, as analysis of the RRV genome for ORFs encoding proteins of 80 amino acids or more failed to identify similar ORFs. From this analysis it was determined that these small ORFs are specific to JMHV and includes proteins such as myosin XV (JM25), myosin IA (JM26), ataxin 7 (JM54), chromatin associated protein (JM71), chromatin remodeling complex (JM85) and Neurexin 1-alpha (JM88) (Del-Favero et al., Hum Mol Genet 7:177-86, 1998; Liang et al., Genomics 61:243-58, 1999; Patzke and Emsberger, Mol Cell Neurosci 15:561-72, 2000). Thus, these proteins could be involved with molecular mimicry or epitope spread leading to the MS-like disease observed in the Japanese macaque.

The sequence of the JMHV ORFs are provided in Table 3.

TABLE 3
ORF sequences

| | |
|---|---|
| JMHV | SEQUENCE |

| | | |
|---|---|---|
| ORF | SEQUENCE | IDENTIFIER |

| | | |
|---|---|---|
| JM 1 | MRWAWFSPFLSHQLGVSNHAPSPGPTSAPLPIPLLPVPSPASMLPAP | SEQ ID NO: 2 |
| | TALSVRSPRATCRADLGRLPGGPGEGSGPVSSCGPATPSHAAGRLPH | |
| | YYQP | |
| JM 2 | MFVLLIFILLQPASLELLPAKLTAVPTWCPPHPGDTYLLTCRGTSTA | SEQ ID NO: 3 |
| | RDQRSTQWFRNNTLMHGSNFYGRLVSVTPNSTISDWYACQTKTTTRS | |
| | NSIDFRVRSSRLTLQERCSSYGYSNANNTRVLRCYSGGNVTLRNVVF | |
| | HLNGTAVINGTTTDIYTFVLTEKTGGTYYCSAFIGTEKLYSQKINVF | |
| | FTSFTFKHTDNVQMGSEFNKTEQIQQTANVQHTANYVVFSVPVFSIG | |
| | VLTGIAISLIMCWLFTLRCNKNSESSNNRHAHQTSYIQPSHNQHSHT | |
| | SESTTHTYRNDHQEESIEELPNQHTRKTNSCQTVLLEVKNVAFDGPQ | |
| | GNLHNTNDEVMEQYDDVVVENIEQTSYDNNIEQMDYSDIIRPNFNYY | |
| | SGLILEEVDEVFYNELANQYHGLILENLDHDEYNHLNKLNMIEQYDW | |
| | LE | |
| JM 3 | MDITVNCIVAVDKQLGIGKNGTMPWPYLKNEMMYFQKMTSTPSVIGE | SEQ ID NO: 4 |
| | KNVVIMGKRTWFSIPEKKRPLANRINIILSRELREPPKGAHFLARTL | |
| | DDAFNFYRQYKLTKQLNTVWVIGGKSVYESVLNYECSLKLYITRIME | |
| | SFDCDVFFPSINFTEYTMLSELPGKDTNFEENGIKYKFQVYEKRLIN | |
| | KAVTQ | |
| JM 4 | MRLIMFWFPVTVCFVCHFILVNAQNETNVTCDKPNFDRFMVAMEKKE | SEQ ID NO: 5 |
| | KYVLGDKVELTCRPGYTLQGKVYVQCLQSGMWTTPNAECHRKKCTNY | |
| | GDILNGQVIVPDSDNAFKFGTNITYKCNTGYLLLGSMVRTCLLTGNS | |
| | NTVNWQPAAPTCEIEKCKKQPDIENGKYYPVQDFYNYLETITFTCNK | |
| | DFSLIGMTTTTCMTMGTWSSPVPKCEQITCSAPNIEHGTLLVGSSRV | |
| | YKHGQSITIGCENGFTLNGHKMCTCEYSSWNPPLPTCVPINKTVPTP | |
| | SEVPSPGTNKQERPTPENPKSHESETTTETPKTGTHKSETPSKKIPN | |
| | PETHKPTTPKSGTSEQTTNRPSKAPSQNPPMEPPMSKWKRHVVLVLF | |
| | ASVASLLFVLVTLYCCFLK | |
| JM 5 | MASKGNAGQPLEDNQGSRAPIGACGYVYAYSKQDFPFAEASILGNRP | SEQ ID NO: 6 |
| | SGSGVFSLPILYGLTVEHEFPLTVKAAHKKVDTTTLAVKVTCFHREV | |
| | IVFHNANLFRPVFEGTGLNELCEEARALFGYTQFIEPGPPHGIWNPL | |
| | ECPQLPDKDEMFLGVVVTEGFKERLWRGCLVPAVFQTQQVQIAGRQA | |
| | FKVPLYDEDLFAPHGHRMPRFYHKDVSAYLYNSLFTSIAQALRLKDV | |
| | TAVIHATEKQFMQDHYKIAKIVQAKQFSTTLPKTADGSSHMTVDSVV | |
| | AELALSYGCMFLECPQDACELLNYDSWPIFDGCDSSEDRVNALERWS | |
| | AEQAVHVAGQLFAANSVLYLTRVQKQAPRGQKGDVNVYNSFFLQHGL | |
| | GFLNEATIKENGSEAFKGVPSNALDGSSFTPYHLAYAASFSPHLLAK | |
| | LCYYMQFLQHHKSSTNQTFNIVHYVGTAANSEMCTLCHGDTPGTCLN | |
| | TLFYRLKDRFPAVTTPQRRDPYVVTGTAGTFNDLEILGNFASFRDRE | |
| | EDGNPADEHPKYTYWQLCQTVTEKLSAIGITEDQDNHVNLITNIQSF | |
| | LRVFKGIDSVVDGEVMKFINSMIKNNFNFREHVKSVHHILQFCCNVY | |
| | WQAPCAVFLNLYYKSLLWIIQDICLPYCMIYEQDNPAMGILPSEWLK | |
| | MHFQTLWTNFKAACLDRGVLTGCELKIVHRDMFCDFFDTDAGSNGLL | |
| | APFKMQVRIARAMMVVPKSIKIKNRIIFSNTAGSEAVQSGFVKPTGT | |
| | RDTYVVAGPYMKFLNSLHRALFPNTKTAALYLWHKISQTNKTPVLKD | |
| | VPDDELAELVSYVKTNSLAFEETNVLDVVPDSLMSYARIKLNGAILR | |
| | ACGQIQFYATTLHCLTPVLQTIDAEEYPHVLGSAAIATPVAYLSEIR | |
| | GRTALTVQTTARQPVAATGRLRPVITVPMVVNKYTGVNGNNNVFHCG | |
| | NLGYFAGRGVDRNLWPESSPFKKTGVSAMLRKRHVMMTPIVDRLIKR | |
| | AAGQTISTFEAESVKRSVQALLEDKDNPNLLKSVILELIRHLGKGCQ | |
| | DLSSEDVQYYLGDYCMLTDEVLFTLDNIAQSGVPWTFEDAGALIEDR | |
| | QNTDDLQFVDSDDIATASCQPPEEQLPTPSAGALLAGKKRKINVLLS | |
| | DLDL | |
| JM 6 | MVLHELFFCGVDDRRHVFESQGLGDTGKKGVVEVRANVFMVKPRHPM | SEQ ID NO: 7 |
| | PVRCEQVFVVQRHFERLTPGDLHLLGLKHCGNETAPPQSLLKPFRHD | |
| | NAKEHLILVR | |
| JM 7 | MAPFSLILAYDMRESGTTSSTFVSSNARLLVLTYDTSSASSSSGTSF | SEQ ID NO: 8 |
| | RTGVLLVWEILCHRYSAAVLVFGNSARWSEFRNFMYGPATT | |
| JM 8 | MQRCGVELNLTARPQNGPVQFNPRVRHEGIRDHVQHVRFLEREAVGF | SEQ ID NO: 9 |
| | DVRHQLRQLVVRDVFQDWGFVGLGDFVPQVQRGGLGVWE | |
| JM 9 | MFLARCSTSSDRLDVSTEFCLRSSGRRSRFRFSICVFVFRMREPSIL | SEQ ID NO: 10 |
| | RGSAKTTRLRSTARADSCAYKAASSLAIVVRRPRFS | |
| JM 10 | MARELAALYAQLSALAVDLSLVVFADPRSIDGSRILKTKTQIENLNR | SEQ ID NO: 11 |
| | DLLPLLRKQNSVETSSLSLEVEHLAKNIEDKLGELERSLRQRYSSRE | |
| | HFETLHLRPECHYHATVTFEFYGGGLIDVNMCLINDVELLCKRLGSV | |
| | FYCIGANEALSGLDRVLAFLSTLRGISPIPHPDLYVTSVPCVQCLRE | |
| | IELVPNQGSSLLAVLADRHCDHLCKKVRAEPIHGLFETELSQLGLKV | |
| | AKGSDATQHGVRSSADQLRESSLAAIQDHNIFKRVSASIMELSNLIY | |
| | WNAGQTGLQTGTENECSQMAKLLTHEAEMHEHRALITPKQSAAHFYD | |
| | CFRPDPIESLFCGGLFNSIDDTINALSRDCSVTFFQQANYTNVMRKQ | |
| | NELFTRLNSILCQGSAGSQKPATPSEPRTATVAATAASDVIKDAQYR | |
| | KEQYMKKVARDGFKKLTECLQTQSAVLANALCMRVWGGVAYGEASEL | |
| | VNHFLLRRRFVALPWEARCRSNQILFENSKYIKNSLYSQRLSREHVE | |
| | IITLQFYGLITGPLTRQSDLFPGPANVVLAQCFEAAGMLPHHKMLVS | |
| | EMIWPQIQPKDWIDQTFNRFYQLPEGDLNAVQKSAWCFIRELVLSVA | |
| | LYNRTWEKTLRIFSLAREKPSISDLDVKSLTPGLYLTYEQNTPLVLI | |
| | SQMTGWIFKDLYALLYHHLQLSDGHDDN | |
| JM 11 | MWGTIFFLNTIRMPSWCVLSSVPGHVFSRLNLKSSPVAEALHTRNLN | SEQ ID NO: 12 |
| | ASALGGFSGGVGVDGPGVDFAVVVTPFGVVTFSPTAAPIAIITTHAR | |
| | SRRRVRLVIIMAV | |
| JM 12 | MMITNRTRRLLRAWVVIIAIGAAVGENVTTPKGVTTTAKSTPGPSTP | SEQ ID NO: 13 |
| | TPPENPPRAEAFKFRVCSASATGELFRFNLEKTCPGTEDKTHQEGIL | |
| | MVFKKNIVPHIFKVRRYRKVATSVTVYRGWTETAVTGKQEVIRPVPQ | |
| | YEINHMDTTYQCFSSMRVNVNGIENTYTDRDFTNQTVFLQPVEGLTD | |
| | NIQRYFSQPVLYTTPGWFPGIYRVRTTVNCEIVDMIARSAEPYSYFV | |
| | TALGDTVEVSPFCLNDSTCSVADKAENGLGVRVLTNYTIVDFATRTP | |
| | TTETRVFADSGEYTVSWKAEDPKSAVCALTLWKTFPRAIQTTHEASY | |
| | HFVANDVTATFTSPLSEVANFTGTYSCLDNVIQKTLNATIKKLSDTH | |
| | AANGSAQYYETEGGLFLLWQPLTQLSLVDEMRGLNDTTPAPPTTSTA | |
| | SRVRRSVDTNSQTAEDLAAPQLQFAYDKLRASINKVLEELSRAWCRE | |
| | QVRDTYMWYELSKINPTSVMTAIYGRPVSAKFVGDAISVTDCVTVDQ | |
| | GSVSIHKSLRTSTPGICYSRPPVTFRFLNSTTLFKGQLGPRNEIILT | |
| | DNQVEACKETCEHYFIASNVTYYYKDYVFVKKINTSEISTLGTFIAL | |
| | NLSFIENIDFRVIELYSRAEKKLSGSVFDIETMFREYNYYTQRLAGL | |
| | REDLDNTIDLNRDRLARDLSDIVADLGDVGRTVVNVASSVITLFGSI | |
| | VSGFINFIKSPFGGMLMILVIVAVILIVFALNRRTNAIAQAPIRMIY | |
| | PDIDKMQPSGGKVDQEQIKNILAGMHQLQQEERRRLDEKQRSAPSLF | |
| | RPASDGLKRRFRGYKPLENEEAQEYEMNK | |
| JM 13 | MGAWAMALVRRFSANTIRMTATITRIMSMPPNGLFIKLMNPLTIDPN | SEQ ID NO: 14 |
| | RVITLLATLTTVRPTSPRSATISDRSRARRSRFRSIVLSRSSRSPAS | |
| | RCV | |
| JM 14 | MDFFNPYLGPRGPRPPSHKCTDAPAPAGAVQPPPDVCRLIPACLRTP | SEQ ID NO: 15 |
| | GAGGMIPVTIPFPPTYFENGARGDVLLAHERSMWTARGQRPVVPDPQ | |
| | DQSITFHAYDVVETTYAADRCAEVPSRFQTDIIPSGTVLKLLGRTEN | |
| | GTSVCVNVFRQQVYFYAKVPAGVNVTHVLQQALKNTAGRAACGFSTR | |
| | RVTKKILKTYDVAEHPVTEITLSSGSMLSTLSDRLVACGCEVFESNV | |
| | DAVRRFVLDHGFTTFGWYSCAPATPRLAARDARTALEFDCSWEDLSV | |
| | QADRSDWPPYRIVAFDIECMGEAGFPCATRDGDAVIQISCVFYTTRE | |
| | GAPNPPNILFSVGTCDPIPDTDVLEFPSEYDMLVSFFAMLRDFEVDF | |
| | LTGYNISNFDLPYLITRASQVYNLRLNEYTKIKTGSIFEVHEPRGGG | |
| | GGFMRSVSKIKIAGIVPIDMYQVCREKLSLSDYKLDTVARQCLGGKK | |
| | EDVSYKDIPPLFRSGPGGRAKVGSYCVMDSVLVMDLLKMFMIHVEIS | |
| | EIAKLAKIQARRVLTDGQQLRVFSCLLEAAARDNFILPVPTPEGQGG | |
| | YQGATVINPIPGFYDEPVLVVDFASLYPSIIQAHNLCYSTMIHGRDL | |
| | HLHPNLTPDDYETFVLSGGPVHFVKKHKRESLLGRLLTVWLEKRRAI | |
| | RRTLAACDDPSLKTILDKQQLAIKVTCNAVYGFTGVASGLLPCINIA | |
| | ETVTLRGRTMLEMSKSYVEALTTDNLRTRLGREVTACHGARFRVVYG | |
| | DTDSLFIACDGYSAEAVSAFCDDLAARITADLFPPPIKLEAEKTFKC | |
| | LLLLTKKRYIGVLLNDKMVMKGVDLIRKTACKFVQERCRSILDLVLH | |
| | NQEVKAAARLLCKRPPHAVYEEGLPAGFIKIVEVLNASYVDLRNSVV | |
| | PIEQLTFSTELSRPVCDYKTTNLPHLAVYQKLASRCEELPQVHDRIP | |
| | YVFVDAPGALKSDLAEHPDYVKQHQIPVAVDLYFDKLVHGAANILQC | |
| | LFGNNADTTVAILYNFLNVPYKLFS | |
| JM 15 | MLRDVVRFENFFGYSSGREAARGPAGGVLEGLLEDVGDVDAGWDLRV | SEQ ID NO: 16 |
| | EIYLLTEHVHAHAGAVLGSPQELEHGSARNNVRLEAARYLGAPVRRV | |
| | RRFNDVVRVKRNGLVLGVRDDWPLAARRPHGPFVGEQHVSARAVFKV | |
| | RRRERDRDRDHPACPRRSEAGGNEPADVWWRLISTGAGGGGRIGAFV | |
| JM 16 | MHRSAAQHERLVVVRRQVGVQVQVSSVYHGGVAQVVRLDDAWVQAGK | SEQ ID NO: 17 |
| | IDHQDRLVVKPRDGVNTHRRALITPLSLGRWDREDKVIPGRGLQQAGE | |
| | HAKLLAVR | |
| JM 17 | MPLHPGPGAPQSGGQGGGAAVVQATAARGIRGGVAGGLYKNCGGPQR | SEQ ID NO: 18 |
| | ELCGPSKQRRAHRAVNVLDRAQPPRLRLQDHQPAPPGGVPEAGEQVR | |
| | GAAPGAR | |
| JM 18 | MLVNELSMVLGDWEVTFHRGKFSFVNLARLQTFKGHGGYAKIRLPFS | SEQ ID NO: 19 |
| | LDQLLHQHFAFGLVTRLNELPPFSDCVALIAPRDSGGDADAARVAPG | |
| | FVLDSSRPLTVWVNANGRHTVRFCLLFLKPIDLERAVTYVFGENGGA | |
| | RSEGAPKPTCNTESLPGGPLRVSGEASQTSPHSFVAYFPTADPVACL | |
| | SLLRLQVRPFSDDAAHRDARISPKYVTFSNAGGNVCKASVHTLSPSR | |
| | CKTAQMEIIYAPGDPNAEIVLGQSGPVLPTHTGGRVLGVYADAEKTI | |
| | QPGSSAEVRVQLIFQQGAAARGDLAFLVAGVAPEPLFIVTPTLLLSG | |
| | CTTHLRLFNPNGTPTTIKRDTLVAAAAPCPVVRVSSADDAPRDLVAS | |
| | PDTGALSIDAFTIPVGLPGVVSAECHVSMRDNGVHERMSH | |
| JM 19 | MESAPVSGDATRSRGASSAELTRTTGQGAAAATRVSLFIVVGVPLGL | SEQ ID NO: 20 |
| | NRRRCVVQPESKSVGVTMNRGSGATPATRNARSPRAAAPCWKIN | |
| JM 20 | MGTPVRFFRGEWQTSSLVDNGTPRYSSLVWAATIHDGYLTLVNRSEL | SEQ ID NO: 21 |
| | CVTERSPCLPACPSIGRLVGKRFPGFAFASATLGDRGTRTVFYAFGH | |
| | RDNPLDIVPAVVERADRELVLRVHAPRTTRVSRYGLKIFVAIVTVVR | |
| | PPGVFLHFPQDRVPIALTDACSQEGSRLTSEEPWIKIQGFPVLSDET | |
| | AHPFLLTQKTKPFTERKFCRLIMDDNQLSAVNTVYLGKQHVRVTVTR | |
| | PPETIVTDGPVTATLSLTGNAPIAFRHNPYFELPWSSTTAIFTPVVY | |
| | VGLTVCIPPNCSKFVRYGNTYVSAFNRKLTAIISNHAHDGGFRIQDC | |
| | EWPPNREIEILVTNVTQAPVYVSTGTQLGRAIFVFAPRFGGPAKLRQ | |
| | LLGHRSRALELPGGVTVDSQKLCRFETMYLFST | |
| JM 21 | MFPVWFVLFYLSCWAASPTLAPPPTAAGINVLPQWAGNRASLDRTRG | SEQ ID NO: 22 |
| | RLSELGLNIQRWFVYLCYHSTLCRVREYPRIMSFINFPILMSNVECQ | |
| | RREFRGAECMNAMVRGLRAYESYLTRSKMLLDDAPGNAGAAAIGSAV | |
| | TVVLSALNSLIEELPVDNKIGGVESNDKIVPALAEQSPGDVILSAFR | |
| | LLEYLQMFLRDGRRAIAMM | |
| JM 22 | MIVLVHLGICYVKKIIPACLSGIAAVRCRVSRGSEVAAARAPDGRGE | SEQ ID NO: 23 |
| | HGELQYLAHLDLIIRHGVQRDDRTGVGTRSVFGLQARYNLRDEFPLL | |
| | TTKRVFWRGVVEELLWFIRGSTDSTELSRRGVKIWDAHGSRAFLAAQ | |
| | GFGDRREGDLGPVYGFQWRHFGAEYRGADADYEGQGVDQLRYVVDLI | |
| | VRRPHDRRIVMCAWNPADLARMALPPCHVLCQFYVARGELSCQLYQR | |
| | SADMGLGVPFNIASYALLTYLIAHVTGLTPGDFVHTLGDAHVYNNHV | |
| | GPLLLQLGRTPRPFPRLKILRKVTRLEDFTRADLSLEGYDPHPHIEM | |
| | EMAV | |
| JM 23 | MCLHGGASTMTLKINVFCGSCTMRVWLMNAGGDSGGDAGGDSGGDAG | SEQ ID NO: 24 |
| | GDSGGDAGGDSGGDAGGDSGGDAGGDSGGDAGEGPRGFTTR | |
| JM 24 | MRVLVIGAFFAVFACVVDYAFPMGSMSGPAPEVCCLGYINKLPPSGA | SEQ ID NO: 25 |
| | VALYYYTSSQCTLDAVILETHRGQKLCANPGDDGVRKLLQKVDNRPK | |
| | RNKGRRTRRSLLDDASDEGLESGSGF | |
| JM 25 | MRRVCLGGGDFPFIAGTGPLLVLEIPFSTGSLLRRKAVYLFGESPFF | SEQ ID NO: 26 |
| | RTPGATVVRHARFGTRDATSGREARAPGGWPRLVSVEADPGRGY | |
| JM 26 | MLFLVAIGDIFRCPPNDPVRRGFFGSFGGHRKMFPRARRATVETIFS | SEQ ID NO: 27 |
| | DPLVTPEPAPPRGAPCLGAEQSVLVRTPNAGLAVPTPARKTRGTPAG | |
| | FYAITLCYFGVGAPRCPC | |
| JM 27 | MSGVSVTRGAEAAGGGVGKNSGRRARVSVMTRLDGGHLAAGQRREHR | SEQ ID NO: 28 |
| | ERPAGGAARDRAPRSVKMADRRRRLGAADVNHGGEERRWSGYTSITE | |
| | GRAGWQII | |
| JM 28 | MAAIQGPPPLPEEENENSLPDDVYAIEGIFLYCGLGQAEYLHHPVFS | SEQ ID NO: 29 |
| | PIKEFISSFLKDSARLYERLLRHTDYRSLRGLNAIGQGMLHINTDGR | |
| | HNWGRALAVLGLGAYVVDKIRDDERLLTFAIAVLPVYAYEALESQWF | |
| | RSHGGWEGLRNYCERILRHRRNARRHMCYGVAAGLLALVALFAIRR | |
| JM 29 | MTSVYVGGYVDVVSLPKIEKDLYLEPSIVATLLPYTNPLPINIEHVP | SEQ ID NO: 30 |
| | EAHVGHTLGLFQVTHGIFCLGKLTSPDFLALASRLAGDSRAAQIQLN | |
| | PMPRDPLLEMLHTWLPELSLSSLHPEELQDPNHPPAFQHVSLCALGR | |
| | RRGSIAVYGPDPTWVVSKFDSLTRDEAGKITSKCLDLCERQVTPPEF | |
| | AAPLETLMAKAIDAGFIRDRTDLLKTDKGVARVARSTYLKASQSPSS | |
| | QHGGNRDTQTMSALPDDNITIPKSTFLTMVQSSLDHMRNQGQRAYVS | |
| | APPSMPATAAYPSWIPPPELTVPSYAPPVAPPFPFQSAFAPQPSPYA | |
| | ATYYSPTYGYAQAPSRHQKRKRDVELSDEPVFPGEEVGIHKDVMALS | |
| | KNILDIQADLRDLKRAASQTSGEKDTDQRPQPPPVQFSWPQTYASAP | |
| | YLAYQPQWYPGTDTHLHASQPYQIAQGIQQTQPPPPQPASHHAGLAT | |
| | QPVAPAPAAQESANSNAVPSASAPRAGACPPLDSDCGQSARAPVEAS | |
| | VQPAPVSQIQKMFCEELLK | |
| JM 30 | MFIGRGFVYGSRVATIEGSKYRSFSIFGRLTTSTYPPTYTEVMLGRC | SEQ ID NO: 31 |
| | LREPKEMSAGLRGLMWRVIRCENLNTFLPGELRFLHLVLCEMYNYGL | |
| | NVYLLKEAIANTGTRDDIVLGRKVPVEFWKIIYDGLREMGVSDATLL | |
| | SETKRGALWLYFNGRPCLLKGLGDYVFCRLGLSHSVRVVPENLTDGN | |
| | YLYNLGSVIPCRLLVALSYCLAFWGHADHEPWVRLFAGKIFILYLII | |
| | SGHIMPRKSILEQVGTSGYGGFVEAVCRDVRAVHGIPAWDFAAAAPA | |
| | LTSRQTDYLFAFNNSVV | |
| JM 31 | MSPASGAELLLGVLGARRPRALGQVVRRQDFYTVLNNIRPHYATKVN | SEQ ID NO: 32 |
| | LRTGGNVRVRWLCRGCVSRRSRGPRHSGVGFCGSGSGVNVSTDGLPV | |
| | CVQ | |
| JM 32 | MRTSEKCCMRYPRKPARQITATFWAPHPNNVLFIHKPSLIEERRNAF | SEQ ID NO: 33 |
| | VMRNQQLALRVHTLRKNLLRLELDNVLQTHQRETEMVMRDLDTIQNM | |
| | VGDLRSPTRETADTQTSINPRPKIAPQTHGDAFVVTIAPGDPGFTVN | |
| | QDLRLELLPSLYMNQNQWLPQYGPWYSSLTDNAMQRRVFPRDLRGTT | |
| | NFQNSTSLKLMSAVISTAASITQDFYADVRNVSDTQAALCLLNGYYC | |
| | HRTGTPLPPTRDGLWDNLGTKLATLISHLKQNTKGLGFEFTYSNPKQ | |
| | RASLAPLNKETKYSADFFTNHVIYATLAQSGLLPGSKNPGTGQPPGP | |
| | DLVYILATTLFSEDVPPFQAYQWNLRAGLSALGCLVLVYVLLELAQI | |
| | TPRSPHRRLNLASLLGGRFSKVEDPSGSKQYLKKGQLFDFLTENYIS | |
| | PLLSRAPDAPTSFLFPGAYLTALEAKAISHLKHTRPFVNLTGSRFNE | |
| | IFDILNQKLTFRDAGSLIQAQTSLRLTAEEGLAAILSHPSPPGLTHE | |
| | IMKSQFGVYDDYDKVYFLVLGYLPVATSVV | |
| JM 33 | MTVANQCKHAATLEALPASRKRAGIRAHLAVYRRLIKHRSLDDILKF | SEQ ID NO: 34 |
| | LSIRPTREATKDVKFRIFFEVTLGRRIADCVLTVDSEHQKTCYVIEL | |
| | KTCLSAAVFPGNAIKISQRWQGLHQLTDSVAYIGRAAPRGHENWSVR | |
| | PWLLFKNQKTLKTIHTESSAFPPTFINTTSVALNGFFSQWEDAHVRK | |
| | MLYEISTKTSAANYRNFLGPTSKQRSVHSQTIADRRKKKRVCDAKST | |
| | AGAKGSHAAKKPAPSRARQRAADAPTGNRNGHARPRHNSKHGRGSAV | |
| | PDQGNRRYPNVNKPTTQNRPSDTWRRVCCHDSPRRPGLHGKPGSPSG | |
| | APAQPVHEPKPMAATIRSVVQ | |
| JM 34 | MAEGGSGFGDELVRQMRDRKPRWDESSDDTDDVDTESTDLEYDDVFP | SEQ ID NO: 35 |
| | VVDTHGLTSPGSQNYDVPTSPSGTPWELLHPDALYAQPRCPPKRAAV | |
| | PGGGARPKVSAFSARLQYVGRQSFGDRETRQLTGAQFSSESEHEYAE | |
| | IPERTTRPVESGDKRHFTSGRRGGISGPSSSKPSNGAGLTRKTKTSL | |
| | SVSLKMLLRIKDDDVKVDVPRPVTVPVHLMQPHPMTEYRNAFLIYLE | |
| | GVMGVGKTTLLNSMTGMVPQENVLSCPEPMKFWTCVYSNCLKEQRSI | |
| | VKQGTHGKSITSARVYACQSKFALPFRATAAGIGRNLQPWLVGNGST | |
| | KPANWIVFDRHLLSATVVFPLVHVKYNRLTPDHLFQILSLFSAHDGD | |
| | VVVLLTLNSSEAHRRIQSRGRKEEKGITQNYLRQVAWAYHAVFCTWV | |
| | MMQYLTPEQMVQLCVQTVSIEDICNMNSRLTHRFLTLTKLHEQSMIP | |
| | MVVEMLAAVKEHVTLMEVCLGLFKELRKLQILIVDAGEHLDDTCGLW | |
| | GNIYGQVMSNEAIKPRAVNWPALESYIQTLTSLESNAAN | |
| JM 35 | MTYPRLRPERRGNYCTRTLCMRSRGAHLKGRPCRAVVRGPRCPRSRL | SEQ ID NO: 36 |
| | DFNMLDDRVLGIERRGSLPGPSFLPRASTNMRKYRSVLHARLKAGTR | |
| | DILPLGVGVEFRDPRRQNLVTVRG | |
| JM 36 | MSLVPAFNRACSTLRYFRIFVLALGRKLGPGKLPRLSIPKTLSSNIL | SEQ ID NO: 37 |
| | KSSRERGHLGPRTTARHGRPFRWAPRLRIQSVRVQ | |
| JM 37 | MFNFKIPFLFCNVSCNVSRIFELAQASCITREVNTSLTFFQLIRYRV | SEQ ID NO: 38 |
| | LLNSSSTVDISLLTGVVVAGVKRTISSPGTFSSLYSYPLITENVITN | |
| | KHKPWLIGCIAFQAG | |
| JM 38 | MQPINHGLCLLVITFSVINGYEYNEENVPGLEIVLFTPATTTPVKSD | SEQ ID NO: 39 |
| | ISTVELEFNRTRYRINWKNVSEVLTSRVIQDAWASSKILETLQETLQ | |
| | KRKGILKLNMAYVSNKEINFCKSFSSSEIINITSDFNYSSLPGFLGS | |
| | FDVINHELTHGVLVSKRELFTNALDIMGLFYAIKLNAETFKITFDES | |
| | KVIISGIMTADWILVSIANSSSEVNGQCVALLFGDPEKFPPLKGYVS | |
| | YRDLVVVRNDNYAIAVIAPMSHGTMGLNFLPQWLTDIFFSILNSPLK | |
| | VINYLKGKLFELESKGACQNPSNEQNILSLFFEVTAIHFLYVRNLNK | |
| | EEPVNVGCVVKHVAALKSLERLFKLCFPAFELHSLNLWTLSHLAASQ | |
| | IANLPPNNVMSLTMDEQEVVFSMFKLADNNNVNEIILNEIINISDQM | |
| | YTMYSDIYQLSNTYRQTVMDIYEVLTTVSLTNVGARAVYPYILFTSM | |
| | CNNVEISYMINQISKPDDITIFRVFSPCFLSLRFDLDENKLRSDAPQ | |
| | TSKRTGSELAQGASGFWRLLHAFHATRINEFSVINCTRLAWKQVTAL | |
| | MPLTNITYVISSVRPDHARVYEVSEVFLNSAMFVSAVYPNCSHFTPP | |
| | GTALHIPILYNFSAPRIGCPLCDSIVLSYDENQGLQTMMYVSMPQVQ | |
| | ANLFSPYSPFFDNDNFHIHYLWLMNNGTVVEIRGLYRRHALSAIAFV | |
| | FAFIGTMSALYFLFKLFSILA | |
| JM 39 | MFIISFKMISFTLLLSASLNIENTTSCSSIVNDITLFGGKLAICDAA | SEQ ID NO: 40 |
| | RCDNVHKLSECNSNAGKHNLNNLSKLFKAATCFTTHPTFTGSSLFKF | |
| | LTYKK | |
| JM 40 | MGSRQPQIGESPITAFNTVTIMQRANNSIMFLPNLKLKPIQHLFLKH | SEQ ID NO: 41 |
| | VLLQRLGLENILFHFKMLYANTCKAAAPYQREYFESMLSRVKQRLED | |
| | MVFCLNSIESHNFQKDFKVTSRAPQQLLTATDKYFLMFPPQNRDLAI | |
| | QVGAEVIESICDGTPLFEVLANLNPRVTIQKETGNNLLKFYALLTV | |
| JM 41 | MIVTVLNAVIGLSPICGWREPMAKNMSASKQSDCTSESLVAGGCLRM | SEQ ID NO: 42 |
| | DRSFLSSDVKGALHKVGGAFGSYSCVVFASSFMVICLLFVRVTGGVL | |
| | TVTWTENGL | |
| JM 42 | MIKIPDLKARLVGGAVQLSNGEYVCHVVYSSALAAMVGLPGPAVPLP | SEQ ID NO: 43 |
| | LLFKKFGTIYSNMMPLYAPKRPELSMLRIMVSPRPYALNSCLCVGTD | |
| | EGERGVSLFRDPVIRSSDFDDTPITVRLKISDRE | |
| JM 43 | MMLLQGPVLLPACPATATADTPSPANSDFKTQLAIFCCLATNNEILE | SEQ ID NO: 44 |
| | NVSLEVLDRANETETTFYACPALRRLVLGEGLYPFIHRQGGIVGKTG | |
| | NEYAGPGLIIDDAIGCTFSHIETHTFLPTVFTYELSDTVWVQRDERI | |
| | LRSLYCSPLMVCGVNYQSMFRILCRYLQIWEFEECFAAFTRTLPEHL | |
| | IGTCYQNYFKLLEPFKTLTLARCPPPCAKLHLNYLKFNILGFTSDWI | |
| | SHPELHRVQTVIIYNIESNPVLLKNLSKQNKFQDIRVASELIIDYQN | |
| | IVNQSLGVNLQVKINKKDPGKKPYKVVVVTPKSTYYLTFPPEVPIFR | |
| | VAMCMSVAEHVCHSCDRLYPNTEFLGPGETPRVLEAMFSRIQYAPKD | |
| | RDHNFIFDANQNPDRHKQVHHDHQTEPLPDMFDPVKHLSLHNFKISV | |
| | FNTNMVINTKITCRSLTGTFESIIDIPRLTNNFVMKKFSVKEPSFTV | |
| | SVFYSDNLCNGAAINVNISGDMLHFMFANGNLRCFLPVKHIFPVSIA | |
| | NWNSTLDLHGLENQYIVRRGRRDVFWTTNFPSVVSSKDGCNVSWFKA | |
| | ATATISKIYGRPLLKKLSDELDPILSVPYARIDQVKNTIFTTLETRN | |
| | KAQIQTLHKRFIECLVECCSFLRLDLGALNRAAJRLGAFDFSKRIISH | |
| | TKSKHECAILGYKKCNLIPKIYVRSKKIRLDELGRNANFMSFIATTG | |
| | HAFSNLKPQVIRHTIRRLGLHWRHKAKI | |
| JM 44 | MYIFPHRNAYVFTHRLHLRTVRHGLGSTRRTNPTVPVLFAINGMWRK | SEQ ID NO: 45 |
| | LSKHVSNSLSLPSNMGIRGMLCRLHAYTAGTSDRHVLSELL | |
| JM 45 | MEAALEVRPFPYMATEANLLRQMKESAASGLFKSFQLLLGKDAREGG | SEQ ID NO: 46 |
| | VQFEGLLGVYTNVIQFVKFLETSLAVACVNTEFKDLKRMTDGKIQFK | |
| | VSVPTIAYGDGRRPTKQKQYIIMKACNKHHIGAEIELSTDDIELLFI | |
| | DRETPLDYTEYAGAVKTITASLQFGVDALERGLVDTVLNVKLRSAPP | |
| | MFILKTLSDPVYTERGLKKAVKSDMVSMFKSYLMDNSFFLDKSDIAV | |
| | KGKQYVLSVLSDMVGAVCHETVFKGTNTYLSASGEPIAGVMETTENV | |
| | MRKLLNMLGQVDGGMSGPASYANYVVRGENLVTAVTYGRVMRTFDQF | |
| | MKRIVDRPNAQPNVDDDRDAVADGQDSLAKTPIAAAVIQIGDKLVAL | |
| | ESLQRMYNETQFPFPLNRRMHYTYFFPIGLHMPRPQYSTSATIKGVE | |
| | NPAEQSVETWIVNKNNVLLSFNYQNALKSICHPRMHNPIPCGQALGQ | |
| | AFPDPGHVHRYGQKSEHPPNMNLYGLVYNYYQGKNVAHVPDIALKAT | |
| | MTTDELLHPTSHETLRLEVHPMFDFFVHQQPGAQAAYRATHRTMVGN | |
| | IPQPLAPNEFQNSRGLQFDRAAAVAHVLDQSTMEIIQDTAFDTSYPL | |
| | LCYVIECLVHGQEDKFLINSPLIALTIETYWNNAGKLAFINSFPMLR | |
| | FICVHLGNGSISKDVYAHYRKVFGELVVLQQALSKIAGHEVVGRRPA | |
| | SELINCLQDPNLLPPFAYNDVFTNLLRQSSRHPMVLIGDEGYETEND | |
| | RDTYINVRGKMEDLVGDMVNIYETRNNADHDGRHVLDVGPFNENEQH | |
| | MAVLEKLFYYVVLPACANGHVCGMGVDFDNVALALTYNGPVFADVVN | |
| | PDDEILDHLENGTLREMLEASDIHPTVDMIRTLCTSFLTCPFVTQAS | |
| | RVVTQRDPAQLLTTHDDGRYVSQTVLVNGFAAFAIADRSRDVAETMF | |
| | YPVPFTKLYSDPLVAATLHPLVANYVTRLPAQRVPVAFNVPPALMAE | |
| | YEEWHKSPILAYANTCPITPTSLSTLTSMHMKLSAPGFICHAKHKIH | |
| | PGFANTAVRTDEVLAENLLFSARASTSMFLGQPSVMRREVRADAVTF | |
| | EVMHELASLDMALGYSSTITPAHVAAITSDMGVHCQDLFLMFPGDSY | |
| | QDRTLNDYVKQKAGCQRFGGPGQIREPVAYVAGVPHSDNIPGLSHGQ | |
| | LATCEIVLTPVTADVTYFQTPNSPRGRASCVISCDAYNNESAERLLF | |
| | DHSIPDSAYEYRTTVNPWASQQGSLGDVLYNSTSRQVAVPGMYSPCR | |
| | QFFHKDAILRNNRGLRTLVTEYAARLTGTPATSATDLQYVVVNGTDV | |
| | FLEQPCQFLQEAFPTLAASHRALLDEYMSNKLTHAPVHMGHYMIEEV | |
| | APMKRLLKIGNKVAY | |
| JM 46 | MSCCTRPRTRRFVWRFIRCLIFLFISSQVRKPRIGLPTGLWWVTFRN | SEQ ID NO: 47 |
| | HWRPTSFKTAEACSLTERRPWLTCWTSQPWKLSKIRRLTRHTHCFVM | |
| | SLNASSTDRKTNF | |
| JM 47 | MIRVVSGFVNVNHVTDEVLHFSSDVNVRIPVIFRFVPLVSYEYHRVP | SEQ ID NO: 48 |
| | RGLPKQVGKNVIVSKGRQKIGVLKTVNQLGRWPASHHLVASNFRERL | |
| | LQNNELAKNFSVMGVHVLRNTTVAQVNADKSQHREAVNKRQFSGVVP | |
| | VGFNG | |
| JM 48 | MTGIRTLTSEEKWRTSSVTWLTFTKPETTRIMTAATSLTSVPLTKTN | SEQ ID NO: 49 |
| | STWRCWKSSFITWSCQPVPTATSAAWASILTTWPWP | |
| JM 49 | MALDKSIVVSVTSRLFADEIANLQSKIGCILPLRDAHRLQNIQALGL | SEQ ID NO: 50 |
| | GNLCSRDSAVDFIQAYHYLDKCTLAVLEEVGPNSLRLTRIDPMDNYQ | |
| | IKNAYQPAFHWDNYSELVVIPPVFGRKDATVSLESNGFDVVFPAVVP | |
| | EPLAQTVLQKLLLYNIYYRVAETTPTDVNLAEVTLYTTNITYMGRNY | |
| | ALDMDPVGSSSANRMLDDLSIYLCVLSALIPRGCVRLLTSLVRHNKH | |
| | ELVEIFEGVVPPEVQALDLNNVSVADDITRMGALMTYLRSLSSIFNL | |
| | GRRLHVYAFSSDTNTASCWCAYN | |
| JM 50 | MSIPKIMTVSRDNEGTVCEVAVDNGRHRAMIYYPKITNSSNERAAQR | SEQ ID NO: 51 |
| | ADVVKEAFDTETPVDIVKQIVNEGLAISKRNCVRLALYLYFYLQYVC | |
| | FAILITWQLNPHIDPPGLVFAVNPMGPKHVSKLPHPAIVAVGCGTDA | |
| | ICKNCSVPDIKTELGVVYNNGSSDSGQSAHYGLALLKAAWLVMGNVC | |
| | PEPVVRQGAELLGPWNRTAWLDFKSAMAATTFCGSRGVLWSPIHEKN | |
| | LCRPTWNDVINTSSFFTNESLCPNVPGVSEIVIVLNGDA | |
| JM 51 | MTAHTNGVLTTTGVSTSQPESFQISPFFRVITKPPIMGLFFCVAMCI | SEQ ID NO: 52 |
| | IALVWYVMRRVYCKGRVVADSCRDPRRPAYEMLNVRLRPHGTNP | |
| JM 52 | MLQKDAKLIFISSSNSSDKSTSFLLNLKDAHEKMLNVVSYVCPDHKD | SEQ ID NO: 53 |
| | DFNLQDTVVACPCYRLHIPAYITIDETVRSTTNLFLDGAFSTELMGD | |
| | AATSAQSMHKIVSDSSLSQLDLCRVESTSQDIQGAMKPFLHVYIDPA | |
| | YTNNTDASGTGIGAVIAVNHKVIKCILLGVEHFFLRDLTGTAAYQIA | |
| | SCAAALIRAIVTLHPQILHVNVAVEGNSSQDAGVAIATVLNEICSVP | |
| | LSFLHHADKNTLIRSPIYMLGPEKAKAFESFIYALNSGTFSASQTVV | |
| | SHTIKLSFDPVAYLIDQIKAIRCIPLKDGGHTYCAKQKTMSDDVLVA | |
| | TVMAHYMATNDKFVFKSLE | |
| JM 53 | MSFLYHNRCKECQMTRVNSPICQFHNVSSLYQCLDCKRYHVCDGGRD | SEQ ID NO: 54 |
| | CVIVYTRENLVCDLTGNCVLDNVQDVCSYGPPERRAPDAFIDPLVSH | |
| | GTRESLKSDILRYFETVGVKSEAYSTVVKNGQLNGIIGRLIDATFNE | |
| | CLPVMSDGEGGRDLAASIYIHIIISIYSTKTVYDNLLFKCTRNKKYD | |
| | HIVKTIRAQWMRMVSTGDPSRVMATGCFT | |
| JM 54 | MLAARSLPPSPSLITGRHSLNVASINLPMMPFSCPFLTTVEYASDFT | SEQ ID NO: 55 |
| | PTVSKYLNISLLRLSLVPCDTRGSMNASGARRSGGPYEHTSCTLSKT | |
| | QFPVKSHTRFSRVYTMTQSRPPSHT | |
| JM 55 | MDAHGLNRRSVAGQCDGLFHVILPRGFILANNITCGGRQRFFAHTRF | SEQ ID NO: 56 |
| | AASERTSKTLYVWGRVFQMTDPGSGDGPSGPWSGLAISLPLFTTNGK | |
| | FHPFDVVILRAETPGSGSSWTVKFLYMSLIAAYRNAMRGLKDKVSQS | |
| | TDAAVDGEVHPLTVLKEALVSPDTATRPVSACNPLQMLTGLLQSRVR | |
| | DDYVTHHRALERPGNVRGQVSAPTRTEMPNGSPSRVRLGFRPPKQAN | |
| | YPKTWAQARHVFSSRTYYVCVYDNEELDTKWQRQDPRPLSLDWSDPV | |
| | AYLLEGDLFLGAKQNAFVDSLEKTCRCQNYTIKQFFPVSINRDNDTV | |
| | DLIKEHFIEACLVIRNQASERSAWVKAALFRNDSNTYWKDVLGLWEH | |
| | GPHKLGTAIKRPTSEPCNADIDWSWLLCDADITRSINGQSTVCLVVS | |
| | PALIAWLVLPGGFVIKGRYDLSSEDLMFVASRYGHPAA | |
| JM 56 | MATQRRDILKSFLNKECIWLRHPGTSAFVRVYTATTAHSAVFDPPVT | SEQ ID NO: 57 |
| | SEDAMSHNCLNVMIMLMKPKEFGPCVTVYINGDILDFCATEYVAIRE | |
| | VPGRADLCLIRFGTLSNAPRSVPIPGPLNPHPRENVPGLTKQEIIYT | |
| | SQTVPPAQIQDAIKGKAFKQINPFVWFDGGAFWQLFLSVDYMLLCPA | |
| | LEIVPSLARIVGLLTQCDKSTCKICTLAHVHVNAYRGYTPPDSQGTS | |
| | PSCPCLISCGARHATDVLVTGHVNLLGLLFDPKVLPKVSRLRLKRNP | |
| | HPVPIEDAMSGVTAEGTEVLPTSQPWALIRLPDLASRVMLYGCQNLK | |
| | TICLRSY | |
| JM 57 | MASCICALGTVWEVYIISCFVSPGTFSRGCGFNGPGIGTLLGALERV | SEQ ID NO: 58 |
| | PKRIKHRSALPGTSLMATYSVAQKSRISPFMYTVTHGPNSFGFMSII | |
| | MTFKQLCDIASSLVTGGSKTAECAVVAV | |
| JM 58 | MPAHGCEVGSTSVPSAVTPDMASSIGTGWGFLFKRSLDTLGSTFGSK | SEQ ID NO: 59 |
| | SRPKRFTCPVTRTSVACLAPHEIRQGHEGEVPCESGGVYPRYALTCT | |
| | WASVHILHVLLSHCVRRPTILARDGTISRAGQSSI | |
| JM 59 | MLLTSYRERLQNNLRAVTDGGCENWFRQPPVIISGNDKTERMANPCL | SEQ ID NO: 60 |
| | GVIHAVNAYSSVLDDYLQTYRRLQEPMPPPTLGKPRISSHATLPRLT | |
| | EELTNYLRQTCCRVRMADAKDQHMEYQSAQRTHEAFLECPVYAELRQ | |
| | FLANLSSFLNGSYVPGVCCLEPFQQQLIMHTFYFIASIKAPEKTHQL | |
| | FATFKQHFGLFETTDDVLQTFKQKASVFVIPRRHGKTWIVVAIISVL | |
| | LSSVENVHVGYVAHQKHVANAVFSEVIATLSRWFPAKNLNIKKENGT | |
| | IVYASPGRRPSSLMCATCFNKNVSRLFLNSGSRIALHDWLNPAGE | |
| JM 60 | MLSGPNGRRQGPAHGIPIGPTDPRSFPRVSCLCRTAAIFSQPVVIFK | SEQ ID NO: 61 |
| | WELRARGLLPRALSATVNTIAHVLFYRVYQSTRKDTPVICHV | |
| JM 61 | MFPSSFLNNENPETGRRFVKGVQLALDLCDNTPGQFKLVETPLNSFL | SEQ ID NO: 62 |
| | LVSNVLPESRPVRDCPQAEGFDFEHIHLPKLTRMQRVLGRYCDHVNN | |
| | DDDMCVNVKARSSNAQGALFYLPYGQDEWNWALTLRKDKLVKMAVEG | |
| | LSDPTTWKGLEPVDPLPLIWLLFYGPRSFCREPECLYERNFGMKGPI | |
| | LLPPHMYAPRKDVMTFVHHVIKYVKFLYVNAGGGLETELSPPFEASR | |
| | LRSAIARLGDVEADDAYLSAKCMLCHLYKQNDTISIHETHVGGVIAL | |
| | GGDGARYITSSVRTQRCTSRGDFVLIPLYNIEGLVSMIREHGLSNS | |
| JM 62 | MWSFGDSSSTGTDSSLSVPRISSSPYSSSLRSRTASVTASNSSIFFL | SEQ ID NO: 63 |
| | LNPFSNPNSLCFSRSDVSSVPTTVARLSFFFRLSYDRSDSLVPLYCA | |
| | LNTALSAPNLSKTEMDSFLLISDSSSDLINNFLAVAEANFPYHAYEP | |
| | FNVVQRD | |
| JM 63 | MASATAKKLLIKSELESEINKKLSISVFDRFGADSAVFNAQYKGTRE | SEQ ID NO: 64 |
| | SLRSYDSLKKKDWLATVVGTLETSLREKQSELGLLKGFNRKKIEEFD | |
| | AVTDAVRDLKDELYGELEILGTLNDESVPVEEESPKDHIIRWKLERL | |
| | PRVCPKSP | |
| JM 64 | MNLFPWKKSPQRTTLLDGNWSVCQECAPKALDPIPKVQTDLDRTALS | SEQ ID NO: 65 |
| | HITVIRTRKTLAQLKIPNTWSQCSHQATDWTAVLGRGSYGVVRSMSL | |
| | GRCVKHFGSRREFFYECIFNDIVRARREKHPLNRGGDRILCFLEPCV | |
| | PCRALIFPQLTGNLLNADFKHVNPERLAVEFSELREGVSFLNNICGI | |
| | VHCDISPENILIKGELTTAYWRLMIGDLGSASLHTGTPWTGVMVTSK | |
| | LGFVQHTYHFKAPARFICKHMYRPSCLLYRCLLSCAGGPQARMLDQP | |
| | FQITPQLGLTIDMSSLGYSLLACLEKYLQPADPFPQQGALADASSES | |
| | AHPLFYLRCMVPRVVIAEIFSVAWDVPLDLGIDSSGHAPAIPLREAY | |
| | RRFFANQCSLYRAQYKEDALENASSRLCNSKLKLVLQKLLVRDYFSH | |
| | CGNCGDHGFFLR | |
| JM 65 | MDFFSDEPMVQEMALLDIDEQQRHLSKMSLANFLKHERVRAFFNDNK | SEQ ID NO: 66 |
| | KKISMPAIRFVYNFYLFAKVGDFIGNTEVYDFYVSCVFRGRRLTLLS | |
| | DVYDACLNMHPHDRHHVCALIEQVTRGQNINPLWDALRDGIISSSKF | |
| | HWAIKQQNSSKKIFNPWPIVNNHFIAGPLAFGLRCEDVVKKILATLL | |
| | HPGEAHCENYGFMQSPHNGVFGVSLDFGINVKSNPKDGLEFHPDCKI | |
| | YEIKCRFKYTFSKMECDPIYAAYAKLYQKPSMQTLKGFLYSISKPAI | |
| | EFVGEDKLPSEADYLVAYDKEWEVCPRKKRRLTAVHHLVKKCMIHNS | |
| | TAPSDVYILSDPQETGGQINIKAHLSANLFINVRHPYYYQVLLQSLV | |
| | VQEYISLSKGTKNLGTQKNFIATGFFRKRQFQDPSNCTIGEFAPLDP | |
| | HVEIPTLLIVTPVYFPSVAKHQLVKQATEFWAASASEAFPELPWDLS | |
| | SLCANAPPTP | |
| JM 66 | MKISRSDSFILSSWVKLLVILGLMFIMSAVVPLTATFPGLGFPCYFN | SEQ ID NO: 67 |
| | TLVNYSALNLTVRSSAKHLTPTLFLEAPEMFVYISWAFLVDGYLLCY | |
| | YAWAILAIFKAKRVHATTMTSLQTWIVLIGSHSVVFMSILRLWTIQL | |
| | FIHVLSYKHILLASFVYCIHFCLSFTHVQAMISCNSATWSLRVLEQQ | |
| | IPENSLLDTLLRYGKPIGANLYLSLIANEMLVFSLGTMMAIGNSFYM | |
| | LVSDIVFGSINLFFVLTVAWYINTELFLVKYLKHQIGFYVGVFVSYL | |
| | ILLLPVVRYDKVFISASLHKVIAVNISMIPITCILAIILRIIRNDWK | |
| | WCAKAPEYAPLPQGSKEKTTKVKYSPELNALYETEEDVSDDEDAYPK | |
| | YI | |
| JM 67 | MYTVYKRGQKYMFVRQHVNKQLNGPQPQNRHKNYAVRTYEHDPGLEA | SEQ ID NO: 68 |
| | GHCRRVHALGLEYGQDGPRVVTQQISVHQKRPRDVNKHFWCFQKQRR | |
| | RQVFSRTSDR | |
| JM 68 | MNAREVALTGHVLHISLHSTHEREKLIIWQVHLLVCQQCGIQGDAAY | SEQ ID NO: 69 |
| | LFVTETLSNTDWGNIPAINRHAPSMNEHGRNYMQWELRTRLRNPIIQ | |
| | SLSRQPGAVNVRVSEPNMVIVSCERALHHSCSVRVTGAYLHCNTTMD | |
| | FRLDSNVSPTREFWFSEMFSKCLVSNIEVYLKTTGGLYYRASSATQC | |
| | RKRAKDGALGILDIFNCESREIQVAGQKYNLSIATATFHVLWVDEAC | |
| | MWNGALAEFFRALHNKLFGDREGVAPTLTYVCPGATPEGTPFPPYFS | |
| | AFPHLLLVFGRPRRLDVTAVQELPKAQIAVHWPPFKNSILGDQLLIP | |
| | GISPKKPGTVPVRWPLWVQDVNLSLCETTESVARIVDPHSIVIIKIS | |
| | TLLCQHLKCHRAFVKNELEYIATICSSDLRLFIQEEYNRLLATIFTW | |
| | AAASGYTWAAIDKTTVFIKAPQLSAAVSGSCPSLNSCRRKQYYKGLK | |
| | ITVHFLSQEQQKVVTRLEAQLGLPVQETSRPPDWLKYEVCSASVFLK | |
| | IPAGVLYAGLAKDPVSEAKRDSWLDCLVEGATLSLNNSVPPIGALAG | |
| | ILPTLFAKRRCVNFWLLPREWVKSAPICPPLPIDCVTPQQFVVTKRG | |
| | PICWYKEWPLPXTDVDFMYYLQEALCVFSVVSNGEGTESHADDIRQLE | |
| | KFEKVLCLF | |
| JM 69 | MNTVVLSMAAQVYPLAAAHVKIVASNRLYSSWMKRRRSLEQMVANYS | SEQ ID NO: 70 |
| | NSFLTNARWHFRCWHNSVEIFMITIECGSTMRATLSVVSQRLKLTSC | |
| | TQSGQRTGTVPGFLGEMPGMRS | |
| JM 70 | MDQILKRLMGEQHRSEAIMPETECASRGPYNYPVLPRLMLEVHKKNS | SEQ ID NO: 71 |
| | ICMASNTPKLCVRGRLNVPDLGVHVRTRLQSATFTGFVFACVVEHED | |
| | MVNTLDIYPHVFSDRVQLFKPASASVTELCCILSMLENYDKPPLSFI | |
| | LSALDRARYLHERYTCNDSAFILYGIEVIASTLAAYHELNPPQGVLR | |
| | VPPLVRFKLHKLLDENADDMKGLLKPIYLESFRLTENVEEDSHGETF | |
| | NIFYCGTIFTRHLHNASVLKYFQITSLHSLPRQTLF | |
| JM 71 | MGFSKPFMSSAFSSNSLWSLNRTRGGTRKTPWGGFNSWYAAKVDAIT | SEQ ID NO: 72 |
| | SIPYNINAESLHVYLSCRYLARSSADRINDRGGLS | |
| JM 72 | MFKMNPGLGSTCLVHPTELSISLFEILQGKYAYVRGQTLHSSLRNPG | SEQ ID NO: 73 |
| | IFGRQLFIHLYKTALGSCTYDNVLKDWTNFETTLKTRWRGVEHLTPE | |
| | FKRSTFESWARTVRLTVDQLLLNTINQVLHTRTVLSYERYVDWVVAL | |
| | GLVPIVRRAPDGETIARIQAHCQQMRKTHASGDVTISRIVDKLAQEI | |
| | TAIMTDVTSIYIPDYAEVSVEFNGDKAAYLGTYRQKDITVEVVSRPI | |
| | IYNGRVSFDSPLYRLFTAIMTCHRTAEHAKLCQLLNTAPLKALVGST | |
| | CNDMYKDILARLEQSSQKTDPKRELLNLLIKLAENKTVSGITDVVED | |
| | FVTDVSQNIVDKNKLFGTGTESTTQGLRKQVSNTVFKCLTNQINEQF | |
| | DTISNLEKERDDYVKKIQCIETQLLQSLPEGGRPRHDINILTQNTLQ | |
| | ALSGLRDPTINLSECHIPKGSSVVNSFFSQYVPPFMEMLRELTSLWE | |
| | GEMFQTYNLTPVVDNQGQRTSIAYSQDTVSILLGPFTYIIAKLTHMD | |
| | LINHSLISLSLHDIADQLYVDSRLFVYINDIGHKYCEQIIQPGTDGP | |
| | NTEAFNGGAAPIGGNNA | |
| JM 73 | MESSVGWTKHVEPSPGFILNMTSDAKVRGVVDHVSRLSNITTSPPEM | SEQ ID NO: 74 |
| | GWYDLAFDPAEDSGPFLPFTVYLITGTAGAGKSTSISALYQNLNCLI | |
| | TGATTIAAQNLSRRLKTFCPTIFSAFGFKSRHINIAVRKAHQTGAVS | |
| | IEQVQQQELSKYWPVIVDIMKEVMAKKPNGMYGTISNADFETLSRMT | |
| | GPCLWTSNIIVIDEAGTLSSYILTTVVFFYWFLNSWLNTPLYRQGAV | |
| | PCIVCVGSPTQTNAFQSTYNHGTQKTEISSCENILTFMIGKKVVSEY | |
| | VNLERNWALFINNKRCTDLQFGHLLKILEYNLPIPDEVMSYVDRFVV | |
| | PKSKIMDPLEYIGWTRLFLSHSEVKAYLTNLHTCLTLGGDTRDTKLF | |
| | TCPVVCEVFVKPFEEYKRAVNLTHLTVTEWVTKNLFKLSNYSQFVDQ | |
| | DMSIVATESTERSTQVTFITKFVKNSHVSLNGKTKKCICGFQGTYFE | |
| | FKRILDSELFVETHSQDRPEYVYGFLNTLLYNAMYSFHAYGVTRAHE | |
| | KYLEDLKFAPLPTALATGRVDFQTVREELNLEDDIFYHVCSPPPPAG | |
| | ITSLQVLVDTYCALKDVFASRIKVACRWFGGEFEKETFSAFTVNMVV | |
| | RDGVDFVSPSERLNGLLAFASTVESYKIKGYTFLPIAFGRCQGLPLS | |
| | DDLRKKMPSLVVQDSSGFIACLENNISKLTETMDDGSVFQVCCAGDY | |
| | GVSSNLAMTIVKAQGMSLERVAVVFGAHKNVQTSHVYVAISRAVNSN | |
| | YLVMDSNPLKTLLREPVDNTSAKIHIVRALHNPNTTLIY | |
| JM 74 | MVSVSLDMLFSRHAIKPLESCTTRDGIFFLRSSLSGNPWQRPNAIGK | SEQ ID NO: 75 |
| | NVYPLILYDSTVDANANNPFRRSEGETKSTPSLTTMLTVNAENVSFS | |
| | NSPPNQRHATFILEANTSFRAQYVSTKTWREVIPAGGGGLHTW | |
| JM 75 | MAMFLSDPPRTPPATPRMLPIPGAPRKKRTRRFLFAGSRTGLPVPPG | SEQ ID NO: 76 |
| | YGGPPVIDMTAPNDVFDADSPPTTPKTPDETDSHSENSNYSDMDEED | |
| | EQPVSSPPRTDPHARDGESFNQSDWRPTVITAAGPAAQPSAPAPLTA | |
| | FGGQRPVAVVTGQHRAPPSSTSDSGDDFFIDDYEDTDESGEDADGFS | |
| | PRASPAWSGDTSRSPAGGGWSSNEEEEPAVTGSAVEQETIIISDDDD | |
| | TDDRGSVETWDESDADEGTGATDVIDLCSSSDSDDDADHVTSGGVRA | |
| | ACKRHASRRDCNGDDDVIYLGTTPAPKRRMTSTTGGGATSNPEGPGV | |
| | SGRQTMAATPPVCGNDISIYPWPWLD | |
| JM 76 | MRVGFVWRFWSGGRRICIKNIVRCSHVNHRGASVTGRYRQPRTGPGE | SEQ ID NO: 77 |
| | QKPSCSLFPRCTGNRQHPGSGRRGPWRVRQEHSHDQNVHL | |
| JM 77 | MDTWLETVVWHKMSMTGPNETPTQMLLISDSWLKFLNLSPFLKKKLA | SEQ ID NO: 78 |
| | ALLRRVMDMSKATVIYPPIDRIMWWSYCCEPEDIKVVILGQDPYHRG | |
| | QATGLAFSVAPDYSIPPSLKNIFKEIANTVPGFTAPSHGCLDCWAKR | |
| | GVLLLNTILTVERGKAGSHSNLGWDWFTSYIISCLSAKLQRCVFMLW | |
| | GRKAIDKAVLINGQRHLVLKARHPSPLAAAHAATGSPWPQFLGCNHF | |
| | KLANDYLVQNQRGAVDWNIN | |
| JM 78 | MYTLSFFTTYFILYIGYSSGLIPNPCCDIVPLTGVNIPAPFEIVSFH | SEQ ID NO: 79 |
| | FTDLAWCQGRCVATLRYKVGTITTELCVNGFHLPAFFIRILSGLDFS | |
| | VHREELDLLNYVRISLEDFLSAFKDTHDNSESVTNLPAVPDLTKKGS | |
| | AAFRTRKVGARRGDLWILGSRQ | |
| JM 79 | MAVSIPVQGVDRETESNWRSIVTTFEQHGNADRAIRSLLRFFKGVDH | SEQ ID NO: 80 |
| | PGFLASLVILKDVAIDSEKTIERTDIIPLLQGVRFVTQQIYMHLKDH | |
| | ASESPVTEIWRDCKERFCLALELACGCQSCTSAARQLRDCQQACRPP | |
| | KLNPHKQQCGAARLLTAVYNQMVLRTRVSVSEFCLNALMCVPREFGF | |
| | VSGDVRVETSRVASCMNLSWLYLILDSYIRTDLTNLEMAMSRACRIR | |
| | GLSTRDPFYSALVWLKNSCACAANTFFFTVNSTRVTTPILMDICASL | |
| | AGPVPDVIKINMLPLVNNQMYPSVCVERANFTGSCPKMSPTHRFDGL | |
| | KLETTSLTLAADSLDDILQALELICDDDEGILDSHISDIDTETEVDE | |
| | STIEEEIVFEELS | |
| JM 80 | MVDPFKKSQKRPDGSIRVSMLFKGSHDGSPVRLGLPVNTLNWNGNSH | SEQ ID NO: 81 |
| | DTVNFLMSPKTNPETELNTNDCHPYPKPRPRTIRPG | |
| JM 81 | MDTDDNQVIKLFFIQDSGGRVYSGVGGSISRRWEWLMNPHLVLGSGA | SEQ ID NO: 82 |
| | LCILLVQLNLNLACLGRTNQKTAWPRLLGALCKL | |
| JM 82 | MSRHYGKDHLLNHMYKFHYPPLGMIVGEMNTLTVNARNPLYQAATLR | SEQ ID NO: 83 |
| | VERALYLSKILQVLMQHRQGERFIVPQCRSNMVYCLKELHKITNDRI | |
| | RGLINSVLPLVDAGCVGFDEELVRILPEILKLEYPHVHELLPPHDPT | |
| | SPLSWCLSHMVGVTKTFKGEVKEMIDTFHDLSVPSFQYLASLVKKFF | |
| | LVEEVIYEDYQDTQFNVFLNLCFFWTTVIKMYQSCIFKDKLLDTIKA | |
| | CIELLKGEAPQFFGWYDLNTPNLGSAALVKYTEHLIRALSVDSSAIP | |
| | IGEICSHLHHCKHALLNLE | |
| JM 83 | MGMADESTLSARIRCSVYFTSAAEPKFGVFRSYQPKNCLASPFRSSM | SEQ ID NO: 84 |
| | QALMVSNSLSLKMQLWYIFMTVVQKKQRFKNTLNCVSW | |
| JM 84 | MECASLGPISGLIADLNLFNLFCLYRGSRVKTRGAATCNVPCAECAQ | SEQ ID NO: 85 |
| | GVVRILTERALCCTEKMFIASACSGVVLPPQLAKVFHDVYAEMKAKC | |
| | LGAWRRLICCRRPIMAIADSVLVTYNTLDAEGKLDLKLKALCKLVFQ | |
| | PIFLQRILAPMQLLANGKMVPDNYFTITGTAEKRRPVVTGSTSGMTC | |
| | PGSSLVPDSLILPVCEPGLLPAPLVDLSNVLENPEIILSAPPLSQFV | |
| | ITNTHPSLPQSVSIITPTQGVVPGQCFMDTWKAVSQSIHHQAHTPIF | |
| | AATLAGSTSAAPGPHIACSPVAGTSRQVEASAGVDCGKPVCAPQPDI | |
| | PPHVPTKRMETVAQSGNAPVKNVHIGGRVYAPLVNIPIIDLTSPSGS | |
| | GQSPVNIANTPESRMAAGSPPFAETAATVPAKRKQPREDVADKRSKG | |
| | DGRGAATVNPPFPGPPANRVREQGLFDLIESSTDVTANASGPKNDDD | |
| | MLAAILQDLYGLQSPPAIDSPSSNSDNEDIFPEVSPPSSGHGSP | |
| JM 85 | MSSLSELLEGESIAGGDCRPYRSCRIAANMSSSFFGPDAFAVTSVEL | SEQ ID NO: 86 |
| | SIKSNKPCSRTRIAGGPGNGGFTVAAPRPSPFDLLSATSSRGCFLLA | |
| | GTVAAVSAKGGEPAAMRDSGVLAILTGLCPEPDGDVKSIIGILTSGA | |
| | YTRPPMWTFFTGAFPDCATVSILLVGTWGGISGCGAHTGFPQSTPAD | |
| | ASTCRDVPATGEHAICGPGAADVEPASVAAKIGVCA | |
| JM 86 | MPRVKTQPKRPQVFDFMPLDLHGETRAEMDSQSLCPDGQDLLGSYIY | SEQ ID NO: 87 |
| | TENNGPFSQMMHNGQSNNGTGESFGSYAAGDGFLGGSVSGMHGNGTV | |
| | DGLCSKKQSACRKRSAALIHAASEASVAEQGTSQGTNAVSDRIGRDG | |
| | GIGNKLLKVSARLPDKTKTLPDPSLNCYF | |
| JM 87 | MRIYGIKGLNFFGIMGFWNIPLGWGLCFMVWVAWIARGRSVCPTWHL | SEQ ID NO: 88 |
| | TDGKYEAVYRHYLEECRKHEGSGSPDGSGKTEGSGTKATTEANISIR | |
| | PNVVTSGQDKEPMKTAPRAESSHDLPRIEQVNALHLSTPELAQPLPV | |
| | VESTPRESQSGGTPWDARPHAFIMHTNDMLNPSVVLSFRAVRARSIR | |
| | DTEQSIRDRNTVTTSYRTPGRPSLVQARPSSYGARLPPSPRTMARYA | |
| | ETRPTDDQN | |
| JM 88 | MDCSVSRMERARTALKDRTTDGLSMSFVCIMNAWGRASQGVPPDCDS | SEQ ID NO: 89 |
| | RGVDSTTGRGCANSGVDKWRAIJTCSMRGRSCDDSALGAVFIGSLS | |
| JM 89 | MSSTRPKTRAPKKELTMEELAAQVQKLSVENKQLKKLINSGDPTRSG | SEQ ID NO: 90 |
| | SDPVISNSEKEAKIAAAVSALCNVATRKIEAKVPAVTAKAVTRGQVE | |
| | EALAGINIRVDVSMDETTRGGITASADGALRRRRAQSRTRNNDAD | |
| JM 90 | MTGSIVLALALTACLYLCLPVCATVTTSSTTGTGTPPVTTTPSAAPS | SEQ ID NO: 91 |
| | VTPSFYDYDCSADTYQPVLSSFSSIWAVINSVLVAVATFLYLTYMCF | |
| | FKFVETVAHE | |
| JM 91 | MAEVTAHTVAYAFDSCKFEIIPKNNSSRIALRNKFPVVVKPGEPLVV | SEQ ID NO: 92 |
| | PLGLRIIRAPQCAFFLSGAPTDEVYYHTGLIDQGYRGEIKIIVLNKT | |
| | KQSVTLYRGEVNVSLIAFMYASPGPLKCPILNLPHYSLDAGFDVTSP | |
| | HAMTIPPTDRTPFTLSLYYKSPQLSTPHVPLIVGRSGLAAKGLTVDA | |
| | TKWTQSLVHLRFYNFTKEPIDIPANSRICQVVFIHEDHVPSGWNILR | |
| | SRVQLGSTLQISWAKIRFTDVATLPATHPLNSRPTQSQTEPETNRGA | |
| | KGLGSSGL | |
| JM 92 | MSGSVYSRRPRPKRVEHSEIPRTTRQHPPDIVGQNQVHRRGHASRNP | SEQ ID NO: 93 |
| | PAQLPPHSKPDRTRDQPRRKGVGVVRVIILKHGIILKLFIFWKTIQI | |
| | NINHQPGHLVLFCLARYYPPPPTGASVVESPDGRGWRTLRYLPAHY | |
| JM 93 | MAAPGSFWTCCGFSPFGRVGCRYRPLPDPLNECPTHWRTEIANGLPP | SEQ ID NO: 94 |
| | GVDMGDVKQAEMCTAALRQTYLLAVQSNKITEYLRRFDAARVPAGCQ | |
| | ETVRIQISKLKSIQNVIWNAMLSLAIGDITVDESAFHALLNKRADET | |
| | VSLLEMEKLATTIASDDSVTWAAEINNVLVDTEASSNPSHPVIRQPT | |
| | PQLAVADNIVPDKIIQDAQADG | |
| JM 94 | MSQVRPLPDERVNEIRAIFSTSGDMAEVITDILTGTQATASFFCVLH | SEQ ID NO: 95 |
| | DRGNVPINTPHAVIKLCLPARRPGGGPRCLPLMVLNLPAWQVHLFLT | |
| | GDAPLTSDNIKDRIDLAQTEEILEPILSVLACKRSAQQTKHDSFKSK | |
| | VAWFRAKFVSALRKVYKMTPSPYWMITLLGSFEASFVLAGTFYFFQS | |
| | YTCTAETLVHLTRLFISSQGQSLVTVNTYDELGRVFGRSDFLEIVPN | |
| | FWAYLKYKMQQDDVESKAIDQTINSIRGGLMLSPQDLVHFIYLSFYE | |
| | CMNAQTFLSYSRTTASLPTPATVNPPQLCRRLEADFKEHVMAYYNKA | |
| | SYLSTYITILTVPAPLPEGYENFQELACQYWCGQSRDVAEIMTRIND | |
| | QYPQLNLTKDLSGLLDLAALDQDSGGPKENLFTVASRIPTYRCEFLN | |
| | KQYFVLMHTDCIDAYWKQNIIVPEDAQLQGLTDQDLTSRIFYCDLGL | |
| | SLPTFKQQIMVSRHEYFNPRLPVYRWVLDFDLKVTEGRRTLNDIYNI | |
| | CVTLRQVILETLQLIGPLKPHHPVYFFKSACPAVTWPDDISDTAFCH | |
| | CDTKIGMRIVTPFPIGYCLVGSAPLVSLTNILNRVVKLDTRLASEYP | |
| | GILEDKGPFDSGIYAKGRCVRVPHCYKVGPGGELSRLLKIIICHPEE | |
| | SDKSAYLKNAFKVSNLLHHAPGDSVTKNGHLVYAIADENEGFLESKT | |
| | KNNLPKTITDLAEKIERTTEKPLIDWAATAVWPKLHDTIQRFFPDDR | |
| | IGQFASVSFMHSGDNIIQVRPQKGNNFFCINHKHRNHTQTVRVFLTL | |
| | HSTKESEVTVTFMSQCFAAKCNHNSPTAHFSFMVPITCT | |
| JM 95 | MLSPECMKLTDANWPMRSSGKKRCMVSCSFGHTAVAAQSISGFSVVR | SEQ ID NO: 96 |
| | SIFSAKSVIVFGRLFLVLLSRKPSFSSAIAYTRWPFLVTESPGA | |
| JM 96 | MAQAMVSLEYMKDILDGKKTSYGSYNFYLKPQLTKRLVLYALHAVRV | SEQ ID NO: 97 |
| | SRTNLFYRGHIIIGLRRLLIGRDGRPVPDAGARTVHGTAQLS | |
| JM 97 | MRYVFHALICFIGGISSSDFDDSSSDEMDDLSPTPEPEPSTAPHSFP | SEQ ID NO: 98 |
| | EGPKSKVVALPKIRKRSRSKTPVKIEHRSPLNRSRSRSRTRSGSGQR | |
| | TSSTYVKRFKPTVDAPRSREPWHRGGKGKAPFIRRNALAERGRRTYG | |
| | NEHRGKSALTRNIKESIKKMHLPSTMLSRPHDKKVFEGLLPRHLEQC | |
| | FQVCLPAPPPLRPEVFTDRQLTAIVKSGGRRDALVAKKVSLAKVMSM | |
| | YKPLLTFVTGRNNQAHWLATRKNTLASAGLEALAAFIEEGLAWAQVC | |
| | VSQNRSLNDSNLDIILDSSQSVCTWFISKIRHLHIQCFLENQGEISL | |
| | VKQLTYLVCINNRLAEAANLAGEVKLNFKFAMLIGFALTLPALLAEQ | |
| | KLSGESLYLFRSFLEKYRPGDVMGLLNSIVVEHYTKCRSAECVITTR | |
| | AMVGSGENNKGLFFFPA | |
| JM 98 | MATWCPPNSGGPSAMGLREWIVTHANLGTYSGLFWADDEKTRVVLAT | SEQ ID NO: 99 |
| | TTAWTVEFDYPRDGKVYEDYCNQRNIPLPSGRPRLCQAKARLLGAIR | |
| | KSAYFVEEKNFLRRSFSFPTVVFRLRSNEEMSCRLCPRASGVAAELR | |
| | GLRFRMFKRKGADDVGRVTEYTVKQLLGLLRTRHAGASTMTAPATEA | |
| | SATTAGEDGRQDESEGGAVALPEEHTPPLSVSSGLSACLAPSVDDPW | |
| | GFMHIQVYYYGFLQSQVFTRTGMGVRLSTRPTDKNEHHVCMAHGPLQ | |
| | LWLPPAPHMDDDVMLSRLVNALNALEDGIVLSSCQYGIMMNGYGFLN | |
| | LWFRGNTLNTLEPTRVPSGVGHRIFDTDDYITKLAQSPRPSDPGPPD | |
| | PFAQIWVAAWSLYEEEDLSQAPICIIVHQREIYRHFE | |
| JM 99 | MAPNTQKDRLIQIAAECVPRVTQPRPLHSRPAYRLHRRWLRTPRWPG | SEQ ID NO: |
| | RSWWRHRRDGYEAARAAALPCIQLPCPHRRRPCA | 100 |
| JM 100 | MRLPVGADVCSRHEIIPPAATNSLSAQSAGYRLISLTSTPPHHLHRS | SEQ ID NO: |
| | CPCPDCPLNLVRSRPALLPGQAIDSSPLRDLPYGRNRRHEPLMFQLS | 101 |
| | RGKNNPRCRTQTRFRTRNAVLWRWVHGRDSATEPTDTGRFVHIJL | |
| JM 101 | MAGRGVDIKAWLVAAVESSEYHGLVWENEDKTVVRVPWNKVTAARSD | SEQ ID NO: |
| | SEKFFDDYCNMRGICQGEKPPHYGRFRKMRFLYDMRHHKSIRELKFI | 102 |
| | NKAYGRSEAPYRLFRLLPEPVVSCANCNLMSSTETQCLGLTSEFRYD | |
| | QWGGFGRERRRVFTATVLARSWMDKNKRVREHRLPGAIQLTFLYFGS | |
| | TVGLERVHAGTRVCSRPYPVLAGHACCFQDERTLFLPPPGVVDCSFA | |
| | REDLRGMHKKCEKGLLIALTDTGISVKNLENREMKVLTNNEEEYKDL | |
| | PSRQPVQVFDMVDYLRALARSPKPGDDPPRDYAQIALCLSVQSPNPA | |
| | HAPIAIRLRYVCETSSVCGTEGCFYPGTIGTSEARVAGSFHMEDPGE | |
| | GTSQSLDPAVELGDSGPDSMDISIPDTGTSGEDDGVACS | |
| JM 102 | MGGIRRLNRQTQGDLCIISRWVVSRLRGPCKSSKIIHHIKHLNGLPG | SEQ ID NO: |
| | RQVLILLLIVSKHFHFPVLQVLYGNASTRKGNQQTLLALFVHAP | 103 |
| JM 103 | MERPVRVTKPSSLRGWLVECCETGRHPGMRWIDDERTLIRIPWNHDR | SEQ ID NO: |
| | GSRGVEESEKNIFIDYCRSRGILHAAGRELTAKECKNWLSSAIRHSQ | 104 |
| | TVSDVSTNDDLSTPYPNRCRTIRLLPITVRSCARCDQASGTTAMLRG | |
| | LREEAVNKFGPVGAGVQYTGAVGAGGEQCWMLRITFYYYGDRFGEVV | |
| | TESPNGTRVLPLSERRAQGHICVAPIAEQALVPEIPSHLVEFQAEAL | |
| | RFLDKDLLRGLAFWADPSGIYIRWLGHSMAFVQGNVESPGAIVVLSC | |
| | ANACRAFNLVDYMKGMARTPRDGTAPPQACVYLYFGGVPTPEGGVQS | |
| | TVPLIIQLWHECLWQALSAANV | |
| JM 104 | MAIRDSNYSHLRSWTLYHLNEKTYSDLTWCDQEKKTFKLSWKKGAAG | SEQ ID NO: |
| | TPAVVAYCAQRGLQVGIDGNVFECKRRFLRGLRENAGFQECEHGVVR | 105 |
| | THGGGWTAFRVKPLMDSGCFACILDEMSEGIINYLEQVCGIGLEPGM | |
| | PLPAPLPTLVPPTRSAYARAHRLGVPEAPLPHQIVPFWRLRIQVFYF | |
| | GVLALDHTSQDRRGVRLHPRPVPRPGHLCFYGTGFTVWFPSPDPGKL | |
| | TPDQITQINTMLVTYNEGIYVHGNETGVYVDNRNRETLYAAGNDCNG | |
| | DIIQREVMFLSKQQIFYFMGFMRKLARSPVPESHAPCNGATLYLSQQ | |
| | PGAQESPQVPISVVVCQDELVQGQMNPSRWCA | |
| JM 105 | MALWYRGAGKLSHETHEIKNLLFREEHDLSLNNITITVVTRSVKRFP | SEQ ID NO: |
| | VPIIDVHSGFIAVYVDALVIRDQHCVYLRYLIWSQFSRIWRWKPNGK | 106 |
| | PGAVKTQMTGTWNRARMQTNTSSILGRMIQCQNAKIKHLNS | |
| JM 106 | MAAGESRRGPSRYGMALREWLISKADSGFYPGLFWADEHKTRLVLAA | SEQ ID NO: |
| | TSPSLPNYDYQRDGQHYDAYCDLRHIPLPSGRGRLCQAKGRLLGAIR | 107 |
| | KSKYFDEDKDFPTDQFPFTAVVFRLRSFEEMSCPVCPRVDALRLELR | |
| | NMRFAMLGRGMLHAFPESSVGDHEARHLEGQEGGDDARDDDAPYSSG | |
| | VLRARLMACAAPSAGDPWGHMHIKIYYYGQLQAELSTATGQGIRLSS | |
| | RPTNKSGHHVCVLDGPLQAWFPPIPQITDTSMGQRLEDALKWLVDGI | |
| | IFCSTSRGIVFTITGAPNVWFQGNTVEPRNFPHRAYTGMHVWAFDTD | |
| | RYLLDIARSPSPHDTDPPAAFVKLWVSGCSLGEERNVSRAPLSMTVH | |
| | QTEIYRHFE | |
| JM 107 | MTQLRPYCDKHRESGTRVLLWPFCQMNPTGNIYFLQYGAKVETEVLP | SEQ ID NO: |
| | VVPEAPPRTPDGPSSPLLLPVALSPVSTDTSSHT | 108 |
| JM 108 | MAEQNMDLKAWFIDAVESRRYPGVVWDDDDRTIIRIPWNRSTDSRID | SEQ ID NO: |
| | EEYNKIFDDFCLARGVCQSGSTAHANKFKKIRMLYAVRSHRYLRELT | 109 |
| | PPSKAGGVSGERYRLFQLLPEVTNGCDLCNLIATTSLHSCSMGSGVR | |
| | DDFFEQAPRPRARLPLRVSIQRRKARLQGSPAQAAPGAIEVSFFYFG | |
| | ENVGVEILRSGCGVRICGLPDPKRPGHLCCADNPLTRFLPSAQLVPC | |
| | EFARADLQALQKTCERGLICVMVESGICVKNLEERNMTALTNYSENY | |
| | YELQPTQQFQVFDLLHYLRALARSPTPGDLPPRDCAWIFLCPSTQSE | |
| | NTWDAPIALKLRYVCDDVSVETGDSATGSNSGDEGPSGVRGGASGTT | |
| | GSTSVSTFAPYWRK | |
| JM 109 | MLRSSRFFTQIPDSTITQIRPLSHVFCRACKSALANSHGTNCADGRK | SEQ ID NO: |
| | RVNGLSAQHRCPGRLGSGRPHIRTPQPERKISTPTFSPK | 110 |
| JM 110 | MRLSVDLFHGIRIMVLSSSSHTTPGYRRDSTASMNQAFKSMFCSAIS | SEQ ID NO: |
| | RVHHLYRAGQLKYRAHRTTSLRDQKNLDLKEAYLYTPTGQLSNK | 111 |
| JM 111 | MAEGRAGSIRVNRPSGLRAWLLDCCDNGKMPGMHWLNEEKTLVRLPW | SEQ ID NO: |
| | NHLKGAGGVSENEKNIFLDYCQFKGIRHTGNRPLSLRECKNWLASAI | 112 |
| | RHSQTVEDVSTEENLSAPAPYRCRVIRLLPIFVRSCPLCNEADATGG | |
| | MLLDLRNEVTARFLYLGAGREYEGAVGSDGVRCWMIRLVIYYYGRLV | |
| | GNMEVGSPNGVRLLPAPKQPLQGHVCAGIKPEQVMLPHNPHNLLPYQ | |
| | TSMLRWLAKEIIRGLMIYADGSGIYVRYMGHVPAFLLGNGGSPEPVD | |
| | MINSGRVARVFSLVQYLSAVSATPPHGTLFPAAYASLHIGGVPTPEG | |
| | EPCPTIPLSIQMWHECLWRACGDAAM | |
| JM 112 | MAESEITHNHLRRWIISNLEANTYPDHLRWCDEEKRSFKLSWHRGMQ | SEQ ID NO: |
| | GMQPVVAYCLDRDLQCGRQHNVSDCRKRLLRVLRENAGFEQDDTPAT | 113 |
| | TTNSGGERFFYLKPAVDPLCYACILDSHSETVINYLEAACVHGLEPW | |
| | TPLPPQAPAEASGAARSVYARAARLAIAAPPHPEQITPFWRLRIQVF | |
| | YFGSLVAEHTGVDRRGVRLHKRQDPKAGHACYYGTAFKMWLPTPHEN | |
| | GPLTPEQRETVCEIINYCEEGIFLHGNELGIYVDNRTRHTLSCAGND | |
| | AEGNHAQRFVRSCAKFQIFYVMGLLRKLALSPVPGDPVPINAVTLYL | |
| | GGRPGSRKRPQVPVILVICQDELTHGDIRPARWIL | |
| JM 113 | MANSWGYMWITEQGTLFPAPGTTQRETTHSVLCDPVPNFRFFMLWVY | SEQ ID NO: |
| | CANSPFPPFPATRFPSTQSLFTLGVALAPVKDPRSLSFW | 114 |
| JM 114 | MDLSLGRPYRLKLRRRLVVRRGPFMPEPPGWQLPRHHTRSRSLHFGG | SEQ ID NO: |
| | YVYKFFILDRWWRNTRAWIVEGYACISAKTQKLANRVIMGQRLRCGF | 115 |
| | QRRMRMDH | |
| JM 115 | MGTYTSEASLAWLSFMSGTVAASPFILCFIYHSLYFLEPLNSVENII | SEQ ID NO: |
| | FSWGAVGLHGLLLLVCVVGPPAWLSRQVDVPCTISAILITAGSMAST | 116 |
| | LGVDLPWVYVSFFVGSCLCLLLCVVVANDVVYLCPTIAHRYYELGFF | |
| | AAFSVYYFLVLKNLFLAPVFLLPLVAFIVGGVCSLRALRSHPLYEAG | |
| | LQRRHAIFSLTSRRYITYSIKQALEVCGWDFYLVTVLIGGAAAGTLS | |
| | VGLTTPLLLGLVHYFFVFHVGLFCCIGLVFRSNVLALVYVLAAAVLL | |
| | TLTHVLGPGTHNLFTRVCVFTVFLLTMFGAIGCELQIIRKKLQRAAN | |
| | SPRIVLGVCACGNLLMAVVFFSLNKVELVAL | |
| JM 116 | MPVSFHYGARVDVDALGNISKVYDHIKGIVKKGVIQISGQGRAPVLS | SEQ ID NO: |
| | VLSSVGDAGVLGLRLKNALAPLMVYSDMTDEVSFSFRNTSLGNTFTH | 117 |
| | TREMFGVNITEMNVAFYHHGDESNAEGKPQFVRTTIAYGDNBTSTVH | |
| | KSVVDEPNLPSFHDRLEQAGTGNRLFLTVKTLTLLSKWLRQQKTRAK | |
| | QVVTVSLSETLAVATFTVDGVSKIIDFKPDTPDAKWTCAKGRKLDVG | |
| | VVSSDLTTHVSLESLVAALNACKIPGFFLPGFRWHANEILEVEGLPL | |
| | TDSLADVKLGVMLLKVDPTDRNNAVPGNLSEGADPEGVPELPSPPRT | |
| | PDLDLKEQCVPNAEDGTEPTDGGAKSLRTSGSRPEKKHGKRKHSSSP | |
| | SRGKAKTKTPRATFNPLF | |
| JM 117 | MLTPNISRVWVNVFPREVFRKLKLTSSVMSEYTISGASAFLSRRPRT | SEQ ID NO: |
| | PASPTLESTLRTGALPWPLICITPFLTMPLMWS | 118 |
| JM 118 | MSGLSIVTAANESPDRFLYASDHPGFLALTQETWQNRWFPSQISLHE | SEQ ID NO: |
| | DSDEVRLLSPTDREFYQFLFTFLGMAESLVNFNIEDLVKEFSNHDVT | 119 |
| | HYYAEQVANENIHGKVYANILNLFFGGNRGDLMTYAKKIVEDATLAK | |
| | KIDWLHSRVRKATTRAEKVLLFLVIEGIYFISSFYSIGLFRLRGVMR | |
| | GVCLANDYISRDELLHTRAAGLLYNTMISRDESPSVAYIHGLFREAV | |
| | EIETLFIRSKSRGVTMVNVGDIEQFLQATADRILKSINVPPLFGARP | |
| | PNACPLSYTSAKSVNFFERDNSEYVTSVHNDL | |
| JM 119 | MNTETSFSAAKSAKPLTLVTNAETGGCSSSLDPERCAESLVNSLKAT | SEQ ID NO: |
| | LGWDVEANSLTGLLWHRIMEDRCLVTVKDYLTVFGEKLSDEVREFNL | 120 |
| | KHEAALDGLLQDFKQSKAYANLVNCGYLSAVRFYDTYVLRTHGSSPI | |
| | FESVAQMFMRVAVFVACQCIKFPCLRKTLRHLVQSETELDEMYLVGY | |
| | AFHYISSQIVCCATPVLRSAGLRGGQLSSCFILKPSMATENKTLKAL | |
| | HEEMSPLLASKSGVGIDVSSFAEHKNITSCLKLINAHVGYFNDNNIR | |
| | PVGASAYMELWHHQICDFLNAKMPENQERCHNLFQGVCVPELFFRLY | |
| | ETNPDGQWHLFAPEVAPNLLKLYGAEFEIEYNRLVAAGKHSSSLPLK | |
| | SMMYALINTVIKTGSPYVLLKEALNKHHWCETQGSAINCSNLCAEIV | |
| | QQPEGQASVCNLANISLPKCLRPPRGKSGVEPGKGDVTFGFELLDDA | |
| | VEAAVIIVNACILGGTAPTESVRRGQKERSMGIGVQGLADVFAELGF | |
| | GYLDAESAKLDVEIFQAMYFAAVHTSHEIVLLGEGTPFHGWERSRLA | |
| | QGVFHWQTWDGVKPSHPPLERWEQLGRSIAQHGIFNSQFLALMPTAG | |
| | TSQLTGYTEAFYPFFANIASKVTSKEEILKPNVTFFKRVKPGDLRTV | |
| | RRYGGDVASFPEPLKDRYKIFLTAFDYCPIKQLERAGAPAPFVDQSQ | |
| | SLNFFLKEEQATRASYIRDLLLTGYRLGLKTMLYYCRIQKQTKLWAL | |
| | QCLDQVVGDNTSSEGAESNRVQKADGEQAEVCLACQ | |
| JM 120 | MTPWRPQLLLSMRAFWAGRRLPRALGAARRNDQWALASKDWPTCLPN | SEQ ID NO: |
| | SALATLTPRAPSWTWRFFRQCILRRCTRVTRLFYLARGLRFMDGSGV | 121 |
| | DWLRVCFTGKRGTALSRPTRPWKDGSSWGAASPSMEFSTASFWR | |
| JM 121 | MERVTNQVHLVQLRFTLHQVSEGLPQTRKFYALTGHKNSHPHKHLGN | SEQ ID NO: |
| | ALKNGRRAMGSQHVRIVKPDGRQVAAVNEICVCLALLKVLQKTVKGR | 122 |
| | FVF | |
| JM 122 | MKTRDANVNKLNDSLMRLLPPPPHRVSLSRGRDFSKGVRDLLSKYVV | SEQ ID NO: |
| | STTTGVEAIKDGFLSVSPKCQTYGDFLIYSQTMSSQEPRGTYLFSFK | 123 |
| | QTDTGSSIDMLFTPTSLARLSRMDADSAPQTNRIACVWYGHESGLLD | |
| | AIPNFEELLETGSLHQFLAPVGPLVQTVHSTFVTKVTSALKGDVVAP | |
| | EPVVTHIGLTLPSDMFVDLDDSCPSSLRDEPLPAHSSIYVCLTYIRA | |
| | NNRPALGLGFFKSGKGYCEIAAQLRDFYSGVIRTKYIQLQNDLYINR | |
| | LAFGVVCRLGSVPSGSQPSFQSLHFKGAALPVLKFTEFVSNPGSWKL | |
| | FL | |
| JM 123 | MTSSISAARADNGDENTGGLYRLIDNLLTCTGSLQQLKLLMEFQLKP | SEQ ID NO: |
| | LPTAHLLSMPTVTRFLMTAFKIDNPLVSFIQKHPVFFLMRVARLPDP | 124 |
| | VITDHQSAETSTGILSEVVNVLNIAIQKPHESPAAKDDDYLDNRAIL | |
| | TMITEYIHHVTSRTPSGIPPTPPMGISHLPCVEQILHETHRQYWNLA | |
| | LPESLFIDTGEVASPLQTWLIISYCKKLQLAPPPLFPPADELARRLV | |
| | TGHHELFVPLSTSLETYITMPVSKRRAFEIYSVFAKSKNIVDGTPIL | |
| | AFTDTELTTFTPELLFLYDFVIESLCKNQAYGCSRNAIEHFIKKGID | |
| | FMAELGAFIEKTCGYRSTVSLSNVRSVKARLASCGLSKEACEDFRTM | |
| | ILMTPHETTPKWENFTDFLEMVNQLTLYGFYFYECLNQYSPTSISLA | |
| | KIQNILNRVDAEQSDRALWRTPLIGSFPFPWKLNNVLAFFKPSAPVA | |
| | TLQKIYKAIPSYLMRSLFEIAANKSWGNIALAERAPLTDIQTAEPDQ | |
| | GPVPTQVIAKYCSRLQISATDYDAVIVSSPGFATEFIRTKLYPILSE | |
| | VLRNTSKKNRSLFQLRWLIVFAAEDAKDLAPIRRSLALAYFQIMDIL | |
| | EEKHSPESFYNLLDYLQDTFRCIRQVIPEATCPQEFLQYLFTFQNLP | |
| | IAASFIQTSMTFVDDLNNGIPGILDLVSLGAAFYNMKLLYDSTLDTV | |
| | EIPTEEGQPIVVSMFVFKSTIRILEKLLQEAVIALAQTSEPMYAAHI | |
| | RLMQHLTYMQKIAGHEIMTTQLPSVFHEIHEGYLQCFKRFKRLMLHV | |
| | TGSCCYSLTRYFGFLYQPPLIPDTIVQKILNFNDKTDTTDDILKSLS | |
| | QPVGQEPLSAENESSSRLSKNDVELLQKLYDDFRNGSTNDNPTSIKL | |
| | EYSGNYNETQVSVDWSTYNLVTYTAPDDTLKFTPVNTEALDRMLAE | |
| JM 124 | MELPPIFSKFKIEGVATTHQADCRFGQYAGSQCLSNCVIYLAQSYFN | SEQ ID NO: |
| | RESPVTDTNDLDDVLRQGATLDFILRRSGTLGYNQYAQLHHIPSFIK | 125 |
| | TNEWSAAIFQSQEYFGLIGLDAAIREPFIESLKSILTRNYAGTVQYF | |
| | LFICGDKAGAVIIKNKTFYLFDPHCVPHVPNSPAHVISSADPTAILE | |
| | YVSPPDREYTGSFLYIMPSEYVNPEHYITNHYRTITFAKVHGPQIDI | |
| | STGIEFCTIEDIPSPPRSPDVTSKSSNLARVPKTTTDTSSAKTTPAP | |
| | LSGLLGVEPPTSYPDPAADDADTKLLTPAPARTAVDHPEFQTTPGAT | |
| | LLLSELSASRGRKRKLSSLQRYSDSDEASSDDEGAPRRPAHDDAISA | |
| | EVIWMDDDISPLYSPSATPSFDDVFDSPPMSPEFTYTNATEDTDGAF | |
| | LEQIVRDAETPFSAFNDLISDHDFSSLDKKIEQLIKYEAPSQHLPNI | |
| | SDKRNGRAVREAAALQAMDKIMINIILEHGLITDAQARGPSACKNVL | |
| | QFFILWGEKLNIPISDAKQVLELDLQLIPLHTALSEGKFKQGAFKKH | |
| | LTTKINRCLASMRATHANAHKKLASAFNIEGSQISSSETKISVRALK | |
| | EQIANHLSPGFLAVYSADELKHLRDKIRDLKTGIEQRNREIQQEELF | |
| | FGAVLTALDTFQPPPKPAYPMEIFPHRKTDVMLNHLASITTRLTDDA | |
| | TEALNNYLETPPDQGTHITNIPNFSSIVANIISTIKILTYAENDMQL | |
| | NVTPMATYRRQLLYLGGELATIFNLEWPYETVPPVQELPLVARAKAK | |
| | MESVTKMEKNQQALDQILGDAETLLDTITATSGNENPVRAMSIPILE | |
| | TYITNAGALIGTSRNPRFEKLKAAIHDLASSESFIIMLLNNTRLDNI | |
| | SDNLAKIDGILTNNTRFLSTATVTKTLQTLGSSLIRECVEALDKRSP | |
| | SSLTDARLLAVQTILGHASVPDHETLTRIVSGVASAQKESAGDDPDR | |
| | WTRVTGHLNELKLAATQSNVDKATRRKLLMIITRDLKEAEVSQETVL | |
| | ETRWQENVLKFQPSTSKEIEDFLQSAPSAKARKFAEKHLRTLIAQFD | |
| | GHEQRPSEATAVPMDYTPTPIPTPQAVSTATAEKGKAAWNKIQQAFQ | |
| | DFNFHLIDASDWQEMASEYSRHGSSLPGTVGPKLVRFMESISNTLDD | |
| | ILTQKLASLLPNGPAFRPPAFDWIAPYQTRVNAFLKTIGLPMVRDLA | |
| | DKIHHQCQTVSHAVQSADLQQATVGTSLERPAAEYCRILSDMQVAFN | |
| | DHGIAVRSEAAAYTDAINSPANVATPPKPNLEAPKKLMTATDVLTVE | |
| | DFPEFLKTSILQQEQRLIALQRAEFQQLEASISAAERLRQSTRNEIA | |
| | GKMATAITQLLPRAPVAISSRPLNLSKPIDFLSSTVYDKILDKEPYE | |
| | TAIAGFAWLETATKSVMVYSHQNQTQQLNVLLSEVEKQSTIAQRLHD | |
| | LEVSARNTDDVKVLKQALDELAPLRVKGGKTTVDAWKQKLESIESLL | |
| | RATRTAGEISSELERIGAQAVGTIAVRDLGTLSDQCREAANFLKQAS | |
| | LPEGFSDIGTKLSELQAYIKYKKQFLEHFETTQPNVFRRFPLSQNIT | |
| | ENVPARLAMDSMARLANHLHVRGSAPHFTTWIETLPTVDPEKPTHVP | |
| | AHGGAPLHRQITYSNVLEALFSLCSTTLTPVPTAPGLEIATRARRGA | |
| | EAATWMDRQWPDIAQTLQDVLDTYEHTTTHANRDATSNAFLAMCVFT | |
| | QIIRGASRAVTLPKLPGTAVDFPEEIVLTPRECTTLVTAMWPTLAAA | |
| | ILRLKSYSEALGLMSRFLPLMFQALPHLTLEAQVQNGPHNTPPQVRC | |
| | FARTEAIPYFPAQWQSANLEQSLWGQTDFLQICDNNQRKARVAAVTW | |
| | ALTTINGVVLDQLWSTFKPMTAASDDTYVDLVKTLHLTTFGPRGPTP | |
| | RRETTTEHPPYEYGQPTGYCISGQSTTPVQASNTPGVRF | |
| JM 125 | MESPTVNIEEIYRRPSRSPRRISHRRVRAYVGPLRRQTTLRRNPNIA | SEQ ID NO: |
| | EGWTACVSDPWMPTVLKEVAWLPVLFGIRGGRRRFALERELRVLCPR | 126 |
| | RRLPGLGSLSVVRRPVDRIAVPVWFWR | |
| JM 126 | MILIIILSIAWRAAASRTARPFRLSEMFGRCCEGASYLINCSIFLSR | SEQ ID NO: |
| | EEKSWSDIRSLNAENGVSASRTICSRNAPSVSSVAFVYVNSGLIGGL | 127 |
| | SNTSSNDGVADGEYRGDISSSIQITSADIASSCARLRGAPSSSDDAS | |
| | SESEYR | |
| JM 127 | VWTARRRASVREDGLLLSSASTHSRIRLLPSVWSVFVTVAVERKRV | SEQ ID NO: |
| | LLVRMPSILARLSDMLSSRVLFSSIMNDSDDARSWMAALSFSNRGF | 128 |
| | REVPIRAPAFVI | |
| JM 128 | MSRCSCWRIDVFRNSGKSSTVRTSVAVISFLGASRLGFGGVATLAGE | SEQ ID NO: |
| | LIASVYAAASDLTAIPWSLKATCMSESIRQYSAAGRSKLVPTVAC | 129 |
| JM 129 | MVPTACAPMRSSSEDISPAVLVARSRDSMLSSFCFHASTVVFPPFTL | SEQ ID NO: |
| | RGASSSSACFSTFTSSVFLADTSRSCRRWAMVLCFSTSLSSTFNCCV | 130 |
| | WFW | |
| JM 130 | MGTGLRGALYRLLNGSTSGTHSRMGVRSPMPPRAWPRRWACRQKDSH | SEQ ID NO: |
| | WYVKHHRSEHRLKSGHPACLKPGPAWSIGPRCSSPWAVRTRTAGARW | 131 |
| | SFLALVLDRADQRWSGVGS | |
| JM 131 | MGLLTPILECVPDVEPFKSLYNAPRKPVPINTLPASLHPHDEQQVFL | SEQ ID NO: |
| | RQAQWLSYRFIPHEAARSSSPPLLVVIDPENLVTATYSSGGPANFES | 132 |
| | RPFYVMPGPYPTDWPKTLSVTSNTSVTHLSHDEICNLFTTLSREHGT | |
| | VQGRDIFAAAPTNVTPERTANHPTWETENQLTTQTEPAEKTHVIPAS | |
| | PKARTDPIAETTAHHSQGQASQHANNVNQPGQITSHASRNKPSTAPQ | |
| | ASSRPEKLNTQTVPRLISQTSETAHINHPASGQVTEPKGIFGTYKPR | |
| | VLTEPAKPAHASIASRQPEATTTVPKLPINAPTAKVFIGTASKFLPD | |
| | VEESHGTTPGAHQSKIDQKQYGESQPHRTPHLEEVPRAPHVDTPTSA | |
| | HINVPSSQGPKTIHARVTPGIQTVTPSAPPPAGQIRAFNTRFPEPTA | |
| | GRSTTNRMWN | |
| JM 132 | MLYFQEFGGVLVDESLRLDRMGRSIYSRPVRACETHFVFWAILLPHW | SEQ ID NO: |
| | LGSIPHPIRRGATGRRFWEACIECADLTGRWGGAGSYCLNTGCYARV | 133 |
| | YCFWTLARGDVYVCAGWSVDVRRPWNLFKVWGAVRLTFAVLFLIYL | |
| JM 133 | MSSLRVKEPIVQGRLEHDYPNHPLVAEMNNLPQGDMSPAQYAIAKRN | SEQ ID NO: |
| | YLVFLTAKHHYDMYTQKKNGILRKDHLRGLRGKKDASSSESSVLSGS | 134 |
| | GSAAPSVAPVASTLGSNSFTTISSGPHSLIGSIGPTPGGGGPGSVAS | |
| | SGIGSTSLSPSDATTLDTRRSSQNKKSK | |
| JM 134 | MPEDATLPGPPPPGVGPIEPINEWGPLEIVVKLFDPRVEATGATLGA | SEQ ID NO: |
| | AEPDPDKTLEILELASFLPRRPRRWSLRKIPFFFCVYMS | 135 |
| JM 135 | MASGRLPDLAEDEAACHGRVPYPVHHWLDCSRLGLDFAASMRAIGLC | SEQ ID NO: |
| | PECYVCFVTYGLGAWEGQPPEWARTLISAPSFQTALNDIATGWMPDN | 136 |
| | PPKNGDVKSRLHDTGRLLLEAYAWVLRCICTGVGYADNEGLSLTAVP | |
| | RGAWSRYLVVSFPRACCFVCKTLNCRQRFPLVTCSPQHALDLPVLRK | |
| | KWNGAGCVAMQLNVPSESRRLGANLDERGLGPSDAGLLASLRELAPT | |
| | VPCGNPFNALLRSLTFRGLLSMSRVVLPIGESTETEISRDLGQKVLA | |
| | YNVLFPCISLPVWSQVVARSVLEKTVPAPRVVVCLECGYCLNFGRGK | |
| | FETVNFPPTNVFFSRDQKEKQLSICATTGRVYCSYCGGSHMRVISLF | |
| | EITCVGDPYLRCVLANNAAHSIRDANSLVSVVVPCLASPDCATGLLK | |
| | HLRVAELFYLTSSISSLSCGKCNRS | |
| JM 136 | MGSTTRDMLNRPLNVRLLRRALNGLPHGTVGASSRSEASSPASEGPR | SEQ ID NO: |
| | PRSSRLAPKRLEMDGTFSCIATQPAPFHFFRKTGKSSACCGEHVTRG | 137 |
| | NRWRQLRVLHTKQHALGNDTTRYLDHAPLGTAVKLRPSLSA | |
| JM 137 | MSSGKRLVDELCDVVVSYLGPSGISLDLERCQDGAPVYAKGGAVPVC | SEQ ID NO: |
| | TVRLQHGCVYHLEFVYKFWLHKLERLAYPFAPCFVITNNGLATTLKC | 138 |
| | FLCKPRDADAQFGKNLPINSDVYLERNSSVFLGQDDFMKFKARLVFS | |
| | GDLNVYSSMVICRTYFTEHRQVLQFLVVTPKSAKRLKTLLRTVFALT | |
| | GHSDGLGALRRTGSVARPSGSELTDIGSGERCGNDRLTDSIGTIGGW | |
| | PRGACLTWLKTKLPVMGAFLILSIIGWIVLGWA | |
| JM 138 | MMDRIRNAPMTGSFVFSQVRQAPRGHPPIVPMESVNRSLPHRSPLPI | SEQ ID NO: |
| | SVNSDPEGRATEPVRLNAPRPSECPVRAKTVLRRVFNRLALFGVTTK | 139 |
| | NCKTCRCSVK | |
| JM 139 | MSGDDLLAFESLLPEDMKIMFPTIYSRLNAINYCQYLKTFLCNRAQT | SEQ ID NO: |
| | RSAHCEHCMVLDAKVNAVKQVIHKIVSTDAVFTGAAHST | 140 |
| JM 140 | MFVPWQLETLMKHWPSLRELVEQSFLPGTPDVAFNSPVLIHTQDSLQ | SEQ ID NO: |
| | PASSCRVCNILFTLVRTFPPPDSFFEDYGWLCLTCLYAPRSWTATLM | 141 |
| | VAADLLELIHMYFPQRVKDGPVYTTQNILGIDVQLHFFATRCFRPID | |
| | REQILHTSHLNFLQTEFIRGMLEGTIPGSFCFKTSWPRTEKDDQQPT | |
| | VACCSVGRGNHANRDNHLPDDLEEAFNSTNAKEKPSILGVFSATWSE | |
| | SQLLGSDTQQATIHSQPSTFPTPEDADQSQGPCLMHPTLNLKTKNHT | |
| | ASICVLCECLAAHPDAGPVLRDLRRDILENNENNVKLVNRISYILND | |
| | PDSLSHVRDEHLRGLIKRCSAQEIHKHFFCDPLCVLNTYSHCPAVLF | |
| | KCPPPEKYKKLKARLATGEFLDCNRIFDCETLQTLAVLFKGSQLAKI | |
| | GKTTSLEIIRELGFQLRRHNIQITHPFQTSNLYI | |
| JM 141 | MYDMRLTSLTLFSMFSRMSRRRSLRTGPASGWAARHSHRTQMDAVWF | SEQ ID NO: |
| | FVLRLSVGCIRQGPCDWSASSGVGKVEGCEWIVACCVSEPRSWDSDH | 142 |
| | VAEKTPRMLGFSLAFVELNASSRSSGRWLSRLA | |
| JM 142 | MFDECPNDERDTHRPGAMVFRFNGNITDFEVHIGVPISLKKSTPSSW | SEQ ID NO: |
| | RRVRAEKMCVSETNFFHCIISLLQLPCLDAGCVDWGPSGRLTLGAYG | 143 |
| | VRDANRRLGCFGIV | |
| JM 143 | MPKQPRRRLASRTPYAPSVKRPDGPQSTQPASRHGSCKSEIMQWKKL | SEQ ID NO: |
| | VSDTQFFSALTRRHELGVDFLREMGTPICTSKSVMLPLNLKTIAPGR | 144 |
| | CVSLSSFGHSSNMGFNCSSCTPTDRSAVSLDANALGEDSARKNSELC | |
| | SVALTFYHHAEKVVQHKGFYLSLLSHSMEVVRRSFTQPGLLYAHLVL | |
| | KTFGHDPLPIFTVDANERLALWAVFHTRDLHLGETSLRLIMDNLPNY | |
| | DITVDCIKQTYIMKFTPSRPDNATVTVPVNSICEAVATLDCTDEFRE | |
| | EIQRGTTIINSQGSL | |
| JM 144 | MLTRAPRLGASVQLTAVRLLLLFLTSSTPTAINCTKPGSVFFWLSWP | SEQ ID NO: |
| | KVFMENHYSFISVNSVSITVVAAGKISSRGKTATNC | 145 |
| JM 145 | MWCLVQRAGPPVGCRSPGGVQVPRWGAGPPVGCRSPGGVQVPRWGAG | SEQ ID NO: |
| | PPVGCRSPGGVQVPRWGAGPPVGCRSPGGVQVPRWGAGPPVGCRSPG | 146 |
| | GVQVPRWGAGPPVGCRSPGGVQVPRWGAGPPVGCRSPGGVQVPRWGA | |
| | GPPVGCRSPGGVQVPRWGAGPPVGCRSPGGVQVPRWGAGPPVGCRSP | |
| | GGVQVPRWGAGPPVGCRSPGGAPGGSGWGGGSGWLRVGWRLRVAPGG | |
| | VAAPGGSGWAQVLKATVNPTANPTQKSPCASRAEARLLTCASGALYI | |
| | GQNTLPK | |
| JM 146 | MGNPRIDRSHSKHVGFTLFGESPLAGPNVPAPCTWVLRNAKLPLPCR | SEQ ID NO: |
| | VPYSCSATFEYTALHGWRAVGRWCANQKLMIHLLVLWLHNNTMLLIL | 147 |
| | GGCFGLYKGRRKHR | |
| JM 147 | MYSWGEFRTMERKMSLRVTRGSQKHITMGLFGAHKRAVGNGLGGAPA | SEQ ID NO: |
| | PPCARNLGRGVRRGSPKHVLMVAARTHRPLFGAGVIRRSAQNVAHGT | 148 |
| | HCTHGAHEGGGVAGRSHRAGRGARRLWHRASDVYGRNPLSRGIKGRR | |
| | QGRFPLARRAVNQTDARRRLVVATVGFLLRCRVPSRDWLGDLSIKNA | |
| | RRPLAQPPVNIHGILAANWPKQTANPRVGFCLQVPLYYIYSIIFFPC | |
| JM 148 | MTPAPKSGRWVRAATIKTCFGDPRLTPRPKLRAHGGAGAPPSPFPTA | SEQ ID NO: |
| | RLWAPNRPMVICFCDPRVTRSDILRSMVLNSPHEYMFR | 149 |
| JM 149 | MVPFGAHGAVFAARGRRRVTETCTHGANSGPWSAKCRSGSPEGHRNI | SEQ ID NO: |
| | SPWAYLVPISGPWEMGSVGRRRHHVPAIWGAGSGGGHRNNF | 150 |
| JM 150 | MSCEHFPPGYNGQESAGKTSTGLPVGEREARRYAACVSEVAPMGRMT | SEQ ID NO: |
| | TPATRRAAGADISQNRPRRQAARLPPPTTLILAFKLLFKARLFVAPA | 151 |
| JM 151 | MFPHKRIVDLGRHLEADDREAVLWLFDRPVSDNTPEGFANGLCPPTG | SEQ ID NO: |
| | EPGIPFPVLLEAVFLVGRLDLVSTFFLLDVGFIVERLRSSPSYFSPY | 152 |
| | KHLMLSINRQLSERDVKNLVFLTGNQLGRKRNQSPTFFQWISQMEKA | |
| | ALVSPSDYLVLKDLLQAVSRRDVAKVVAANAPG | |
| JM 152 | MAFVGPVPTGTIDPVLYQDRAISNLLAHETSFVTSTACYGTVQTEVT | SEQ ID NO: |
| | LGMRVILGTWMRSVARAHQADASVFPLAVSILDRYLECRSIPRRRFQ | 153 |
| | RLGAACLFLAGKIRDLNPFKAAFLCFCAADDFSVADLLKQEKSVLKA | |
| | LRWKLEAVLPTDAIGPALFKSGFTKEQLFALHSRVVESVHKAIVNPV | |
| | TGGLSPSLVAAACALFSLGAAAPPLDKLAEAIGVSAATLTAAAESVA | |
| | TTLRELDEDRILNNARGSS | |
| JM 153 | MWGSRQHRSGIVSGHGLRSSCRGHCGRRGGTREQAGGRSRGRGRGAA | SEQ ID NO: |
| | APASASAASASPTPPGPQVLVVVEQGHGSDTETATESGHGSSQGSPS | 154 |
| | GSGSESVIVLGSPTPSPSGSAPVLASDLSPRNTSGSSPGSPASHSPP | |
| | PSPPSHPAPLSPAPPSSHHPSPDPQPPSFLQPLPHDSPEPPGPPTSS | |
| | PPPNSPGPPQSPTPTSSPPPQSPPDSPGPQQSPTPQQAPSPNTQQAV | |
| | SHTDHPAGPSRPGPPFPGHTSHTYTVGGWGPPRRPGGVPCLRLRCTS | |
| | HNSHEDEVPERQREQEGEERQQQPARPPRPPRYPIPIPYPHPRRRYQ | |
| | ENTAHKEDFIVRRWETGSTPLDRARGVTESSFVTQTPIHCIAWQGAY | |
| | SFPGLEHPASAFCRTHLEVPSSRLFASRCSVKVAEPPRPSKKPVAGG | |
| | NNFTHPPPTSMLA | |
| JM 154 | MGPVEALAIAVVQVSRAGIAEGARTLALAIVVVQVSWEGARTLEAQA | SEQ ID NO: |
| | VQVSRGATVAKTTEAADRAMDDATTAVQVRGGRGAMGVMVGANAMRG | 155 |
| | IPGTTLMCFEVTGLKLTPAQTRLAMALEIPTR | |
| JM 155 | MTDSDPDPEGDPCDDPCPDSVAVSVSDPCPCSTTTRTCGPGGVGEAE | SEQ ID NO: |
| | AADAEAGAAAPRPLPRLRPPACSLVPPRLPQCPLQELRNPCPDTMPE | 156 |
| | RCCRLPHMIAVYCV | |
| JM 156 | MRARPDQNSPPPSGHNRKRTKHRFCVRALRWLRVVERAIYLICRFLH | SEQ ID NO: |
| | AVNRDHVGQPATSFRHSIGTRVAKFLQGTLRKTRRH | 157 |
| JM 157 | MALRVGGNLFEKDLLPPGVKHRHRPCVFNHVGRNYINAAAGDARHGS | SEQ ID NO: |
| | VRSSNALCGGPRALYRVPWVRVNNSPQRSYRYLAKTGIA | 158 |
| JM 158 | MLGGITLTLLLATLATVRCALQTHYAAVPVHSTASLGCVLTTAHNVL | SEQ ID NO: |
| | IVTWQKQESPSPVNVATYSSEAGTVVQPPFAGRVDIPEHKLTRTTLK | 159 |
| | FFNATMEDEGCYLCIFNAFGVGKLSGTACLTVYVPLSMSVTFYPPIN | |
| | PTQLVCRAEASPAPSVNWTGVPPELCSEPEVFPRPNGTTLVVGRCNV | |
| | TSVDPEDLRNATCLVTHIGGLASARPLGPVFSDPLEGTSHYVVGVVA | |
| | AVAVLGIFLTGVFLYRSM | |
| JM 159 | MSDQARGVSEVFGIHRRYVAATHDQGCSVGSGKHFGFAAQLGWHAGP | SEQ ID NO: |
| | VNRRCGTGLGPTDKLRRVNWGVKRDGHGQGDVNRQAGGSRQLSHSKR | 160 |
| | VKDTQVAPLVLHSGIKKLQGCSGQLVFGDVNPTGERGLNHRARFGTI | |
| | CGHVNGARRFLFLPGNDKNVVGCC | |
| JM 160 | MDALNNNLNLLMDFLSNYSNSYSSYDDNISYTLDTESTLCRLTIIFP | SEQ ID NO: |
| | PTIYAIICFFIFCITLLGNALVLYIFFKFKALANSVDVLMAGLCCNS | 161 |
| | LFLCASFLFSWLLYVAPQILTPATCKVEIFFFYLYTYFGVYIVVCIS | |
| | LIRCLLVVFSRRPWVKHWASGFLCVCVSLIVALALSANASLYRTALR | |
| | HPETSEWICYEDAGEDTVNWKLRIRTISAICGFLVPFGLLVLFYGLT | |
| | WCIVKSTKLARKGAVRGVIVTVVVLFLIFCLPYHLCNFFDTLLRTGF | |
| | VTETCYIRDVISVAMHICSLLQSMYSAFVPVVYSGLGSLFRRRVRDT | |
| | WSMFRCFSTSGSL | |
| JM 161 | MAQRTNPRWAAAALSADEEAFIHDNSDAESVLALVPEQCFSEFLLWL | SEQ ID NO: |
| | VTRPSDNFDNDDDDPTLGVIWQLLAPLVNYAPLETRSAHLQGHHTIS | 162 |
| | LPYGPDLLRQPTTRSSELVQCLRDSGFDTALRLELARHLSCQTRRFV | |
| | ADRVPPGTFAALTLGALVEYDVRVQRQLPVTVQSTAWRPLPERDPIC | |
| | AAVMLPLQRNILPLAVQASNGNSYTVSRYAVMARRSYGCVFQRLPCE | |
| | NVTHIADSFTHLHSAIRTGAGALQDILFHSTLLPGGDIRSALCGFYA | |
| | TTPSVGAFSRARHRAINTTTTLHCQQLARTGTPVLGGFLKTVHSATT | |
| | SEANVITTTSLLSCVPQAYTFLRRSLFNQPIICLGSFEPVDGDGNQR | |
| | SLYLGSAAGINRINQTLSLAYEILEGPLFTSINRAHEPASTISHLGA | |
| | LVSRGGLRLFVSQLPPTILSQLTATPDISRETVNDILLNKFLNVSAF | |
| | VVFAVLPRDTEPEPGPLDAIRRAARICGCPFAVVGETCEEPGIQFVN | |
| | DLELWNPGAWPIRQPTSAEVIATFGFDEQPVSSNWLVRPEEPEEGGE | |
| | QAPSPTDWGLFRLASVVDQLLRCPTVGSKEFVTRHVDRCSNGLVAQQ | |
| | CEVGPLGRPLSDYHIVNHTAVFTDRMARVPIHRPQPITRQDATERLG | |
| | SPETWVTQGRGRLRWVGQCVAHGEQAYKMGIDAAVGARYAICEAVTN | |
| | IMLAHVRRLSDITLTASVGWNPEEDQAWLLQHALFACKELCRDLSVN | |
| | FAITSAGSTPCLSEELISATQQHQTVAPVPFNAVIITATAEVKSSRR | |
| | RVTPDLKATGNLLVLVTFPGPHLTQGSTFEHLCLLPSPTLPDVQATH | |
| | LANLFMLTESMLSRGLVVSGHDVSDGGVVVTAIEMALAGNRGLQICI | |
| | PSEETPLPWLVSETPGVIFEILPQHVDEVRQACQNFDCQATVCGTVG | |
| | QEGLSERIVISHNNEEVYSQTLTSVAANWTSFSDEQWYSWGPSFTPA | |
| | QELYRKDYGCNRHNLGHLADVCRNSELTLFAVPSRPPAVAALIAPGA | |
| | PLPRALMPAFTNVGFDVAAVSTNDLRGGNILRGFSGLTIGGNVGIED | |
| | SYVGARCAIMGLLNDPGCYGGLMAFFRRADTFSLCCGEFGFQLLGAL | |
| | GLLRETPHDTPGPKTPDQWDIHLEENASGNBECLWLNLHIPQTTISI | |
| | MFRVLRGLVLPGWANGRYLGVRYPRDAIEYHLNQQQRIAUJFHTGNP | |
| | DPRMFAQHYPRNPSANSAVAAITSPDGRHLASLVDPAVVFHPWQWAY | |
| | VPPELADMTISPWAIJAFQSLFLWCVRNRQ | |
| JM 162 | MRNHGSSQRPWVLWGPYGILPPSGYIFTVLRGVWVPAPGGARSTQRN | SEQ ID NO: |
| | PSRHAPAQNPRPVGHPPGGERIREPRVPLAKPPHPPDHHQHNV | 163 |
| JM 163 | MPTFPPMVKPEMPRRMFPPLRSLVETAATSNPTLVNAAISALGSGAP | SEQ ID NO: |
| | GAIRAATAGGLDGTANSVSSLFLQTSARWPRLCRLHP | 164 |
| JM 164 | MTILSDRPSWPTVPHTVAWQSKFWHAWRTSSTCWGRISKITPGVSDT | SEQ ID NO: |
| | SHGRGVSSDGMQICRPRLPAPAISIAVTTTPPSLTSWPDTTRPRESI | 165 |
| | DSVSIKRLARCVA | |
| JM 165 | MAKFTLRSRHSSLQANSACCSSQAWSSSGFQPTDAVSVMSLRRRTCA | SEQ ID NO: |
| | SMMLVTASQMAYLAPTAASMPILYACSPWATHCPTHLSLPLPWVTQV | 166 |
| | SGLPRRSVASCLVIGWGRCMGTRAILSVNTAVWLTMW | |
| JM 166 | MPFCRRRGNLRRAGNPVRERPRAVEPGSVADQTADLGRSDRNFWVRR | SEQ ID NO: |
| | AARFLQLAGAPRRTGGGRRTGTLADRLGPIPPGLRGRSASAMSNRRQ | 167 |
| | QRVCHATCGQMLQRTRSSAVRSGTPGPTAVRLPHRQPHGGVYGQNGA | |
| | GAHTSPPTDHQAGRDGTPG | |
| JM 167 | MFRCNGSITAAHIGSLSGNGLQAVDCTVTGSWRCTRTSYSTSAPSVR | SEQ ID NO: |
| | AANVPGGTRSAThRLVWQLRCLASSNRNAVSKPLSLRHCTSSLDLVV | 168 |
| | GWRSRSGP | |
| JM 168 | MFVSFATMGNTYDFYNNNIMEWTLQNYTLNTANIYSNGILWICMVKF | SEQ ID NO: |
| | TNKHCKNNWIVVCNTCRYVAILLLFIINRGNIYEEINCLFFVTALIG | 169 |
| | MYAVTEASTTSSLTMALAYSIITANTGIF | |
| JM 169 | MACNCPFFVWCTWLFSNILTGTFWLISLAQYACDNNKDLYIVAVSTV | SEQ ID NO: |
| | ACFFFLWKSLGLYFYQSRSQRLNTPLLKLIPWITGMTL | 170 |
| JM 170 | MPGGGRPASGAARRGLWGPGLGGRRGAAPPGSAGPGRSRESAGPGGA | SEQ ID NO: |
| | GPGGGPGPPRDPRPREPKAARRAARGGRGRRPRARAGPAEGREPGEA | 171 |
| | GGAPLGGPGAARETLAPGARWRPRPRAAAGRAPGGPRRAPPLGALAW | |
| | RRC | |
| JM 171 | MPGGRRPAWRSSAGLPSPTSPRKVVSAPPGGGLGPGAPRRGRPRGAT | SEQ ID NO: |
| | EGPGRTGDRENARGGGGQRGTCACLWNTMACWPARLLGSLQACC | 172 |

Several JMHV ORFs have been identified that can impact this model system.

For example, JM25 is homologous to collagen and myosin. Collagen is found in the arterial walls and during MS disease there is a break down of the blood brain barrier. Some forms of myosin are found in neurons and is involved in vesicular transport.

JM26 is homologous to myosin and sphingosine kinase. Some forms of myosin are found in neurons and is involved in vesicular transport. Sphingosine kinase is involved in cell cycle progression and is a major component of oligodendroglia. Oligodendroglia is the non-deural cfells of ectodermal orgin forming part of the advential structure (neuroglia) of the central nervous system; projections of the surface membrane of each of these cells (oligodendrocytes) fan out and coil around the axon of many neurons to form myelin sheaths in the white matter. With micorglia, they form the perineuronal satellites in the gray matter.

JM39 is homologous to succinate dehydrogenase. Succinate dehydrogenase is found in the mitochondria and is an enzyme of the oxidoreductase class that catalyzes the oxidation of succinate to fumarate, using a variety of hydrogen acceptors. Succinate dehydrogenase is down regulated in patients with multiple sclerosis.

JM41 shows homology to alpha-1a adrenergic receptor. Adrenergic receptors are involved in secreting epinephrine or related substances, particularly in sympathetic nerve fibers that liberate norepinephrine at a synapse when a nerve impulse passes.

JM48 shows homology to mucin. Mucin is defined as any group of proteins-containing glycoconjugates with high sialic acid or sulfated polysaccharide content. Lipmatosis is a condition characterized by abnormal localized or tumor-like, accumulations of fat as well as an accumulation of mucin in the tissues. Lipomatous metaplastic changes occur with people diagnosed with multiple sclerosis.

JM54 is homologous to collagen and myosin. Collagen is found in the arterial walls and during MS disease there is a break down of the blood brain barrier. Some forms of myosin are found in neurons and is involved in vesicular transport.

JM57 has homology to calcium channel proteins. Mitochondrial calcium membrane potential is a component of cellular survival. Once membrane potential is lost a cell will progress towards apoptosis.

JM71 has homology to a chromatin associated protein. Chromatin is a deoxyribonucleic acid attached to a protein structure base and is the carrier of the genes in inheritance. A characteristic of apoptosis is chromatin condensation and DNA fragmentation. Therefore, JM71 may have an anti-apoptotic function.

JM76 has homology to plexin. Plexins have been shown to promote axon repulsion. Recent evidence has shown that if plexin expression is inhibited in carcinoma cell migration and when it is overexpressed migration is inhibited.

JM80 has homology to NADH-ubiquinone dehydrogenase. NADH-ubiquinone dehydrogenase is a protein involved in the mitochondrial electron transport chain. Generation of reactive oxygen species is a byproduct of this pathway and is important in the aging process and in neurodegenerative diseases.

JM85 has homology to a chromatin remodeling complex protein. Chromatin is a deoxyribonucleic acid attached to a protein structure base and is the carrier of the genes in inheritance. A characteristic of apoptosis is chromatin condensation and DNA fragmentation.

JM87 has homology to mucin. Mucin is defined as any group of proteins-containing glycoconjugates with high sialic acid or sulfated polysaccharide content. Lipmatosis is a condition characterized by abnormal localized or tumor-like, accumulations of fat as well as an accumulation of mucin in the tissues. Lipomatous metaplastic changes occur with people diagnosed with multiple sclerosis.

JM88 has homology to neurexin. Neurexins link the pre- and postsynaptic compartments of synapses by binding extracellularly to post-synaptic cell adhesion molecules and intracellularly to pre-synaptic PDZ domain proteins. These proteins are essential for Ca2+ triggered neurotransmitter release.

JM132 has homology to c-myc promoter binding protein. C-myc has been shown to be involved in cell proliferation and tumorgenesis.

JM152 has homology to cyclin D. Cyclin D is a key regulator of cell cycle. Cell cycle events that are regulated by cyclins play a major role in the loss of neurons in diseases such as Alzheimer's disease.

JM166 has homology to collagen. Collagen is found in the arterial walls and during MS disease there is a break down of the blood brain barrier JM167 has homology to myosin phosphatase. Some forms of myosin are found in neurons and is involved in vesicular transport.

JM168 has homology to a CC chemokine receptor. Recent evidence has shown CC chemokine receptor expression by mononuclear phagocytes in multiple sclerosis lesions.

### Example 3

### Non-Human Primate Model for MS

MS is a devastating disease affecting an estimated 1 million people worldwide, or 0.1% of the U.S. population. Current research is directed at elucidating potential viral/bacterial causes of MS, as well as determining the involvement of the host immune system in the disease. Rhesus macaques have provided a nonhuman primate experimental autoimmune encephalomyelitis (EAE) model of MS since the 1930's (Ferraro and Cazzull, J. Neuropathol Exp. Neurol. 7:235-260, 1948; Rivers, and Schwentker, J. Exp. Med. 61:689-705, 1935). Recently, the common marmoset has been used to develop an animal model for MS (Brok et al., J Immunol 165:1093-101, 2000; Genain and Hauser, J Mol Med 75:187-97, 1997). The marmoset model relies on myelin oligodendrocyte glycoprotein-specific antibodies for demyelination (Genain et al., Nat Med 5:170-5, 1999; Genain et al., J Clin Invest 96:2966-74, 1995; Raine et al., Ann Neurol 46:144-60, 1999). While EAE studies have provided data into how anti-myelin T cells and antibodies can cause demyelination, there are questions about their relevance. Even more important is that EAE induced in these animal models is due to active immunization with myelin antigens or passive transfer of myelin specific T cells, neither of which occurs in MS.

Until now there has not been a nonhuman primate model in which a naturally occurring chronic viral infection results in an MS-like demyelinating disease. The availability of the complete JMHV sequence, the ability to grow JMHV in vitro and the association of the virus with a demyelinating disease in vivo can be used in a relevant nonhuman primate model for MS in the Japanese macaque.

It will be apparent that the precise details of the methods or compositions described may be varied or modified without departing from the spirit of the described disclosure. We claim all such modifications and variations that fall within the scope and spirit of the claims below.

1. An isolated viral nucleic acid comprising a nucleic acid sequence at least 90% identical to the nucleic acid sequence set forth as SEQ ID NO: 1.

2. A host cell transformed with the virus of claim 1.

3. A polypeptide at least 95% identical to a polypeptide encoded by the nucleic acid of SEQ ID NO: 1.

4. The polypeptide of claim 3, wherein the polypeptide is 100% identical to the polypeptide encoded by SEQ ID NO: 1.

5. The polypeptide of claim 3, wherein the polypeptide encoded by nucleic acid 21845 to nucleic acid 22120 of SEQ ID NO: 1 (JMHV25), nucleic acid 22363 to nucleic acid 22701 of SEQ ID NO: 1 (JMHV26), nucleic acid 33254 to nucleic acid 33553 of SEQ ID NO: 1 (JMHV39), nucleic acid 35301 to nucleic acid 35687 of SEQ ID NO: 1 (JMVH41), nucleic acid 40188 to nucleic acid 40439 of SEQ ID NO: 1 (JMHV48), nucleic acid 45836 to nucleic acid 46195 of SEQ ID NO: 1 (JMHV54), nucleic acid 47768 to nucleic acid 48136 of SEQ ID NO: 1 (JM57), nucleic acid 57325 to nucleic acid 57573 of SEQ ID NO: 1 (JM71), nucleic acid 62823 to nucleic acid 63086 of SEQ ID NO: 1 (JM76), nucleic acid 65629 to nucleic acid 65880 of SEQ ID NO: 1 (JM80), nucleic acid 67920 to nucleic acid 68594 of SEQ ID NO: 1 (JM85), nucleic acid to nucleic acid of SEQ ID NO: 1 (JM87) nucleic acid 70328 to nucleic acid 70606 of SEQ ID NO: 1 (JM88), nucleic acid 75447 to nucleic acid 75722 of SEQ ID NO: 1 (JM95), nucleic acid 105581 to nucleic acid 106003 of SEQ ID NO: 1 (JM132), nucleic acid 117501 to nucleic acid 118265 of SEQ ID NO: 1 (JM152), nucleic acid to nucleic acid of SEQ ID NO: 1 (JM159), nucleic acid to nucleic acid of SEQ ID NO: 1 (JM166), and nucleic acid to nucleic acid of SEQ ID NO: 1 (JM167).

6. The polypeptide of claim 5, wherein the polypeptide comprises an amino acid sequence set forth as one of SEQ ID NOs: 1-171.

7. An antibody that specifically binds the polypeptide of claim 3.

8. The antibody of claim 7, wherein the antibody is a monoclonal antibody.

9. A nucleic acid encoding the polypeptide of claim 3.

10. The nucleic acid of claim 9, operably linked to a promoter.

11. A vector comprising the nucleic acid of claim 10.

12. The vector of claim 11, wherein the vector is a viral vector.

13. A method for testing the efficacy of an agent for the treatment a symptom associated with multiple sclerosis, the method comprising:
(a) administering the agent to a non-human primate infected with a virus comprising the nucleic acid sequence of claim 1; and
(b) observing the non-human primate to determine if the drug inhibits the presentation of one or more symptoms associated with Japanese macaque herpesvirus (JMHV) infection as compared to a non-human primate not administered the agent.

14. The method of claim 13, wherein the agent is a drug used to treat multiple sclerosis.

15. The method of claim 13, wherein the non-human primate is a Japanese macaque monkey.

16. The method of claim 13, wherein the symptom is associated with multiple sclerosis.

17. A method of detecting the presence of a virus in a biological specimen, comprising:
(a) amplifying by polymerase chain reaction a Japanese macaque herpesvirus (JMHV) nucleic acid sequence, if such sequence is present in the sample, using two or more oligonucleotide primers comprising 20 contiguous nucleotides of the nucleic acid sequence of claim 1 to form an amplified sequence; and
(b) determining whether an amplified sequence is present.

18. The method of claim 17, wherein the step of determining whether an amplified sequence is present comprises one or more of:
(a) electrophoresis and staining of the amplified sequence; or
(b) hybridization the amplified sequence to a probe.

19. The method of claim 18, wherein the probe comprises a detectable non-isotopic label comprising a fluorescent molecule, a chemiluminescent molecule, an enzyme, a co-factor, an enzyme substrate; or a hapten.

20. The method of claim 17, wherein the biological specimen is a non-human primate specimen.

21. A method of detecting the presence of Japanese macaque herpesvirus (JMHV) in a biological specimen, comprising:
(a) exposing the biological specimen to a probe that hybridizes to a Japanese macaque herpesvirus (JMHV) nucleic acid sequence of claim 1, if the sequence is present in the sample to form a hybridization complex; and
(b) determining whether the hybridization complex is present, thereby detecting the presence of the Japanese macaque herpesvirus (JMHV).

22. The method of claim 21, wherein the primate specimen is a non-human primate specimen.

23. A method of detecting the presence of Japanese macaque herpesvirus (JMHV) in a biological specimen, comprising:
(a) contacting the biological specimen with the antibody of claim 5,
(b) detecting binding of the antibody to the biological specimen or a component thereof, wherein binding of the antibody to the biological specimen indicates the presence of a Japanese macaque herpesvirus (JMHV).

24. The method of claim 23, wherein the antibody comprises a detectable label.

25. The method of claim 24, wherein the detectable label comprises a fluorescent molecule, a chemiluminescent molecule, an enzyme, a co-factor, an enzyme substrate; or a hapten.

26. A kit comprising a container means comprising an oligonucleotide primer comprising at least 15 contiguous nucleotides of the nucleic acid sequence of claim 1.

27. A kit comprising a container means comprising an antibody of claim 7.

28. A non-human primate model for multiple sclerosis, comprising a non-human primate infected with a virus comprising the nucleic acid sequence of claim 1, wherein the non-human primate exhibits a symptom or a pathological feature of multiple sclerosis.

29. The non-human primate model of claim 28, wherein the symptom is acute onset paresis or paralysis involving one or more limbs.

30. The non-human primate model of claim 28, wherein the pathological feature is myelin destruction in a central nervous system.

31. The non-human primate model of claim 28, wherein the non-human primate is a Japanese macaque.

32. An isolated virus comprising the nucleic acid of claim 1.
